SEQUENCE LISTING (10 sequences)

SEQ ID NO: 1 - DNA, 22 bp, Synthetic construct
  source 1..22: mol_type = other DNA
gccaaacttcagccttctcttc

SEQ ID NO: 2 - DNA, 23 bp, Synthetic construct
  source 1..23: mol_type = other DNA
ctctggcaacatttcacactaca

SEQ ID NO: 3 - DNA, 21 bp, Synthetic construct
  source 1..21: mol_type = other DNA
ggctggcttcctgagcttatt

SEQ ID NO: 4 - DNA, 21 bp, Synthetic construct
  source 1..21: mol_type = other DNA
acttcctgaacagctcatcgg

SEQ ID NO: 5 - DNA, 20 bp, Synthetic construct
  source 1..20: mol_type = other DNA
aatgatacggcgaccaccga

SEQ ID NO: 6 - DNA, 21 bp, Synthetic construct
  source 1..21: mol_type = other DNA
caagcagaagacggcatacga

SEQ ID NO: 7 - DNA, 20 bp, Synthetic construct
  source 1..20: mol_type = other DNA
acggaccagagcgaaagcat

SEQ ID NO: 8 - DNA, 20 bp, Synthetic construct
  source 1..20: mol_type = other DNA
tgtcaatcctgtccgtgtcc

SEQ ID NO: 9 - DNA, 15 bp, Synthetic construct
  source 1..15: mol_type = other DNA
cagcmgccgcggtaa

SEQ ID NO: 10 - DNA, 20 bp, Synthetic construct
  source 1..20: mol_type = other DNA
ggactachvgggtwtctaat

(19) United States
(12) Patent Application Publication
(10) Pub. No.: US 2024/0084360 A1
(43) Pub. Date: Mar. 14, 2024

(54) HOST DEPLETION AND MICROBIAL ENRICHMENT OF A BIOLOGICAL SAMPLE AND RELATED METHODS AND SYSTEMS

(71) Applicant: California Institute of Technology, Pasadena, CA (US)

(72) Inventors: Rustem F. Ismagilov, Altadena, CA (US); Natalie Wu-Woods, Pasadena, CA (US); Jacob T. Barlow, Cambridge, MA (US); Anna E. Romano, Pasadena, CA (US)

(21) Appl. No.: 18/351,461

(22) Filed: Jul. 12, 2023

Related U.S. Application Data
(60) Provisional application No. 63/388,623, filed on Jul. 12, 2022.

(51) Int. Cl. C12Q 1/6806

(52) U.S. Cl. CPC C12Q 1/6806

(57) ABSTRACT

Provided herein are methods and systems to selectively deplete a biological sample of host compartments and/or host nucleic acid while enriching the sample microbial compartments and/or related microbial nucleic acid. In addition, provided herein are compositions, methods and systems related to said host depletion and microbial enrichment methods and systems.

### CROSS-REFERENCE TO RELATED APPLICATION

The present application claims priority to U.S. Provisional Application No. 63/388,623, entitled “Nucleic Acid Analysis from Host Rich Samples” filed on Jul. 12, 2022, with docket number CIT 8851-P, the contents of which is incorporated by reference in its entirety.

### STATEMENT OF GOVERNMENT GRANT

This invention was made with government support under Grant No. W911NF-17-1-0402 awarded by the Army. This material is also based upon work supported by the National Science Foundation Graduate Research Fellowship under Grant No. DGE-1745301 and the National Institute of Health under Grant No. 1RC2DIK1133947-01. The government has certain rights in the invention.

### FIELD

The present disclosure relates generally to microbial nucleic acid and related applications such as qualitative and/or quantitative detection of microbes and/or microbial nucleic acid in sample from a host. In particular, the present disclosure relates to host depletion and microbial enrichment of a biological sample and related methods and systems.

### REFERENCE TO SEQUENCE LISTING

Further, the computer readable form of the sequence listing of the XML text file P2925-US-2023-12-01-Seq-Listing.xml, created on Dec. 1, 2023, with a size of 9,964 bytes measured on Windows Server 2019 Datacenter, is incorporated herein by reference in its entirety.

### BACKGROUND

Many methods for detecting and profiling nucleic acids are currently available in particular in connection with studies of samples comprising nucleic acid from different sources.

Challenges however remain for developing accurate and robust methods that enable the quantification of microbial nucleic acids with wide dynamic range and broad microbial diversity with minimized interference from contaminant nucleic acids and potential biases in complex nucleic acid mixtures.

### SUMMARY

Provided herein is a host depletion and microbial enrichment methodology (herein also MEM) as well as related methods and systems, which selectively targets the host cells and related nucleic acid of a biological sample to selectively deplete host cells and increase host NA accessibility, while minimizing depletion of microbial compartments and related nucleic acid possibly present in the sample. The MEM and related methods and systems herein described, allow a robust enrichment of microbes and related nucleic acid in the sample as well as related detection and analysis with minimized impact from presence of cells and/or nucleic acids of the host.

According to a first aspect, a host depletion method and systems are described and a disrupted biological sample obtained thereby. The host depletion method is a method to deplete a biological sample of a host comportment and increase accessibility of a host nucleic acid (NA), the host comportment having an elastic modulus Eₕₒₛₜ and a host diameter Hd in at least one dimension, the host compartment encapsulating the host NA. The host depletion method comprises providing a set of beads configured to selectively disrupt the host compartment with respect to a microbial compartment having an elastic modulus E_{microbe} and a microbial diameter Md in at least one dimension and, the microbial compartment encapsulating a microbial NA, the target microbial NA having a mass equal or lower than the host NA, the providing performed by
a) selecting a bead radius to determine for each of the host compartment and the microbial compartment, a compartment crush volume (V_{crushed compartment}) as a function of compartment diameter and bead radius;
b) selecting bead parameters including number, bead collision frequency and bead beating time to determine, for each of the host compartment and the microbial compartment, a compartment effective crush volume (V_{total_effective-compartment}) as a function of said compartment crush volume (V_{crushed compartment}), the compartment elastic modulus (E_{compartment}) and said bead parameters; and
c) determining, for each of the host compartment and the microbial compartment, a percentage of total sample volume that is bead beaten for a given compartment (BB%_{compartment}) as a ratio between the compartment effective crush volume (V_{total_effective-compartment}) and a total volume of the sample,
d) providing a selected set of beads having a bead radius and set of bead parameters selected to obtain a percentage of total sample volume of at least 100% for the given host compartment and a percentage of total sample volume of up to 50% for the given microbial compartment.

The method further comprises disrupting the biological sample by contacting the biological sample with the selected set of beads and selected beads parameter to provide a disrupted biological sample comprising disrupted host compartments and accessible host NA, the biological sample further enriched with the target microbial compartment.

The host depletion system comprises at least one set of beads having a radius R, a an elastic modulus E_{beads}, in combination with a lookup table connecting i) R and ii) E_{beads} of at least one set of beads, iii) suitable numbers of beads of the at least one set of beads, iv) frequency of collision and/or speed of the disrupting and v) duration of disrupting, with one or more percentage of host compartment depletion and/or one or more increased accessibility of host NA for one or more type samples. In the host depletion system the connecting is performed according to the host depletion method of the present disclosure.

According to a second aspect a method and a system are described and a nuclease treated sample obtained thereby. The method and system are directed to enrich a biological sample with a microbial compartment and related target microbial nucleic acid (NA), the biological sample comprising a host NA encapsulated within a host compartment having a host diameter Hd equal to or higher than 8 um in at least one dimension, the host NA having a mass equal to or greater than the target microbial NA.

The enrichment method of the disclosure comprises contacting a disrupted sample of the present disclosure with a nuclease for a time and under conditions to break the nucleotide chain of nucleic acid accessible to the nuclease within the disrupted sample, to obtain a nuclease treated biological sample depleted of the host compartment and depleted of the accessible host NA, the nuclease treated sample thus enriched with the target microbial compartment and the target microbial NA as will be understood by a skilled person. Optionally, the enrichment method of the disclosure can comprise providing the disrupted sample with the host depletion method of the present disclosure.

The enrichment system according to the second aspect comprises one or more nucleases in combination with a lookup table indicating amount of nuclease, temperature and duration of nuclease degradation in connection with one or more set percentage enrichment of the target microbial compartment and/or target microbial NA. The enrichment system can optionally comprise at least one set of beads and/or lookup table to perform host depletion for a simultaneous, combined, or sequential use in the method to enrich a biological sample of the present disclosure.

According to a third aspect a method is described to isolate a target microbial compartment from a biological sample. In the method of the fourth aspect, the target microbial compartment encapsulates a target microbial NA and has a microbial diameter Md, the biological sample further comprising a host NA encapsulated within a host cell having a host diameter, the host NA having a mass equal to or greater than the microbial NA.

The method to isolate a target microbial compartment of the disclosure comprises separating the target microbial compartment from a disrupted biological sample and/or a nuclease treated biological sample to obtain a microbial fraction comprising the target microbial compartments of the biological sample and the related target microbial NA.

The method to isolate a target microbial compartment of the disclosure can optionally further comprise contacting the microbial fraction with a nuclease for a time and under conditions to degrade accessible host NA in the microbial fraction thus obtaining a nuclease treated microbial fraction of the disrupted biological sample, which is enriched with the target microbial compartment and the target microbial NA. The method to isolate a target microbial compartment of the disclosure can further optionally comprise, isolating the microbial compartment from the microbial fraction and/or nuclease treated microbial fraction to obtain an isolated microbial compartment.

In embodiments wherein the isolated target microbial compartment is an isolated replication competent target compartment, the method to isolate a target microbial compartment of the disclosure can further optionally comprise, contacting the isolated replication competent target microbial compartments with reagents for a time and under conditions allowing replication of the isolated replication competent target microbial compartments to obtain isolated amplified target microbial compartment.

The system to isolate a target microbial compartment of the disclosure comprises reagents and/or devices for isolating the microbial compartment from the disrupted sample and/or the nuclease treated sample optionally in combination with one or more nucleases and/or reagents to amplify the replication competent target microbial compartment. The system to isolate a target microbial compartment of the disclosure can optionally further comprise at least one set of beads and lookup table to perform host depletion and reagents to enrich the biological sample with the target microbial compartments, for a simultaneous, combined, or sequential use in the method to isolate the host NA of the present disclosure.

According to a fourth aspect, a method and a system are described to detect and/or a target microbial compartment of a biological sample. The method comprises detecting and/or analyzing the target microbial compartment in a disrupted biological sample, a nuclease treated biological sample, a microbial fraction, a nuclease treated microbial fraction, an isolated microbial compartment and/or an isolated amplified microbial compartment of the present disclosure to obtain a detected target microbial compartment, detected features of the target microbial compartment and/or a modified microbial compartment.

The system according to the fourth aspect comprises reagents for detecting and/or analyzing the target microbial compartments in optionally in combination with reagents and/or devices for isolating the microbial compartment from the disrupted sample and/or the nuclease treated sample optionally in combination with one or more nucleases and/or reagents to amplify the replication competent target microbial compartment. The system to isolate a target microbial compartment of the disclosure can optionally further comprise at least one set of beads and lookup table to perform host depletion and reagents to enrich the biological sample with the target microbial compartments, for a simultaneous, combined, or sequential use in the method to isolate the host NA of the present disclosure.

According to a fifth aspect, a method and a system are described to detect and/or analyze a target microbial community possibly present in a biological sample. The method comprises detecting and/or analyzing the target microbial community in an isolated microbial fraction, an isolated microbial compartment and/or an isolated amplified microbial compartment of the present disclosure to obtain a detected microbial community, detected features of the microbial community, and/or a modified microbial community.

The system to analyze a target microbial community according to the fifth aspect of the disclosure comprises reagents and/or devices for detecting and/or analyzing the target microbial community optionally together with reagents and/or devices for isolating the microbial community from the disrupted sample and/or the nuclease treated sample optionally in combination with one or more nucleases and/or reagents to amplify the replication competent target microbial compartment. The system to isolate a target microbial community of the disclosure can optionally further comprise at least one set of beads and lookup table to perform host depletion and reagents to enrich the biological sample with the target microbial community, for a simultaneous, combined, or sequential use in the method to analyze a microbial community of the present disclosure.

According to a sixth aspect a method is described to isolate a target microbial NA from a biological sample. In the method, the target microbial NA is encapsulated within a target microbial compartment, the biological sample further comprising a host NA encapsulated within a host compartment, the host NA having a mass equal to or greater than the microbial NA. The method to isolate a target microbial NA from a biological sample comprises isolating the target microbial NA from a nuclease treated sample, a microbial fraction, an isolated target microbial compartment and/or an amplified target microbial compartment of the present disclosure, to obtain an isolated target microbial NA.

The system to isolate a target microbial NA from a biological sample comprises reagents and/or devices for isolating the target microbial NA from the nuclease treated sample optionally in combination with one or more nucleases and/or reagents to amplify a replication competent target microbial compartment comprising the target microbial NA. The system to isolate a target microbial NA of the disclosure can optionally further comprise at least one set of beads and lookup table to perform host depletion and reagents to enrich the biological sample with the target microbial compartments, for a simultaneous, combined or sequential use in the method to isolate a target microbial NA from a biological sample of the present disclosure.

According to a seventh aspect, a method is described to detect and/or analyze a target microbial nucleic acid in a biological sample comprising a host cell. The method comprises, detecting and/or analyzing the target microbial nucleic acid in a disrupted biological sample, a nuclease treated sample, a microbial fraction, a nuclease treated microbial fraction, an isolated microbial compartment and/or an amplified microbial compartment of the present disclosure, to obtain a detected target microbial nucleic acid, detected feature of the target microbial nucleic acid and/or a modified target microbial nucleic acid.

The system to detect and/or analyze a target microbial nucleic acid in an biological sample according to the seventh aspect comprises reagents for detecting and/or analyzing the target microbial nucleic acid in optionally in combination with reagents and/or devices for isolating the target microbial nucleic acid from the disrupted sample and/or the nuclease treated sample a microbial fraction, a nuclease treated microbial fraction, an isolated microbial compartment and/or an amplified microbial compartment of the present disclosure, optionally in combination with one or more nucleases and/or reagents to amplify the replication competent target microbial compartment. The system to detect a target microbial nucleic acid in a biological sample of the disclosure can optionally further comprise at least one set of beads and lookup table to perform host depletion and reagents to enrich the biological sample with the target microbial compartments, for a simultaneous, combined or sequential use in the method to detect and/or analyze a target microbial nucleic acid in a biological sample of the present disclosure.

According to an eighth aspect, a method and a system are described to detect and/or analyze a target plurality of microbial nucleic acid of a biological sample. The method comprises detecting and/or analyzing the target plurality of microbial nucleic acid in a disrupted biological sample, a nuclease treated sample, a microbial fraction, a nuclease treated microbial fraction, an isolated microbial compartment and/or an amplified microbial compartment of the present disclosure, to obtain a detected target plurality of microbial nucleic acid, a detected feature of the target plurality of microbial nucleic acid and/or a modified target plurality of microbial nucleic acids.

The system to detect and/or analyze a target plurality of microbial nucleic acids in an biological sample according to the eighth aspect comprises reagents for detecting and/or analyzing the plurality of microbial nucleic acids optionally in combination with reagents and/or devices for isolating the target plurality of microbial nucleic acids from the disrupted sample and/or the nuclease treated sample a microbial fraction, a nuclease treated microbial fraction, an isolated microbial compartment and/or an amplified microbial compartment of the present disclosure, optionally in combination with one or more nucleases and/or reagents to amplify replication competent target microbial compartment. The system to detect a target microbial nucleic acid in a biological sample of the disclosure can optionally further comprise at least one set of beads and lookup table to perform host depletion and reagents to enrich the biological sample with the target microbial compartments, for a simultaneous, combined or sequential use in the method to detect and/or analyze a target plurality of microbial nucleic acids in a biological sample of the present disclosure.

According to a ninth aspect, a host NA isolation method and systems are described. The host NA isolation method is a method to isolate a host NA of a biological sample, the host NA encapsulated in in a host comportment. The host NA isolation method comprises isolating accessible host NA from a disrupted sample herein described. The isolating can be performed by separating the disrupted sample to provide a microbial fraction and a host fraction and extracting the host NA from the host fraction of the disrupted sample. The isolation method can optionally further comprises providing the disrupted sample with a host depletion method of the present disclosure.

The host NA isolation system of the ninth aspect comprises reagents and/or devices for isolating the host NA from the disrupted sample optionally in combination with at least one set of beads and/or a lookup table to perform host depletion for a simultaneous, combined, or sequential use in the method to isolate the host NA of the present disclosure.

According to a tenth aspect a method and system is described to detect and/or analyze a target microbial nucleic acid in combination with a host nucleic acid of a biological sample. The method comprises detecting and/or analyzing the target microbial NA in a microbial fraction, nuclease treated microbial fraction of the biological sample, and/or in isolated microbial compartments and/or amplified microbial compartments of the present disclosure The method further comprises detecting and/or analyzing the host NA from a host fraction of the disrupted sample of the present disclosure.

The method can optionally further comprise separating the disrupted sample to obtain a microbial fraction and a host fraction of the disrupted sample, The method can also further comprises providing a disrupted sample with a host depletion method of the present disclosure.

The system comprises components to detect and/or analyze the target microbial NA from the microbial fraction, nuclease treated microbial fraction, in isolated microbial compartments and/or amplified microbial compartments of the present disclosure, and components of a system to isolate detect and/or analyze the host NA from a host fraction of the disrupted sample of the present disclosure Optionally in the system those components are comprised in combination with components of the system to provide a microbial fraction, nuclease treated microbial fraction, in isolated microbial compartments and/or amplified microbial compartments of the present disclosure.

According to an eleventh aspect a method and system is described to detect and/or analyze a target plurality of microbial nucleic acids and a target plurality of host nucleic acid of a biological sample. The method comprises detecting and/or analyzing the target plurality of microbial nucleic acids in a microbial fraction, in a nuclease treated microbial fraction, in isolated microbial compartments and/or amplified microbial compartments of the present disclosure, to provide microbial detected features of the target plurality of microbial nucleic acids. The method further comprises detecting and/or analyzing the target plurality of host nucleic acids from a host fraction of the biological sample, to provide host detected features of the target plurality of microbial nucleic acids. The method can further comprise comparing the data microbial detected features and the host detected features.

The method can optionally further comprise separating the disrupted sample to obtain a microbial fraction and a host fraction of the disrupted sample, The method can also further comprises providing a disrupted sample with a host depletion method of the present disclosure.

The system comprises components of the system to detect and/or analyze a the target plurality of host nucleic acids from a host fraction of the biological sample, optionally in combination with components to of the systems to obtain a microbial fraction, nuclease treated microbial fraction, in isolated microbial compartments and/or amplified microbial compartments of the present disclosure, and components of a system to isolate detect and/or analyze the host NA from a host fraction of the disrupted sample of the present disclosure.

The host depletion and microbial enrichment methodology and related methods and systems herein described, allow in several embodiments to enrich and possibly quantify microbial compartments and/or microbial nucleic acid in biological samples with low microbial loads down to 10² compartments/mg of tissue with an increased precision and accuracy and with an increased limit of detection due to the effective depletion of host nucleic acid performed minimizing the loss of microbial nucleic acid. Accordingly, the host depletion and microbial enrichment methodology and related methods and systems herein described allow in several embodiments to isolate, qualitatively and/or quantitatively detect and/or analyze target microbial nucleic acid present in amounts from about 10 to about 10¹⁰ microbial compartments.

The host depletion and microbial enrichment methodology and related methods and systems herein described allow in several embodiments to enrich and thus also isolate, detect and/or analyze microbial compartments and/or microbial nucleic acid in samples with low microbial abundance, e.g. due to the presence of high levels of host biological DNA (e.g at concentrations up to 10⁵ host cells/mg of sample or ˜600 ng DNA/mg of sample), as is common in human clinical samples.

In particular, the host depletion and microbial enrichment methodology and related methods and systems herein described allow in several embodiments also isolate, detect and/or analyze microbial compartments and/or microbial nucleic acid in samples with microbial target loads varying from a 10² cells/mg of sample or 2 pg NA/mg of sample up to 10⁹ cells/mg of sample or 20 ug NA/mg of sample and containing high contaminant host polynucleotides, such as host DNA background at concentrations up to 600 ng DNA/mg of sample.

Notably, the host depletion and microbial enrichment methodology and related methods and systems herein described allow in several embodiments to improve the limit of detection of target microbial compartment and/or target microbial nucleic acid in a manner proportional to the improvement in percentage of microbial compartments comprised in the disrupted sample and/or nuclease treated sample. For example, MEM processing of a biological sample allows improvements in % microbial reads by shotgun sequencing—from 0.01% microbial reads to 10% microbial reads, corresponding to a 1000-fold improvement in limit of detection as will be understood by a skilled person.

Accordingly, the increased limit of detection of microbial compartments and/or microbial nucleic acid of the host depletion and microbial enrichment methodology and related methods and systems herein described, allows the related detection and/or analysis with an increased resolution as would be understood by a skilled person.

In particular, the increased limit of detection of microbial compartments and/or microbial nucleic acid of the host depletion and microbial enrichment methodology and related methods and systems herein described, allows detection and/or analysis of targets plurality of microbial compartments (e.g. a target microbial community such as a microbiome) and/or target plurality of microbial NA (e.g. target genomes of a microbiome) with increased resolution due to inclusion of microbial compartments and/or microbial NA of the target plurality which are present at low loads and/or at a low abundance, compared to existing methods.

Accordingly, the host depletion and microbial enrichment methodology and related methods and systems herein described allow in several embodiments to expand microbial coverage of microbial isolation, detection and/or analysis from a host sample while significantly reducing non-specific host nucleic acid, thus achieving a wide dynamic range in microbial quantification and broad coverage for capturing high microbial diversity in samples with or without high host NA background in the target environment. For example, method and systems of the disclosure allow detection in animal samples of target plurality of microbial nucleic acids comprising components with a concentration of equal or lower 2 pg NA/mg of sample in the presence of the host NA at a maximum of 4 ug NA/mg of sample.

Therefore, the host depletion and microbial enrichment methodology and related methods and systems herein described allow in several embodiments to perform analysis and in particular profiling of microbial communities such as profiling of the microbiome of a biological sample such as an intestinal sample, a biopsy or saliva sample with increased resolution and therefore accuracy and precision.

The host depletion and microbial enrichment methodology and related methods and systems herein described allow in several embodiments to enrich and possibly quantify microbial nucleic acid in variety of sample types with an approach that is robust also in biological samples with low microbial abundance and/or low microbial loads.

The host depletion and microbial enrichment methodology and related methods and systems herein described allow in several embodiments to reduce the amount of sample needed and/or time and reagent costs due to the effective enrichment of host samples.

The host depletion and microbial enrichment methodology and related methods and systems herein described can be used in connection with various applications wherein enrichment detection and/or analysis of microbial nucleic acid and/or microbial compartment in complex samples is desired, in particular in target environment including contaminant polynucleotides. For example, the microbial enrichment approach and related methods and systems herein described allow in several embodiments herein described can be used for qualitative and/or quantitative microbiome profiling in human and animal microbiome research, or detection of monoinfections and profiling of polymicrobial infections in tissues, stool, and bodily fluids in human and veterinary medicine, or environmental sample analyses (e.g., soil and water); or broad-coverage detection of microbial food contamination in products high in mammalian DNA, such as meat products, oil industry, bioburden monitoring (e.g. maintenance of clean rooms, surgery rooms and additional applications identifiable by a skilled person.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1A shows schematics illustrating estimated percentages of bacterial reads in an exemplary human sample sequenced without sample processing.

FIG. 1B shows a schematic illustration of an exemplary two-step selective-lysis and nucleic-acid removal techniques used in MEM processing according to the present disclosure.

FIG. 2 shows a schematic illustration of an exemplary microorganism enrichment/a host depletion workflow in accordance with the present disclosure.

FIGS. 3A to 3F shows schematic illustration of exemplary MEM methods and systems of the present disclosure. In particular FIG. 3A schematically illustrate a workflow in which a disrupted sample and/or a nuclease treated sample of the disclosure are further processed for isolation, detection and/or analysis of microbial compartments, microbial nucleic acid, and/or host nucleic acid. FIGS. 3B to 3F shows schematic illustrations of methods and systems to perform the further processing of the disrupted sample and/or a nuclease treated sample of the disclosure encompassed by FIGS. 3A-3F.

FIG. 4. Shows a schematic of two beads colliding. In this model of bead beating, two beads are depicted as half circles come into contact to crush a microbial compartment of characteristic size Md, which defines the volume between the two beads that are capable of crushing a microbial compartment of characteristic size Md.

FIG. 5A to 5E show schematics, diagrams and charts reporting data illustrating the effectiveness of an exemplary microbial enrichment approach of the disclosure on stool, saliva, and intestinal samples in comparison with existing methods.

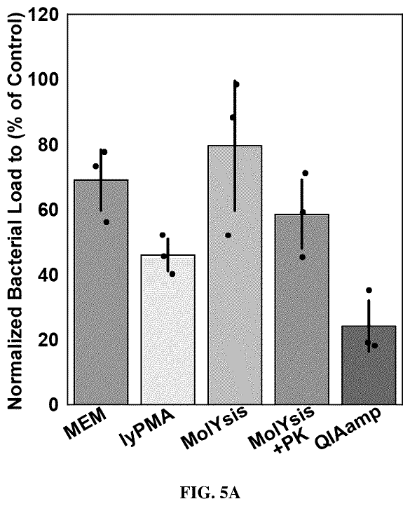

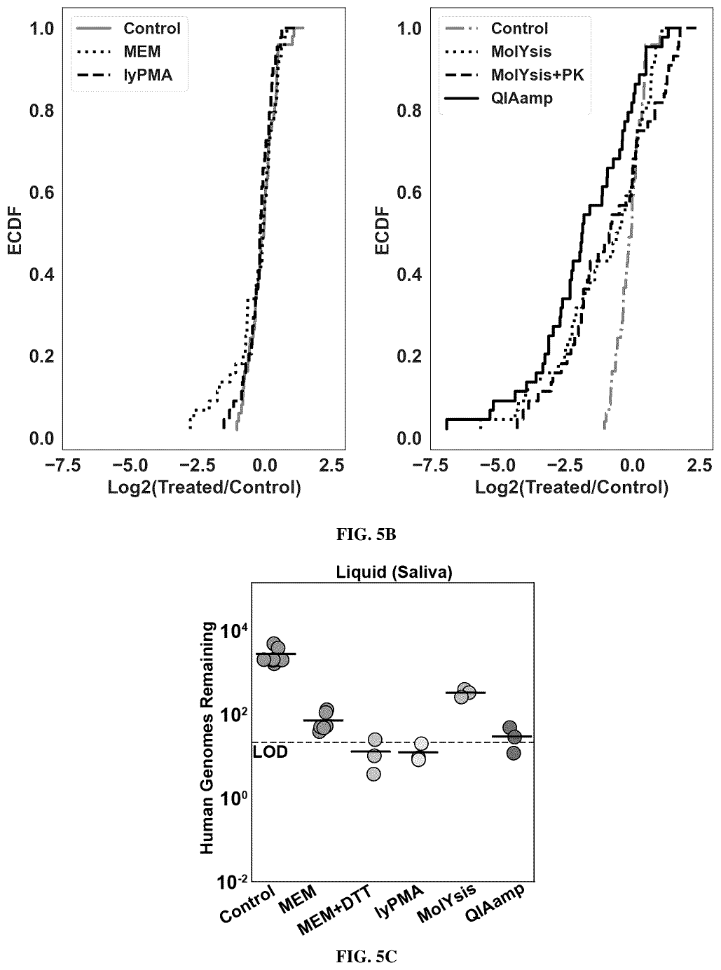

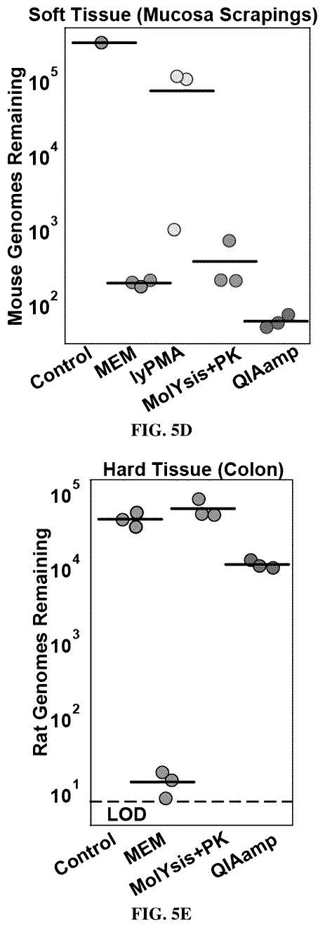

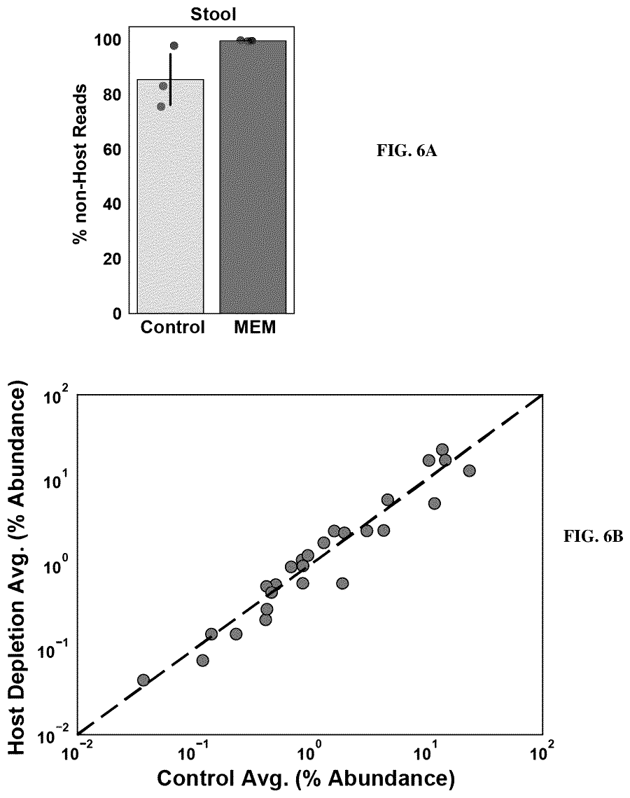

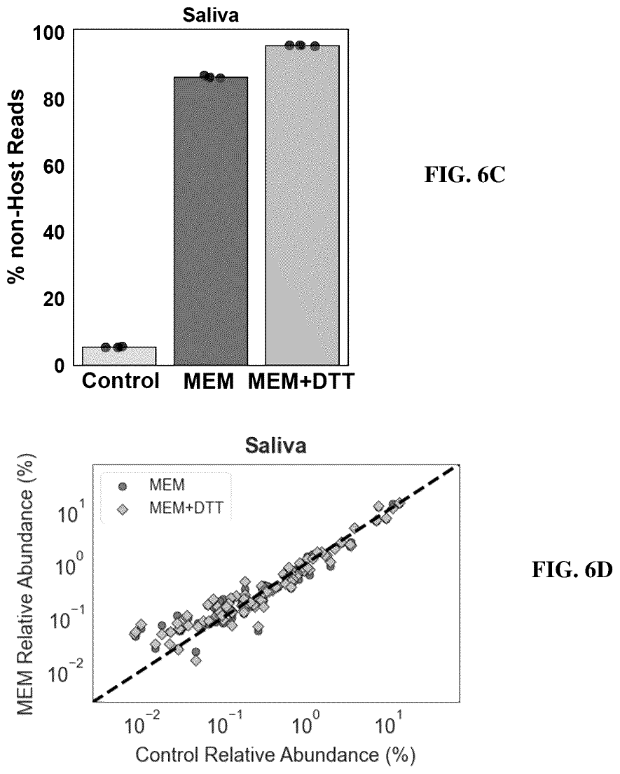

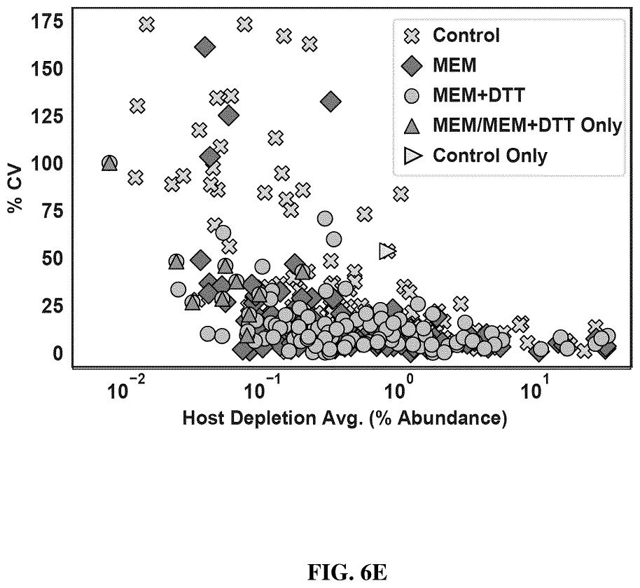

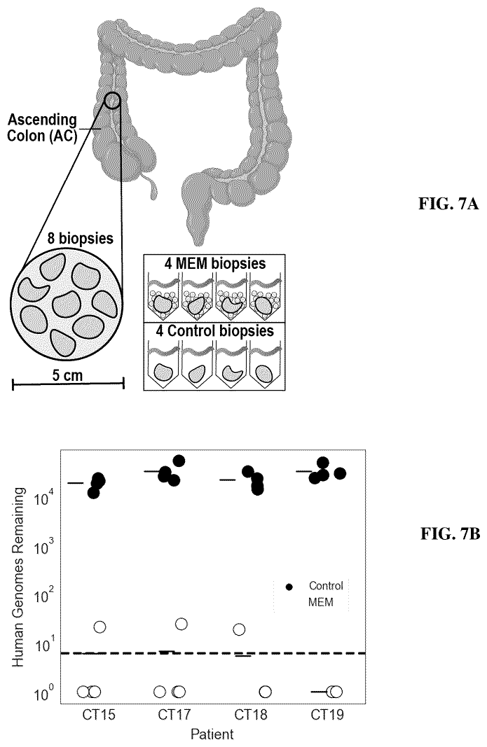

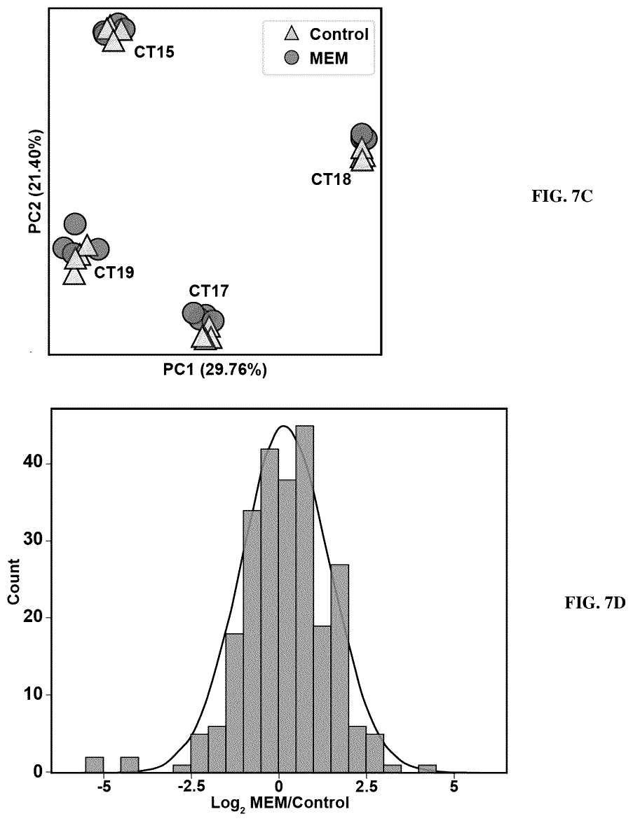

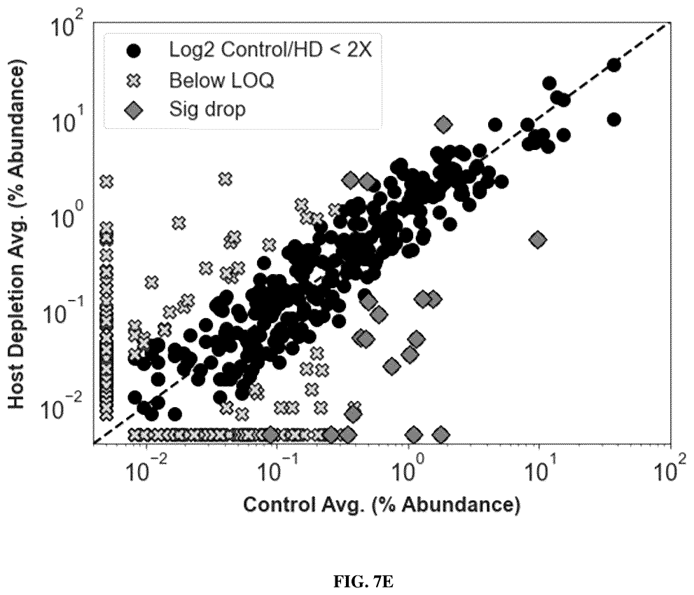

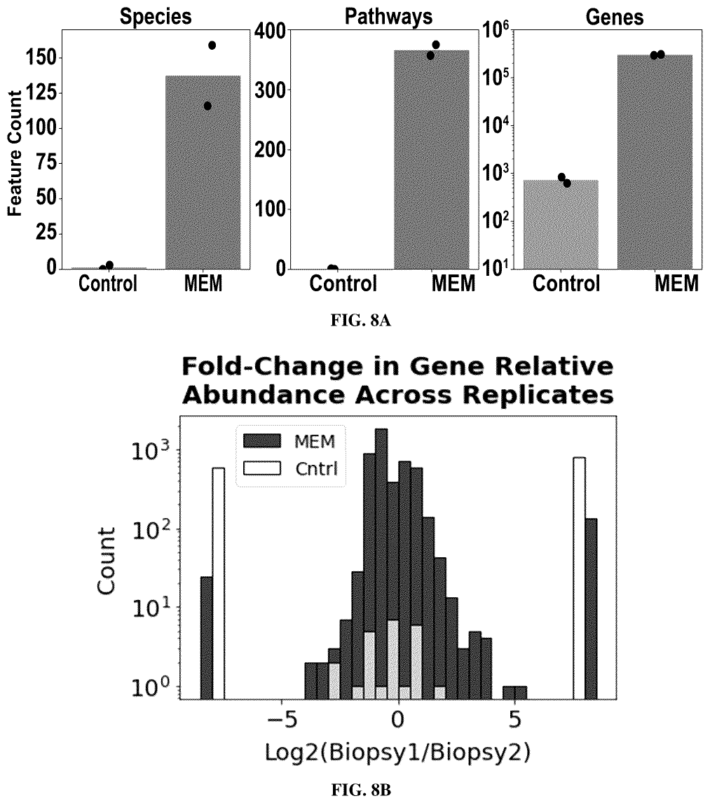

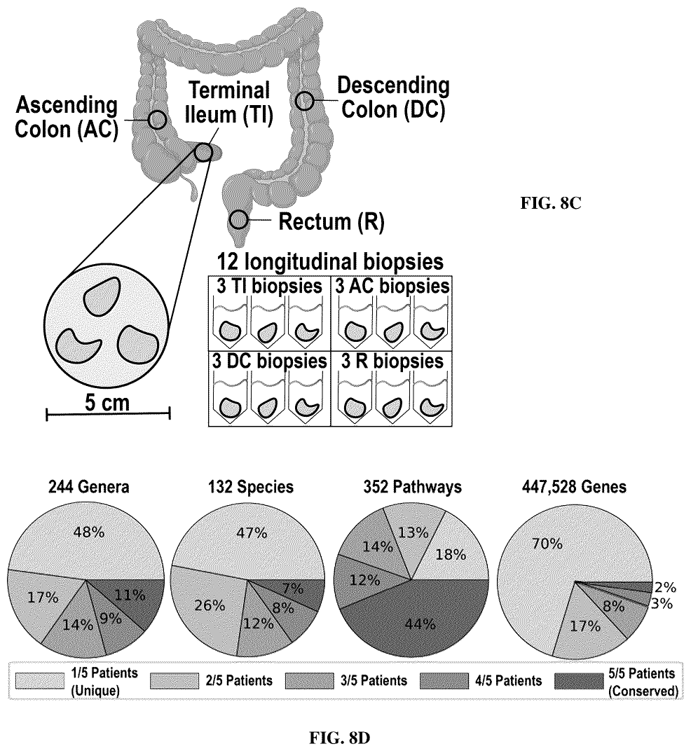

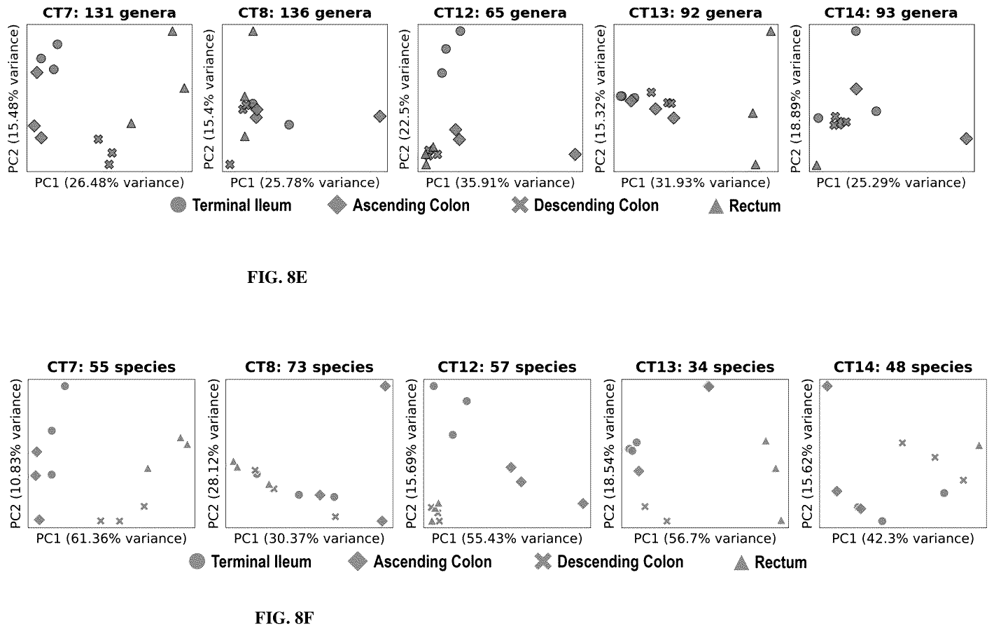

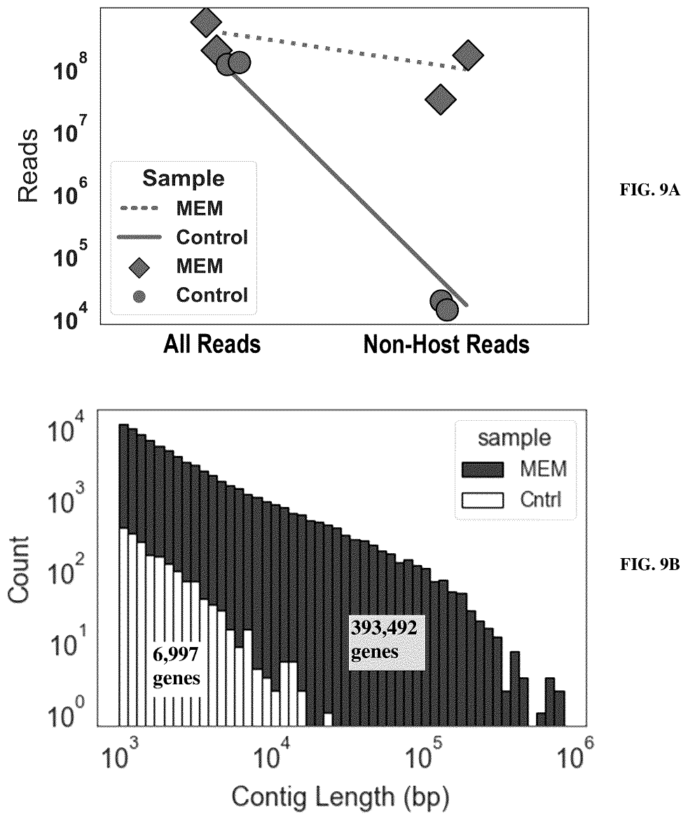

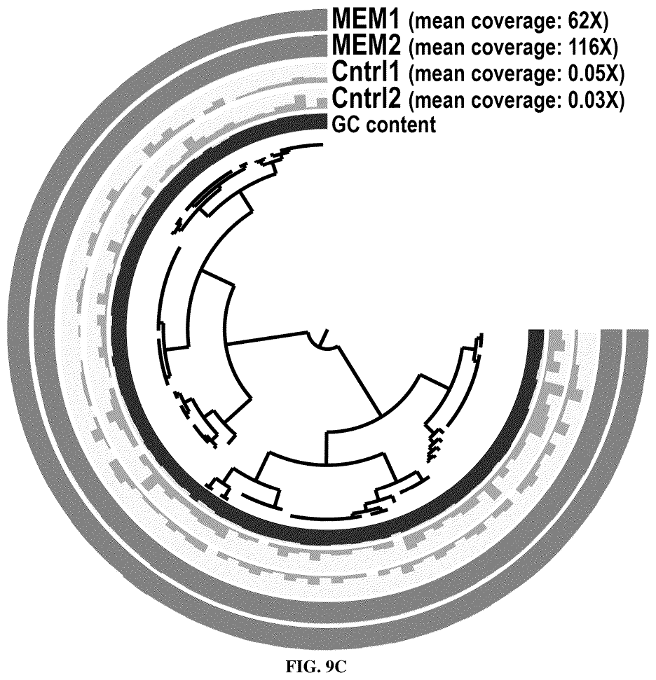

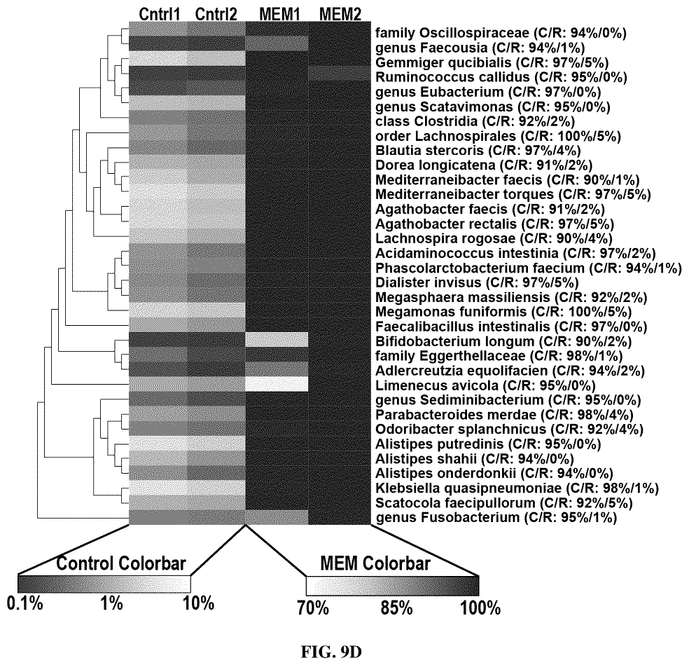

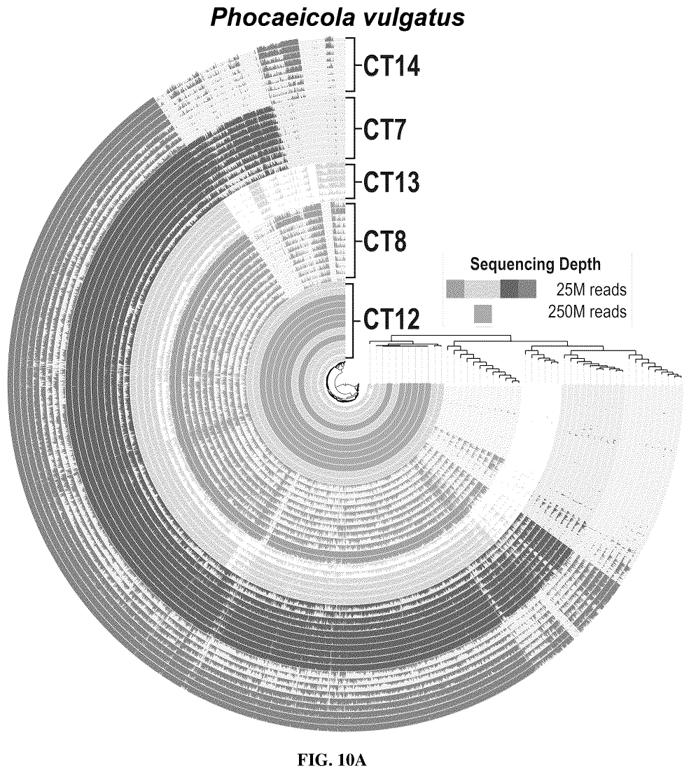

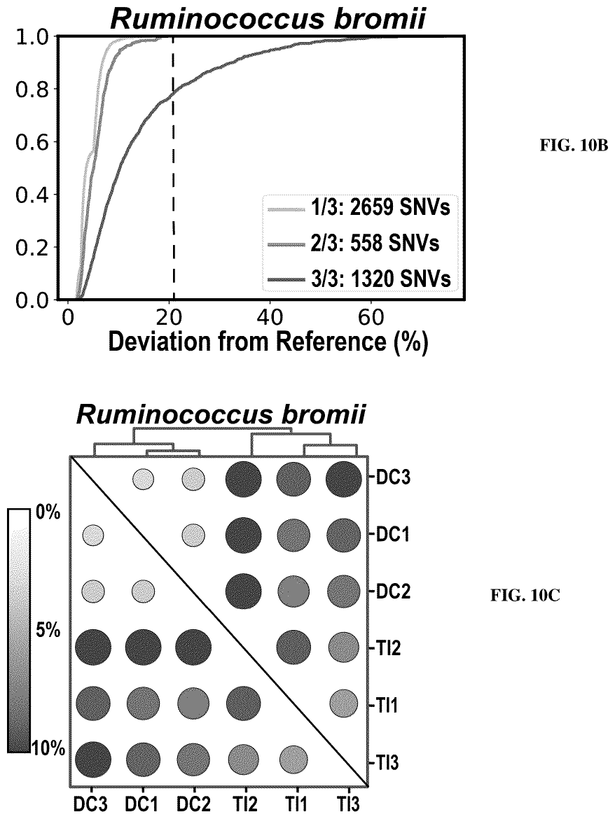

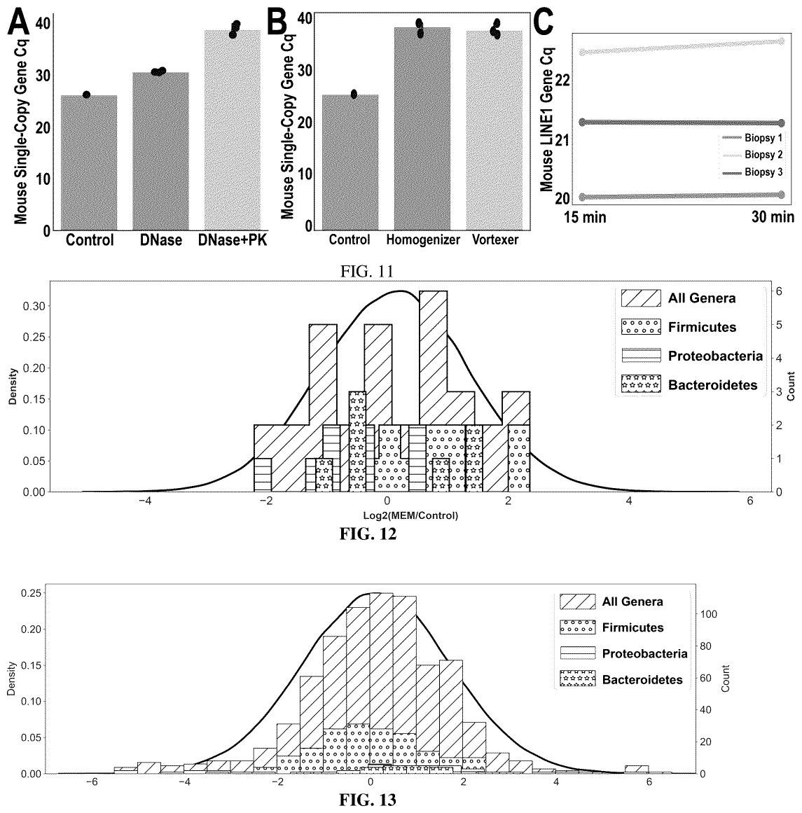

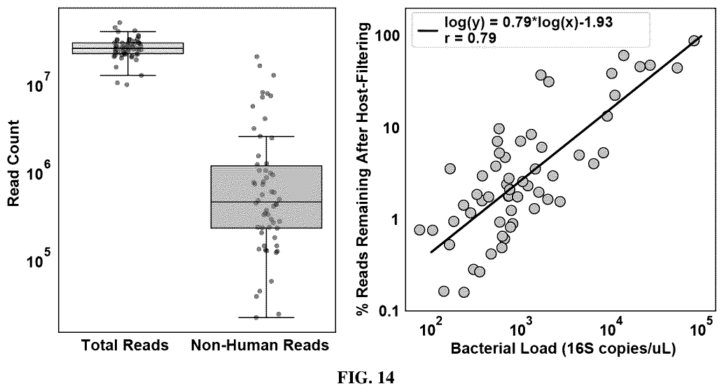

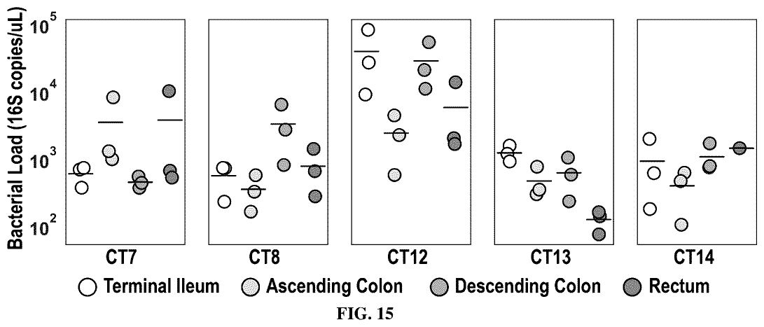

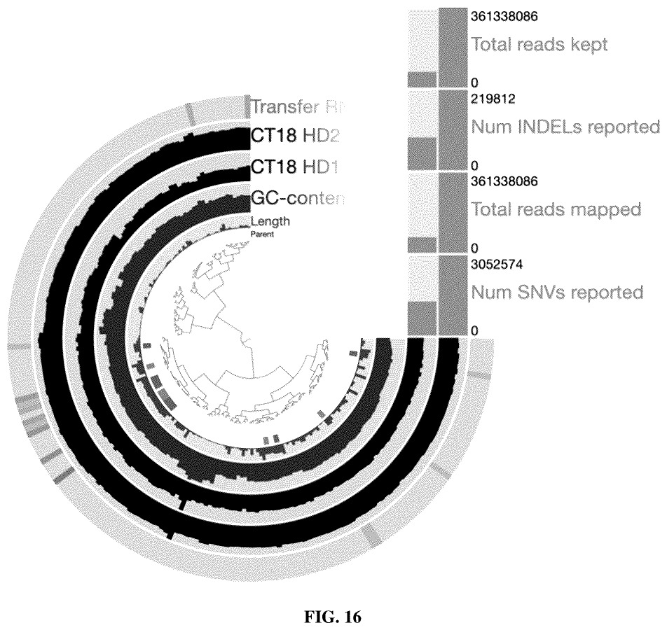

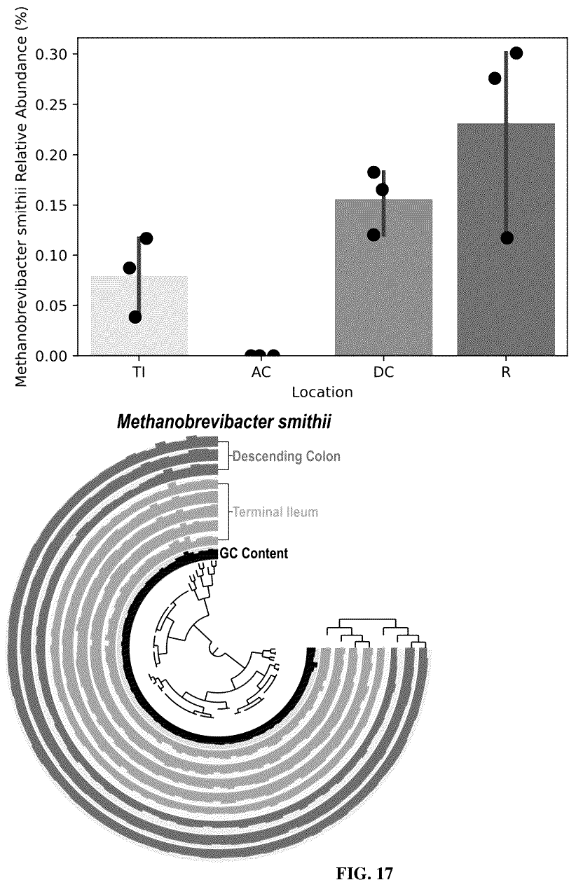

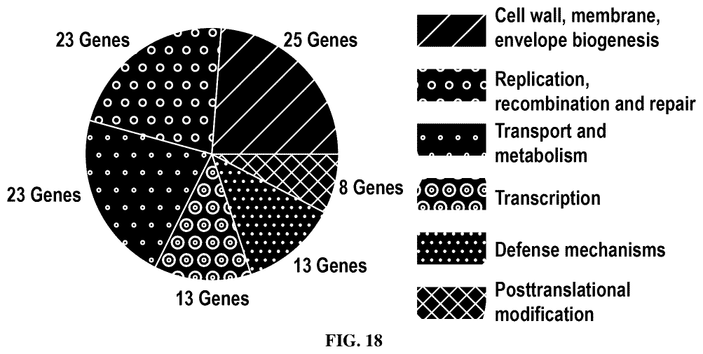

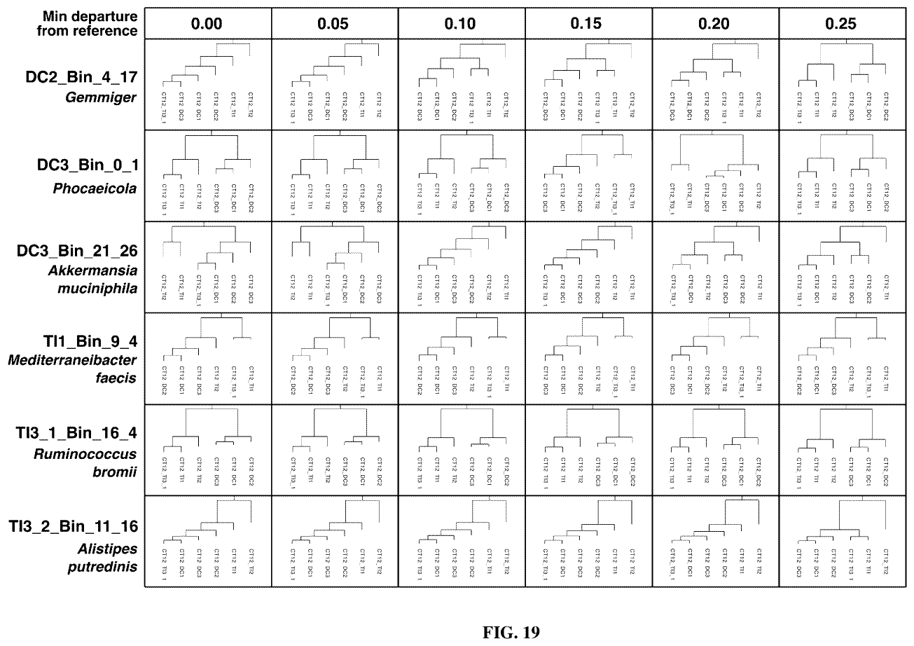

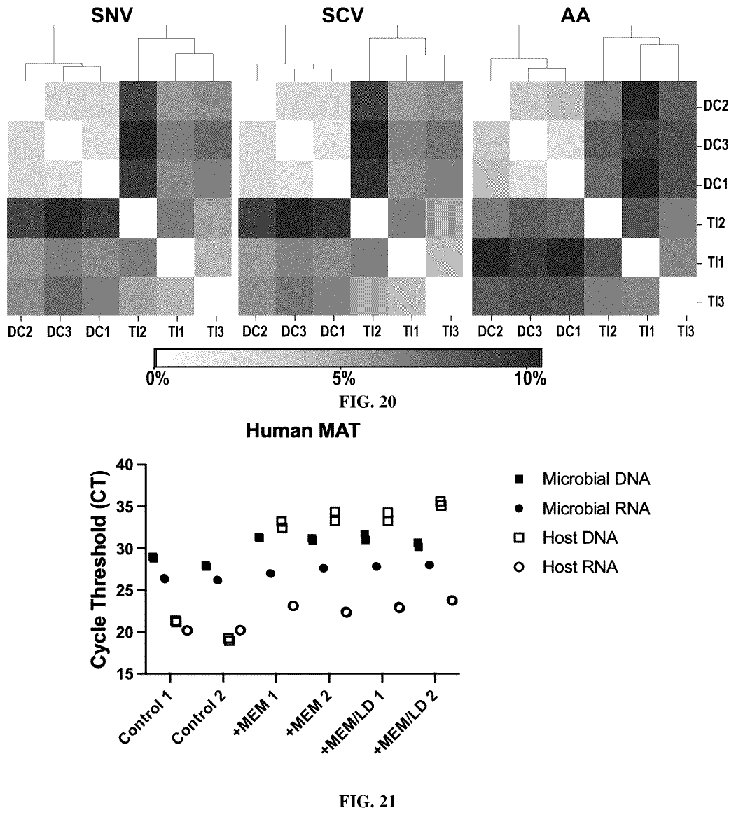

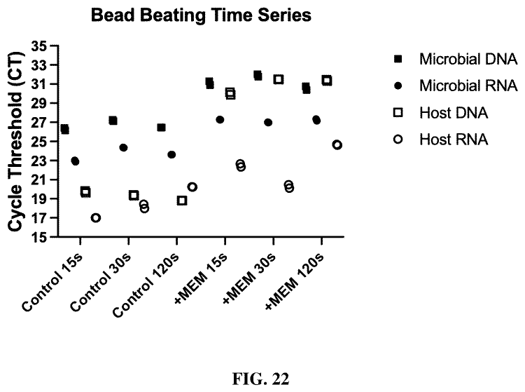

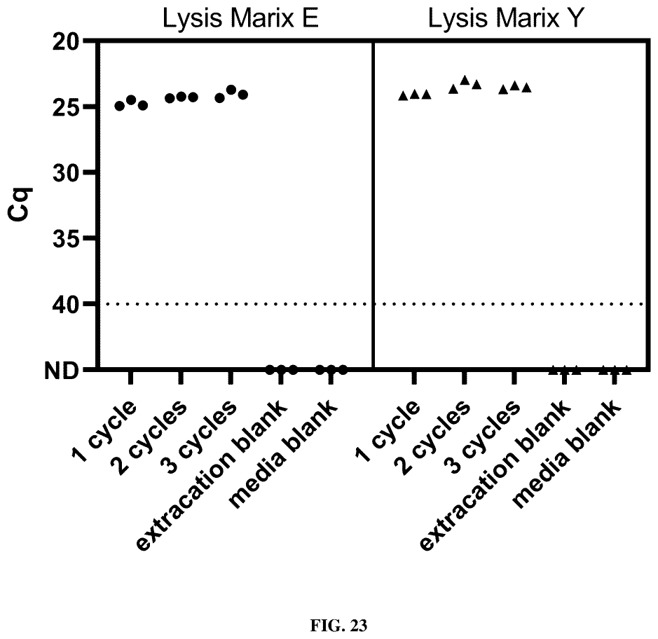

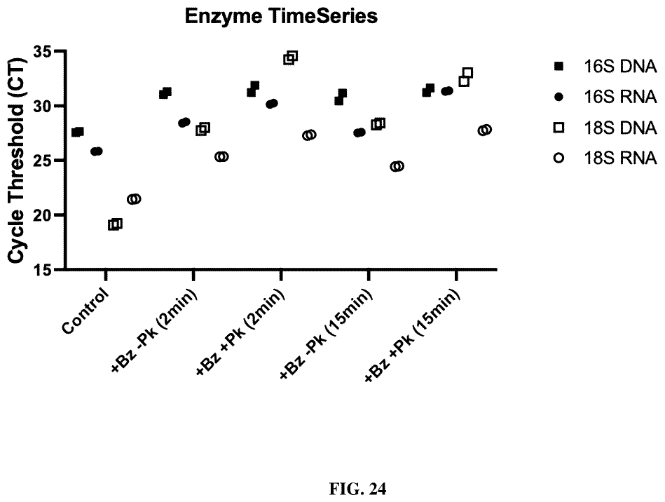

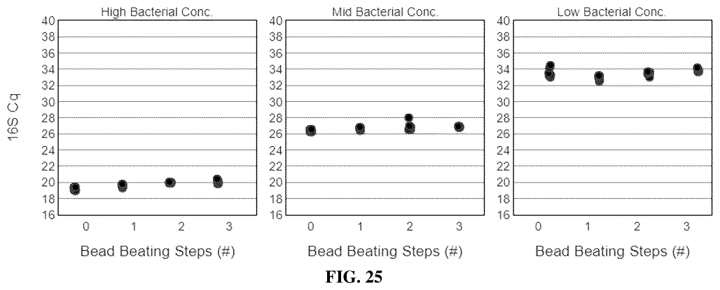

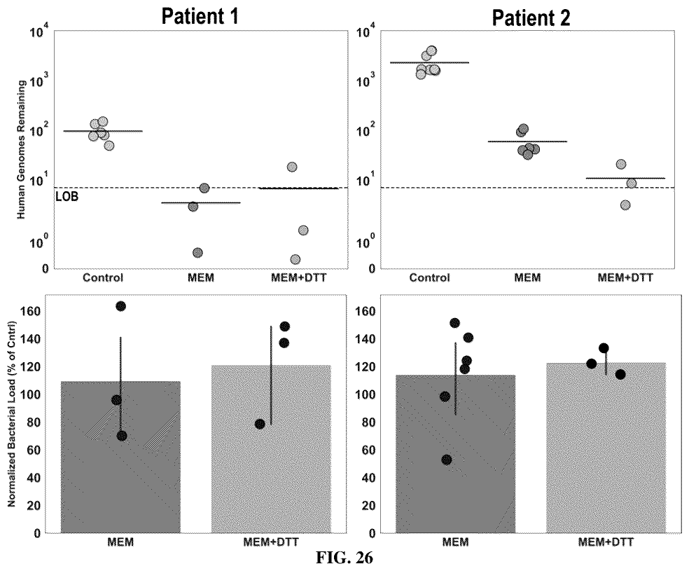

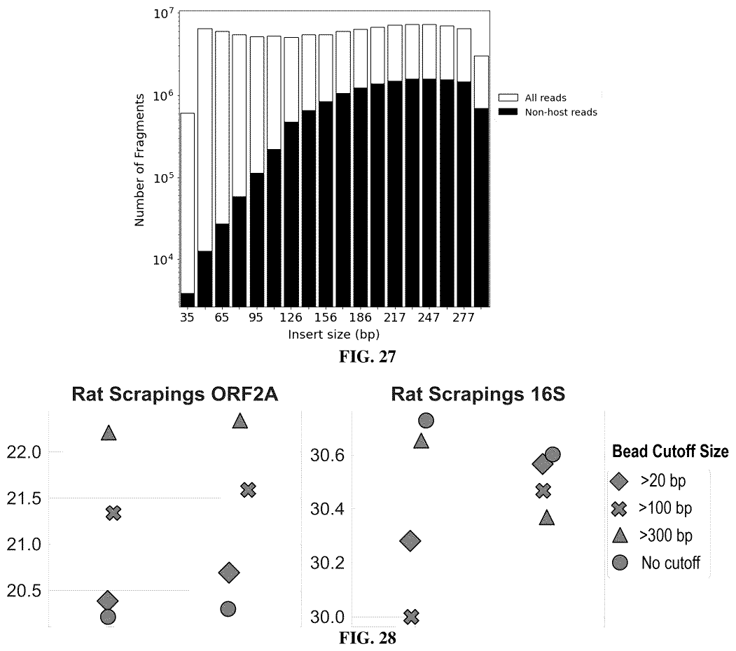

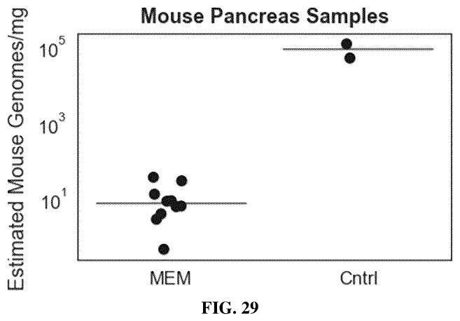

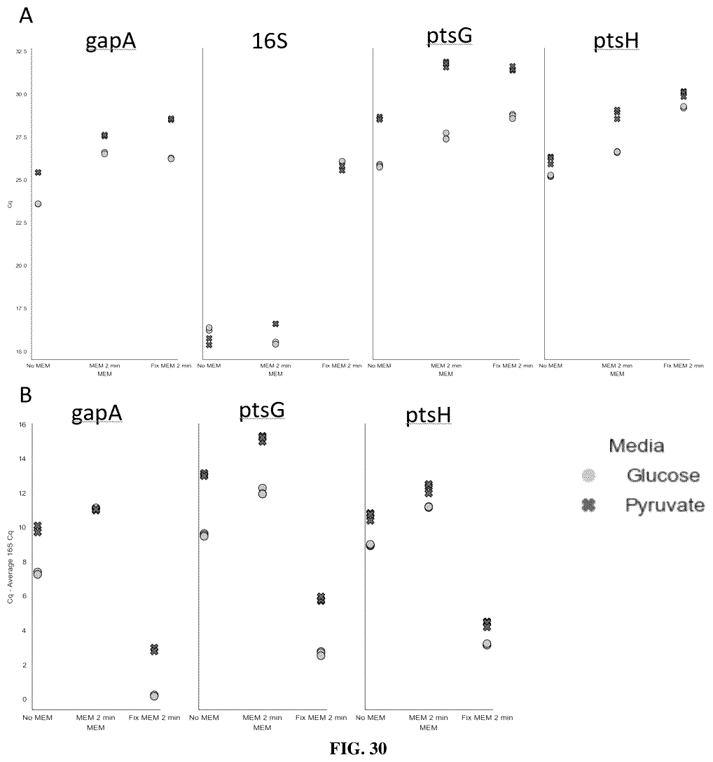

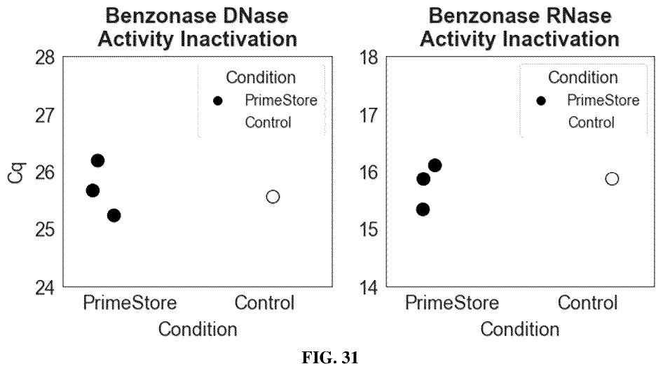

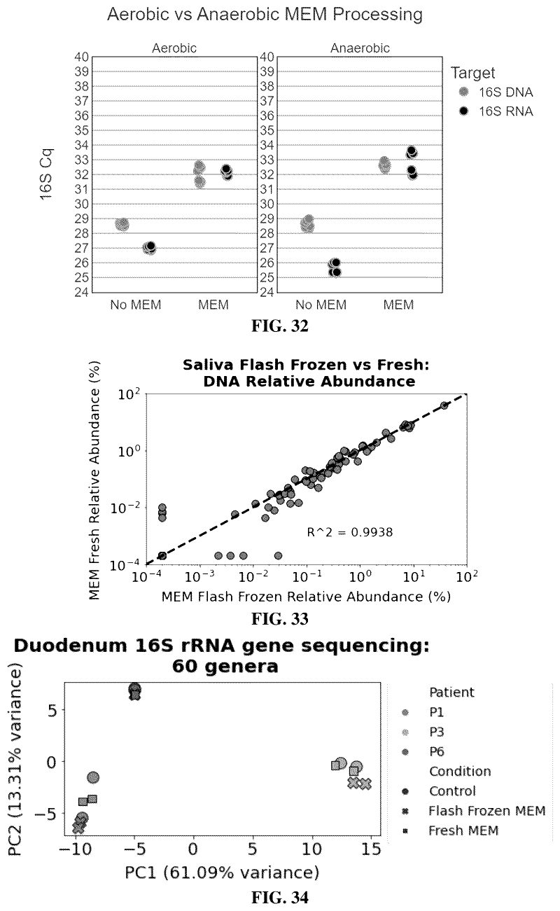

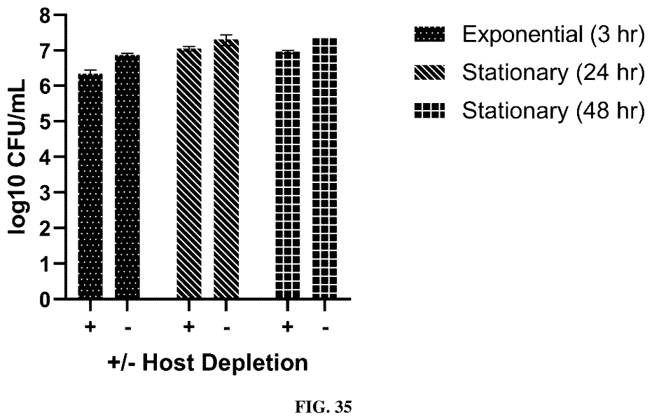

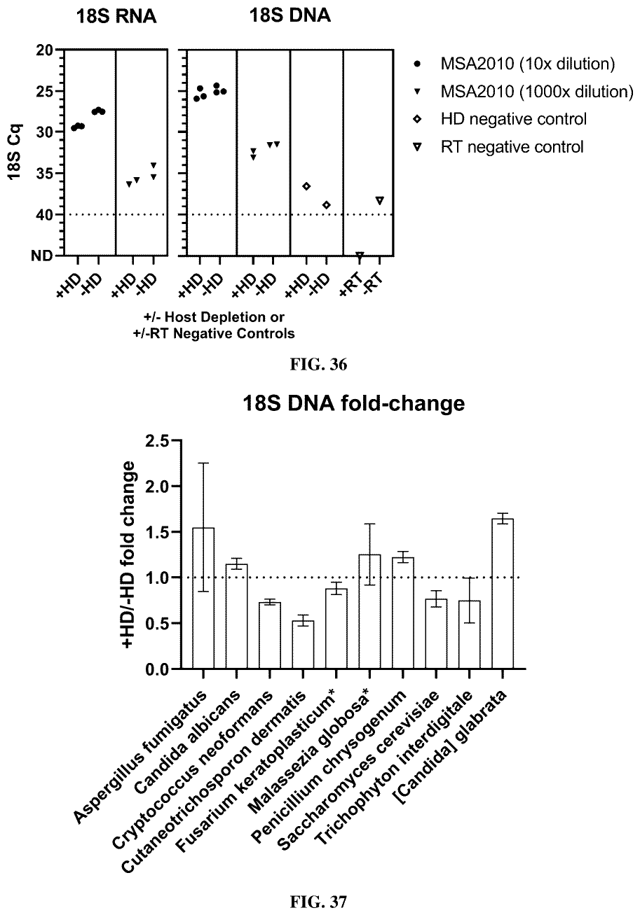

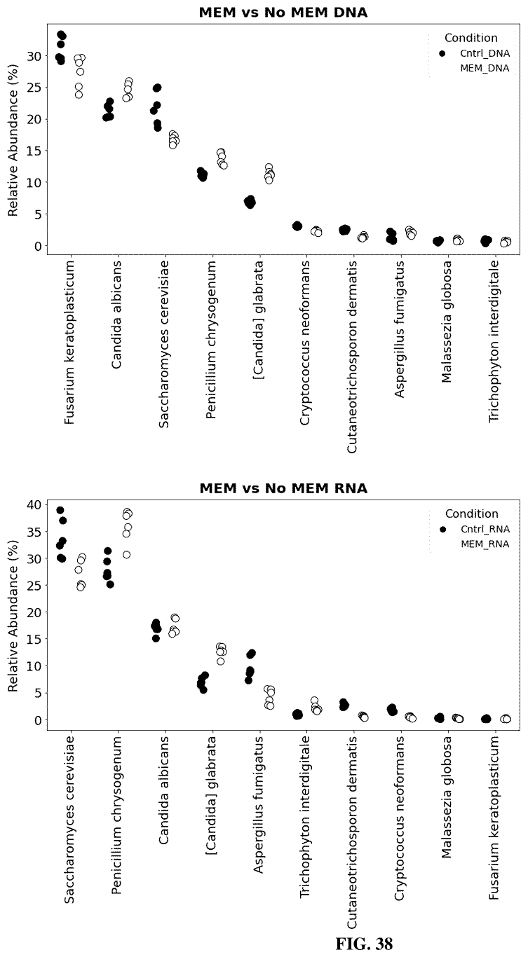

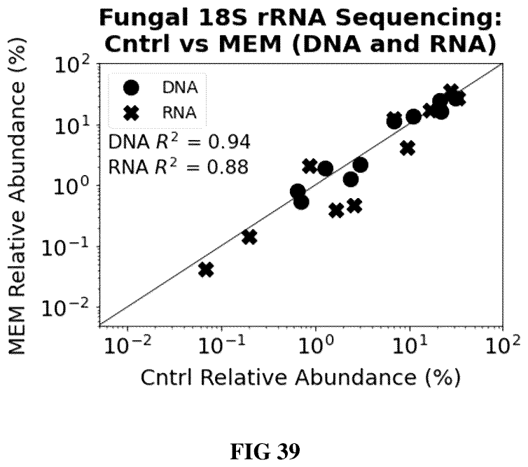

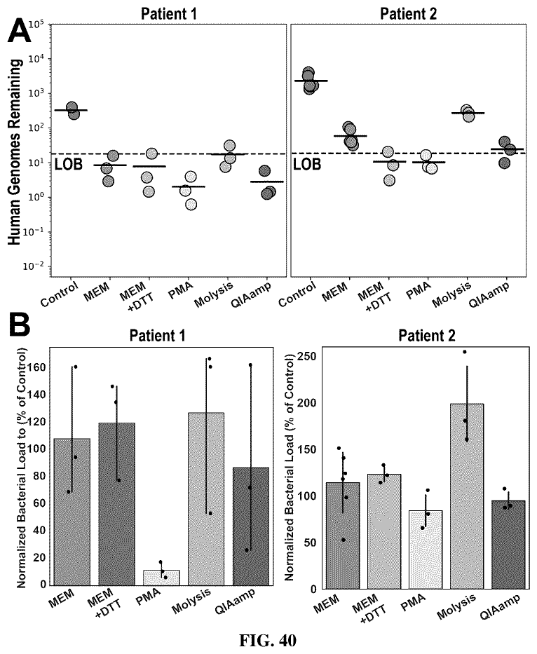

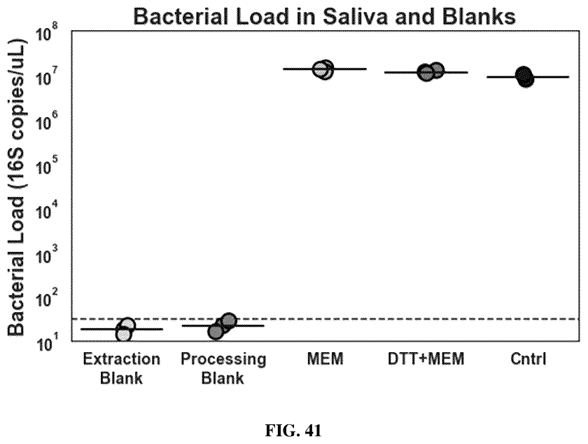

In particular, FIGS. 5A and 5B show diagrams and charts reporting data illustrating the effectiveness of an exemplary microbial enrichment approach of the disclosure on stool mouse stool samples in a comparison with published host-depletion methods. More particularly, FIG. 5A shows a diagram reporting results of experiments where bacterial loads from mouse stool samples treated with five different host-depletion methods. Loads are normalized to the control (no host depletion) stool samples (N=3; error bars are 95% CI). FIG. 5B shows a chart with an empirical cumulative distribution function (ECDF) of 16S rRNA gene amplicon sequencing results from mouse stool samples normalized to the control stool samples (N=3). Curves shifted to the left of the control indicate a greater percentage of taxa with lower abundance than the control samples following host depletion.

FIGS. 5C to 5E show diagrams and charts reporting data illustrating the effectiveness of an exemplary microbial enrichment approach of the disclosure on saliva, and intestinal samples in comparison with the same published host-depletion method analyzed in FIGS. 5A and 5B. In particular, FIG. 5C to 5E show charts reporting results of quantification of remaining host DNA through ddPCR of a single-copy host specific primer (see Methods). Reported genomes remaining refers to the abundance of this single-copy gene present in 1 μL of elution. More particularly, FIG. 5C shows the results of quantification of remaining human genomes in fresh human saliva after treatment with each host-depletion method and in untreated controls (N=3). FIG. 5D shows the results of host-depletion methods tested on mouse intestinal mucosal scrapings as a representative of soft tissue and remaining mouse genomes were quantified (N=3; biologic replicates from one mouse). FIG. 5E shows results of host-depletion methods tested on rat colonic sections as a representative hard tissue (including connective tissue, muscle, and mucosa) and remaining rat genomes were quantified (N=3; biologic replicates from one rat).

FIGS. 6A to 6E show diagrams and charts reporting data proving that the microbial enrichment approach and related method and systems of the disclosure enables accurate capture of stool and saliva microbiome. In particular, FIG. 6A to 6E illustrates results of microbial enrichment of stool (FIG. 6A and FIG. 6B) and saliva (FIG. 6C and FIG. 6E) after host-depletion performed with exemplary methods in accordance with the present disclosure as confirmed by shotgun sequencing. More particularly FIG. 6A shows, the percentages of non-host reads in control and MEM-treated mouse stool samples were calculated bioinformatically through alignment to a mouse reference genome (N=3; error bars are 95% CI). FIG. 6B shows species-level taxon relative abundances detected and plotted for control and MEM-treated mouse stool and overlaid on a dashed line showing 1:1 correlation. FIG. 6C shows results of shotgun sequencing performed on control and MEM-treated fresh human saliva. The percentages of non-host reads were calculated bioinformatically by identifying the percent of reads aligning to a human reference genome divided by the total number of reads passing QC. One saliva sample was evenly split nine ways for this comparison (N=3). FIG. 6D Species-level taxon relative abundances were plotted for control and MEM-treated fresh human saliva and overlaid on a dashed line showing 1:1 correlation. An additional DTT pre-treatment was performed prior to MEM treatment for a subset of MEM-treated samples (MEM+DTT) (N=3). FIG. 6E Coefficient of variation was plotted against relative species abundance and colored based on treatment types the taxa were detected in. Each point represents a species; crosses (X), diamonds ( [symbol] ) and circle (o) points indicate taxa that were present in all three treatments (control, MEM, and MEM+DTT). MEM/MEM+DTT only (vertical triangles (\hat{ })) indicate the 10 taxa found only in the MEM-treated samples. Control only (horizontal triangles (>)) indicates the single taxon that was found only in the control samples, which was identified as Haemophilus.

FIGS. 7A to 7E show schematics, diagrams and charts reporting data showing successful translation the microbial enrichment approach and related method and systems of the disclosure to clinical intestinal biopsies. In particular, FIGS. 7A to 7E, shows results of an analysis of microbial enrichment in paired human intestinal biopsies processed with and without microbial enrichment methods of the instant disclosure. More particularly, FIG. 7A shows a sampling graphic illustrating the collection from four participants (CT15, CT17, CT18, and CT19) each with eight ascending colon biopsies. Four biopsies from each participant were MEM-treated and four were untreated controls. FIG. 7B shows a chart reporting results of detection of host DNA quantified for each biopsy using ddPCR of a single-copy host primer. Human genomes remaining refers to the abundance of this single-copy gene present in 1 μL of elution (* indicate measurement was below limit of blank [LoB] defined as LoB=mean_{blank}+1.645[SD_{bank}] based on three processing blanks). FIG. 7C shows a chart reporting detection and analysis of biopsies characterized with 16S rRNA gene sequencing and principal coordinates analysis (PCA) on microbial genus-level relative abundances were performed to visualize microbial population variation. FIG. 7D shows a diagram reporting Log 2-fold differences in relative abundances of taxa between control and MEM-treated biopsies detected and plotted with a standard normal distribution overlaid in black. FIG. 7E shows a chart reporting the relative abundance of taxa measured in control vs MEM-treated biopsies were plotted and overlaid on a dashed line showing 1:1 correlation. Taxa that were below the assay limit of quantification (LOQ) are represented as grey x's. Taxa with greater than 4-fold changes between control and MEM biopsies are represented as grey diamonds.

FIG. 8A to FIG. 8F show schematics, diagrams and charts reporting data reporting results of shotgun sequencing of human intestinal biopsies treated with an exemplary MEM processing in accordance with the disclosure. In particular, FIG. 8A shows a diagram reporting results of shotgun sequencing of four biopsies from participant CT18 (two MEM-treated and two control) wherein the number of microbial species, pathways, and genes identified in each sample are plotted (N=2; error bars are 95% CI). FIG. 8B shows a diagram plotting for the top 5,000 abundant genes, the log 2 fold-change in relative abundances between the two MEM-treated biopsies and the two control biopsies. FIG. 8C shows a sampling graphic for collection of 12 biopsies from each of 5 participants (3 biopsies taken from 4 separate regions of the GI tract). Biopsies within one region were sampled within one field of view (5-cm diameter). FIG. 8D shows pie charts reporting results for all 60 biopsies processed with MEM, followed by 16S rRNA gene sequencing (for genera) and shotgun sequencing (for species, pathways, and genes). The number of features for genera, species, pathways, and genes were grouped based on whether they were present in at least one biopsy sample from only one participant (1/5), two participants (2/5), three participants (3/5), four participants (4/5), or from all participants (5/5). Analysis was performed on annotated genes, and separately on all genes (annotated+unannotated). FIGS. 8E to 8F show charts reporting PCA performed on all 60 longitudinal samples grouped by participant. In particular, FIG. 8E shows a chart reporting PCA on relative abundance of 16S rRNA gene sequencing genera assignments for each participant separated by sampling location Terminal lleum (circle (o)) Ascending Colon (diamond ( [symbol] )), Descending Colon (cross (x)), and Rectum (triangle (\hat{ })). FIG. 8F shows a chart reporting PCA on relative abundance of shotgun-sequencing species assignments for each participant separated by sampling location Terminal lleum (circle (o)) Ascending Colon (diamond ( [symbol] )), Descending Colon (cross (x)), and Rectum (triangle (\hat{ })).

FIGS. 9A-9D show schematics, diagrams and charts reporting data illustrating MAG construction with human intestinal biopsies treated with an exemplary enrichment method of the present disclosure, the MAG construction performed from shotgun metagenomic sequencing. In particular: FIG. 9A shows a chart reporting shotgun-sequencing of two control and two MEM-treated biopsies from the same participant (CT18) and intestinal region (ascending colon). Number of non-host reads were determined after alignment to a human reference genome. FIG. 9B, shows a diagram reporting contigs constructed from co-assembly of the two samples from each condition and the distribution of contig lengths was plotted. The number of prokaryotic genes identified in these contigs is shown. FIG. 9C, shows a MAG of Alistipes putredinis constructed from co-assembled MEM biopsies. Bar heights represent mean coverage and are scaled independently for each sample. FIG. 9D, shows 34 high-quality MAGs (>90% complete, <5% redundant) constructed de novo from co-assembly of MEM biopsies, 34 high-quality MAGs (>90% complete, <5% redundant). Heatmap shows the percentage of each genome that is covered at least 1× by the sample (i.e., detection or breath of coverage), with a maximum of 3.7% in control samples and 99.999% in MEM samples. The average detection for MEM1, MEM2, Cntrl1, and Cntrl2 were 99.8% (SD: 0.7%), 97.3% (SD: 6.4%), 0.8% (SD: 0.7%), and 1.2% (SD: 1.1%) respectively across all MAGs. Taxonomy was assigned for each MAG and listed to the right along with completion/redundancy (C/R). The phylogenetic tree to the left of the heatmap highlights taxonomic grouping of each MAG.

FIGS. 10A to 10C shows charts and schematics, illustrating biodiversity present along GI tract detected following microbial enrichment approach and related method and systems of the disclosure. In particular: FIGS. 10A to 10C shows data illustrating Interindividual and intraindividual bacterial biodiversity present along GI tract detected in sample following microbial enrichment with an exemplary method of the present disclosure. FIG. 10A shows a chart reporting the results of a gene-level analysis performed on Phocaeicola vulgatus for all five participants. Samples were grouped by gene detection, defined as percentage of each gene with at least 1× coverage, and showed strong participant-dependent grouping but lacked grouping by GI location. FIG. 10B, shows a chart reporting the ECDF of the occurrence of single-nucleotide variants (SNVs) in a MAG of Ruminococcus bromii and the deviation of these SNVs from the reference across three technical replicates. 1/3, 2/3, and 3/3 indicates the number of technical replicates that had an SNV at that location followed by the total number of SNVs in each of these categories. A black dashed line is drawn at 21% deviation from reference; above this value, all observed SNVs were present in all three technical replicates. FIG. 10C, shows a chart reporting the results of nucleotide-level analysis performed on MAGs with a mean coverage above 50× across all samples. Shown here is the fixation index from SNVs analyzed within the coding region of R. bromii with a minimum deviation from reference set at 21%. Samples were clustered based on fixation index and strong region-dependent groupings can be seen. DC, descending colon; TI, terminal ileum.

FIG. 11 shows charts reporting the results of experiments performed to obtain MEM protocol optimization. In particular, FIG. 11 panel A, reports results of MEM protocol tested with and without Proteinase K (PK) treatment on mammalian mouse cell culture. In particular the host was quantified after MEM treatment and DNA extraction with single-copy mouse primers with qPCR. FIG. 11 panel B, reports results of MEM protocol performed on mammalian mouse cell culture to compare the effectiveness of a homogenizer or vortexer in host cell disruption. A comparison between homogenizer (4.5 m/s for 30 sec) vs vortexer adapter (1 min at max speed) was performed and remaining host DNA after MEM treatment was quantified using qPCR on single-copy mouse primers. We concluded homogenizers are not necessary and this may be important for field work for future environmental samples. FIG. 11 panel C reports results of MEM performed on 3 rat biopsies split into two different reactions with 15-min or 30-min incubation at 37° C. Host load was quantified through LINE1 transposon primers with qPCR. Minimal differences in host DNA removal with incubation of 15 or 30 minutes in nuclease. Final protocol used 15-minute incubation to minimize processing times.

FIGS. 12 and 13 show diagrams illustrating the Impact of host depletion on specific bacterial phyla and in particular detection of Log 2 fold-change between relative abundance of genera within each phylum in treated and control samples from 16S rRNA gene sequencing data for FIG. 12, mouse stool with a variety of host depletion methods, FIG. 12, human saliva on paired MEM-treated and untreated controls, The histograms in FIGS. 12-and 13 are overlaid with a normal distribution (black line).

FIG. 14 shows a diagram illustrating the correlation between bacterial load and non-host reads. Shotgun sequencing was performed on longitudinally sampled intestinal biopsies after processing with host depletion. Roughly 25 million reads on average were obtained for each biopsy and all samples fit on a single NovaSeq S1 flowcell. After host-filtering an average of 2 million reads were remaining with a range from 2E4 reads to 2E7 reads. The variability in non-host reads remaining had a strong correlation (Spearman, r=0.79) with the total microbial load as measured by digital PCR. This strong correlation indicated that our process was achieving a relatively uniform depletion across all samples. Additionally, the strong correlation indicates that the majority of non-human reads in our samples come from bacteria picked up by the 16S primers used for total microbial load quantification.

FIG. 15 shows a diagram of bacterial loads following MEM treatment on longitudinally collected intestinal biopsies from 5 individuals, where the individuals are labeled CT7, CT8, CT12, CT13, CT14. 16S rRNA gene copies were quantified as a proxy for bacterial load for all biopsies. Samples were plotted by participant and then by location.

FIG. 16 shows a diagram illustrating MAG of Fusobacterium performed after an microbial enrichment performed with an exemplary method of the present disclosure. From two MEM-treated ascending biopsies from CT18, a MAG of Fusobacterium was constructed (completeness: 94%, redundancy: 1.4%).

FIG. 17 shows a diagram illustrating Archaeon Methanobrevibacter smithii found along the lower GI tract in a sample treated with exemplary microbial enrichment method in accordance with the present disclosure. From shotgun sequencing, we detected participant CT12 had low levels of Methanobrevibacter smithii present in the terminal ileum, descending colon, and rectal biopsies. MAG construction was performed on co-assembly of all biopsies taken from the terminal ileum and descending colon to reconstruct a full Methanobrevibacter smithii genome (completeness: 100%, redundancy: 0%).

FIG. 18 shows diagrams illustrating genes unique to the strains of Phocaeicola vulgatus detected in CT12 biopsies that were treated with exemplary microbial enrichment method of the present disclosure. 100 annotated genes found in only CT12 were sorted based on COG20 Category and the number of genes in each category are shown.

FIG. 19 shows a diagram illustrating Fixation index across MAGs with varying deviation from reference, detected following an exemplary enrichment method of the present disclosure. Six MAGs with greater than 50× mean coverage were selected for SNPs analysis. Fixation index analysis was performed on each MAG for various thresholds of minimum departure from reference nucleotide. Clustering of fixation index by location can be seen for some MAGs.

FIG. 20 shows a diagram illustrating Ruminococcus bromii strain variants at the nucleotide (SNV), codon (SCV), and amino acid (AA) level. SNVs present in R. bromii above the threshold of 21% deviation from reference were analyzed at the codon and translated-level to determine if SNVs may indicate a functional change. The fixation index for each level of analysis were plotted.

FIG. 21 shows a chart illustrating results of experiments where human mesenteric adipose tissue (MAT) was homogenized using bead beating and processed with MEM either with or without lipid depletion (LD). Control samples represent non-MEM treated samples (tissue homogenate that proceeded directly to extraction). Numbers indicate processing replicates. Microbial nucleic acids were measured using primers targeting the V4 region of the microbial 16S rRNA gene, and host nucleic acids were measured using primers specific to the mammalian 18S rRNA gene. Filled icons represent microbial NA measurements, while open icons represent measurements of host NAs. Squares represent DNA measurements, while circles represent RNA measurements.

FIG. 22: shows a chart illustrating results of experiments with Human mesenteric adipose tissue (MAT) was homogenized for 15, 30, or 120 seconds preceding either direct sample extraction (Control) or MEM treatment (+MEM). Microbial nucleic acids were measured using primers targeting the V4 region of the microbial 16S rRNA gene, and host nucleic acids were measured using primers specific to the mammalian 18S rRNA gene. Filled icons represent microbial NA measurements, while open icons represent measurements of host NAs. Squares represent DNA measurements, while circles represent RNA measurements.

FIG. 23 shows a chart illustrating different lysis efficiency of Saccharomyces boulardii with three different lysis beads for 3 different durations. Saccharomyces boulardii was quantified with qPCR using the TEF gene.

FIG. 24 show a chart illustrating different enzyme inputs and incubation times on host depletion and microbial recovery using the MEM protocol. The samples were proceeded directly to extraction (Control), mixed with 2 ul of nuclease benzonase (+Bz −Pk), or mixed with 2 ul of nuclease benzonase and 5 ul of Proteinase K (+Bz +Pk) and incubated at 37 C with 600 RPM shaking for either 2 minutes or 15 minutes. Following extraction the samples quantified using RT/qPCR detecting 18S (Host) or 16S (Bacteria) gene. Filled icons represent microbial NA measurements, while open icons represent measurements of host NAs. Squares represent DNA measurements, while circles represent RNA measurements.

FIG. 25: shows charts illustrating the impact of non-specific bacterial cell lysis when bead beating using large (1.4 mm) zirconium silicate beads across high, mid and low bacterial loads. Samples were then MEM treated then incubated at 37 C for 15 minutes at 600 RPM shaking and then recovered by centrifuging at 10,000 g for 2 minutes, removing the supernatant, and resuspending cell pellets with 800 ul of lysis buffer. DNA loads were quantified with qPCR detecting 16S rRNA gene.

FIG. 26: shows charts illustrating the effect of DTT in saliva on efficiency of host removal. Two healthy patients donated stimulated saliva and were split to be processed with and without MEM (with and without DTT). Microbial nucleic acids were measured using primers targeting the V4 region of the microbial 16S rRNA gene, and host nucleic acids were measured using primers targeting a single copy gene.

FIG. 27: shows a chart reporting data illustrating shot-gun DNA sequencing of a human colonic biopsy treated with MEM generates a large population of libraries with very small inserts (eg. between 30-100 bp) that are host derived.

FIG. 28: shows schematics, diagrams and charts reporting data illustrating exemplary method to remove partly degraded host DNA (i.e. defined as below 5000 bp) is through SPRI bead clean-ups. To test this, rat scrapings (RS) that were previously MEM treated were SPRI cleaned at various ratios of beads to elution (see legend) to remove partly degraded host DNA. From this experiment, we see additional removal of ORF2A host signal with bead clean-up above 100 bp, with minimal impact on 16S bacterial signal. We would expect additional removal of host signal may be possible by increasing the cutoff size of the SPRI beads.

FIG. 29 shows a chart illustrating MEM application to remove host signal on a soft organ tissues type. Pancreatic tissue from a mouse was MEM treated and host was quantified using qPCR detecting a single copy mouse gene normalized to tissue input in mg.

FIG. 30 shows a study inducing transcriptional expression of bacteria cells grown in media containing Glucose (Grey circle) or Pyruvate (grey x) treated with different embodiments of MEM. Transcriptional responses were quantified using two step RT-qPCR to detect gapA, 16S rRNA, ptsG, ptsH reporting raw abundance per target (Part A) or mRNA levels were normalized to 16S levels to account for different amounts of total RNA extracted from the samples (Part B).

FIG. 31 shows a chart illustrating a method that can inactivate the nucleases added during the MEM during microbial lysis. To test this, we added 2 uL of Benzonase into inactivation (PrimeStore MTM) buffer and a sample of DNA/RNA from Neisseria gonorrhoeae. We then quantified the amount of DNA and RNA. From this experiment no DNA or RNA losses were detected when exposed to PrimeStore inactivated Benzonase

FIG. 32 shows charts containing data illustrating MEM conducted in aerobic and anaerobic conditions yields similar recovery of bacterial DNA and RNA. Human mesenteric adipose tissue (MAT) was processed with direct sample extraction (No MEM) or MEM under aerobic or anaerobic conditions. Microbial nucleic acids were measured using primers targeting the V4 region of the microbial 16S rRNA gene.

FIG. 33 shows a chart containing data that illustrates MEM treatment is compatible with fresh and flash freezing in liquid nitrogen. Human saliva specimen was split 6 ways (3 frozen immediately and 3 processed immediately as fresh with MEM). Using 16S rRNA sequencing, relative abundance of taxa present in both MEM treated flash frozen and fresh were plotted against each other.

FIG. 34 shows the feasibility of MEM treatment on both fresh and flash frozen duodenal biopsies by extracting and quantifying DNA of paired control (untreated), fresh, and flash frozen duodenal biopsy samples. Using Principal coordinates analysis, one can see that any differences in microbial relative abundances introduced by MEM were less than the differences observed between participants.

FIG. 35 Shows a chart containing data illustrating MEM treatment of different growth phases of S. boulardii at different time points after culture inoculation. Colony counts of S. boulardii are plotted on the y axis (log 10 CFU/mL) for samples that were processed before (−) and after (+) host depletion at exponential growth phase (3 hours after culture inoculation) and stationary phases (24 hours and 48 hours after the initial inoculation). Each bar is calculated from the average of four plating replicates, except for the (−) host depletion condition at stationary (48 hr) growth, where only one plate was counted. Error bars depict standard deviation.

FIG. 36 shows 18S quantification of RNA and DNA of a fungal community before and after MEM host depletion. 18S rRNA Rt-qPCR reveals losses of under 2 Cq upon MEM treatment of a fungal community. The Cq of 18S RNA (first two columns) and 18S DNA (third and fourth columns) before (−HD) and after (+HD) host depletion is plotted on the y axis for a complex ten-taxa mix that has been either 10× diluted (solid circle) or 1000× diluted (solid upside down triangle) from stock.

FIG. 37 shows community composition of a ten-taxa fungal mix before and after host depletion supporting that fungal communities treated with MEM does not bias towards a specific taxon. The ratio of the relative abundances of each fungal taxa after and before host depletion (+MEM/−MEM) are plotted on the y axis for each fungal taxa in MSA2010 for 18S. Each condition is calculated from the average of three extraction replicates. Error bars depict the standard deviation.

FIG. 38 shows a chart containing data that illustrates application of MEM to isolate replication viable microbes is feasible by comparing no MEM and MEM treated fungal ten taxonomic cell mixture DNA:RNA ratios. Using 18S rRNA DNA and RNA sequencing specific for fungal taxa, relative abundances of MEM treated (white circles) or no treatment (black circles) fungal taxa were compared.

FIG. 39 shows a chart containing data that illustrates application of MEM to isolate replication viable microbes is feasible by comparing no MEM and MEM treated fungal ten taxonomic cell mixture DNA:RNA ratios. Using 18S rRNA DNA and RNA sequencing specific for fungal taxa, relative abundances of MEM treated (white circles) or no treatment (black circles) fungal taxa were compared.

FIG. 40 shows diagrams illustrating saliva host depletion variation across participants. All commercial host depletion protocols and MEM were performed on two separate saliva donors a total of three times per donor. After host depletion, host load and bacterial load were quantified with qPCR of single-copy human primers and 16S rRNA gene primers. In particular, FIG. 40 Panel A shows Host loads in participant 1 saliva (shown in FIG. 5C) was 10-fold lower compared to participant 2 we tested. The addition of DTT did not have an effect on the participant with lower host load but caused higher reduction in host in the participant with higher initial host load. Dashed line indicates limit of blank (LOB) defined as LoB=mean_{blank}+1.645(SD_{blank}) based on three processing blanks. FIG. 40 Panel B, shows diagrams reporting minimal loss of microbial DNA from PMA treatment in participant 2 saliva (right) but dramatic microbial losses in participant 1's saliva. This loss in low host saliva may be due to excess PMA that was incompletely inactivated. Disadvantage of lyPMA is it will need to be reoptimized for each sample's biomass.

FIG. 41 shows a diagram illustrating: Bacterial load present in saliva and extraction/processing blanks. Bacterial load measurements for saliva samples that were shotgun sequenced compared to processing and extraction blanks. Dashed black line represents the measurement LOB.

### DETAILED DESCRIPTION

Provided herein is a microbial enrichment methodology and related methods and systems which selectively targets the host cells of an biological sample to obtain depletion of host cells and related nucleic acid while minimizing depletion of any microbial compartment and related nucleic acid possibly present in the sample.

The term “microbial” “microbe” or “microorganism”, as used herein indicates a microscopic organism selected from viruses and living organisms which can exist in a single-celled form or in a colony of cells form. Accordingly, microorganisms in the sense of the disclosure, viruses and an extremely diverse unicellular organisms, including prokaryotes and in particular bacteria, but also including fungi (yeast and molds), and protozoal parasites as will be understood by a skilled person.

The term “virus” and “viruses” as used herein indicates a submicroscopic microbe capable of replicating only inside the living cells of an organism. A complete virus particle, known as a virion, consists of nucleic acid surrounded by a protective coat of protein called a capsid. These are formed from protein subunits called capsomeres. [1]Viruses can have a lipid “envelope” derived from the host cell membrane. Viruses can have a lipid “envelope” derived from the host cell membrane. The capsid is made from proteins encoded by the viral genome and its shape serves as the basis for morphological distinction. [2] [3]

Viruses can have various shapes and dimensions and encompass round shaped viruses having a diameter from 20 nanometers to 300 nanometers, as well as filamentous viruses, having a total length of up to 1400 nm; and a diameters of about 80 nm [4]. Viruses can consist of a membranous and/or protein-derived compartment monolayered or multilayered barrier. Accordingly, viruses in the sense of the disclosure comprise non-enveloped viruses which consist of a compartment defined by a monolayer proteinaceous compartment barrier of the capsid.

Exemplary non-enveloped viruses comprise DNA viruses such as Adenoviruses, Parvoviruses Polyomaviruses and Anelloviruse and RNA viruses such as Caliciviruses, Picornaviruses, Reoviruses, Astroviruses, Hepeviridae and additional viruses identifiable by a skilled person. [5] Viruses in the sense of the disclosure also comprise enveloped viruses which further include the membrane bilayer of the envelope possibly presenting one or more proteins. Exemplary enveloped viruses comprise DNA viruses such as Herpesviruses, Poxviruses, Hepadnaviruses, Asfarviridae and RNA viruses such as Flaviviruses Alphaviruses, Togaviruses Coronaviruses, Hepatitis D, Orthomyxoviruses, Paramyxoviruses, Rhabdovirus—Bunyaviruses, Filoviruses as well as Retroviruses and additional viruses identifiable by a skilled person. [5]

Viruses in the sense of the disclosure can also be categorized in view of the related viral NA according to the Baltimore classification as double-stranded viruses (dsDNA viruses), single-stranded DNA viruses (ssDNA), double-stranded RNA viruses (dsRNA viruses), positive-strand RNA viruses (+ssRNA viruses), negative-strand RNA viruses (−ssRNA viruses), single-stranded RNA-reverse transcriptase viruses (ssRNA-RT viruses), and double-stranded DNA-reverse-transcriptase viruses (dsDNA-RT viruses).

The term “prokaryote” is used herein interchangeably with the terms “prokaryotic cell” and refers to a microbial species which contains no nucleus or other membrane-bound organelles in the cell. Exemplary prokaryotic cells include bacteria and archaea.

The term “bacteria” or “bacterial cell”, as used herein indicates a large domain of prokaryotic microorganisms. Typically a few micrometers in length (from 0.5 to 6 um), bacterial cell can have a diameter from 1 to 10 um or be as large as 750 um as will be understood by a skilled person. Bacteria have a number of shapes, ranging from spheres to rods and spirals, and are present in most habitats on Earth, such as terrestrial habitats like deserts, tundra, Arctic and Antarctic deserts, forests, savannah, chaparral, shrublands, grasslands, mountains, plains, caves, islands, and the soil, detritus, and sediments present in said terrestrial habitats; freshwater habitats such as streams, springs, rivers, lakes, ponds, ephemeral pools, marshes, salt marshes, bogs, peat bogs, underground rivers and lakes, geothermal hot springs, sub-glacial lakes, and wetlands; marine habitats such as ocean water, marine detritus and sediments, flotsam and insoluble particles, geothermal vents and reefs; man-made habitats such as sites of human habitation, human dwellings, man-made buildings and parts of human-made structures, plumbing systems, sewage systems, water towers, cooling towers, cooling systems, air-conditioning systems, water systems, farms, agricultural fields, ranchlands, livestock feedlots, hospitals, outpatient clinics, health-care facilities, operating rooms, hospital equipment, long-term care facilities, nursing homes, hospice care, clinical laboratories, research laboratories, waste, landfills, radioactive waste; and the deep portions of Earth's crust, as well as in symbiotic and parasitic relationships with plants, animals, fungi, algae, humans, livestock, and other macroscopic life forms. Bacteria in the sense of the disclosure refers to several prokaryotic microbial species which comprise Gram-negative bacteria, Gram-positive bacteria, Proteobacteria, Cyanobacteria, Spirochetes and related species, Planctomyces, Bacteroides, Flavobacteria, Chlamydia, Green sulfur bacteria, Green non-sulfur bacteria including anaerobic phototrophs, Radioresistant micrococci and related species, Thermotoga and Thermosipho thermophiles as would be understood by a skilled person. Taxonomic names of bacteria that have been accepted as valid by the International Committee of Systematic Bacteriology are published in the “Approved Lists of Bacterial Names” [6] as well as in issues of the International Journal of Systematic and Evolutionary Microbiology. More specifically, the wording “Gram positive bacteria” refers to cocci, nonsporulating rods and sporulating rods that stain positive on Gram stain, such as, for example, Actinomyces, Bacillus, Clostridium, Corynebacterium, Cutibacterium (previously Propionibacterium), Erysipelothrix, Lactobacillus, Listeria, Mycobacterium, Nocardia, Staphylococcus, Streptococcus, Enterococcus, Peptostreptococcus, and Streptomyces. Bacteria in the sense of the disclosure refers also to the species within the genera Clostridium, Sarcina, Lachnospira, Peptostreptococcus, Peptoniphilus, Helcococcus, Eubacterium, Peptococcus, Acidaminococcus, Veillonella, Mycoplasma, Ureaplasma, Erysipelothrix, Holdemania, Bacillus, Amphibacillus, Exiguobacterium, Gracilibacillus, Halobacillus, Saccharococcus, Salibacillus, Virgibacillus, Planococcus, Kurthia, Caryophanon, Listeria, Brochothrix, Staphylococcus, Gemella, Macrococcus, Salinococcus, Sporolactobacillus, Marinococcus, Paenibacillus, Aneurinibacillus, Brevibacillus, Alicyclobacillus, Lactobacillus, Pediococus, Aerococcus, Abiotrophia, Dolosicoccus, Eremococcus, Facklamia, Globicatella, Ignavigranum, Carnobacterium, Alloiococcus, Dolosigranulum, Enterococcus, Melissococcus, Tetragenococcus, Vagococcus, Leuconostoc, Oenococcus, Weissella, Streptococcus, Lactococcus, Actinomyces, Arachnia, Actinobaculum, Arcanobacterium, Mobiluncus, Micrococcus, Arthrobacter, Kocuria, Nesterenkonia, Rothia, Stomatococcus, Brevibacterium, Cellulomonas, Oerskovia, Dermabacter, Brachybacterium, Dermatophilus, Dermacoccus, Kytococcus, Sanguibacter, Jonesia, Microbacteirum, Agrococcus, Agromyces, Aureobacterium, Cryobacterium, Corynebacterium, Dietzia, Gordonia, Skermania, Mycobacterium, Nocardia, Rhodococcus, Tsukamurella, Micromonospora, Propioniferax, Nocardioides, Streptomyces, Nocardiopsis, Thermomonospora, Actinomadura, Bifidobacterium, Gardnerella, Turicella, Chlamydia, Chlamydophila, Borrelia, Treponema, Serpulina, Leptospira, Bacteroides, Porphyromonas, Prevotella, Flavobacterium, Elizabethkingia, Bergeyella, Capnocytophaga, Chryseobacterium, Weeksella, Myroides, Tannerella, Sphingobacterium, Flexibacter, Fusobacterium, Streptobacillus, Wolbachia, Bradyrhizobium, Tropheryma, Megasphera, Anaeroglobus, Escherichia-Shigella, Klebsiella, muribaculum, alloprevotella, paraprevotella, oscillibacter, candidatus arthromitus, aeromonas, romboutsia, campylobacter, salmonella, faecalibacterium, roseburia, blautia, oribacterium, ruminococcus.

The term “Archaea” or “Archaea cell” as used herein refers to prokaryotic microbial species of the division Mendosicutes, such as Crenarchaeota and Euryarchaeota, which comprises methanogens (prokaryotes that produce methane); extreme halophiles (prokaryotes that live at very high concentrations of salt (NaCl); extreme (hyper) thermophiles (prokaryotes that live in extremely hot environments), Methanobrevibacter, and Methanosphaera. Archaea are single-celled organisms that lack a nucleus (prokaryotes), may have morphology including but not limited to coccus, bacillus, square, and triangular. Archaea lack a peptidoglycan cell wall and Md range from 0.1 um to 100 um. Archaea in the disclosure refer to archaea within the genera: Halostagnicola (pleiomorphic, 1.0-3.0 μm length, non-motile), Caldisphaera (coccus, 0.8-1.1 μm diameter, non-motile), Cenarchaeum (rod-shaped, 0.5-0.9 μm diameter), Caldococcus (coccus, 0.7-2.1 μm size), Ignisphaera (coccus, 1-1.5 μm diameter), Acidilobus (coccus, 1-2 μm diameter, non-motile), Acidococcus, Aeropyrum (coccus, 0.8-1.2 am diameter), Desulfurococcus (coccus, 0.5-15 am diameter), Ignicoccus (coccus, 1-3 μm diameter, motile), Staphylothermus (coccus, 0.8-1.3 μm diameter), Stetteria (coccus, 0.5-1.5 μm diameter), Sulfophobococcus (coccus), Thermodiscus (coccus, 0.2-3 μm diameter), Thermosphaera (coccus, 0.5-1.5 μm diameter), Geogemma (coccus, ˜1 μm diameter), Hyperthermus (coccus, ˜1.5 μm diameter), Pyrodictium (coccus, 0.3-2.5 am diameter), Pyrolobus (coccus, 0.7-2.5 am diameter), Nitrosopumilus (candidatus) (rod-shaped, 0.15-0.27 μm diameter and 0.49-2.00 μm length, some motile), Acidianus (spindle-shaped, 900×24 nm), Metallosphaera (coccus, ˜1 μm diameter), Stygiolobus (cocci, 0.5-2 μm diameter, carries Stygiolobus rod-shaped virus), Sulfolobus (cocci, 0.5-2 μm diameter, carries virus), Sulfurisphaera (cocci, 1.2-1.5 μm diameter), Thermofilum (rod-shaped, 0.17-0.35 μm diameter and 4-100 μm length), Caldivirga (rod-shaped, 0.4-0.7 μm diameter and 4-100 μm length), Pyrobaculum(rod-shaped, 0.4-0.5 μm diameter and 4-100 μm length), Thermocladium (rod-shaped, 4-100 μm length), Thermoproteus (rod-shaped, 0.4-0.5 μm diameter and 4-100 μm length), Vulcanisaeta (rod-shaped, 0.4-0.6 μm diameter and 4-100 μm length), Aciduliprofundum (pleiomorphic coccus, 0.6-1.0 μm diameter), Archaeoglobus (triangular, 0.4-1.2 μm wide), Ferroglobus (coccoid), Geoglobus (coccoid), Haladaptatus (coccus, 1.0-1.2 μm diameter, motile), Halalkalicoccus (pleiomorphic, ˜5 μm), Haloalcalophilium (pleiomorphic, ˜5 μm), Haloarcula (pleiomorphic, 1.0-2 μm diameter 2.0-3.0 μm length), Halobacterium (rod-shaped, 2-5 μm length), Halobaculum (rod-shaped, 0.4 μm diameter and 0.6 μm length), Halobiforma (pleomorphic, 0.5-2 μm diameter), Halococcus (cocci, 0.6-1.5 μm diameter), Haloferax (pleiomorphic, 1.1-2.0 μm), Halogeometricum (pleomorphic), Halomicrobium (rod-shaped, 1.80-2.25 μm diameter and 2.25-2.80 μm length, non-motile), Halopiger (rod-shaped, ˜3.75 μm diameter and ˜0.75 μm length), Haloplanus (rod-shaped, ˜1.5 μm length), Haloquadra (square, 40×40 μm), Halorhabdus (pleiomorphic, 3-5 μm), Halorubrum (pleiomorphic, 44×55 nm), Halosarcina (pleiomorphic, 0.8-2 μm diameter), Halosimplex, Haloterrigena (coccoid, 1.5 μm-2.0 μm diameter), Halovivax (rod-shaped, 0.4-0.5 μm diameter and 4-5 um length), Natrialba, Natrinema(pleomorphic, 0.5-2.0×1.5-11.0 μm), Natronobacterium (rod-shaped), Natronococcus (coccoid, 1-2 μm diameter), Natronolimnobius(rod-shaped), Natronomonas(pleomorphic), Natronorubrum(pleomorphic, 0.8-3.6 μm), Methanoregula (candidatus)(rod-shaped, 0.2-0.8 μm in diameter or coccoid, 0.2-0.3 μm diameter and 0.8-3.0 μm length), Methanocalculus (coccoid, ˜1 μm diameter), Methanobacterium)(rod-shaped, 2.5-5 μm in diameter), Methanobrevibacter(rod-shaped, 0.34 to 1.6 μm), Methanosphaera (coccoid), Methanothermobacter(rod-shaped, 7 μm length), Methanothermus (rod-shaped, 2-5 μm length), Methanocaldococcus (coccoid, 0.1-100 μm length), Methanotorris (coccoid, 0.1-100 μm length), Methanococcus (coccoid, 0.9-1.3 μm diameter), Methanothermococcus (coccoid), Methanocorpusculum (cocci, <2 μm diameter), Methanoculleus (cocci, 0.5 to 2.0 μm diameter), Methanofollis (cocci, 0.8-1.8 μm diameter), Methanogenium (cocci, 1.2-2.5 μm diameter), Methanolacinia (rod-shaped, 0.6 μm diameter and 1.5-2.5 μm length), Methanomicrobium(rod-shaped, 0.6-0.7 diameter 1.5-2.5 length), Methanoplanus (cocci, 1-3.5 μm diameter), Methanospirillum(rod-shaped, 2-5 μm length), Methanosaeta(rod-shaped, 2.5-6 μm length), Methanimicrococcus (cocci, 0.8 μm diameter), Methnococcoides (cocci, 0-1.8 μm diameter), Methanohalobium (cocci, 1.0-1.2 μm), Methanohalophilus(rod-shaped), Methanolobus (cocci, 1.0-1.25 μm diameter), Methanomethylovorans (cocci), Methanosalsum(rod-shaped), Methanosarcina(rod-shaped, 2.3±0.2 μm), Methanopyrus(rod-shaped, 2-14 μm length and 0.5 μm diameter), Palaeococcus, Pyrococcus (cocci, 0.8-2 μm diameter), Thermococcus (cocci, 0.6-2 μm diameter), Ferroplasma(pleomorphic or cocci, 0.66±0.18×0.57±0.20 μm), Picrophilus(pleomorphic), Thermoplasma (cocci, ˜1 μm diameter), and Nanoarchaeum (cocci, 0.4 μm diameter).

The term “fungi” or “fungal cells” as described herein, indicates eukaryotes such as yeasts and molds that exist in single unicellular forms (yeast) or multicellular forms (molds such as hyphae and mycelium) which are characterized by a cell wall that contains of glucans, glycoproteins, and chitin. By weight, fungal cell walls typically contain up to 60% glycans, up to 30% glycoproteins, and up to 20% chitin. [5] Fungi can typically range from about 0.5 to 50 um and in particular 0.5 to 20 um 5-50 um in size. Fungi in the disclosure refer to fungi within the genera: Aaosphaeria, Acaromyces, Agaricus, Alternaria, Amorphotheca, Annulohypoxylon, Antrodia, Apiotrichum, Aplosporella, Arthroderma, Ascochyta, Ascoidea, Aspergillus, Aureobasidium, Babjeviella, Bacidia, Batrachochytrium, Baudoinia, Beauveria, Bipolaris, Blastomyces, Boeremia, Botrytis, Brettanomyces, Brettanomyces, Candida, Cantharellus, Capronia, Ceraceosorus, Cercospora, Chaetomium, Chaetomium, Cladophialophora, Clavispora, Coccidioides, Colletotrichum, Coniophora, Coniosporium, Coprinopsis, Cordyceps, Cryptococcus, Cucurbitaria, Cutaneotrichosporon, Cyberlindnera, Cyphellophora, Dacryopinax, Daldinia, Debaryomyces, Diaporthe, Dichomitus, Didymella, Diplodia, Dissoconium, Diutina, Dothidotthia, Drechmeria, Drepanopeziza, Emericellopsis, Endocarpon, Epithele, Eremomyces, Eremothecium, Exophiala, Fibroporia, Filobasidium, Fomitiporia, Fomitopsis, Fonsecaea, Fulvia, Fusarium, Gaeumannomyces, Geosmithia, Glarea, Gloeophyllum, Grosmannia, Guyanagaster, Heterobasidion, Hirsutella, Histoplasma, Hyaloscypha, Hyphopichia, Ilyonectria, Jaminaea, Kalmanozyma, Kazachstania, Kluyveromyces, Kockovaella, Komagataella, Kuraishia, Kwoniella, Laccaria, Lachancea, Lachnellula, Laetiporus, Lasiodiplodia, Lentinula, Leptosphaeria, Letharia, Linderina, Lindgomyces, Lobosporangium, Lodderomyces, Macroventuria, Malassezia, Marasmius, Meira, Melampsora, Metarhizium, Metschnikowia, Meyerozyma, Microdochium, Microsporum, Mitosporidium, Mixia, Moesziomyces, Mollisia, Morchella, Mycena, Mytilinidion, Nannizzia, Naumovozyma, Nematocida, Neohortaea, Neurospora, Ogataea, Orbilia, Paecilomyces, Paracoccidioides, Paraphaeosphaeria, Parastagonospora, Penicilliopsis, Penicillium, Pestalotiopsis, Phaeoacremonium, Phanerochaete, Phialophora, Phycomyces, Pichia, Pleurotus, Pneumocystis, Pochonia, Podospora, Postia, Protomyces, Pseudocercospora, Pseudogymnoascus, Pseudomassariella, Pseudomicrostroma, Pseudovirgaria, Pseudozyma, Pseudozyma, Psilocybe, Puccinia, Punctularia, Purpureocillium, Pyrenophora, Pyricularia, Ramularia, Rasamsonia, Rhinocladiella, Rhizoctonia, Rhizophagus, Rhizopus, Rhodotorula, Saccharomyces, Saitoella, Saprochaete, Scedosporium, Scheffersomyces, Schizophyllum, Schizosaccharomyces, Sclerotinia, Serpula, Sodiomyces, Sordaria, Sparassis, Spathaspora, Sphaerulina, Spizellomyces, Sporisorium, Sporothrix, Stereum, Sugiyamaella, Suhomyces, Suillus, Synchytrium, Talaromyces, Tetrapisispora, Thermothelomyces, Thyridium, Tilletiaria, Tilletiopsis, Torulaspora, Trametes, Trematosphaeria, Tremella, Trichoderma, Trichophyton, Truncatella, Tuber, Uncinocarpus, Ustilaginoidea, Ustilago, Vanderwaltozyma, Venustampulla, Verruconis, Verticillium, Wallemia, Westerdykella, Wickerhamiella, Wickerhamomyces, Xylaria, Xylona, Yamadazyma, Yarrowia, Zasmidium, Zygosaccharomyces, Zygotorulaspora, Zymoseptoria.

Microbes in the sense of the present disclosure are thus structured as compartments defined by an outer biological barrier which can be monolayered (see non-enveloped viruses such as norovirus, enterovirus, adenovirus, and rhinovirus.) or multilayered (see e.g. enveloped viruses such as and HIV-1 and SARS-CoV-2 and archaea such as Mycoplasma pneumoniae) Gram-negative bacteria such as Escherichia coli, Pseudomonus aeruginosa and Klebsiella pneumoniae, or Gram-positive bacteria such as Staphyloccocus aureus, Streptoccocus pneumoniae, and Mycobacterium tuberculosis or fungi consisting of a chitin derived cell wall and membranous barriers).

Accordingly, a “microbial compartment” in the sense of the disclosure indicates the structure defined by a biological barrier that separates and protects the interior of the microbe from the outside environment. The biological barrier of a microbial compartment can be formed by at least one sheet of protein, lipid (e.g. phospholipid and/or glucolipid), and/or polysaccharides molecules, and possibly further comprising additional compounds and molecules such as lipopolysaccharides, lipoteichoic acid, flotillins, carotenoids, and hopanoids as will be understood by a skilled person.

Accordingly, a microbial compartment can have a Md ranging from 200 nm of certain viruses to 20 um, 50 um or even 750 um diameter of certain fungi and bacteria.

Microbial compartments are also characterized by a stiffness which primarily depends on the composition of the barrier of the biological compartment < and can be measured by measuring the Young's modulus.

The wording “elastic modulus” “Young's modulus” or “E” as used herein refers to a mechanical property that measures the tensile or compressive stiffness of a solid material when the force is applied lengthwise. It quantifies the relationship between tensile/compressive stress a (force per unit area) and axial strain F (proportional deformation) in the linear elastic region of a material and is determined using the formula: E=σ/ε Young's moduli can be expressed in pascals [7].

Accordingly a microbial compartment typically has a microbial Young's modulus Em ranging from 0.01 MPa to 100 GPa.

Microbial compartments in the sense of the disclosure encapsulate microbial nucleic acid (NA).

The term “nucleic acid” or “polynucleotide” as used herein indicates an organic polymer composed of two or more monomers including canonical or noncanonical nucleotides, and nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. Accordingly, the term “polynucleotide” includes nucleic acids of any length, and in particular DNA, RNA, and fragments thereof. A polynucleotide of three or more nucleotides is also called “nucleotidic oligomer” or “oligonucleotide.”

The term “DNA” or Deoxyribonucleic acid” as used herein indicates a polynucleotide composed of deoxiribonucleotide bases or an analog thereof to form an organic polymer. The term “deoxyribonucleotide” refers to any compounds that consist of a deoxyribose (deoxyribonucleotide) sugar joined to a purine or pyrimidine base and to a phosphate group, and that are the basic structural units of a deoxyribonucleic acid, typically adenine (A), cytosine (C), guanine (G), and thymine (T). In an DNA adjacent ribose nucleotide bases are chemically attached to one another in a chain typically via phosphodiester bonds. The term “deoxyribonucleotide analog” refers to a deoxyribonucleotide in which one or more individual atoms have been replaced with a different atom with a different functional group. For example, deoxyribonucleotide analogues include chemically modified deoxyribonucleotides, such as methylation hydroxymethylation glycosylation and additional modifications identifiable by a skilled person.

The term “RNA” or “Ribonucleic acid” as used herein indicates a polynucleotide composed of ribonucleotide bases: or an analog thereof linked to form an organic polymer. The term “ribonucleotide” refers to any compounds that consist of a ribose (ribonucleotide) sugar joined to a purine or pyrimidine base and to a phosphate group, and that are the basic structural units of a ribonucleic acid, typically adenine (A), cytosine (C), guanine (G), and uracil (U). In an RNA adjacent ribose nucleotide bases are chemically attached to one another in a chain typically via phosphodiester bonds. The term “ribonucleotide analog” refers to a ribonucleotide in which one or more individual atoms have been replaced with a different atom with a different functional group. For example, ribonucleotide analogues include chemically modified ribonucleotides, such as methylation hydroxymethylation glycosylation and additional modifications identifiable by a skilled person. Examples of chemical modifications of RNA comprise dynamic modifications to RNA identified in the transcriptome, including N⁶-methyladenosine (m⁶A), inosine (I), 5-methylcytosine (m⁵C), pseudouridine (Ψ), 5-hydroxymethylcytosine (hm⁵C), and N¹-methyladenosine (m¹A), and related epitranscriptome which are described in Song and Yi 2017.

Additional chemical modifications of transfer RNA (tRNA) are described in Jackman and Alfonzo 2013 [9]Accordingly, the term RNA includes ribonucleic acids of any length including analogs or fragments thereof.

Microbial nucleic acids in the sense of the disclosure comprises DNA and RNA enclosed within a microbial compartment. Microbial nucleic acids can be double stranded and/or single stranded, can possibly include non-canonical base pairs and/or nucleic acid modifications (such as DNA methylation) and or be complexed with proteins.

In the microbial enrichment methodology and related methods and systems herein described enrichment of a biological sample with microbial compartment and related nucleic acid is obtained by selectively targeting the host cells of the animal sample.

The term “sample” as used herein indicates a limited quantity of something that is indicative of a larger quantity of that something, including but not limited to fluids from a specimen such as biological environment, cultures, tissues, commercial recombinant proteins, synthetic compounds or portions thereof.

In particular, a “biological sample” in the sense of the disclosure indicates a sample comprising cells and/or other biological compartments wherein the term “cell” in the sense of the disclosure indicates the basic structural and functional units of life, as will be understood by a skilled person. Accordingly a biological sample can comprise one or more cells of any biological lineage such as cells of an individual, microbial cells and in particular prokaryotic cells as well as viral compartments, as being representative of the total population of similar cells or viral compartments in the biological environment which can be a sampled individual or a portion thereof such as a tissue or an organ. Biological sample may comprise a host sample combined with a reagent, a buffer, a dilutant.

The term “individual” or “host” as used herein indicates any multicellular organism that can comprise microorganisms, thus providing a biological environment for microbes and in in particular an environment for microbial communities, in any of their tissues, organs, and/or biofluids. Exemplary individual in the sense of the disclosure includes plants, algae, animals, fungi, and in particular, vertebrates, mammals more particularly humans. Exemplary biological samples from an individual comprise the following: whole venous and arterial blood, capillary blood, blood plasma, blood serum, dried blood spots, cerebrospinal fluid, interstitial fluid, sweat, lumbar punctures, nasal secretions, sinus washings, tears, corneal scrapings, saliva, sputum or expectorate, bronchoscopy secretions, transtracheal aspirate, endotracheal aspirations, bronchoalveolar lavage, vomit, endoscopic biopsies, colonoscopic biopsies, subcutaneous and mesenteric adipose tissue biopsies, bile, vaginal fluids and secretions, endometrial fluids and secretions, urethral fluids and secretions, mucosal secretions, synovial fluid, ascitic fluid, peritoneal washes, tympanic membrane aspirate, urine, clean-catch midstream urine, catheterized urine, suprapubic aspirate, kidney stones, prostatic secretions, feces, mucus, pus, wound draining, skin scrapings, skin snips and skin biopsies, hair, nail clippings, cheek tissue, bone marrow biopsy, solid organ biopsies, surgical specimens, solid organ tissue, cadavers, breast milk, or tumor cells, among others identifiable by a skilled person. Biological samples can be obtained using sterile techniques or non-sterile techniques, as appropriate for the sample type, as identifiable by persons skilled in the art. Depending on the type of biological sample and the intended analysis, biological samples can be used freshly for sample preparation and analysis, or can be fixed using fixative.

Accordingly, in general all individually comprise host cells which are eukaryotic cells with membrane-bound organelles suspended in the cytoplasm enveloped by a plasma membrane.

In some embodiments, an individual in the sense of the disclosure can be a plant which indicates a eukaryote multicellular organism predominantly photosynthetic, of the biological kingdom Plantae. [10] Exemplary plants comprise green algae, bryophytes, pleidophytes and spermatophytes. Plant cells have dimension from 10 uM to 100 uM as will be understood by a skilled person. The s distinctive features of plant include primary cell walls containing cellulose, hemicelluloses and pectin, in particular the plant cell wall is a natural nanoscale network structure that primarily consists of polysaccharide polymers such as cellulose, hemicellulose, and pectin, but often includes glycoproteins and lignin as well. the plant cell characteristic extracellular matrix is primarily composed of cellulose as will be understood by a skilled person. [10]

In some embodiments the individual is an animal which indicates a eukaryotic multicellular organism of the biological kingdom of Animalia Exemplary animal comprise bird, mammals, fish, reptiles, amphibians, porifera, platyhelminthes, cnidaria, annelida, echinodermata, mollusca, crustacea, arachnida, insect, and myriapoda. Individual of the kingdom animalia. are composed of cells having dimension ranging from 8 um to 23 um and being surrounded by a characteristic extracellular matrix composed of collagen and elastic glycoproteins. [11] Contrary to animal plants Animal cells do not have a cell wall or chloroplasts as will be understood by a skilled person.

Mammalian cells usually range in diameter from 10 to 100 μm. Some examples of mammalian cells include Human cells (7 μm), HeLa cells (20-40 μm), cells (12-13 μm), mouse cells (10-20 μm), and monkey cells (17-25 μm). Fish cell diameter usually ranges from 7 to 25 μm. Some examples include Spermatogonia (12-16 μm), embryonic cells (16-17 μm), and Trout red blood cells (11.5-16 μm). Insect cells usually have a diameter from 11 to 18 μm). For example, fly spermatids have a diameter of 12 μm. Bird cells have a diameter range of 10 to 15 μm. Reptiles have a cell diameter range of 20 to 7 μm. Amphibians have a cell diameter ranging from 5.1 to 24.17 μm as will be understood by a skilled person.

Accordingly, also the host cells are structured as compartments defined by an outer biological barrier which is formed by a biological membrane. The term, “host cell”, and “host compartments” are thus used interchangeably in the present disclosure and for the purpose of the present disclosure are characterized by their dimension and their stiffness that can be expressed in terms of elastic modulus.

Accordingly, a host compartment typically has a host diameter Hd equal to or higher than 8 um in at least one dimension and an elastic modulus 10 kPa to 100 GPa.

Animal cells do not have a cell wall, while fungal, bacteria, archaea, and plant cells do. A cell wall provides the cell with more rigidity and makes a cell harder to lyse. However, animal cells have nuclei and therefore multilayered membrane structure encapsulating the host NA.

Accordingly, animal compartments in the sense of the disclosure typically have a Young's Modulus E of 10 kPa to 1 GPa.

Plant compartments in the sense of the disclosure typically have a Young's Modulus E of 10 kPa to 1 GPa.

Host compartments in the sense of the disclosure encapsulate host nucleic acid (NA) where host NA may be DNA and/or RNA. Host NA can possibly be protein bound to histone or other NA-binding proteins. Host NA can be possibly single and/or double-stranded. Host NA can also be encapsulated within a single or multilayered barrier as will be understood by a skilled person.

In a host sample the host NA is encapsulated within the host compartment comprised in the sample together with a sample matrix. A “sample matrix” or “host matrix” in the sense of the disclosure indicates all the components of a sample excluding the NA analytes of interest. These include but are not limited to cellulose, fibrous or non-fibrous proteins a of the extracellular matrix (ECM) such as collagens, elastins, fibronectins, and laminins, as well as peptidoglycans of the ECM such as mucins, perlecan, decorin, biglycan, lumican, hyaluronic acid, and tenascin. The matrix may also include cellular proteins, including but not limited to structural, housekeeping, and cell type-specific proteins, extracellular proteins, including but not limited to immunolobulins, globins, albumin, and transferrin, and enzymes including but not limited to nucleases, proteases, amylase, and lipases. Proteins in the matrix may have post-translational modifications including but not limited to glycosylation, phosphorylation, acetylation, hydroxylation, methylation, ubiquitination, deamidation, and lipidation. The matrix may also be composed of nucleic acids excluding the nucleic acid analytes of interest, including DNA and RNA that may or may not include chemical modifications including but not limited to methylation and acetylation. Additional matrix components include but are not limited to lipids, phospholipids, glycans, peptides, and ions (including those that may be inhibitory to nucleic acid analysis such as Mg2+ and Ca2+).

The host matrix thus binds the host cells and imparts the sample with a certain viscosity which can be measure by a skilled person can impact the sample process as will be understood by a skilled person upon reading of the present disclosure.

Some host samples can be obtained by contacting a swab with a surface on the host body and removing some material from said surface, for examples in human being include throat swab, nasal swab, nasopharyngeal swab, oropharyngeal swab, cheek or buccal swab, tongue swab, urethral swab, vaginal swab, cervical swab, genital swab, anal swab, rectal swab, conjunctival swab, skin swab, any tract swab, and any wound swab. Some animal samples can be obtained by utilization of the entire and/or part of the organism (e.g. insects processed whole/coral processed in chunks). Some animal samples can be obtained by taking a section of tissue, such as during an endoscopy or colonoscopy or during a surgical procedure including GI tract scrapings and/or biopsies.

Accordingly, exemplary animal samples according to the instant disclosure and in particular human samples comprise tear fluid, saliva, nasal, oral, tonsillar, and pharyngeal swabs, sputum, bronchoalveolar lavage (BAL), gastric, small-intestine, and large-intestine contents and aspirates, feces, bile, pancreatic juice, urine, vaginal samples, semen, skin swabs, tissue and tumor biopsy, blood, lymph, cerebrospinal fluid, amniotic fluid, mammary gland secretions/breast milk. Examples of environmental and industrial samples: soil and other media for (agricultural) plant growth, water, sediment, oil well samples, bioreactors (e.g., complex/mixed probiotics). Samples can also include clean room swabs, hospital surfaces, and mucosal brush biopsies.

The microbial host depletion and enrichment methodology and related methods and systems herein described is based on selective targeting of the host cells to obtain depletion of host cells and related nucleic acid while minimizing depletion of any microbial compartment and related nucleic acid possibly present in the sample.

The term “selective” as used herein in connection with an action indicates that the action that is highly specific in activity and/or effect. Accordingly, selectively targeting in the sense of the disclosure when referred to compartments of a sample indicates a targeting that is specific for the referenced item (here the host cell) among the other items present in the sample. The wording “specific” “specifically” or “specificity” when referred to an action indicates that the action is directed to a referenced item with minimized activity and/or effect towards other items present in the sample.

The term “depletion” as used herein indicates reduction a reduction in the number of a referenced items in a substance. Therefore, the wording host depletion in a sample indicates the reduction of host cells and/or host nucleic acid in the sample. Reduction can also refer to reduction of non-target cells and/or non-target nucleic acid in subset of the sample, such as splitting the sample into host and microbial fractions which would reduce host cells within the microbial fraction/subset of the sample as will be understood by a skilled person.

Conversely the term “enrichment” as used herein indicates an increase in the number of referenced items in a substance therefore the wording microbial enrichment in a sample indicates an increase of microbial compartments and/or microbial nucleic acid in the sample compared with other compartments and nucleic acid According enrichment can be detected my detecting increase in the number of referenced items (microbial compartment and/or microbial NA) relative to the number of a corresponding item in the sample (host compartment and/or host NA). In particular in the enriched sample with a microbial compartment and/or microbial NA the proportion (and in particular the ratio) of microbial compartments/NA is increased even if the absolute count does not change as will be understood by a skilled person upon reading of the present disclosure.

In some embodiments of the microbial enrichment methodology of the disclosure, enrichment of an animal sample with a microbial compartment is performed by selectively targeting the animal cells over microbial compartments leveraging the differences in the related dimensions. In particular, in those embodiments, beads are selected so that the related use in disrupting of the animal sample targets host compartments selectively over microbial compartments of the sample.

The term “disrupt” and “disrupting” as used herein indicates the treatment of a referenced item to release one or more of the related components from the item. Accordingly, disruption of a sample indicates any processing of the sample directed to release cells, biological molecules and/or additional components of the sample as will be understood by a skilled person.

Sample disruption in the sense of the disclosure can be performed chemical and mechanical/physical methods identifiable by a skilled person. In particular chemical methods rely on the use of detergents chaotropes and additional chemical compounds identifiable by a skilled person. Mechanical and physical methods instead rely on grinding, shearing, beating and shocking as well as additional mechanical actions directed to separate the sample components which are identifiable by a skilled person. Related devices comprise mechanical homogenizers, manual homogenizers, mortar and pestles, sonicators, mixer mills, and vortexers are several of the more common tools used for mechanical and physical disruption. [12]

Disruption of a sample performed with the use of beads is also generally indicated as the mechanical disruption of a sample resulting from application of mechanical forces resulting from the contact of the beads with the sample. Mechanical disruption with beads can be performed by grinding, beating, and/or sharing as will be understood by a skilled person.

The term “grinding” as used herein indicates an action resulting in the creation of friction by sandwiching the sample between two hard surfaces that slide against each other, which can be performed by mortars and pestles including cryo-grinders. [12]

The term “shearing” as used herein indicate disruption performed by creating a tangential force being applied to the sample resulting in the disruption of the sample. Tangential forces can be applied with homogenizers such as blenders, rotor-stators, and some of the glass homogenizers, ad additional devices identifiable by a skilled person. [12]

The term “beating” as used herein involves crushing a sample with a projectile. Most beating methods rely on placing a sample and projectile in a tube and rapidly shaking them back and forth. The most common method is bead beating which uses grinding balls or small beads, though cylinders and irregular shapes have also been used. Bead beating has been in practice for years for the disruption of microorganisms, as will be understood by a skilled person. [12]

The term “beads” as used herein indicates a small-shaped body made of a material configured to disrupt a sample which differ by size, shape, material, and composition as will be understood by a skilled person. Bead sizes range from microns to centimeters, and the range can vary depending on the bead material. Beads can be used in one size or multiple sizes as will be understood by a skilled person. With respect to shapes beads are classified as spherical—utilizing impaction and hammering as the leading force—and angular such as such as satellites, generating mechanical shear forces to chop and cut samples. With respect to the material, beads' material determines density, hardness, durability, sample compatibility, and chemical resistance, which influence lysis efficiency and the integrity of target analytes. The lysing bead's hardness must be greater than the sample, and the set of beads can comprise a combination of bead materials. Exemplary materials comprise Silica, Ceramic, Silicon Carbide Glass, Zirconium Silicate, Zirconium Oxide Stainless Steel—and garnet as well as other materials as will be understood by a skilled person. A combination of grinding media sizes, shapes, and materials within a single sample can help optimize cell disruption. As will also be understood by a skilled person [5] at the filing date of the present disclosure.

Mechanical disruption of a sample through the use of beads is typically performed in a container which has definite dimensions including a container base, a container height and a container volume as will be understood by a skilled person.

In particular, mechanical disruption with beads can be performed in tubes, vials, plates, or deep well plates that contain the sample and beads. Tubes and deep well plates can be made of metal or plastic, such as polycarbonate, to maximize durability and compatibility with beads and sample. Tubes and plates are closed to prevent the beads from being pushed outside the container during disruption. In particular, those contained are usually sealed the with a gasket such as an O-ring or seal to prevent leakage and contamination. The tube, or microwell plate, used in bead beating is not just a sample holder, but actively participates in the homogenization. [12]

Mechanical disruption of a sample with beads can be performed using devices that can shake, grind and shear the samples with beads alone or in combination also in view of the type of sample. Some examples include amalgamators, homogenizers, and mixer mills, as will be understood by a skilled person.

In some embodiments, bead beating is performed by placing a sample in a tube or a deep well plate with projectiles and rapidly shaking, such as in a linear motion or FIG. 8 tridimensional motion. The goal of bead beating is to collide the sample between the projectiles or between a projectile and the wall. Projectiles for this application include the use of grinding balls, small beads, cylinders, satellites, and other irregular shapes (garnet beads) for the mechanical disruption of a sample as will be understood by a skilled person.

For the of disruption samples can be categorized as liquid samples, and solid samples. The liquid samples are defined as “a substance or mixture of substances that flows readily, but, unlike a gas, does not expand indefinitely (i.e., a substance with constant volume but not constant shape)” Examples of liquid samples can include saliva, individual cells, swabs resuspended in liquid, sputum inclusive in a liquid sample comprising individual host cells, culture and/or after single cell isolation such as FACS). the Solid samples are defined as a substance with constant shape and volume that does not flow readily. Solid sample can be categorized as i) Soft samples defined as samples with a maximal Young's Modulus between 1 Pa to 20 kPa Examples of soft samples can include: brain, glandular tissue, adipose tissue (low strain rates), liver, uterine tissue, and lamina propria; ii) Hard samples are defined as samples with a maximal Young's Modulus between 0.1 GPa to 500 GP Examples of hard samples can include bone, coral, insect sclerotized cuticle, teeth, hair, nail and :Intermediate samples (or medium softness sample) defined as samples with a maximal Young's Modulus between 20 kPa and 100 Mpa. Examples of intermediate samples can include: muscle, heart, skin, mucosa, and adipose tissue (high strain rates).

In embodiments herein described, the mechanical disruption of samples using beads is performed by selecting the bead dimensions and material as well as bead parameters including number of beads, bead collision frequency and bead beating time to selectively disrupt the host compartment with respect to the microbial compartments based on dimensions and/or stiffness of the compartments as measure by the elastic modulus.

In particular, in the host depletion method of the disclosure the bead radius and beads parameters being obtained by
a) selecting the bead radius to determine, for each of the host compartment and the microbial compartment, a compartment crush volume as a function of compartment diameter and bead radius;
b) selecting the bead parameters including number of beads, bead collision frequency and bead beating time to determine, for each of the host compartment and the microbial compartment, a compartment effective crush volume as a function of said compartment crush volume, the compartment elastic modulus and said bead parameters; and
c) determining, for each of the host compartment and the microbial compartment, a percentage of total sample volume that is bead beaten for a given compartment as a ratio between the compartment effective crush volume and a total volume of the biological sample.

In the host depletion method of the disclosure the set of beads is then selected having the bead radius and the bead parameters to obtain a percentage of total sample volume of at least 100% for the given host compartment preferably more than 300%, preferably more than 1000 and a percentage of total sample volume of up to 70% for the given microbial compartment preferably less than 50%, more preferably 30%, even more preferably less than 10%, (see Example 3).

The dimension of the beads are then selected to have the Radius of beads R, the disrupting conditions are dictated by the bead parameters comprising the number of beads used, the disrupting time are a frequency of agitation in a disrupting device which correspond to the frequency of collision of the beads times 300 as will be understood by a skilled person (Example 3).

The dimension and material of the beads are further selected so that the Stokes number is maintained at a value of more than 3, preferably more than 10, (see Example 3).

In embodiments of the present disclosure, the set of beating selected with host method herein described are configured to selectively disrupt the host compartment with respect to a microbial compartment when then the microbial nucleic acid has a mass equal to or lower than the host nucleic acid, as shown by the data reported in the Examples section.

In particular, as shown by the data in the Examples section, in some embodiments the selective depletion of host compartments from the sample can be obtained based on a difference in dimension between host compartments and microbial compartments. In particular in some embodiment selective depletion of host compartments can be obtained when the host compartment has a host compartment diameter of 8 um or higher and the microbial compartment has a microbial compartment diameter not greater than 5 um. In particular, in some of those embodiments the microbial compartment has a microbial compartment diameter not greater than 3 um.

In some embodiments the selective depletion of host compartments from the sample can be obtained based on a difference in stiffness of the host compartment with respect to the microbial compartment. In particular in some embodiments selective depletion of host compartments can be obtained when the host compartment has an elastic modulus equal to or lower than 10\hat{ }5 Pa and the microbial compartment has an elastic modulus equal to or higher than 10\hat{ }7 Pa.

In some embodiments, the selective depletion of host compartments from the sample can be obtained based on a combination of dimensions and stiffness of the host compartment with respect to the microbial compartment. In particular in some embodiments selective depletion of host compartments can be obtained when the microbial compartment has a microbial compartment diameter higher than 5 um and an elastic modulus equal to or higher than 10\hat{ }8 Pa. in some of those embodiments, the host compartment has an elastic modulus not greater than 10\hat{ }5 Pa. in some of those embodiments, selective disruption of a host compartments with Hd of 8 um or greater and elastic modulus less than 10\hat{ }5 Pa), with respect to microbial compartment with M_{d} of up to 10 um and elastic modulus more than 10\hat{ }7 Pa).

In some embodiments of the method where enrichment is performed by selecting size of beads based on differential dimension of the microbial compartments and host compartments, the beads can have a size of 1-2 mm for the purpose of enrich the animal sample with microbial compartment having Md of Sum or less.

In those embodiments disrupting the sample with a set of beads and using beads parameters selected with the methods herein described has been surprisingly found to be effective in selectively targeting host cells while minimizing disruption of microbial compartments in the sample irrespective of the type of animal sample which is disrupted as also shown by the Examples section.

In some embodiments, the beads are made of material that does not fragment into structures larger than a threshold value selected to o minimize the chances that beads can generate a fragment that will cause lysis of microbial compartment in the sample. In some embodiments the beads material is selected to minimize chances that fragment of dimension of 800 um or lower, 50 um or lower down to 10 um or lower. In some preferred embodiments the material is selected so to minimize the likelihood fragment are generated during the contacting having a dimension of 10 nm or higher. Accordingly in some embodiments the beads of the set of beads have an elastic modulus of at least 30 GPa.

In some embodiments the beads have a substantially spherical shape and are thus spheroidal. In some embodiments beads of the set of beads have a bead radius selected from 1.0 to 1.8 mm. In particular, in some embodiments ceramic beads of size 1.4 mm.

In some embodiments, the disrupting can be performed by bead beating performed at any speed from 4 m/s to 8 m/s for anywhere from 30 seconds to 2 minutes. Optional time is 30 seconds to minimize potential microbial lysis and loss.

In some embodiments, wherein the target microbes are bacteria the microbial compartment has diameter is less than 5 um and is a membranous compartment with membranous compartment barriers, the disrupting can be performed for a time from 5 seconds to 10 minutes. In some of those embodiments, wherein the target microbes are viruses the microbial compartment has diameter less than 2 um. In some of those embodiments, wherein the target microbes are fungi the microbial compartment can have a diameter from 5 um to 50 um and the disrupting can be performed from 5 seconds to 1 minute.

In some embodiments where selective disruption can be performed with the following bead radius and sent of bead parameters

For bacteria, archaea, fungi, and virus with Md below 1 um: in a liquid sample comprising
2R: 1.0 mm to 1.8 mm
Number of beads: 5-60% by volume of the tube (before packing)
Frequency: 16 Hz to 150 Hz
Time: 15 s to 300 s

For bacteria, archaea, fungi, and virus with Md between 1-2 um: in a liquid sample
2R: 1.0 mm to 1.3 mm
Number of beads: 5-30% by volume of the tube (before packing)
Frequency: 16 Hz to 60 Hz
Time: 15 s to 60 s

For bacteria, archaea, fungi, and virus with Md between 1-2 um: in a liquid sample
2R: 1.3 mm to 1.8 mm
Number of beads: 5-60% by volume of the tube (before packing)
Frequency: 16 Hz to 100 Hz
Time: 15 s to 300 s

For bacteria, archaea, fungi, and virus with Md above 2 um: in a liquid sample
2R: 1.2 mm to 1.6 mm
Number of beads: 5-30% by volume of the tube (before packing)
Frequency: 16 Hz to 351 Hz
Time: 15 s to 60 s

For bacteria, archaea, fungi, and virus with Md above 2 um: in a liquid sample
2R: 1.4 mm to 1.8 mm
Number of beads: 5-50% by volume of the tube (before packing)
Frequency: 16 Hz to 40 Hz
Time: 15 s to 60 s

For bacteria, archaea, fungi, and virus with Md less than or equal to 10 um: in a liquid sample
2R: 1.2 mm to 1.6 mm
Number of beads: 5-30% by volume of the tube (before packing)
Frequency: 35 Hz to 60 Hz
Time: 15 s to 60 s

For bacteria, archaea, fungi, and virus with Md less than or equal to 10 um: in a liquid sample
2R: 1.4 mm to 1.8 mm
Number of beads: 5-50% by volume of the tube (before packing)
Frequency: 40 Hz to 70 Hz
Time: 15 s to 60 s

For bacteria, archaea, fungi, and virus with Md less than or equal to 5 um: in a liquid sample
2R: 1.4 mm to 1.8 mm
Number of beads: 5-50% by volume of the tube (before packing)
Frequency: 70 Hz to 100 Hz
Time: 15 s to 60 s

For bacteria, archaea, fungi, and virus with Md below 1 um: in saliva classified as liquid sample
2R: 1.0 mm to 1.8 mm
Number of beads: 10-60% by volume of the tube (before packing)
Frequency: 30 Hz to 150 Hz
Time: 30 s to 300 s

For bacteria, archaea, fungi, and virus with Md between 1-2 um: in saliva classified as liquid sample
2R: 1.0 mm to 1.3 mm
Number of beads: 10-30% by volume of the tube (before packing)
Frequency: 30 Hz to 60 Hz
Time: 30 s to 60 s

For bacteria, archaea, fungi, and virus with Md between 1-2 um: in saliva classified as liquid sample
2R: 1.3 mm to 1.8 mm
Number of beads: 10-60% by volume of the tube (before packing)
Frequency: 30 Hz to 100 Hz
Time: 30 s to 300 s

For bacteria, archaea, fungi, and virus with Md above 2 um: in saliva classified as liquid sample
2R: 1.2 mm to 1.6 mm
Number of beads: 10-30% by volume of the tube (before packing)
Frequency: 30 Hz to 60 Hz
Time: 30 s to 60 s

For bacteria, archaea, fungi, and virus with Md above 2 um: in saliva classified as liquid sample
2R: 1.2 mm to 1.6 mm
Number of beads: 10-30% by volume of the tube (before packing)
Frequency: 30 Hz to 40 Hz
Time: 30 s to 60 s

For bacteria, archaea, fungi, and virus with Md above 2 um: in saliva classified as liquid sample
2R: 1.4 mm to 1.8 mm
Number of beads: 10-50% by volume of the tube (before packing)
Frequency: 30 Hz to 50 Hz
Time: 30 s to 60 s

For bacteria, archaea, fungi, and virus with Md less than or equal to 10 um: in saliva classified as liquid sample
2R: 1.2 mm to 1.6 mm
Number of beads: 10-30% by volume of the tube (before packing)
Frequency: 40 Hz to 60 Hz
Time: 30 s to 60 s

For bacteria, archaea, fungi, and virus with Md less than or equal to 10 um: in saliva classified as liquid sample
2R: 1.4 mm to 1.8 mm
Number of beads: 10-50% by volume of the tube (before packing)
Frequency: 50 Hz to 80 Hz
Time: 30 s to 60 s

For bacteria, archaea, fungi, and virus with Md less than or equal to 5 um: in saliva classified as liquid sample
2R: 1.4 mm to 1.8 mm
Number of beads: 10-50% by volume of the tube (before packing)
Frequency: 80 Hz to 100 Hz
Time: 30 s to 60 s

For bacteria, archaea, fungi, and virus with Md below 1 um: in intestinal biopsies: classified as medium solid sample
2R: 1.0 mm to 1.8 mm
Number of beads: 10-60% by volume of the tube (before packing)
Frequency: 30 Hz to 150 Hz
Time: 30 s to 300 s

For bacteria, archaea, fungi, and virus with Md between 1-2 um: in intestinal biopsies: classified as medium solid sample
2R: 1.0 mm to 1.3 mm
Number of beads: 10-30% by volume of the tube (before packing)
Frequency: 30 Hz to 60 Hz
Time: 30 s to 60 s

For bacteria, archaea, fungi, and virus with Md between 1-2 um: in intestinal biopsies: classified as medium solid sample
2R: 1.3 mm to 1.8 mm
Number of beads: 10-60% by volume of the tube (before packing)
Frequency: 30 Hz to 100 Hz
Time: 30 s to 300 s

For bacteria, archaea, fungi, and virus with Md above 2 um: in intestinal biopsies: classified as medium solid sample
2R: 1.2 mm to 1.6 mm
Number of beads: 10-30% by volume of the tube (before packing)
Frequency: 30 Hz to 40 Hz
Time: 30 s to 60 s

For bacteria, archaea, fungi, and virus with Md less than or equal to 10 um: in intestinal biopsies: classified as medium solid sample
2R: 1.2 mm to 1.6 mm
Number of beads: 10-30% by volume of the tube (before packing)
Frequency: 40 Hz to 60 Hz
Time: 30 s to 60 s

For bacteria, archaea, fungi, and virus with Md above 2 um: in intestinal biopsies: classified as medium solid sample
2R: 1.4 mm to 1.8 mm
Number of beads: 10-50% by volume of the tube (before packing)
Frequency: 30 Hz to 50 Hz
Time: 30 s to 60 s

For bacteria, archaea, fungi, and virus with Md less than or equal to 10 um: in intestinal biopsies: classified as medium solid sample
2R: 1.4 mm to 1.8 mm
Number of beads: 10-50% by volume of the tube (before packing)
Frequency: 50 Hz to 80 Hz
Time: 30 s to 60 s

For bacteria, archaea, fungi, and virus with Md less than or equal to 5 um: in intestinal biopsies: classified as medium solid sample
2R: 1.4 mm to 1.8 mm
Number of beads: 10-50% by volume of the tube (before packing)
Frequency: 80 Hz to 100 Hz
Time: 30 s to 60 s

For bacteria, archaea, fungi, and virus with Md below 1 um: in glandular tissue: classified as soft solid samples
2R: 1.0 mm to 1.8 mm
Number of beads: 10-60% by volume of the tube (before packing)
Frequency: 30 Hz to 150 Hz
Time: 20 s to 300 s

For bacteria, archaea, fungi, and virus with Md between 1-2 um: in glandular tissue: classified as soft solid samples
2R: 1.0 mm to 1.3 mm
Number of beads: 10-30% by volume of the tube (before packing)
Frequency: 30 Hz to 60 Hz
Time: 20 s to 60 s

For bacteria, archaea, fungi, and virus with Md between 1-2 um: in glandular tissue: classified as soft solid samples
2R: 1.3 mm to 1.8 mm
Number of beads: 10-60% by volume of the tube (before packing)
Frequency: 30 Hz to 100 Hz
Time: 20 s to 300 s

For bacteria, archaea, fungi, and virus with Md above 2 um: in glandular tissue: classified as soft solid samples
2R: 1.2 mm to 1.6 mm
Number of beads: 10-30% by volume of the tube (before packing)
Frequency: 30 Hz to 40 Hz
Time: 20 s to 60 s

For bacteria, archaea, fungi, and virus with Md less than or equal to 10 um: in glandular tissue: classified as soft solid samples
2R: 1.2 mm to 1.6 mm
Number of beads: 10-30% by volume of the tube (before packing)
Frequency: 40 Hz to 60 Hz
Time: 20 s to 60 s

For bacteria, archaea, fungi, and virus with Md above 2 um: in glandular tissue: classified as soft solid samples
2R: 1.4 mm to 1.8 mm
Number of beads: 10-50% by volume of the tube (before packing)
Frequency: 30 Hz to 50 Hz
Time: 20 s to 60 s

For bacteria, archaea, fungi, and virus with Md less than or equal to 10 um: in glandular tissue: classified as soft solid samples
2R: 1.4 mm to 1.8 mm
Number of beads: 10-50% by volume of the tube (before packing)
Frequency: 50 Hz to 70 Hz
Time: 20 s to 60 s

For bacteria, archaea, fungi, and virus with Md less than or equal to 5 um: in glandular tissue: classified as soft solid samples
2R: 1.4 mm to 1.8 mm
Number of beads: 10-50% by volume of the tube (before packing)
Frequency: 70 Hz to 100 Hz
Time: 20 s to 60 s

For bacteria, archaea, fungi, and virus with Md below 1 um: in adipose tissue: classified as soft solid samples
2R: 1.0 mm to 1.8 mm
Number of beads: 10-60% by volume of the tube (before packing)
Frequency: 30 Hz to 150 Hz
Time: 15 s to 300 s

For bacteria, archaea, fungi, and virus with Md between 1-2 um: in adipose tissue: classified as soft solid samples
22R: 1.0 mm to 1.3 mm
Number of beads: 10-30% by volume of the tube (before packing)
Frequency: 30 Hz to 60H z
Time: 15 s to 60 s

For bacteria, archaea, fungi, and virus with Md between 1-2 um: in adipose tissue: classified as soft solid samples
2R: 1.3 mm to 1.8 mm
Number of beads: 10-60% by volume of the tube (before packing)
Frequency: 30 Hz to 100 Hz
Time: 15 s to 300 s

For bacteria, archaea, fungi, and virus with Md above 2 um: in adipose tissue: classified as soft solid samples
2R: 1.2 mm to 1.6 mm
Number of beads: 10-30% by volume of the tube (before packing)
Frequency: 30 Hz to 40 Hz
Time: 15 s to 60 s

For bacteria, archaea, fungi, and virus with Md less than or equal to 10 um: in adipose tissue: classified as soft solid samples
2R: 1.2 mm to 1.6 mm
Number of beads: 10-30% by volume of the tube (before packing)
Frequency: 40 Hz to 60 Hz
Time: 15 s to 60 s

For bacteria, archaea, fungi, and virus with Md above 2 um: in adipose tissue: classified as soft solid samples
2R: 1.4 mm to 1.8 mm
Number of beads: 10-50% by volume of the tube (before packing)
Frequency: 30 Hz to 45 Hz
Time: 15 s to 60 s

For bacteria, archaea, fungi, and virus with Md less than or equal to 10 um: in adipose tissue: classified as soft solid samples
2R: 1.4 mm to 1.8 mm
Number of beads: 10-50% by volume of the tube (before packing)
Frequency: 45 Hz to 80 Hz
Time: 15 s to 60 s

For bacteria, archaea, fungi, and virus with Md less than or equal to 5 um: in adipose tissue: classified as soft solid samples
2R: 1.4 mm to 1.8 mm
Number of beads: 10-50% by volume of the tube (before packing)
Frequency: 80 Hz to 100 Hz Time: 15 s to 60 s

For bacteria, archaea, fungi, and virus with Md below 1 um: in whole insect organisms: classified as hard solid samples
2R: 1.0 mm to 1.8 mm
Number of beads: 10-60% by volume of the tube (before packing)
Frequency: 30 Hz to 150 Hz
Time: 60 s to 300 s

For bacteria, archaea, fungi, and virus with Md between 1-2 um:
2R: 1.0 mm to 1.3 mm
Number of beads: 10-30% by volume of the tube (before packing)
Frequency: 30 Hz to 60 Hz
Time: 15 s to 60 s

For bacteria, archaea, fungi, and virus with Md between 1-2 um: whole insect organisms: classified as hard solid samples
2R: 1.3 mm to 1.8 mm
Number of beads: 10-60% by volume of the tube (before packing)
Frequency: 30 Hz to 100 Hz
Time: 15 s to 300 s

For bacteria, archaea, fungi, and virus with Md above 2 um: whole insect organisms: classified as hard solid samples
2R: 1.2 mm to 1.6 mm
Number of beads: 10-30% by volume of the tube (before packing)
Frequency: 30 Hz to 60 Hz
Time: 15 s to 60 s

For bacteria, archaea, fungi, and virus with Md above 2 um: whole insect organisms: classified as hard solid samples
2R: 1.2 mm to 1.6 mm
Number of beads: 10-30% by volume of the tube (before packing)
Frequency: 30 Hz to 40 Hz
Time: 15 s to 60 s

For bacteria, archaea, fungi, and virus with Md less than or equal to 10 um: whole insect organisms: classified as hard solid samples
2R: 1.2 mm to 1.6 mm
Number of beads: 10-30% by volume of the tube (before packing)
Frequency: 40 Hz to 50 Hz
Time: 15 s to 60 s

For bacteria, archaea, fungi, and virus with Md above 2 um: whole insect organisms: classified as hard solid samples
2R: 1.4 mm to 1.8 mm
Number of beads: 10-50% by volume of the tube (before packing)
Frequency: 30 Hz to 50 Hz
Time: 15 s to 60 s

For bacteria, archaea, fungi, and virus with Md less than or equal to 10 um: whole insect organisms: classified as hard solid samples
2R: 1.4 mm to 1.8 mm
Number of beads: 10-50% by volume of the tube (before packing)
Frequency: 50 Hz to 80 Hz
Time: 15 s to 60 s

For bacteria, archaea, fungi, and virus with Md less than or equal to 5 um: whole insect organisms: classified as hard solid samples
2R: 1.4 mm to 1.8 mm
Number of beads: 10-50% by volume of the tube (before packing)
Frequency: 80 Hz to 100 Hz
Time: 15 s to 60 s

In some embodiments, selective disruption can be performed with the following beads radius and beads parameter

Full lysis of host non detectable effect on bacteria in many tissues
R: 1.2 mm to 1.6 mm
Number of beads: 25%
Frequency: 40 Hz
Time: 30 s

Fat MEM Short lysis: full lysis of host non detectable effect on bacteria in many tissues
R: 1.2 mm to 1.6 mm+one 4 mm bead
Number of beads: 30%
Frequency: 50 Hz
Time: 15 s

Fat MEM Long lysis: full lysis of host non detectable effect on bacteria in many tissues
R: 1.2 mm to 1.6 mm+one 4 mm bead
Number of beads: 30%
Frequency: 50 Hz
Time: 120 s

Fungal lysis Conditions: lysis of host non detectable effect on bacteria in many tissues
R: 0.5 mm beads
Number of beads: 30%
Frequency: 60 Hz
Time: 180 s

Fungal lysis Conditions: full fungal lysis
R: 0.5 mm beads
Number of beads: 30%
Frequency: 60 Hz
Time: 60 s

Bacterial resistance to lysis: minimal bacterial lysis
R: 1.2 mm to 1.6 mm
Number of beads: 25%
Frequency: 40 Hz
Time: 180 s

Additional, combinations of beads radius and beads parameter are identifiable by a skilled person upon reading of the present disclosure (see in particular the Examples section).

In embodiments of the host depletion methods the temperature during the disrupting does not exceed 60 C and can be performed at temperatures down to −100 C if samples are flash frozen prior to the disrupting. The disrupting is performed at near atmospheric pressure where pressure can range from 101 kPa+/−20 kPa as will be understood by a skilled person upon reading of the present disclosure.

In some embodiments the sample is pre-treated before the disrupting with reagents suitable to be used to break down bonds between host cells and sample matrix and/or to decrease viscosity of the sample. For examples in some embodiments biological samples containing sample matrix such as mucous with range in viscosity from 10 mPa*s to 100 Pa*s can be subjected with treatment with Dithiothreitol (DTT), TCEP, beta-Mercaptoethanol, or other reducing agents to decrease viscosity and increase accessibility of the host compartments to the beads during the disrupting and improve host removal.

In some embodiments, where the host is a plan and the host cells comprises a cell wall, pretreatment of the host sample is performed to remove disrupt the sample matrix primary formed by cellulose and to remove the plant cell wall to increase accessibility of the plant cells to the beads during the disrupting. Cell walls can be been removed using a variety of techniques including enzymatic treatment with enzymes like cellulase [1] enzymatic treatment with pectinase and cellulase (either at the same time [6] or one after the other [7]) which are used for example for the isolation of protoplasts in many plant species, as well as additional treatment indefinable by a skilled person.

For example, in some embodiments an procedure can be used for pretreatment of plant sample that is used for preparing protoplasts from the leaves and branches of tea plant suitable s involves treatment with 1.5% (w/v) cellulase and 0.4-0.6% (w/v) marerozyme (a pectinase) in a solution containing 0.4 M mannitol, enzymatic treatment for over 10 hours, and an iodixanol concentration of 65% [8].

Additionally, procedures suitable herein and developed for the isolation of protoplasts from wheat leaves that use 1% (w/v) cellulase and 0.25% (w/v) macerozyme in a solution (pH 5.7-6.0) containing 20 mM MES, 0.4M D-Mannitol, 10 mM KCl, 10 mM CaCl₂), and 0.1% (w/v) Bovine serum albumin [9]. Further, protoplasts exemplary procedures comprise procedures used to isolate protplast from species of seaweed (like Dictyopteris pacifica and Scytosiphon lomentaria) using different enzyme combinations due to the different composition of their cell walls; For example, one protocol performs enzymatic treatment with 1% cellulase onozuka RS and alginate lyase (4 U/mL) with a short incubation time of 3-4 hours [10], while others have also included 1% driselase during the enzymatic treatment step when isolating protoplasts from brown kelp species like U. pinnatifida [11]. Protoplast isolation protocols have also been developed for protoplast isolation from the roots of model legume species, like L. japonicus and M. truncatula, using 1.5% (w/v) cellulase R-10 and 2% (w/v) macerozyme R-10 in a 10 mM MES (pH 5.7) solution containing 0.4M D-sorbitol, 10 mM CaCl₂) solution, 5% viscozyme (a mixture of carbohydrases like arabanases, cellulase, β-glucanse, hemicellulase, and xylanase from Sigma Aldrich), and 1% BSA [12].

In some embodiments, pretreatment of plant sample can be performed in order to improve viable protoplast yields. Some protocols include pre-treatment steps like physical disruption of the tissue (e.g. cutting leaf samples), vacuum infiltration of the enzyme treatment used for the protoplast isolation, or preplasmolysis [13]. For example, Calcium chelation pre-treatment has been shown to increase the number of isolated protoplasts from D. pacifica [10].

In some embodiments the pre-treatment of plant cells can be performed with e cellulases used at an industrial scale, that have been isolated from fungal species [2] and are effecting because plant cell walls primarily consist of polysaccharide polymers, like cellulose, hemicellulose, and pectin [3]. Cellulases in particular can be used to specifically remove plant cell walls and leave fungal cell walls because the fungal cell wall is primarily composed of glucans, chitin, and glycoproteins [4] and, thus, are not targeted for degradation by cellulases. Furthermore, cellulases, pectinases, and xylanases (some of the most common enzymes used in protoplast isolation) are also unlikely to have major effects on bacteria and viruses because bacterial cell walls are primarily made up of peptidoglycan [14] and viral capsids are primarily made of repeated protein structures [15].

In embodiments comprising pretreatment of plant sample, the increased accessibility of host compartments to beads is expected since Isolated protoplasts are known or to be in lysed through exposure to a hypotonic solution, as protoplasts from some plant species have been documented to rupture when placed in hypotonic solutions [5].

In embodiments of the host depletion methods herein described comprising a disrupting treatment of a sample, possibly pretreated, thus results in a disrupted sample comprising disrupted host membranous compartments and accessible host NA.

The wording “accessibility” in the sense of the disclosure refers to the capability of a referenced molecule (e.g. nucleic acid and/or protein) comprised in a referenced mixture, to react with a referenced reagent in the referenced mixture (typically a sample) under referenced conditions (the conditions of a steps or assay of a method).

Accordingly, “an accessible nucleic acid” in the sense of the disclosure is a nucleic acid capable of reacting with a referenced reagent capable within a referenced mixture under referenced conditions. Conversely an “inaccessible nucleic acid” in the sense of the disclosure is a nucleic acid incapable of reacting with a referenced reagent within a referenced mixture under referenced conditions.

Additionally, “an accessible protein” in the sense of the disclosure is a protein capable of reacting with a referenced reagent capable within a referenced mixture under referenced conditions. Conversely an “inaccessible protein” in the sense of the disclosure is a protein incapable of reacting with a referenced reagent within a referenced mixture under referenced conditions.

Reagents added in samples or other referenced mixtures of methods of the instant disclosure typically include degrading reagents (including exonucleases and endonucleases, RNases, DNases, restriction enzymes, proteases, and other enzymes that cleave or degrade nucleic acids and/or proteins. Including reagents that prevent nucleic acid and/or proteins for use in downstream processing (e.g. Propidium Monoazide for nucleic acids)) detecting reagents (including enzymes used in detection of nucleic acids, including reverse transcriptases, polymerases, primers, probes), or binding reagents (including primers, probes, affinity reagents, nucleic acid binding proteins, surfaces for binding nucleic acids such as silica surfaces). Reacting includes binding as will be understood by a skilled person.

In order to increase host NA accessibility in some embodiments the disrupted animal sample can be further subjected to chemical or mechanical treatment to further increase accessibility of the host NA in the disrupted sample. Additional treatment directed to increase accessibility of host NA comprise protease treatment, such as proteinase K to degrade protective proteins from host NA are such as histones as well as other NA bound proteins and/or complexes which can be bound to host NA as will be understood by a skilled person. Proteases enzyme that can be used in protease treatment in the sense of the disclosure comprise proteinase K, TEV protease, trypsin, pepsin, alpha-lytic protease, and other proteases identifiable by a skilled person. Protease treatment can also increase accessibility by removal of proteins associated with barrier integrity to assist in removal of host NA protective features thus further increasing the disrupting as will be understood by a skilled person.

In some embodiments herein described wherein the increase of host NA accessibility is part of microbial enrichment methods of the disclosure, the disrupted sample can be contacted with one or more nucleases for a time and under condition to degrade the accessible host nucleic acid in the disrupted sample and thus obtain a nuclease treated sample enriched with the target microbial compartment and related target microbial nucleic acid.

The term “nucleases” in the sense of the disclosure refer to any enzyme configured to cleave a chain of nucleic acid, Nucleases in the sense of the disclosure encompass enzyme capable of cleave and thus degrading DNA and/or RNA either double-stranded and/or single-stranded and/or DNA-RNA hybrids. Exemplary nuclease include Benzonase and/or DNase I+RNase A. Additional DNA nucleases that may be combined with any RNA nucleases: DNase I, DNase1 L1(DNaseX), DNase 1L2, DNase I L3, DNase II, DNase alpha, DNase beta (lower pH), Cas12a (ssDNA after binding to target site), DNase I XT (NEB), Exonuclease I (ssDNA), Exonuclease III (dsDNA), Exonuclease V, Exonuclease VII (ssDNA), Exonuclease VIII (truncated?), Lambda exonuclease (dsDNA-->ssDNA), mismatch nuclease (T mismatches in dsDNA), Msz exonuclease (ssDNA), RecJ (ssDNA), T5 Exonuclease (dsDNA exonuclease and ssDNA endonuclease), T7 exonuclease (dsDNA), T7 endonuclease I (mismatches and non-beta structures), Thermolabile exonuclease I (ssDNA), Nuclease micrococcal from Staphylococcus aureus (active at pH 8.8), thermophilic DNase, Additional list of RNA nucleases that may be combined with any DNA nucleases: RNAse A. RNAse H. and RNAse III, and exoribonucleases such as PNPase and Exoribonuclease I, Additional nucleases that may be combined with or without any DNA or RNA nucleases (i.e. contain both DNA and RNA cleavage activity): Benzonase, Turbonuclease, Nuclease S1, Nuclease S7. Nuclease P1, and Micrococcal nuclease.

In some embodiments, where the nuclease treated sample is used for detecting and/or analyzing target microbial DNA the nuclease can be selected to degrade host DNA only. Nucleases in the sense of the disclosure comprise any enzyme with activity degrading double-stranded and/or single-stranded DNA. These nucleases may include DNase I, DNase1 L1(DNaseX), DNase 1L2, DNase 1 L3. DNase II, DNase alpha, DNase beta (lower pH), Cas12a (ssDNA after binding to target site), DNase I XT (NEB), Exonuclease I (ssDNA), Exonuclease III (dsDNA), Exonuclease V, Exonuclease VII (ssDNA), Exonuclease VIII (truncated?), Lambda exonuclease (dsDNA-->ssDNA), mismatch nuclease (T mismatches in dsDNA), Msz exonuclease (ssDNA), RecJ (ssDNA), T5 Exonuclease (dsDNA exonuclease and ssDNA endonuclease), T7 exonuclease (dsDNA), T7 endonuclease I (mismatches and non-beta structures), Thermolabile exonuclease I (ssDNA), Nuclease micrococcal from Staphylococcus aureus (active at pH 8.8), thermophilic DNase.

In some embodiments where the nuclease treated sample is used for detecting and/or analyzing target microbial RNA, the nuclease can be selected to degrade host RNA only. These include but are not limited to endoribonucleases such as RNAse A, RNAse H. and RNAse III, and exoribonucleases such as PNPase and Exoribonuclease I or any combination thereof.

In some embodiments, where the nuclease treated sample is used for detecting and/or analyzing target microbial DNA and RNA, one or more nucleases can be selected to degrade both host DNA and host RNA.

In particular, in embodiments herein described the time of degradation is selected based on the mass of host nucleic acids, units of nuclease, and efficiency of nuclease. Parameters that further dictate the time of degradation include incubation temperature and buffer/pH conditions. These parameters can be tuned based on desired time of degradation as well as desired reaction conditions that maintain analyte integrity for a particular sample. For example in some embodiments the time can be from 2 minutes to 30 minutes using an incubation temperature of 37 C and 5 mM Tris and 2 mM MgCl2 buffer.

The temperature of degradation is based on activity of nuclease and origin of biological sample and the desired results For example in some embodiments the temperature can be chosen to minimize degradation of the target microbial compartments. In some embodiments, this includes incubating samples with nucleases at low temperature (room temperature or lower) in order to reduce degradation of the desired analyte. In some embodiments, this may include incubating at a low temperature to optimize enzyme performance. In some embodiments, samples will be incubated at high temperature (>42 C) in order to maximize enzyme activity and minimize the duration of incubation. In some embodiments, samples will be incubated at 37 C to balance enzyme performance with preservation of desired analytes.

In some embodiments the time and temperature of degradation are selected to obtain short enough NA fragments that they can be separated before or after subsequent processing of the nuclease treated sample. In some embodiments, NA size selection is achieved using commercial magnetic bead-based kits such as SPRI or AMPure, which can remove NA fragments smaller than 100-2000 bp. In some embodiments, size selection is achieved using gel electrophoresis. In some embodiments, size selection is used to target either DNA, RNA, or both types of NAs specifically. In some embodiments, size selection targets single or double stranded NAs or both.

Accordingly, in some embodiments methods herein described comprise degrading extracellular nucleic acid in the sheared sample by performing nuclease treatment, proteinase treatment and/or heat treatment of the sheard sample for a time and under condition to minimize lysis of microorganism cells thus obtaining a treated host sample depleted of host cells.

In some embodiments the degrading can be performed by contacting the disrupted sample with an among from 1 U to 1000 U of nucleases such as benzonase and/or other nuclease with a comparable enzymatic activity. In embodiments where the nuclease is benzonase this concentration of nuclease can remove host NA from up to 1E5 cells. Incubation time can range from 2 minutes to 60 minutes as will be understood by a skilled person.

In particular, in some embodiments the nuclease treatment can be performed for incubation at any time from 2 to 60 minutes. Considering Benzonase as a representative example one unit of Benzonase is defined as the amount of enzyme necessary to digest 37 ug of DNA in 30 minutes, in an average sample where up to 1E5 human cells per mg of sample are expected which corresponds to 60 ug of DNA per mg of sample. Accordingly a minimum of 2 U of Benzonase removes host 10,000-fold in samples consisting of more than 1E5 human cells within 30 minutes at 37 C. An excess of Benzonase can be added in order to quickly degrade these host NA with minimal impacts on the compartmented microbial NA. A range of 1 U to xxU or 125 U per sample. A skilled person will be able to identify incubation times and concentrations for additional nucleases upon reading of the present disclosure.

In embodiments herein described the degradation temperature during nuclease treatment is selected from any temperatures where the chosen nuclease has sufficient enzymatic activity and up to physiological conditions of the sample. The degradation step is performed at near atmospheric pressure where pressure can range from 101 kPa+/−20 kPa as will be understood by a skilled person.

In some embodiments the nuclease treated sample can be further treated with additional reagents capable of removing accessible host NA from the nuclease treated sample.

For example, in some embodiments, methods of the disclosure can further comprise contacting the nuclease treated sample with a capture reagent capable of specifically bind and capture extracellular NAs and/or animal NAs or their complexes, to further remove host NA from the nuclease treated sample.

The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions and additional interactions identifiable by a skilled person.

In some embodiments capture agents in the sense of the disclosure comprise anti-histone antibody configured to specifically bind host NAs according to methods and approaches identifiable by a skilled person.

In some embodiments capture agents in the sense of the disclosure comprise nucleic acid probes specific for and capable of complementarily binding to the target animal NAs for removal.

Nucleic acid probes in the sense of the disclosure refer to polynucleotides composed of two or more monomers including nucleotides, nucleosides or analogs thereof. The term “nucleotide analog” or “nucleoside analog” refers respectively to a nucleotide or nucleoside in which one or more individual atoms have been replaced with a different atom or a with a different functional group. Exemplary functional groups that can be comprised in an analog include methyl groups and hydroxyl groups and additional groups identifiable by a skilled person. Exemplary monomers of a polynucleotide comprise deoxyribonucleotide, ribonucleotides, LNA nucleotides and PNA nucleotides as will be understood by a skilled person.

The wording “specific” when used in connection with a primer and a target sequence indicates a primer capable of complementary bind the target sequence forming a duplex polynucleotide more thermodynamically stable under a reaction condition than other duplex polynucleotides resulting from complementary binding of the primers with additional polynucleotides possibly present.

Nucleic acid probes and/or primer specific for host NA suitable to be used in method herein described can be identified in silico using computer supported tools as will be understood by a skilled person. The wording “specific” “specifically” or “specificity” as used herein with reference to a computer supported tool, such as a software indicates a tool capable of identifying a target sequence (such as the nucleic acids of the target organism herein described) among a group of sequences e.g. within a database following alignment of the target sequence with the sequences of the database. Exemplary software configured to specifically detect target sequences comprise Primer-3[13]. [14]. [15].

In some embodiments, the degrading step can be replaced or combined with a chemical treatment of the disrupted sample to chemically modify the host NA to make the host NA inaccessible to reagents that prevent host NA from being compatible with downstream methods. This can include propidium monoazide treatment that may crosslink to NA to prevent downstream amplification (such as described in [5] as will be understood by a skilled person.

In embodiments herein described the disrupted sample and/or the nuclease treated sample can be advantageously used to isolate, detect and/or analyze target microbial compartments and/or target microbial nucleic acid, alone or in combination with detection and/or analysis of the host nucleic acid as will be understood by a skilled person upon reading of the present disclosure.

In particular a skilled person will understand that the increased host depletion and microbial enrichment obtained by the host depletion method alone or in combination with the enrichment method of the disclosure will results in an increased yield increased, limit of detection, increased accuracy, increased precision and/or increased resolution of the detected features in outcome of the detection and/or analysis compared to existing methods, as will be understood by a skilled person upon reading of the present disclosure (see in particular Examples section).

Accordingly, in some embodiments of the disclosure the increase of host NA accessibility and/or microbial enrichment in the disrupted sample and/or nuclease treated sample can be used in combination with methods directed to isolate a target microbial compartment in the host sample. In those embodiments, the disrupted animal sample and/or the nuclease treated sample, can be further processed to isolate one or more target microbial compartments.

In particular in those embodiments, methods of the disclosure further comprise separating the target microbial compartment from the disrupted animal sample and/or the nuclease treated sample to obtain a microbial fraction of the disrupted animal sample comprising the target microbial compartments of the animal sample and the related target microbial NA.

In some embodiments, the separating can be performed by centrifugation, filtration and/or size exclusion chromatography and additional techniques identifiable by a skilled person.

In some embodiments, the separating can comprises separating the target microbial compartments from the disrupted animal sample and/or the nuclease treated the microbial fraction based on density, size, and/or combination thereof of the target microbial compartments with respect components of the biological sample such as lipids, connective tissue, and other components released in the disrupting.

In some embodiments the separating can comprise separating target microbial compartments based on density, size, and/or combination thereof of the target microbial compartments with respect to other microbial compartments possibly present in the biological sample. For example the further separating can be performed to isolate fungi from excess of bacteria, or separate viruses from bacteria.

In some of those embodiments, the method can optionally further comprise contacting the microbial fraction, with a nuclease for a time and under conditions to degrade accessible host NA to obtain a nuclease treated microbial fraction of the disrupted animal sample, enriched with the target microbial compartment and the target microbial NA.

In embodiments of the disclosure the increase of host NA accessibility and/or microbial enrichment in the disrupted sample and/or nuclease treated sample can be used in combination with methods directed to detect and/or further analyze target microbial compartment and/or a target microbial nucleic acid.

The terms “detect” or “detection” as used herein indicates the determination of the existence, presence or fact of a target item in a limited portion of space, such as a sample, a reaction mixture, a molecular complex and a substrate as well as one or more biological features thereof. The term “detect” or “detection” as used herein can comprise determination of chemical and/or biological properties of the target, such as the ability to interact, and in particular bind, other compounds, ability to activate another compound and additional properties identifiable by a skilled person upon reading of the present disclosure. The detection can be quantitative or qualitative. A detection is “qualitative” when it refers, relates to, or involves identification of a quality or kind of the target or signal in terms of relative abundance to another target or signal, which is not quantified, such as presence or absence. A detection is “quantitative” when it refers, relates to, or involves the measurement of quantity or amount of the target or signal (also referred as quantitation), which includes but is not limited to any analysis designed to determine the amounts or proportions of the target or signal. A quantitative detection in the sense of the disclosure comprises detection performed semi-quantitatively, above/below a certain amount of nucleic acid molecules as will be understood by a skilled person and/or using semiquantitative real time isothermal amplification methods including real time loop-mediated isothermal amplification (LAMP) (see e.g. semi quantitative real-time PCR). For a given detection method and a given nucleic acid input, the output of quantitative or semiquantitative detection method that can be used to calculate a target nucleic acid concentration value is a “concentration parameter”.

In embodiments of the present disclosure where the methods and systems are directed to the analysis of a referenced items, one or more detected biological features are further examined with analytical tools to obtain an increased understanding of biological relationships and processes known to be connected to experimental observations, and the translation of that understanding to actionable insights and concrete hypotheses, as will be understood by a skilled person. [5]

In particular in some embodiments, methods directed to detect and/or further analyze target microbial compartment and/or a target microbial nucleic acid herein described can comprises isolating a replication competent target microbial compartment from a microbial fraction and/or a nuclease treated microbial fraction of the present disclosure; and contacting the isolated replication competent target microbial compartments with a reagents for a time and under conditions allowing replication of the isolated replication competent target microbial compartments to obtain isolated amplified target microbial compartment.

The term “replication competent” as used herein in connection with a microbial compartment indicates a microbial compartment capable to undergo amplification process under proper conditions identifiable by a skilled person. The term “amplify” or “amplification” as used herein with respect to target microbial compartments indicates a process of replication of the target microbial compartments which create new compartments which in some embodiments are able of further replication, in particular, in some embodiments, the amplification of isolated compartments can comprise bacterial cell culturing, sequence-Independent, Single-Primer Amplification (SISPA) of viruses.

In embodiments of the disclosure comprising isolation of one or more target microbial compartments, methods and systems herein described can further comprise performing detection and/or analysis of one or more target microbial compartments from a microbial fraction, nuclease treated sample, isolated microbial compartment and preferably an isolated amplified target microbial compartment as will be understood by a skilled person.

In some embodiments, the detection and/or analysis of one or more target microbial compartment can be performed by detecting and/or analyzing the microbial compartment without detection and/or analysis of the target microbial nucleic acid. Those embodiments comprise for example detection and/or analysis of: i) macroscopic features of the compartment (overall appearance of a microorganism, including its shape, size, color, and smell e.g. in cultures such an agar culture) ii) microscopic features of the compartment (overall appearance of a microorganism, under a microscope) possibly preceded and/or followed by staining (such as gram staining, endospore staining Ziebel-Nielsen staining, as well as staining for Fungi and yeast); iii) biochemical tests (such as catalase testing, oxidase testing, substrate utilization testing,) iv) immunological identification, (e.g. ELISA based method); v) chemical analytical identification (such as fatty acid profiling and metabolic profiles/chemo profile, e.g. through gas chromatography and mass spectrometry) as well as other methods identifiable by a skilled person upon reading of the present disclosure.

In some embodiments methods for detection and/or analysis of one or more target microbial compartment are performed through modification, detection and/or analysis of the related target microbial NA.

In particular in some embodiments, isolated microbial cells obtained with MEM methods and systems can be used in Single Cell Barcoding Methods. In those embodiments using MEM processed samples cell upsteam of these methods (eg. scSPRITE, MicroSPilT RNA) will significantly improve the performance of these applications as described e.g. by Arrastia (scSPRITE) and Kunchina (MicroSPLiT RNA) and in 2019-0144854 incorporated herein by reference in its entirety.

In some embodiments, isolated microbial cells obtained with MEM methods and systems can be used in NA Proximity Barcoding methods. In those embodiments using MEM processed samples cell upstream of these methods (eg. MetaHiC, meta3C, SPRITE, Hi-C, ProxiMeta, XRM-seq) will also significantly improve the performance of these applications described by Marbouty (MetaHiC), Quinodoz (SPRITE), Beitel et al., 2014, Yaffe and Relman, 2020, Kent et al., 2020)(Hi-C), Marbouty et al., 2014, Marbouty et al., 2017 (Meta3C), —Press et al., 2017 preprint, Stalder et al., 2019, Bickhart et al., (2019)(ProxiMeta), Ignacio-Espinoza et al., 2020 preprint)(XRM-seq).

In some embodiments, isolated microbial cells obtained with MEM methods and systems can be used in Cell Isolation Methods (eg. FACS, Microfluidics methods. In those embodiments using MEM processed samples cell upstream of these methods ds isolating single microbial cells (eg. Microfluidics, Fluorescence Activated Cell Sorting (FACS) as well as other single cell methods described by (Oluwaseun Adetunji)) for whole genome sequencing will dramatically improve the performance of these applications, Microbe-seq (Zheng et al., 2022), SiC (Lan et al., 2017) SAG-Gel (Chijiiwa) PCR-Activated Cell Sorting (Lim), as stated by Ma isolation of single cells for whole genome sequencing includes “serial dilution (Zhang et al., 2006), microfluidics (Chen et al., 2011), flow cytometry (Raghunathan et al., 2005), micromanipulation (Ishoy et al., 2006), or encapsulation in droplets (Tolonen and Xavier, 2017).”

In some embodiments analysis of microbial compartments of the disrupted sample, nuclease treated sample, microbial fraction and isolated compartments can be performed through isolation detection and/or analysis of the related target microbial NA as will be understood by a skilled person.

In particular in some embodiments the increase of host NA accessibility and/or microbial enrichment is directed to isolate a target microbial NA from the host sample. In those embodiments, the nuclease treated sample and/or the microbial fraction, can be further processed to isolate detect and/or analyze target microbial nucleic acid.

In some of embodiments, methods herein described further comprise extracting nucleic acids from the disrupted sample, nuclease treated sample and/or microbial fraction to provide nucleic acids extracted from the sample. As a person skilled in the art will understand, the extraction process generally comprises mechanical lysis via bead beating, capturing nucleic acids either on a silica column or magnetic beads, purifying nucleic acids by washing with ethanol, and eluting nucleic acids off of column or beads with water.

In some embodiments, mechanical lysis of isolated microbial compartments can be supplemented/enhanced or substituted with chemical lysis (e.g., phenol/chloroform, etc.). Nucleic acids can be also precipitated or phase-separated without the use of a column. Washing can be done with ethanol and many other solvents Elution/dissolution of washed nucleic acid can be done with water or many different stabilizing buffers (e.g., TE buffer).

In some embodiments, the nucleic acids extracted from the sample can be a total DNA extracted and purified from samples such as feces, gastrointestinal contents or aspirates, intestinal mucosa biopsy, using commercially-available kits validated for uniform DNA extraction from complex microbiota (e.g., ZymoBIOMICS) and for quantitative recovery of microbial DNA from samples with microbial loads across multiple orders or magnitude as will be understood by a person skilled in the art.

In some embodiments, the compositions methods and systems directed to further perform nucleic acid detection and/or analysis methods of the disclosure comprise: performing cell lysis and extract nucleic acids from the microbial fraction and/or a nuclease treated microbial fraction of the present disclosure, the cell lysis performed for a time and under condition to maximize extraction of nucleic acid having a length greater than 2000 bp.

In preferred embodiments, the cell lysis is performed for a time and under condition to further degrade nucleic acid having dimension equal to or less than 200 bp and in particular between 1 bp and 200 bp but having dimension lower than 1,000 bp.

In preferred embodiments, the cell lysis is performed for a time and under condition to further degrade nucleic acid having dimension equal to or less than 200 bp and in particular between 1 bp and 200 bp but having dimension lower than 1,000 bp.

In some embodiments, the methods of the disclosure directed to isolate target microbial NA from encapsulated microbes in the sample allow a recovery between 1-100% of the target microbial NA of the sample and in particular more than 10%. In some embodiments, the methods of the disclosure allow remove of 50-100% host NA %.

In embodiments of methods of the disclosure directed to detect and/or further analyze the target microbial NA detection and/or analysis of target microbial NA the method comprises amplifying the target microbial NA.

The term “amplify” or “amplification” as used herein with respect to target NA indicates a usually massive replication of a polynucleotide in particular of a gene or DNA sequence. Accordingly, amplifying indicated in connection with a reference polynucleotide indicates the replication of the referenced polynucleotide to provide a greater number of the referenced polynucleotide and increase representation of the reference polynucleotide in a target environment. Amplification can be conducted through methods such as: Polymerase Chain Reaction, ligase chain reaction, transcription-mediated amplification, methods and additional methods identifiable by a skilled person. Copies of a particular nucleic acid sequence generated in vitro in an amplification reaction are called amplicons or amplification products.

In some embodiments of the disclosure amplification as well as detection and/or analysis can comprise performing Polymerase Chain Reaction (PCR) on nucleic acids extracted from the sample. The term “polymerase chain reaction” as used herein indicates a reaction amplifying copies a polynucleotide in a series of cycles of temperature changes. In particular, in various PCR methods repeated cycles of heating and cooling exposes reactants of temperature-dependent reactions which result in amplification of the polynucleotide. PCR can amplify polynucleotides of up to 40 kilo base pairs (kbp) and typically amplifies between 0.1 and 10 kbp in length, as will be understood by a skilled person. In all PCR methods the amplification is performed by using primers and a polymerase. The term “polymerase” as used herein indicates an enzyme capable of synthesizes long chains of polymers or nucleic acids, replicating a target polynucleotide or template strand using base-pairing interactions. Exemplary polymerase comprises heat stable DNA polymerase such as Taq polymerase or high fidelity polymerases such as Pfu polymerase. Commercial modification of these base polymerases and their associated master mixes work well (e.g., Bio-Rad SsoFast EvaGreen Supermix (Bio-Rad Laboratories, Hercules, CA), 5PRIME HotMaster Taq DNA Polymerase and 5PRIME HotMasterMix (Quantabio, Beverly, MA), KAPA HiFi polymerase (KAPA Biosystems, Woburn, MA), JumpStart Taq DNA Polymerase (Sigma-Aldrich, St. Louis, MO).

In some embodiments, amplification detection and/or analysis of target microbial NA can comprise performing, “digital PCR” assay that provides an end-point measurement that provides the ability to quantify nucleic acids without the use of standard curves, as is used in real-time PCR. Digital PCR includes a variety of formats, including droplet digital PCR, BEAMing (beads, emulsion, amplification, and magnetic), and microfluidic chips. As a person skilled in the art will understand, digital PCR (dPCR) builds on traditional PCR amplification and fluorescent-probe-based detection methods to provide a sensitive absolute quantification of nucleic acids without the need for standard curves.

In some embodiments, detection and/or analysis of target microbial NA can comprise NA sequencing where the word “sequencing” as used herein indicates massive parallel sequencing performed via spatially separated, clonally amplified polynucleotide templates or single polynucleotide molecules, as will be understood by a skilled person. The term sequencing thus encompasses all sequencing methods including the basic methods as well as large scale and high throughput methods such as shotgun sequencing next-generation “short-read” and third-generation “long-read” sequencing methods as will be understood by a skilled person.

A skilled person will understand that methods and systems herein described which are directed to detection and/or analysis of one or more target nucleic acid per se and/or in connection with detection and/or analysis of a microbial compartment can be advantageously performed in connection with detection and/or analysis of a plurality of target microbial NA and/or related microbial compartments.

A skilled person will understand that the MEM host depletion and/or enrichment methods herein described will allow a significant improvement up to 1000-fold in the limit of detection of the microbial NA and/or target microbial compartment, which will result in an increased resolution of detection and/or analysis directed to a plurality of referenced items. This feature of the MEM sample processing of the disclosure makes such processing particularly suitable to detect and/or analyze microbial communities and/or additional target plurality of microbial compartments as well as related target plurality of microbial nucleic acid as will be understood by a skilled person.

The term “microbial community” as used herein refers to a group of microbes sharing an environment which can comprise one or more prokaryotes or individual genera or species of prokaryotes. A microbial community in the sense of the disclosure can thus include two or more microorganisms two or more strains, two or more species. two or more genera, two or more families, or any mixtures of microorganisms in the sense of the disclosure with additional life form such as viruses, comprised in the shared environment. The interaction between the two or more community members may take different forms and can be in particular commensal, symbiotic and pathogenic as will be understood by a skilled person. An exemplary microbial community is the ‘microbiome” of an individual which is an aggregate of all microbiota (all microorganisms found in and on all multicellular organisms) residing on or within tissues and biofluids of the individual.

Microbial communities can and are usually part of a host in the sense of the disclosure including the tissues, organs, and/or biofluids of the host.

In particular, in individual having a digestive tract (e.g. all vertebrates and in particular humans, as well as most invertebrates including sponges, cnidarians, and ctenophores) the microbiome residing in or within the digesting tract, (generally comprising bacteria and archaea), is also indicated as “gastrointestinal microbiome” or gut microbiome.

Additional fluids hosting a microbial community in individuals such as vertebrates and human comprise tear fluid, saliva, nasal, oral, tonsillar, and pharyngeal swabs, sputum, bronchoalveolar lavage (BAL), gastric, small-intestine, and large-intestine contents and aspirates, feces, bile, pancreatic juice, urine, vaginal samples, semen, skin swabs, tissue and tumor biopsy, blood, lymph, cerebrospinal fluid, amniotic fluid, mammary gland secretions/breast milk and tumor tissues.

Accordingly in a human individual, in addition to gastrointestinal microbiome, further microbiomes comprise eye microbiome, skin microbiome, mammary glands microbiome, placenta microbiome, seminal fluid microbiome, uterus microbiome, ovarian follicles microbiome, lung microbiome, saliva microbiome, oral mucosa microbiome, conjunctiva microbiome, biliary tract microbiome, tumor microbiome and additional microbiomes.

Additional exemplary microbiome in individuals comprise insect microbiome plant root microbiome (rhizosphere), aquaculture (fisheries, clam farms) and others identifiable to a person skilled in the art.

in this connection, preferred embodiments of MEM methods directed to detection and/or analysis of a plurality of target microbial NA and/or related microbial compartments, are performed to detect and/or analyze one or more microbiome and/or additional target pluralities of micro compartments and/or related microbial nucleic acid.

Accordingly in some embodiments herein described sequencing of MEM processed samples can be advantageously performed with large scale and high throughput sequencing methods in particular when directed to detection of a plurality of target nucleic acid which particularly benefit from the enrichment of the sample in target microbial NA as will be understood by a skilled person.

In particular, in embodiments herein described sequencing can be performed by Next Generation Sequencing (NGS) performed by generating sequencing libraries by clonal amplification of a target polynucleotide by PCR in vitro to provide amplified templates or providing single target polynucleotides; spatially segregating, amplified templates or single target polynucleotide; and sequencing the spatially segregated target polynucleotide by synthesis, such that the sequence is determined by the addition of nucleotides to the complementary strand rather than through chain-termination chemistry. While these steps are followed in most NGS platforms, each utilizes a different sequencing approach such as Pyrosequencing, Sequencing by reversible terminator chemistry, Sequencing-by-ligation mediated by ligase enzymes, and Phospholinked Fluorescent Nucleotides or Real-time sequencing as will be understood by a skilled person. Exemplary NGS kits commercially available include Illumina™ sequencing, Roche 454™ sequencing, Ion torrent: Protein/PGM™ sequencing, Nanopore sequencing, and SOLiD™ sequencing. Next generation sequencing methods are known in the art, and are described, e.g., in [16].

In some embodiments, the next-generation sequencing approach used herein is amplicon sequencing. “Amplicon sequencing” as used herein refers to a targeted sequencing method in which a discrete region of a genome is first amplified from the entire genome using PCR and the generated amplicons are used as templates for subsequent sequencing. Sequencing can be carried out in a sample containing amplification products of a single amplicon. Alternatively, the sample can contain mixtures of multiple amplicons pooled together, as will be understood by a skilled person. Amplicon Sequencing is a method where multiple amplicons are pooled together and co-sequenced.

“Amplicons” as used herein are defined as replicated DNA (or ribonucleic acid—RNA) strands that are formed by polymerase chain reaction (PCR), ligase chain reactions (LCR), or other DNA duplication methods, where the strands are copies of a target region of a genome. In order to multiplex PCR amplification, each amplicon has to be unique and independent (no overlapping amplicons), which requires careful selection of the primers used to tag the regions to be amplified. Amplicons for sequencing have a length typically in the range between 100 bp and 500 bp.

The processing and sequencing of amplicons with different sequencing platforms can be flexible and allows for a range of experimental designs. A variety of options regarding design parameters can be selected, such as the length of amplicons, the number of amplicons pooled together, the number of reads desired for a given amplicon or a pool of amplicons, whether to read from one end (unidirectional sequencing) or both ends (bi-directional sequencing) of the amplicon and other factors identifiable to a skilled person in the art.

In some embodiments herein described, the target microbial amplicon samples generated from real-time PCR are quantified, pooled, purified, and sequenced with, NGS sequencing which results provide the sequence read and count data.

The terms “read” or “reads” used herein are defined as a sequenced range of DNA or RNA. A read can be a sequence that is output by a sequencing instrument, where the read attempts to match a range of DNA that was input to the instrument. Each set of reads maps to a particular amplicon, with a read being a sequence for the complete amplicon or, typically, a range of bases comprising a subset of the amplicon. The total set of reads in the input data for the filter pipeline can include multiple amplicons, each having multiple reads mapped to them. The range of the read lengths depends upon the primers chosen for a given library. The mapping of reads to an amplicon can be determined by overlapping paired-end reads (generally shorter than the length of the amplicon) for each sequenced amplicon to obtain the complete 16S amplicon sequences. Complete 16S amplicon sequences are used in downstream analysis to identify their proportions in the entire 16S amplicon pool and to identify their taxonomic origin. The mapping of reads to an amplicon can also be determined during alignment/assembly using a sequencing alignment tool, for example the Bowtie™ 2 read alignment tool from Johns Hopkins University (see Ref “Fast gapped-read alignment with Bowtie 2” by Ben Langmead and Steven L. Saizberg, Nat Methods, Author manuscript; PMC 2013-Apr.-1).

In some embodiments, methods directed to perform analysis of the target microbial nucleic acid comprises performing metagenomic analysis and/or meta transcriptomic analysis of the extracted nucleic acid from the treated host sample depleted of host cells herein described.

In some of embodiments metagenomic analysis and/or meta transcriptomic analysis comprises Short Read DNA Sequencing which results in a digital file containing multiple reads of genetic sequences ranging from 2M-2.2 billion reads with each read consisting of 50-300 bases (Illumina ShotGun guide), Long Read Sequencing (Trigodet et al), and/or RNA-Sequencing transcriptomics (Wang).

In some of embodiments metagenomic analysis and/or meta transcriptomic analysis comprises Epigenetics Sequencing to study of stable changes in cell function (known as marks) that do not involve alterations in the DNA sequence.

In some embodiments the metagenomic analysis is selectively performed on extracted DNA from the treated host sample depleted of host cells herein described.

Methods and systems and related composition herein described in some embodiments result in microbial enrichment enabling Metagenome Assembled Genome (MAG) construction from low-biomass sample types including human intestinal biopsies. In particular, methods and systems and related composition herein described in some embodiments result in microbial enrichment enabling high-throughput metagenomic characterization from host-rich samples.

In some embodiments, the metagenomic sequence preparation can be performed by separating the DNA and RNA libraries to allow adjustments in the host depletion protocol or the depletion protocol and library preparation. In particular in some embodiments for DNA, kit can be used that allow sample processing with very low input. After host depletion with MEM process of the disclosure low prokaryotic biomass that total DNA is largely undetectable by traditional measures (Qubit or nanodrop). In those embodiments, an optimized a protocol for library preparation of low input sample types and corresponding kit can provided as will be understood by a skilled person. For RNA, the host depletion protocol can be performed in conjunction with a series of ribo-depletion kits for removal of host and microbial rRNA. Additional optimization can be performed such as a poly-A pulldown to remove host mRNA if remnant host signals are present. By using other kits in conjunction with our own, the host removal is increased.

In some embodiments, methods herein described and related systems and compositions, allow performing high-throughput metagenomic characterization from intestinal biopsies, with over 70 samples on a small NovaSeq6000 SP flowcell.

In some embodiments, MEM processing herein described and related methods, systems and compositions, allow performing MAG reconstruction for bacterial and archaea genomes from intestinal biopsies and we have constructed more than 65 novel MAGs without culturing.

In some embodiments, methods directed to perform analysis of the target microbial nucleic acid comprises performing Gene Expression applications to improve RNA-Seq metatranscriptomics gene expression detection with ribosomal RNA (eukaryotic or microbial) depletion for increased detection of microbial mRNA transcripts as describe by (Gifford)(NEB)(Illumina)(Zymo)(thermofisher) and Characterization of microbial RNA relative and absolute gene expression changes and patterns as described by (Gifford)(Munro) for bacteria, (Aylward) for viruses.

In some embodiments, methods directed to perform analysis of the target microbial nucleic acid comprises performing Non-Coding RNA-Seq applications directed to detect and/or analyze Transfer RNAs (tRNAs) as described by Eren “Transfer RNAs (tRNAs) in Eren and Zhang, microbial community structure and taxonomy can be inferred, characterization of expression of tRNA's and tRNA modifications.

In some embodiments, methods directed to perform analysis of the target microbial nucleic acid comprises performing Single Molecule Direct RNA Sequencing (Tilahun) to combine structural probing with native RNA sequencing to provide non-amplified, structural profiles of individual molecules with novel analysis methods for fungi.

In some embodiments, methods directed to perform analysis of the target microbial nucleic acid comprises performing Assembly of dsRNA and ssRNA viral genomes, RNA Epigenetics and/or Normalization of RNA expression or metaproteinomics dataset with DNA metagenomic dataset to measure microbial functionality (gene expression or metabolites) of a particular genus or species to disentangle from community functionality as described by (Franzosa)(Shaffer).

In some embodiments, methods directed to perform analysis of the target microbial nucleic acid comprises using machine learning to identify biomarkers or impactful features of multiple types (e.g. Multi-omics of host/microbial of and large datasets. Such as Metagenomics identification of biomarkers) Shaffer et al. Multi-omics Microbial interactions (Lloyd-Price)_Holo-omics (Host—microbial interactions using proteinomic, metagenomics, and metatranscriptomics) as described by (Nyholm, Wang, Yan, Lloyd-Price, Pedersen), host-pathogen interactions as described e.g. (Singh, Beltran).

In some embodiments, methods directed to perform analysis of the target microbial nucleic acid can be performed multiple times and over time to produce longitudinal data. Exemplary applications that can benefit from parallel processing of host NA's by integrated analysis of data produced from DNA or RNA Sequencing and/or proteomics comprise Increasing human transcripts detection in high bacterial:host ratios specimen such as human saliva by enriching host transcriptome with MEM isolated host NA and rRNA bacterial depletion techniques as described by Li. Characterizing host protein and transcriptional responses between different disease states as described by (Birgitta) Holo-omics (Host—microbial interactions using proteinomic, metagenomics, and metatranscriptomics) as describe by (Nyholm, Wang, Yan, Lloyd-Price, Pedersen), host-pathogen interactions as described by (Singh, Beltran) and additional applications identifiable by a skilled person.

In some embodiments methods and systems of the disclosure to perform profiling of a microbial community of in a host sample. In some of those embodiments, the method can comprise quantitatively detecting an absolute abundance and/or relative abundance of one or more target microbial NA of a microbial compartment of the microbiome, in a nuclease treated a disrupted animal sample of the present disclosure, a microbial fraction of the present disclosure and/or a nuclease treated microbial fraction of the present disclosure.

The wording “absolute quantification” as used herein in connection with a nucleic acid such as 16S rRNA indicates detecting absolute numbers of copies of the nucleic acid within a target environment such as a sample. Accordingly, absolute quantification of a 16S rRNA as used herein indicates the total number of 16S rRNA ribonucleotide or 16S rRNA gene within a target environment, herein also indicated as “absolute abundance”. Absolute quantification of a nucleic acid can be provided by direct detection of the nucleic acid (by a digital amplification method such as digital PCR which directly detect absolute copy numbers of a target nucleic acid) and/or based on a comparative quantification of the nucleic acid in combination with a standard measurement (herein also “anchor” and/or by detecting fold differences between sample (e.g. by real-time/qPCR).

Absolute quantification of a nucleic acid can also be provided using a fluorescence or spectrophotometric based method (e.g., Nanodrop or Qubit) which is considered to be proportional to the levels of the nucleic acid to be quantified. Absolute quantification of a nucleic acid can be provided by cell counting based methods such as flow cytometry, optical density, plating which is also considered to be proportional to the desired 16S nucleic acid levels. Absolute quantification of a nucleic acid can be provided by sequencing spike-in (adding a 16S sequence not in the sample at a known level, usually determined by dPCR/qPCR and then use the relative abundance after sequencing and the known abundance level that was inputted as the anchor) as will be understood by a skilled person.

Absolute quantification of a nucleic acid can also be directed to quantify a fold difference between a first quantity of the target nucleic acids and one or more second quantities of the same target nucleic acid in a different environment (e.g. a sample) or in a same environment at different times. In particular, absolute fold difference quantification can indicate a fold change in the nucleic acid abundance between two samples taken from a same environment at different times.

The wording “relative quantification” of a nucleic acid such as 16S rRNA quantification indicates a quantity of a target nucleic acid relative to a quantity of a different nucleic acid. In particular, relative quantification can indicate a quantity of the target nucleic acids relative to the quantity of one or more nucleic acids (typically a plurality of nucleic acids) in a same environment (e.g. a sample).

In relative quantification of a target microbial nucleic acid, a relative abundance of the target microbial nucleic acid is determined (e.g. within a group of microbial nucleic acid), but the absolute amount of the target microbial nucleic acid is not necessarily known.

Relative abundances obtained by relative quantification can be multiplied with a standard herein also identified as an “anchor”, to obtain absolute quantification value as will be understood by a skilled person. Suitable anchors comprise a measure of an unchanging parameter in the target environment where the detection is made (e.g. a sample or samples) such as the total concentration of cells, DNA, or amplicons as determined by flow cytometry or qPCR or dPCR.

Methods and systems and related composition herein described in some embodiments result in microbial enrichment enabling analysis of bacterial RNA. Shorter incubations allowed by the methods and systems herein described are expected to allow to capture physiologically relevant profiles for the enriched microorganism to be analyzed, since RNA expression changes with time in microbial cells possibly present in the host sample as will be understood by a skilled person.

In some embodiments, MEM methodology herein described and related systems and compositions, allow identification of individualized gene clusters within the most abundant intestinal microbe, Phocaeicola vulgatus, to highlight the need for entire genome characterization.

In some embodiments, MEM herein described and related systems and compositions, allow identification niche microbiomes present along the SI to the colon, highlighting the need for high-resolution characterization of biopsies.

In some embodiments, MEM herein described and related systems and compositions, have also been demonstrated to be faster than other existing methodology yet more effective at enriching the host sample with microorganism cells and/or nucleic acids associated thereto as will be understood by a skilled person.

In some embodiments, MEM processing herein described and related systems and compositions, allow performing genome reconstruction which enables the study of unculturable microbes as well metagenomic sequencing for future mechanistic understanding of host-microbial gut interactions.

In some embodiments, methods and systems herein described herein described further comprise sample preparation configured to minimize loss of microorganism cells possibly present in the host sample.

In some embodiments, preparing a sample can comprise providing an anaerobic sample or preferably an aerobic samples which provide a reduced loss of microorganisms (and resulting microorganism enrichment) in particular sample types regularly exposed to the air (e.g. saliva, nose swabs, skin swabs, etc.) are expected to be better non-fresh samples than samples from an anerobic environment, while sample types in a typical anaerobic environment are expected to more likely see larger microbial losses. in saliva, negligible microbial loss can be detected by the protocol but processing of our protocol on intestinal biopsies shows about an order of magnitude loss of bacterial load.

In preferred embodiments, sample preparation can be performed by: i) Flash freezing of fresh samples is a suitable method for future host depletion of non-fresh sample ii) Minimizing any preservatives or bactericides and any other materials that can result in significant losses and/or damage to the microbial fraction that would result in losses during sample processing of the microbial fraction as will be understood by a skilled person.

In preferred embodiments, sample preparation can be performed with an amount of starting sample material with as few as 10\hat{ }1 microbes per uL of elution for successful host depletion, defined as below 99% host reads. This number are expected to be possibly lower after remnant host nucleic acid removal, which we did not perform on these samples.

In some embodiments, disruption of the sample releases intracellular microbes for further processing. Examples of microbes that may exist stably intracellularly prior to disruption include but are not limited to Salmonella enterica, Listeria monocytogenes, Francisella tularensis, Legionella lumophila, actively replicating virions including SARS-CoV-2, HIV-1, HIV-2 Hepatitis B, Hepatitis C, Human papilomavirus, and other microbes that have been phagocytosed into immune cells.

In some embodiments, samples are processed prior to or after disruption to remove enzyme or analytic inhibitors. This may include but is not limited to the physical or chemical removal of lipids in lipidaceous samples including adipose tissue, brain tissue, milk, and other lipidaceous samples.

Even if described with reference to host cells and microorganism cells methods and systems and related composition herein described in some embodiments can be used to selectively deplete a sample comprising cells having different dimensions by adjusting the selective mechanical lysis of the cells to be depleted in a target sample as will be understood by a skilled person upon reading of the present disclosure.

In particular although the methods and systems of the disclosure have been designed in the context of microorganisms, the related procedure is expected to be applicable to any cell types where the depletion target cell has a larger cell size than the enrichment target. This is expected to comprise mixed mammalian cell types for downstream study of a specific cell type. Additionally, the target of enrichment can also refer to viral particles to the extent they can also be selectively enriched according to methods herein described as will be understood by a skilled person upon reading of the present disclosure.

Methods and systems herein described can be applied to any method where host and target have a difference in cell or capsule size and including bacteria, fungi, archaea, virus, different cell types of microbial compartments Target and host can also be from the same organism. For example, if a target cell type has a smaller size than surrounding host cells, this cell type could likely be enriched with the correct bead size.

Methods of the present disclosure can be performed with a corresponding system comprising at least one set of beads, and one or more look up table connecting the features of the at least one set of beads, such as material dimensions and elastic modulus with i) one or more set of beads parameters for MEM disruption a) of one or more type of host compartments selectively with respect to one or more microbial compartments possibly present within a host sample, b) in one or more sample types, of c) one or more hosts.

A look-up table as used herein is an N-dimensional array of data indexed by one or more input parameters, such that providing the input parameters provides the system with the data required for the solution (either the final solution, or an intermediate value used to derive the solution). Look-up tables can be stored in firmware or software. Look-up tables can be stored in memory locally, or they can be stored in a remote server where a request is sent to the remote server with the input parameters and the remote server returns the data accessed in the table. The look-up table can be populated by pre-calculating equations using the methods described herein.

In some embodiments, the system further comprises at least one nuclease for performing MEM degradation according to the present disclosure alone or in combination with additional reagents to perform any one of the methods and reactions described herein such us polymerase chain reaction, and amplicon sequencing and additional reactions alone or in combination for simultaneous combined and/or sequential use to according to anyone of the MEM methods herein described.

In some embodiments, the systems further comprise buffers, enzymes having polymerase activity, enzymes having polymerase activity and lacking 5′-3′ exonuclease activity or both 5′ to 3′ and 3′ to 5′ exonuclease activity, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or detection of target nucleic acid sequences herein described.

In some embodiments, the systems of the disclosure to be used in connection with methods herein described, the reagents comprise DNA extraction, RNA extraction kit and amplification mix. The system can also include reagents required for preparing the sample, such as one or more of buffers e.g. lysis, stabilization, binding, elution buffers for sample preparation, enzyme for removal of DNA e.g. DNase I, and solid phase extraction material for sample preparation, reagents required for quantitative detection such as intercalating dye, reverse-transcription enzyme, polymerase enzyme, nuclease enzyme (e.g. restriction enzymes; CRISPR-associated protein-9 nuclease; CRISPR-associated nucleases as described herein) and reaction buffer. Sample preparation materials and reagents may include reagents for preparation of RNA and DNA from samples, including commercially available reagents for example from Zymo Research, Qiagen or other sample preparations identifiable by a skilled person. The system can also include means for performing DNA or RNA quantification such as one or more of: container to define reaction volume, droplet generator for digital quantification, chip for digital detection, chip or device for multiplexed nucleic acid quantification or semi-quantification, and optionally equipment for temperature control and detection, including optical detection, fluorescent detection, electrochemical detection.

In some embodiments the system can comprise a “standard” (anchor)—sample containing either single or complex microbial 16S DNA of known concentration (copy number), such as the one (ZymoBIOMICS Microbial Community DNA Standard, Zymo Research, Irvine, CA, USA) described in [17]) and additional standards identifiable by a skilled person. In some exemplary embodiments, the standard can consist of 10 microorganisms, 8 of which are bacteria (Listeria monocytogenes, 12%; Pseudomonas aeruginosa, 12%; Bacillus subtilis, 12%; Escherichia coli, 12%; Salmonella enterica, 12%; Lactobacillus fermentum, 12%; Enterococcus faecalis, 12%; Staphylococcus aureus, 12%) with 16S genes. These taxa are mixed together at defined concentrations so that the expected outcome of extraction and sequencing is known. The absolute concentration of 16S copies in such standard can be either estimated from the total DNA concentration (e.g., 10 ng/microL) and the approximate genome size of the members of this defined community. Alternatively, the absolute concentration of 16S copies in such standard can be directly measures by digital PCR as will be understood by a skilled person. Additional exemplary standard comprise samples of nucleic acids extracted from other complex mixtures of microorganisms (e.g., stool) or from pure microbial cultures (e.g., E. coli) can be quantified using digital PCR and serve as absolute quantification standards for qPCR and BC-qPCR assays described herein.

The systems herein describe can also include other necessary reagents to perform any of the NGS techniques disclosed herein. For example, the systems can further comprise one or more of: adapter sequences, barcode sequences, reaction tubes, ligases, ligase buffers, wash buffers and/or reagents, hybridization buffers and/or reagents, labeling buffers and/or reagents, and detection means. The buffers and/or reagents are usually optimized for the particular amplification/detection technique for which the system is intended. Protocols for using these buffers and reagents for performing different steps of the procedure can also be included in the system.

In some embodiments the system can comprise cooling bead beating tubes and containers to perform the disrupting as well as any additional components identifiable by a skilled person upon reading of the present disclosure.

The systems herein disclosed can be provided in the form of kits of parts. In kit of parts for performing any one of the methods herein described the set of beads, look up tables, the reagents for the MEM processing of a sample possibly inclusive of downstream processing can be included in the kit.

In a kit of parts, the components of the combinations of the various systems in accordance with the present disclosure can be comprised in the kit independently possibly included in a composition together with suitable vehicle carrier or auxiliary agents. For example, one or more probes can be included in one or more compositions together with reagents for detection also in one or more suitable compositions.

Additional components can include labeled polynucleotides, labeled primer such as barcoded with an adapter sequence for next generation sequencing, labels, microfluidic chip, reference standards, and additional components identifiable by a skilled person upon reading of the present disclosure.

The terms “label” and “labeled molecule” as used herein refer to a molecule capable of detection, comprising to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemoluminescence, production of a compound in outcome of an enzymatic reaction and the like.

In embodiments herein described, the components of the kit can be provided, with suitable instructions and other necessary reagents, in order to perform the methods here disclosed. The kit will normally contain the compositions in separate containers. Instructions, for example written or audio instructions, on paper or electronic support such as tapes, CD-ROMs, flash drives, or by indication of a Uniform Resource Locator (URL), which contains a pdf copy of the instructions for carrying out the assay, will usually be included in the kit. The kit can also contain, depending on the particular method used, other packaged reagents and materials (i.e. wash buffers and the like).

Further details concerning the identification of the suitable carrier agent or auxiliary agent of the compositions, and generally manufacturing and packaging of the kit, can be identified by the person skilled in the art upon reading of the present disclosure.

### EXAMPLES

The host depletion and microbial enrichment approaches (MEM) and related methods and systems herein described are further illustrated in the following examples, which are provided by way of illustration and are not intended to be limiting.

In particular, the following examples illustrate exemplary methods and protocols for performing the methods.

The following materials and methods were used to retrieve the data discussed in the examples section.

### Sample Collection

Mice (stool samples). All animal husbandry and experiments were approved by the Caltech Institutional Animal Care and Use Committee (IACUC protocol #21-1769). Male and female wild-type, non-transgenic surplus mice were used for stool collection. These animals were being fed a standard chow (LabDiet Picolab 5053) prior to stool collection. The stool was freshly collected in the afternoon by gently handling the mice. A total of 3 stool pellets from 3 different mice were collected at a time and were transferred to clean microfuge tubes with sterile tweezers. Samples were stored on ice for up to 30 min before being processed in the laboratory. A total of 1 mL of saline was added to each stool pellet and the samples were homogenized by pipetting up and down with a P1000. After samples appeared homogenous, stool samples were diluted 3-fold in saline and 100 μL from each diluted stool sample was processed with various host-depletion methodologies (see “MEM” and “Methodological Comparisons”).

Rat (small intestine and colonic samples). Tissue collection was performed post-mortem through an institutional tissue sharing program that does not require IACUC approval. One wild-type Syngap surplus rat was euthanized with C02 and the small intestine was removed with sterilized tweezers. The rat was being fed a standard chow (LabDiet Picolab 5053) but was fasted for 6 hours prior to sample collection.

A portion of the small intestine that appeared clear of content was cut and placed on a petri dish on ice. Any remnant lumenal contents were removed by squeezing the intestine with tweezers. The intestine was then cut and opened longitudinally with the mucosa facing upwards. A sterile tweezer was then used to scrape the mucosal contents along the small intestine and placed into a clean microfuge tube on ice. Samples were stored on ice for up to 30 min before being processed in the laboratory. Mucosal scrapings were mixed slightly with sterilized tweezers and separated into 13 clean microfuge tubes, each tube containing a couple milligrams of tissues (see “MEM” and “Methodological Comparisons”).

The large intestine was placed on a separate petri dish on ice and any luminal contents were removed by squeezing the intestine with tweezers. The entire large intestine was then cut into 14 evenly sized pieces with a sterile scalpel. Sterile tweezers were used to transfer each intestinal piece into a clean microfuge tube on ice. Samples were stored on ice for up to 30 min before being processed in the laboratory (see “MEM” and “Methodological Comparisons”).

Human (saliva samples). Human saliva samples were acquired from three healthy adult volunteers under California Institute of Technology Institutional Review Board (IRB) protocol #21-1092. All participants provided (digital) written informed consent prior to donation. No personal identifying information was collected at the time of consent and participant specimens were coded. Volunteers were asked not to eat, drink, chew gum, brush their teeth, or smoke 30 min prior to collection. No volunteers had taken systemic antibiotics for at least 2 weeks prior to donation. Volunteers were instructed to pool saliva in their mouths and spit 2 mL of saliva, ignoring bubbles when estimating volume, into a 15 mL conical tube through a plastic funnel. Prior to undergoing MEM, saliva samples underwent a DTT (dithiothreitol) pre-treatment in some experiments. Saliva was mixed at a 1:1 ratio with fresh DTT (10 mM DTT in 1×PBS) through brief vortexing. Saliva and DTT mixed samples were vortexed briefly and incubated for 1 min at room temperature before undergoing host-depletion processing (see “MEM” and “Methodological comparisons”).

Human (tissue samples). All activities related to enrollment of participants, collection of samples, and sample analysis were approved by the University of Chicago IRB and performed under IRB protocols #15573A and #13-1080. De-identified samples were received at Caltech and analyzed under Caltech IRB protocol #21-1083. Adults scheduled for routine colon cancer screenings via colonoscopy at the University of Chicago Medicine (UCM) were screened for diagnosis and eligibility criteria for enrollment in the study on a weekly basis. Exclusion criteria included: participants with chronic infectious diseases such as human immunodeficiency virus (HIV) or hepatitis C (HCV); active, untreated Clostridium difficile infection; active infection with severe acute respiratory syndrome coronavirus 2 (SARS-CoV-2); intravenous or illicit drug use such as cocaine, heroin, non-prescription methamphetamines; active use of blood thinners; severe comorbid diseases; participants on active cancer treatment; and participants who were pregnant. Approaching prospective participants was at the discretion of their treating physician and was not done in cases that would put participants at any increased risk, regardless of reason. Participants were approached the day of their procedure and informed, written consent was obtained before any samples were acquired.

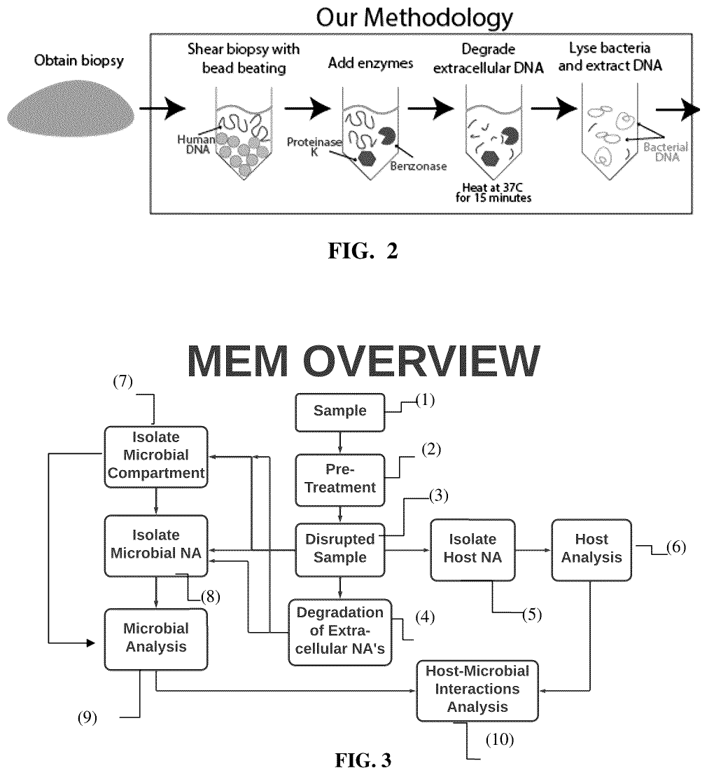

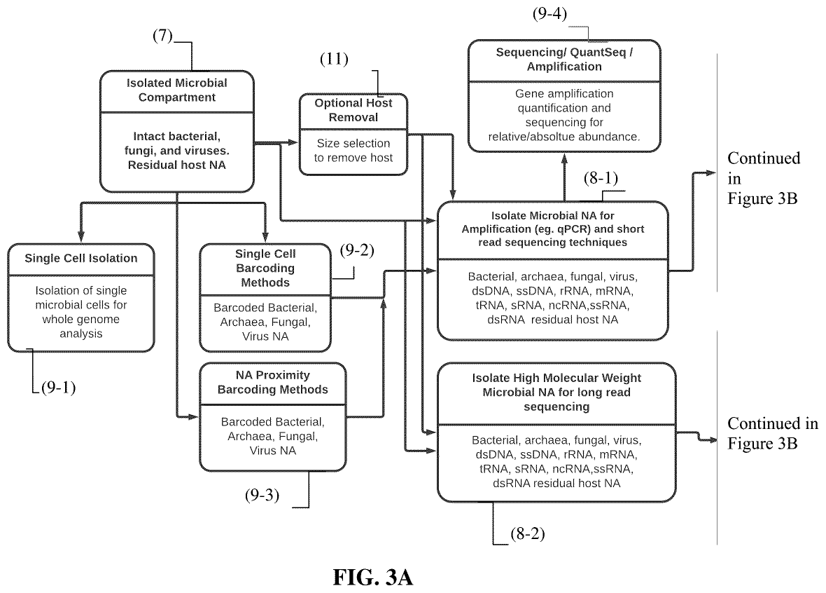

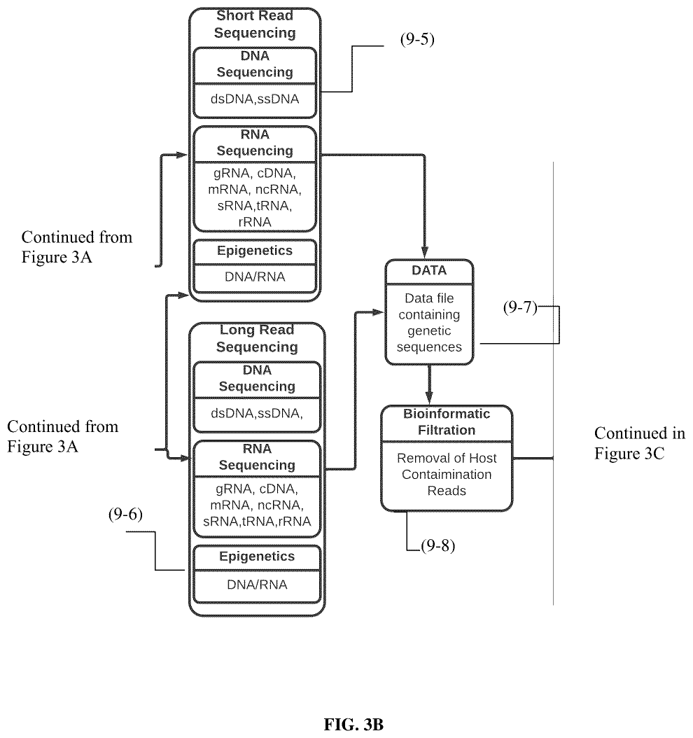

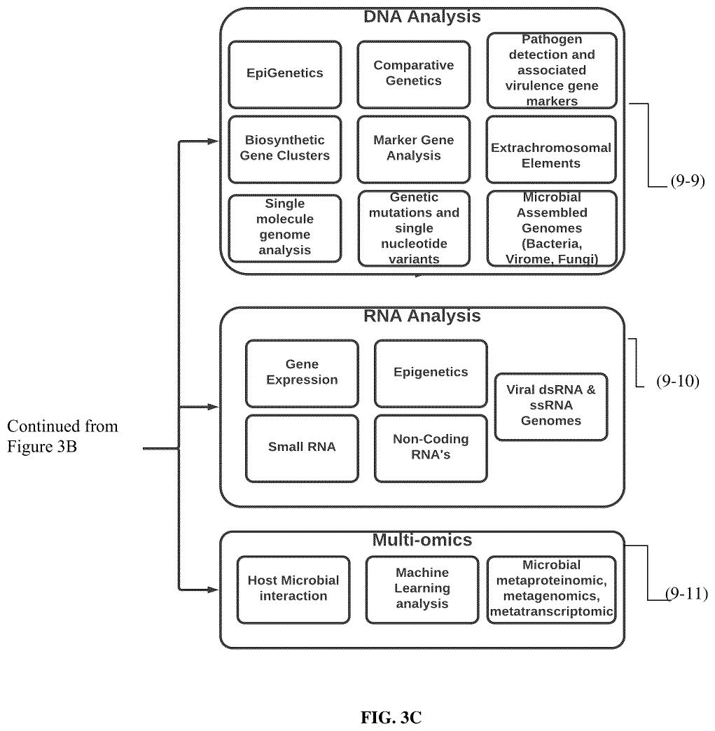

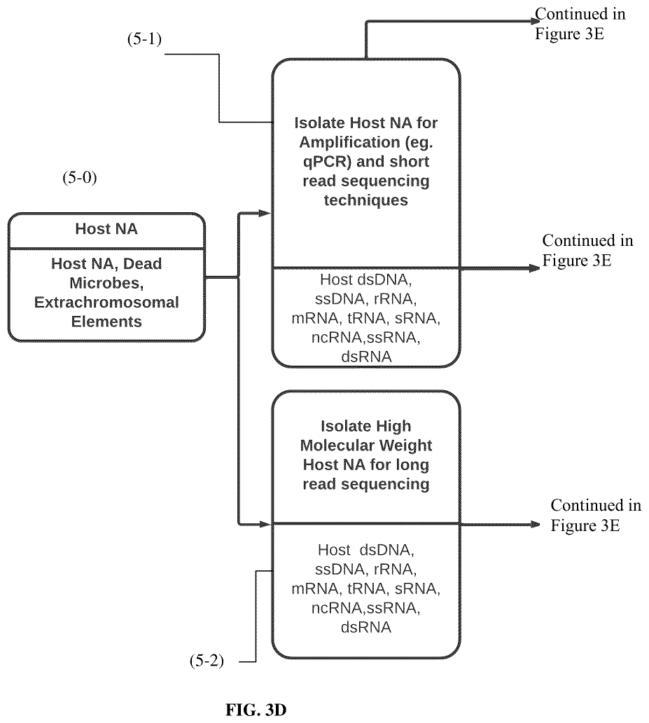

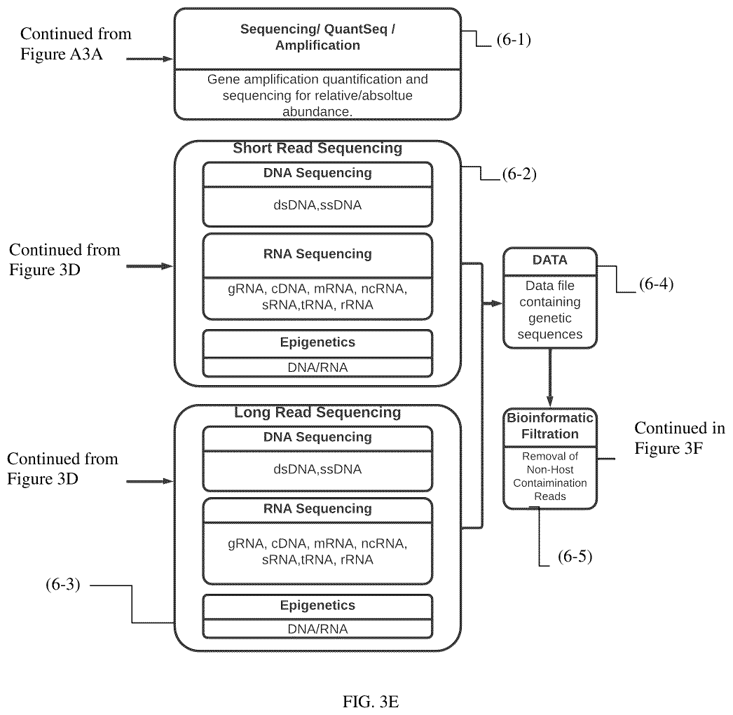

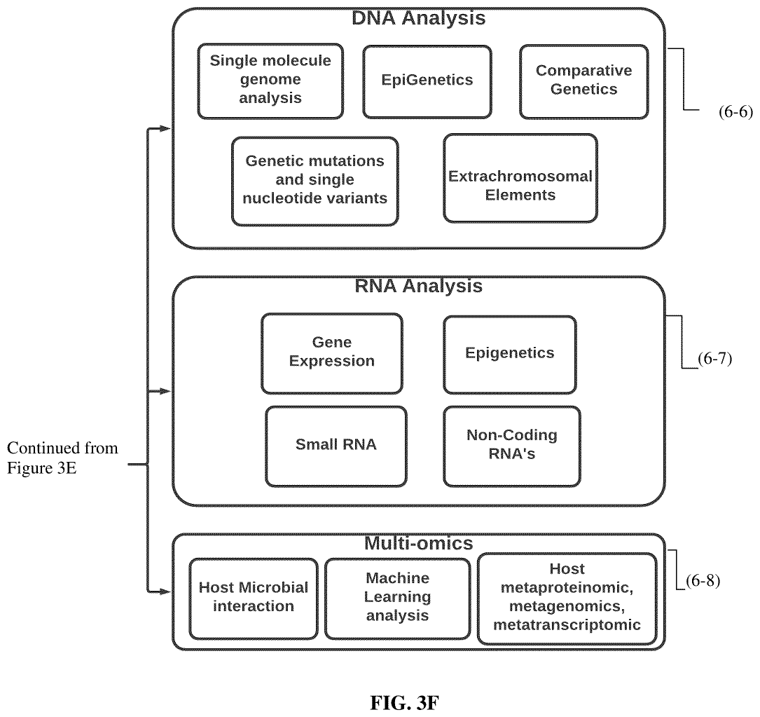

Human Ascending Colon Paired MEM and Control (FIG. 3D). To assess the impact of our MEM on intestinal microbes, 8 ascending colon biopsies, designated as 10 cm distal to the ileocecal valve, were collected from a single field of view (5 cm diameter) for five different participants. Biopsies were collected in a total of 2-3 passages with 3-4 biopsies per passage using a pair of 2.8-mm biopsy forceps. Biopsies from the same passage were stored together on ice in a dry microfuge tube for an average of 28 min (ranging from 15 min to 36 min). After samples were transferred to the lab, biopsies from the same passage were split into control and depleted groups for a total of 4 biopsies per condition with evenly sized biopsies present in each group. Biopsy size ranged from 0.2 mg to 4.8 mg with an average weight of 2.49 mg. Non-host-depleted biopsies were processed individually by adding 150 μL of PrimeStore MTM inactivation buffer to each biopsy and vortexing briefly before storing at −80° C. until DNA extraction. Depleted samples were processed individually at University of Chicago (see “MEM”) before shipment on dry ice to Caltech for DNA extraction.

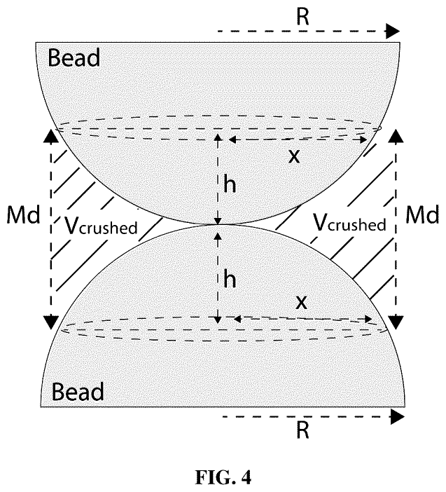

Longitudinal Sampling of the Human Intestinal Tract (FIG. 4C). For longitudinal sampling, a total of five participants were sampled 12 times from 4 different locations during a routine colonoscopy. The 4 locations sampled were the terminal ileum, ascending colon (designated as 10 cm distal to the ileocecal valve), descending colon, and rectum. From a single field of view (5 cm diameter) from each location, 3 biopsies were collected in one passage with 2.8 mm biopsy forceps and stored dry on ice in a microfuge. For participant CT14, only one rectal sample was obtained. On average, biopsies were 2.5 mg with a minimum size of 0.1 mg and a maximum of 5.9 mg. All biopsies were then processed individually in the laboratory at University of Chicago (see “MEM”) before shipment on dry ice to Caltech for DNA extraction. Time between specimen collection and processing ranged from 10 min to 52 min. Samples were processed individually in the laboratory at University of Chicago (see “MEM”) before shipment on dry ice to Caltech for DNA extraction. Additionally, three microfuge tubes of 400 μL saline were opened and closed in the laboratory to serve as clinical processing blanks.

### Depletion Protocols

Microbial-Enrichment Method (MEM). Samples for MEM treatment were placed into 2-mL 1.4-mm ceramic bead-beating tubes (Lysing Matrix D from MP Biomedical, Cat #116913050-CF) with a maximum volume of 400 μL. For solid sample types (stool and intestinal tissue), up to 400 μL of saline (0.9% NaCl, autoclaved) was added into the bead-beating tube. Samples were homogenized using FastPrep-24 for 30 sec at 4.5 m/sec and then immediately placed on ice. A total of 150 μL of homogenized tissue was removed and placed into a clean microfuge tube containing 10 μL of buffer (100 mM Tris+40 mM MgCl₂, pH 8.0 and 0.22 μm sterile filtered), 33 μL of saline (0.9% NaCl, autoclaved), 2 μL of EMD Millipore Benzonase Nuclease HC (Cat #71205), and 5 μL of NEB Proteinase K (Cat #P8107S). Samples were mixed lightly by manually pipetting up and down 5-10 times and spun briefly to pool (1,000×g for 5 seconds). Tubes were placed on a dry block incubator for 15 min at 37° C. with shaking at 600 rpm. Samples were then pelleted at 10,000×g for 2 min and the supernatant was removed and discarded. Pellets were resuspended in 150 μL of PrimeStore MTM (Longhorn), a transport medium, to inactivate residual enzymatic activity and stored at −80° C. until nucleic-acid extraction. The initial MEM protocol utilized DNase I treatment in place of Benzonase. However, we noted continuous microbial lysis during DNase I heat inactivation. Benzonase was used to remove high heat steps as it is fully inactivated by PrimeStore MTM.

Methodological Comparisons with Published Host-depletion Protocols. For all mouse, rat, and human saliva samples, the following published protocols were conducted to compare with MEM.

MolYsis. Host removal was performed with MolYsis Basic5 (Molzym Cat #D-301-050) following the manufacturer's protocol. A proteinase K pre-treatment (10 μL of NEB Proteinase K (Cat #P8107S)) was performed on solid-tissue samples (stool and intestinal samples) based on Molyzm's recommendations. The entire protocol was performed, including the additional BugLysis step before nucleic-acid extraction (see “DNA Extraction”).

QIAamp Microbiome. Host removal was performed with QIAamp DNA Microbiome Kit (Qiagen Cat #51704) following the manufacturer's protocol. Buffer AHL was aliquoted upon kit arrival and was not freeze-thawed more than once. In order to remove confounding factors from different DNA-extraction kits, the QIAamp DNA Microbiome Kit protocol was followed until the proteinase K incubation and the sample was then processed for nucleic-acid extraction (see “DNA Extraction”).

lyPMA. A previously published protocol known as lyPMA, was tested according to the paper's specifications. Liquid samples (diluted stool and saliva) were pelleted at 10,000×g for 8 min. Supernatant was removed and the pellet was resuspended in 200 μL of nuclease-free (NF) water with a light vortex. Samples were left at room temperature (RT) for 5 min. After samples were covered with foil, 10 μM of PMA (Propidium monoazide) was added and mixed by lightly vortexing each tube for a few seconds. Samples were incubated for 5 min in the dark at RT before being placed on ice <20 cm from a fluorescent bulb. Samples were incubated under light for 25 min with a quick centrifugation and rotation every 5 min. All samples were then processed for nucleic-acid extraction (see “DNA Extraction”). The lyPMA method was not tested on rat colonic sectionals due to the limited efficacy of osmotic lysis on solid tissues seen from mouse mucosa samples.

DNA Extraction. Nucleic acids were isolated following Qiagen's AllPrep PowerViral DNA/RNA Kit (Cat #28000-50). Samples were homogenized in 0.1 mm glass beads for 1 min at 6 m/s using FastPrep-24 (MP Biomedical) to ensure complete microbial lysis.[18] A maximum of 24 clinical samples were processed at a time and at least 3 processing blanks were run on each extraction kit. Samples were eluted into 100 μL of NF water. It should be noted that standard microbial bead-beating with 0.1 mm beads was not sufficient to completely lyse intact (control) biopsies in this study. A modified protocol using a mixture of 0.1 and 1 mm beads with extended BB times was utilized (samples were homogenized for 1 min at 6 m/sec three times with a 5-min incubation on ice between each bead-beating).

Quantification of Host DNA. Host load present in extracted DNA was characterized by droplet digital PCR (ddPCR) of a single-copy gene. For human saliva and tissue samples, the gene EIF5B was amplified based on primers found from literature[19] (F: GCCAAACTTCAGCCTTCTCTTC (SEQ ID: 1) and R: CTCTGGCAACATTTCACACTACA (SEQ ID: 2)). For samples originating from rodents, the gene Cyp8b1 was amplified based on primers found from literature[20] (F: GGCTGGCTTCCTGAGCTTATT (SEQ ID: 3) and R: ACTTCCTGAACAGCTCATCGG (SEQ ID: 4)). Samples were amplified on the C-1000 thermocycler (Bio-Rad) and quantified using the Bio-Rad QX200 droplet digital PCR system. The concentrations of the components in the ddPCR mix used in this study were as follows: 1× QX200 ddPCR EvaGreen SuperMix (Bio-Rad Cat #1864035), 500 nM forward primer, and 500 nM reverse primer for a total reaction volume of 25 μL. Thermocycling was performed as follows: 95° C. for 5 min, 40 cycles of 95° C. for 30 sec, 60° C. for 30 sec, and 68° C. for 30 sec, followed by a dye-stabilization step at 4° C. for 5 min and 90° C. for 5 min. All ramp rates were 2° C. per sec.

Quant-Seq. Microbial characterization and quantification was performed using the quantitative sequencing (“Quant-Seq”) pipeline we have described previously. [21] Due to the low bacterial loads present in intestinal biopsies, Quant-Seq was also performed on three MEM processing blanks. If a taxon was detected at a higher absolute abundance in any of the processing blanks compared to the intestinal biopsies, the taxon was removed from downstream analysis.

Shotgun Sequencing. Extracted DNA was prepared for sequencing using the Illumina DNA Prep (Cat #20018704). Based on Illumina DNA Prep specifications, a maximum input of 500 ng of DNA was used for library prep. After processing with the MEM protocol, almost all human biopsy samples had less than Illumina's recommended minimal DNA input amount of 1 ng and were below the limit of detection of the Qubit double-stranded DNA (dsDNA) High Sensitivity assay. Estimations of input DNA were made using 16S rRNA gene ddPCR (see “Quant-Seq” section) and host quantification (see “Quantification of Host DNA” section and Equation 1).

$$30 μ L \cdot Pr okaryotic load ( 16 S \frac{copies}{μ L} ) \cdot \frac{1 cell}{4 \; 16 S copies} \cdot \frac{3 fg DNA}{1 cell} = Pr okaryotic DNA ( fg ) \qquad Equation 1$$

$$Prokaryotic DNA + Host DNA = Total DNA$$

For these calculations, we assumed the 16S rRNA gene copy number (4 per cell), total DNA per microbial cell (3fg based on average genome size of 3 Mb), and lack of non-host/non-prokaryotic DNA. Host DNA was estimated from ddPCR (see “Quantification of Host DNA”).

For samples with DNA concentrations below Illumina's recommended input, additional PCR cycles were added to the amplification step based on DNA input (Table 1).

| DNA Input (pg) | Total PCR cycles |
|---|---|
| 500 | 13 |
| 100 | 15 |
| 10 | 19 |

Finished libraries were quantified through Qubit's dsDNA High Sensitivity assay and a High Sensitivity D1000 TapeStation Chip. If additional peaks were seen around the 45 bp or 120 bp marks, indicating the presence of primer dimers or adapter dimers, we performed an additional clean-up step with AMPureXP beads (Beckman Coulter) at a ratio of 0.8:1 of beads to library volume. For quantification, finalized libraries were amplified on the CFX-96 qPCR (Bio-Rad) with primers targeting the Illumina adapter sequence (F: AAT GAT ACG GCG ACC ACC GA (SEQ ID: 5) and R: CAA GCA GAA GAC GGC ATA CGA (SEQ ID: 6)). Libraries were diluted 1:40,000 in NF water prior to amplification to fall within the range of KAPA standards concentrations (Roche Cat #07960387001) for quantification. The concentrations of the components in the qPCR mix used were as follows: 1× SsoFast EvaGreen Supermix (Bio-Rad Cat #1725201), 125 nM forward primer, and 125 nM reverse primer for a total reaction volume of 10 uL. Thermocycling was performed as follows: 95° C. for 5 min, 40 cycles of 95° C. for 30 sec and 60° C. for 45 sec, followed by a melt-curve step at 95° C. for 15 sec, 50° C. for 15 sec, 70° C. for 1 sec, and 95° C. for 5 sec. Pooled samples were quantified through Qubit's dsDNA High Sensitivity assay and a High Sensitivity D1000 TapeStation Chip before submitting the samples for sequencing. Sequencing was performed by Fulgent Genetics using the Illumina NovaSeq6000 platform. Sequencing batch 1 was performed on the NovaSeq6000 SP flow cell and 2×100 bp reagent kit for paired-end sequencing with an average sequencing depth of 23M reads. Sequencing batch 2 was used for MAG assembly and was performed on one NovaSeq6000 S4 lane and 2×150 bp reagent kit for paired-end sequencing with an average sequencing depth of 223M reads. Samples were demultiplexed on the NovaSeq6000 and raw fastq files for read 1 and read 2 were provided along with fastqc files for each sample.

The number of non-host reads obtained from each sample can be accurately predicted based on a single qPCR measurement of bacterial load (16S rRNA gene copies) (Figure S8). This can be utilized to inform necessary sequencing depth (Figure S4).

Marker Gene Analyses. Sequencing data was processed using the KneadData v0.10.0[22]. Through KneadData, quality control (QC) and host removal were performed with Trimmomatic v0.39[23]. Human derived sample types were aligned to KneadData's default human reference genome (a combination of hg38 human genome reference (GenBank assembly accession #GCA_000001405.29) and small contaminant sequences) and aligned reads were removed. Samples acquired from mice were processed using the reference genome GRCm39 constructed from C57BL/6J mouse-strains (GenBank assembly accession #GCA_000001635.9). After bioinformatic host removal, the percentages of host reads were calculated by dividing reads remaining after host filtering by the total reads that passed QC. To assign species, non-host reads from read 1 and read 2 were then concatenated and processed using the Metaphlan 3.0 workflow outlined in bioBakery [5] under default settings (Database: mpa_v30_CHOCOPhlAn_201901) [22]. For stool, nearly 90% of the non-host reads did not align to known bacteria in the Metaphlan databases, likely due to the bias toward human microbiome datasets.

HUMAnN Pathway and Gene Alignment. Non-host read 1 and read 2 outputted from KneadData were concatenated and processed using the HUMAnN 3.0 workflow outlined in bioBakery (github.com/biobakery/biobakery) under default settings.[22] Taxonomic profiles obtained from MetaPhlan (see “Marker Gene Analyses”) were merged within patients and used as taxonomic inputs using the “--taxonomic-profile” flag in HUMAnN. Reported pathway abundances and gene abundances were normalized to relative abundances and concatenated.

MAG Assembly. Sequencing data was processed using the metagenomic workflow[24],[25] outlined in anvi'o[26, 27] v7.1 (https://anvio.org). QC filtering of short reads was performed using the Illumina-utils library[28] v2.12. Host reads were removed by alignment to the hg38 human genome reference (GenBank assembly accession #GCA_000001405.29). Assembly was performed on each sample individually using MEGAHIT[29] v1.2.9 unless co-assembly was explicitly stated as in FIG. 4, with default setting except setting a minimum contig length of 1000 bp. Short reads generated from each sample were then aligned to contigs generated from all assemblies using Bowtie2[30]. Contigs were processed using anvi'o to generate a contig databases with the command “anvi-gen-contigs-database” with default settings and with Prodigal[31] to identify open reading frames. Single-copy core genes were detected with “anvi-run-hmm” to (bacteria n=71 and archaea n=76, modified from Lee, 2019[32], ribosomal RNAs (rRNAs) (n=12, modified from github.com/tseemann/barrnap) using HMMer [33, 34] v3.3.2. Genes were annotated using using ‘anvi-run-ncbi-cogs’ for NCBI's Clusters of Orthologous Groups (COGs) database[35] and ‘anvi-run-kegg-kofams’ from the KOfam HMM database of KEGG orthologs (KOs)[36]. BAM files were profiled with “anvi-profile” and merged with “anvi-merge” for samples originating from the same participant. Automatic binning was performed by CONCOCT[37] v1.1.0 by specifying a maximum number of bins based on the estimated number of bacterial genomes computed from each sample's contigs. The maximum number of bins was set to 1/3 the number of expected genomes to limit the likelihood of fragmentation. Bins generated with CONCOCT were imported in the anvi'o profile database and were then manually refined and summarized to obtain fasta files of individual MAGs. Once manual binning of all samples from the same participant was complete, MAGs above 50% complete were dereplicated to generate a unique list of genomes using anvi'o and pyani v0.2.11. Representative genomes were chosen based on quality scores and clustered based on >95% ANI. The final list of MAGs were taxonomically assigned with GTDB-Tk (Genome Taxonomy Database Toolkit; v2.1.0[38, 39]) using classify_wf with default settings.

Pangenome Analysis. A Phocaeicola vulgatus MAG from the terminal ileum of CT12 was selected as a reference genome based on genome length. Open reading frames (ORFs) were identified through Prodigal for the P. vulgatus reference genome. Non-host reads from each participant (CT7, CT12, CT13, and CT14) were mapped onto the P. vulgatus reference genome by following anvi'o's metagenomics workflow using reference mode. For each sample and each gene present in the P. vulgatus reference genome, gene detection was calculated. Gene detection refers to the percentage of each gene sequence with at least 1× coverage. The average detection across all genes present within the P. vulgatus MAG was calculated and samples with a mean detection below 0.25 were removed from the final analysis. Pangenome visualization was performed in anvi'o interactive interface using the gene-mode flag with sorting of samples and genes by detection.

Analyses of SNVs. One TI sample from CT12 was split into 3 technical replicates prior to library preparation and each replicate was sequenced at a depth of 150M to 250M reads in sequencing batch 2 (see “Shotgun Sequencing”). SNV analyses across these samples were performed with anvi'o after dereplication (see “MAG Assembly”) using the command “anvi-gen-variability-profile” with a minimum mean coverage of 50× in all samples. Biological SNVs were classified as being present in all three technical replicates. SNVs present in only 1 or 2 technical replicates were classified as sequencing, PCR, or input errors. A threshold for minimum deviation from consensus was set based on the deviation required for all SNVs to be present in all technical replicates. This analysis was repeated for each MAG of interest (min mean coverage of 50×, n=6). After a threshold for minimum deviation from consensus was established, longitudinal samples from patient CT12 were analyzed using “anvi-gen-variability-profile” at the nucleotide, codon and amino acid level with the same minimum mean coverage of 50× and filtering out SNVs occurring in only one sample. The fixation index was computed using “anvi-gen-fixation-index-matrix” to describe the population structure between samples.

### Example 1: Host Depletion/Microbial-Enrichment Methodology (MEM) and Exemplary MEM Workflow

The host depletion and microbial enrichment methodology of the present disclosure has been developed to address in particular the challenges posed by samples including microbial compartments and related microbial NA in proportions relative to host compartments and related host NA.

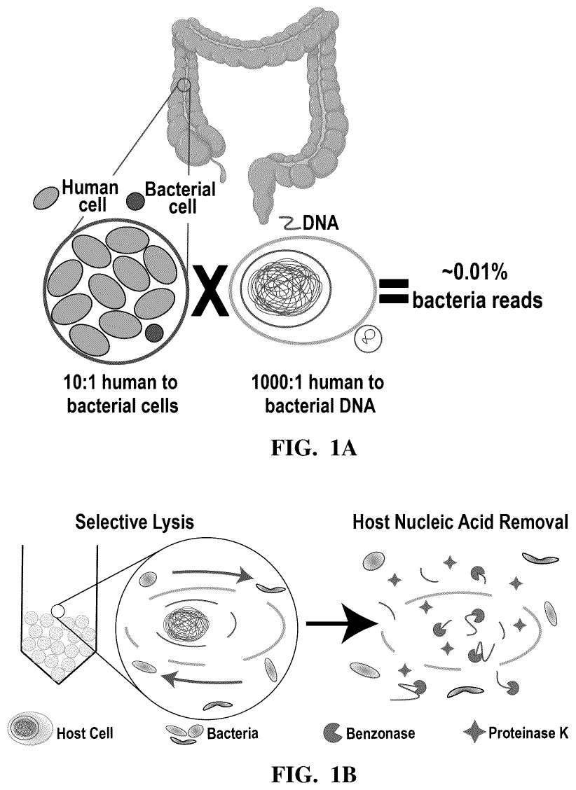

Reference is made in this connection to the exemplary schematics of FIG. 1A which shows a schematic illustrating the example of human colon. As shown in the schematic of FIG. 1A, a sample from human colon has the host cells and bacterial cells comprised in a proportion of 10:1, which converts in a proportion of host DNA to bacterial DNA of 1000:1 and in percentage bacterial reads of approximately 0.01%.

Such a percentage makes the isolation of microbial NA from a human colon sample as well as related detection and analysis particularly challenging because it preferably calls for a removal of the host compartments of at least 1000-fold as will be understood by a skilled person.

The MEM approach of the present disclosure addresses the challenge by performing a disruption of the sample in a manner that increases hot NA accessibility and then proceed with the degradation of the accessible host NA in a degradation step which can be performed simultaneously or sequentially as will be understood by a skilled person.

A schematic illustration of such an approach in a sample such as the human colon sample of FIG. 1A, is shown in the schematics of FIG. 1B. In particular, FIG. 1B shows how, after the disruption performed with the selective shearing of FIG. 1A, the use of a benzonase as an exemplary nuclease would serve the purpose of degrading the host NA made accessible by the lysis of the disrupting step. In the illustration of FIG. 1B the nuclease treatment is performed in combination with the optional use of enzymes such as proteinase K which can degrade proteins of the disrupted samples, such as histones of the host DNA, host membrane proteins and thus further increase accessibility of host NA as will be understood by a skilled person.

In MEM of the present disclosure, the combined selective disruption of host cells disruption and degradation of accessible host NA can be combined with many more steps directed to perform a further processing of the sample which is functional to the experimental design.

An exemplary workflow directed to isolation of target microbial NA is provided in FIG. 2 which schematically shows a workflow which comprises in addition to an initial disruption step (FIG. 2 first panel from the left) a nuclease degradation step (FIG. 2 second) panel from the left) which comprises placing the disrupted sample including the nuclease at a temperature to maximize activity of the nuclease (FIG. 2 third panel from the left) to increase the degraded amounts of accessible host NA possibly present in the disrupted sample, as will be understood by a skilled person.

In the schematics of FIG. 2 the heath degradation step is followed by a lysis of bacterial cells and extraction of related bacterial NA from the degraded sample (FIG. 2 fourth panel from the left) to obtain isolated bacterial NA.

Different workflow starting from the disrupted sample, nuclease treated degraded sample, and/or the heath treated degraded sample with or without proteinase K treatment, are shown in the additional exemplary workflows discussed in the following Example 2.

The results obtained in this example confirm the ones expected for the used configuration of beads and beads parameters according to the host depletion method of the present disclosure. Reference is made in this connection to the data reported in the tables below. Examples 0,4-13, 20,23,25-31 utilize this configuration of beads and bead parameters.
2R: 1.2 mm to 1.6 mm
Number of beads: 25%
Frequency: 40 Hz
Time: 30 s

| | | | | | |
|---|---|---|---|---|---|
| Radius of Bead (mm) | 0.6 | Frequency (Hz) | 40 | Bead Fill Volume % | 25.00% |
| Number of beads | 553 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 5E−10 | 0.06% |
| bacteria | 1 | 9E−16 | 1.00E+08 | 100 | 2E−09 | 0.25% |
| bacteria | 2 | 4E−15 | 1.00E+08 | 100 | 7E−09 | 1.00% |
| fungi | 5 | 2E−14 | 1.00E+08 | 100 | 5E−08 | 6.23% |
| fungi | 10 | 9E−14 | 1.00E+08 | 100 | 2E−07 | 24.86% |
| fungi | 20 | 4E−13 | 1.00E+08 | 100 | 7E−07 | 98.89% |
| virus | 0.1 | 9E−18 | 1.00E+09 | 100 | 2E−11 | 0.00% |
| archaea | 1 | 9E−16 | 1.00E+10 | 100 | 2E−09 | 0.25% |
| host | 8 | 6E−14 | 1.00E+04 | 1 | 1E−05 | 1592.89% |
| host | 15 | 2E−13 | 1.00E+04 | 1 | 4E−05 | 5578.13% |
| host | 50 | 2E−12 | 1.00E+04 | 1 | 5E−04 | 60763.89% |

| | | | | | |
|---|---|---|---|---|---|
| Radius of Bead (mm) | 0.8 | Frequency (Hz) | 40 | Bead Fill Volume % | 25.00% |
| Number of beads | 233 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 3E−10 | 0.04% |
| bacteria | 1 | 1E−15 | 1.00E+08 | 100 | 1E−09 | 0.14% |
| bacteria | 2 | 5E−15 | 1.00E+08 | 100 | 4E−09 | 0.56% |
| fungi | 5 | 3E−14 | 1.00E+08 | 100 | 3E−08 | 3.51% |
| fungi | 10 | 1E−13 | 1.00E+08 | 100 | 1E−07 | 14.00% |
| fungi | 20 | 5E−13 | 1.00E+08 | 100 | 4E−07 | 55.78% |
| virus | 0.1 | 1E−17 | 1.00E+09 | 100 | 1E−11 | 0.00% |
| archaea | 1 | 1E−15 | 1.00E+10 | 100 | 1E−09 | 0.14% |
| host | 8 | 8E−14 | 1.00E+04 | 1 | 7E−06 | 897.00% |
| host | 15 | 3E−13 | 1.00E+04 | 1 | 2E−05 | 3144.29% |
| host | 50 | 3E−12 | 1.00E+04 | 1 | 3E−04 | 34423.83% |

### Example 2: Additional Exemplary MEM Workflows

The MEM selective host depletion accompanied by possible MEM host NA degradation can be used in combination with various existing isolation, detection and/or analysis methodologies for microbial NA, microbial compartments and/or host NA.

In particular, various isolation, detection and/or analysis methodologies for microbial NA and/or microbial compartments can be advantageously performed from a MEM disrupted host sample and/or a MEM degraded host sample as will be understood by a skilled person upon reading of the present disclosure.

Exemplary workflows are discussed in Example 1. Additional exemplary workflows are discussed in this example in connection with the schematic illustration of FIGS. 3A, FIGS. 3B to 3D and FIGS. 3E to AG.

In particular, in the exemplary MEM workflow of FIG. 3A, the disruption resulting in disrupted sample (3) and the related degradation performed in the schematics with the exemplary heath treatment (4) are preceded by providing a sample (1) obtained from an environment of interest (1) and pretreating the sample with an optional pre-treatment (2) to removal of viscosity to assist in host removal and/or removal of inhibitors from downstream processing steps. Pretreatment can include the incorporation of a reducing agent, such as DTT, TCEP, and/or beta-Mercaptoethanol and/or the centrifugation for removal of sample inhibitors, such as those present in blood and/or urine.

The pretreatment (2) is however optional as will be understood by a skilled person upon reading of the present disclosure and also the degradation step (4) may not be included in some exemplary MEM workflows as will also be understood by a skilled person and schematically described in FIG. 3A.

In particular, in the schematic illustration of FIG. 3A MEM workflows are illustrated that do not require degradation (4) and can performed from the disrupted sample (3). In particular the disrupted sample (3) can undergo a variety of processing steps in the alternative or in combination which can be performed in parallel. For example, the host nucleic acids can be isolated through removal of the supernatant of the disrupted sample (FIG. 3A (8)). In addition or in the alternative, host nucleic acid can also be isolated (FIG. 3A (5)) and then detected and analyzed (FIG. 3A (6)) e.g. using qPCR, ddPCR, sequencing, and/or other methodologies identifiable by a skilled person upon reading of the present disclosure

In additional exemplary workflows shown in FIG. 3A, the disrupted sample (3) can also be processed to analyze the microbial compartment by isolation of the microbial compartment (7) which can then be analyzed through various techniques such as detection of microscopic features following amplification of replication competent compartments, staining, biochemical tests, chemical analytical tests, immunological identification and additional testing identifiable by a skilled person. In addition or in the alternative the isolated microbial compartments (7) can be analyzed through isolation of microbial nucleic acids (8) for microbial analysis (9), which may include qPCR, ddPCR, sequencing, single-cell and/or other methodologies as described in herein.

In an additional exemplary workflow shown in FIG. 3A, the results of the microbial analysis (9) and host analysis (6) can be combined in a host-microbial analysis interaction (10) at the level of community and/or subsets of a community and/or individual taxon interactions.

The microbial analysis (9) according to the exemplary workflow of Figure A1, performed on isolated microbial compartment (7) and/or isolate microbial NA (8) is further illustrated in the additional exemplary workflow of FIGS. 3B to 3D.

In particular, in the schematic illustration of FIG. 3B, it is shown that once an isolated microbial compartment (7) is obtained, various processing and analysis steps may be performed alone or in parallel. In particular, the whole cells can be utilized for single-cell analyses such as isolation of individual cells (FIG. 3B (9-1)) through FACS for sequencing, mass spectrophotometry, imaging, labelling, and additional techniques as described herein or identifiable by a skilled person. Additionally, single cells analysis can be performed by barcoding cell contents prior to nucleic acid extraction (FIG. 3B (9-2)) as described herein. While cells are intact, analysis of cellular organization can also be performed through NA proximity barcoding methods (9-3) as described herein. In addition or in the alternative, the analysis of FIG. 3B (9) can be performed following isolation of microbial NA (8). An optional step that can be performed on the microbial compartments prior to extraction is removal of additional extracellular host nucleic acid (FIG. 3B (11)). This step can be performed by mechanical approaches such as centrifugation and/or filtration and/or by a further degradation step (e.g. through nucleases as will be understood by a skilled person, to improve sequencing results of the microbes by removal of undegraded host NA as described in herein. Microbial compartments can then be extracted using either long or short processing methods (8-1) and (8-2) as described herein depending upon the preferred downstream applications. The extracted microbial NA (8) can be detected and/or analyzed e.g. by sequencing/QuantSeq/amplification. Such a detection can be part of a microbial analysis (9-4) or be focused on detection and/or analysis of target microbial NA per se as will be understood by a skilled person.

Additional method for detection/analysis of the microbial NA (9-5) to (9-11) are schematically illustrated in FIGS. 3C and 3D and can be used for a microbial analysis and/or for a detection and analysis of target microbial genes sequences and/or biomarkers.

In particular, in the schematics of FIG. 3C it is illustrated how samples processed with short read compatible extraction methods that yield fragments of low molecular weight can be processed for various analysis metrics outlined herein. (9-5) Samples processed with long-read compatible extractions (high molecular weight products) can be utilized with both short and long-read sequencing methodologies (9-6) as described in herein. After sequencing, sequencing files, such as fastq/fasta, can be generated containing the microbial genetic sequences (9-7). Sequences can be bioinformatically processed (9-8) for host removal using alignment to a reference genome as performed in Example 6.

The schematic of FIG. 3D shows, after host filtering, analysis of the microbial nucleic acids can be divided into DNA (9-9), RNA (9-10), and Multi-omic analysis (9-11) as described herein.

The NA isolation methods and NA detection/analysis methods can also be applied to host NA for the purpose of performing the host NA isolation (5) and host NA detection and/or analysis (6) as will be understood by a skilled person upon reading of the present disclosure.

An exemplary host NA workflow illustrating exemplary isolation, detection and analysis method for host NA is reported in FIGS. 3E to 3G.

In particular, in the illustration of FIG. 3E: exemplary methods are schematically shown to perform processing of Host Nucleic Acids. After removal of the microbial compartment, extracellular nucleic acids (5-0) can be derived from host, dead microbes, extrachromosomal elements, and additional sources in a disrupted sample identifiable by a skilled These nucleic acids can be isolated using high molecular weight techniques (5-2) or short molecular weight techniques (5-1). If high molecular weight techniques (5-2) are desired the disrupting step is performed by selecting the MEM bead sizes and number, as well as related speed, duration and frequency of the disrupting, to retain nucleic acids above 10 kbp as will be understood by a skilled person upon reading of the disclosure.

In the illustration of FIG. 3F Samples processed with short read compatible extraction methods that yield fragments of low molecular weight can be processed for various analysis metrics including Sequencing QuantSec and amplification (6-1) outlined herein. Samples processed with long-read compatible extractions (high molecular weight products) can be utilized with both short and long-read sequencing methodologies (6-21) and (6-3) as described in herein. After sequencing, sequencing files, such as fastq/fasta, will be generated encoding sequences. (6-4)) Sequences can be bioinformatically processed for non-host read removal by alignment to a reference genome. (6-5)

In the illustration of FIG. 3G shows that after non-host filtering, analysis of the host nucleic acids (6-5) can be divided into DNA (6-6), RNA (6-7), and Multi-omic analysis (6-8) as described herein.

A skilled person will understand each and all the techniques and methodologies illustrated in FIGS. 3E to A3G can be performed advantageously alone or in various combination using a MEM disrupted sample and/or a MEM degraded sample in the sense of the disclosure in view of the increased enrichment in microbial compartment and/or microbial NA resulting from MEM processing of the sample.

A skilled person will also understand that methodologies and approaches directed to detection and/or analysis of microbial compartment and/or target microbial NA which are based on detection and/or analysis of a target plurality of microbial compartments (e.g. microbial community such as a microbiome) and/or a target plurality of microbial NA (e.g. metagenomic analysis such as metagenomic data assembly) will particularly benefit from the use of MEM processed samples which are known or expected to provide an increased resolution as shown by the representative examples reported below.

### Example 3: MEM Parameters

The use of bead beating for the selective mechanical disruption of compartment samples (which may contain some combination of bacterial, fungal, archaeal, and mammalian cells, as well as viruses) can be tuned based on: 1) decreasing the number (or increasing the size) of beads to preferentially target larger compartments types (host cells), or by 2) decreasing the bead beating severity (i.e. by decreasing the duration of beating, bead beating frequency, bead beating speed, etc.) to preferentially lyse softer compartments.

During sample bead beating, temperatures can range from −100 C, if samples are flash frozen prior to homogenization, to temperature no higher than 60 C. Typically the protocol can be at physiological temperature of the target microbes, or at 37 C, whichever is lower. The protocol can be carried out at further reduced temperatures to reduce microbial degradation.

Decreasing the number of beads in a bead container such as a beating tube (which can be achieved by increasing the size of the beads while approximately maintaining their total volume) will decrease the number of collisions that occur during a bead beating cycle for a given bead beating duration and frequency. One can estimate the effective volume of the sample that is being bead beaten by considering the finite space between two colliding beads in which the gap between the two beads is smaller than the size of the compartments.

The crushed volume between two collided beads can be approximated as a spherical cap geometry as shown in FIG. 4, where M_{d} is the characteristic size of the microbial compartment (in) being disrupted (used here for as an exemplary compartment of the sample being crushed for exemplary purposes), R is the radius of the bead (in), h is the height of the cap (m), and x is the radius of the cap (m).

The dimensions in the collision of two beads of FIG. 40, can be represented as spheres with a cap of radius x and height h,

$$R = \frac{{x}^{2} + {h}^{2}}{2 h}$$

$$x = \sqrt{2 Rh - {h}^{2}}$$

Per the diagram, the height of the cap h can be estimated in terms of the microbial compartment size:

$$h = \frac{1}{2} {M}_{d}$$

The following general considerations apply:

This estimate assumes that any deformation of a cell (compartment) caught between the two beads would lead to lysis. In reality, cells may tolerate some level of deformation, perhaps 10-20%, and therefore h would be smaller.

This estimate assumes that compartments are caught between beads randomly.

Since crushed volume is set by a pair of beads, note that this equation assumes that each bead makes two contacts, so number of crushed compartments is equal to the number of beads. At high concentrations of beads, as is typical in bead beating experiments, this contact number could be higher than 2 per bead. However, perfect packing would not be expected under these conditions, as beads are more likely to form an irregular (possibly jammed) packing structures, therefore 2 contacts per bead, while possibly a bit low, is a reasonable estimate.

Effective tissue disruption and microbial cell lysis relies on energetic collisions of beads. In a liquid, the collisions are cushioned by the viscous forces acting on the moving beads, which slow down the movement of the beads. The Stokes number measures the relative effects of viscous relaxation time and a characteristic time based on collision velocity and bead radius. Gers et al (Gers et al, (2010). Numerical modelling of grinding in a stirred media mill: Hydrodynamics and collision characteristic. Chemical Engineering Science. 65. 2052-2064. 10.1016/j.ces.2009.12.003.) estimate the Stokes number of beads colliding in a mill chamber as

$$St = \frac{R \cdot ρ \cdot Δ U}{9 μ} ,$$

where R is the diameter of the beads (m), p is the density of the beads in

$$\frac{kg}{{m}^{3}} , Δ U$$

is the relative velocity of the beads (in other words, the velocity at which they are approaching each other) in

$$\frac{m}{s} ,$$

and μ is the viscosity of the particle suspension in Pa·s.

For the MEM example for 1.4 mm beads in a suspension with viscosity of water, with bead beating velocity of 4.5 m/s, if we take the collision velocity to be half of beat beating velocity

$$Δ U = 2.25 \frac{m}{s} ,$$

density of beads

$$ρ = 4500 \frac{kg}{{m}^{3}} ,$$

R=1.4-10⁻³ m, and =1-10⁻³ Pa·s, we estimate that St=1,580. This indicates that viscous forces do not dominate this collision.

However, the same calculation performed on a high viscosity mucus sample (e.g. saliva sample with high viscosity mucus), where by maintaining

$$ρ = 4500 \frac{k g}{{m}^{3}}$$

and adjusting the viscosity such that μ=1 Pa·s, a St=1.58 can be estimated.

The number falling to around 1 indicates that viscous forces significantly cushion the bead collisions, and predicts bead beating to be less effective in such viscous samples. When 0.1 mm beads are used in the same experiment, Stokes number is about 113 in the non-viscous solution and 0.113 in the viscous solution. Therefore, for such highly viscous samples, reducing their viscosity (e.g. by addition of DTT as done with some saliva samples) would significantly increase Stokes number and would facilitate bead beating.

For bead beating to be effective, Stokes number should be maintained above 1, preferably above 10.

In order to ensure that bead beating occurs, one wants to maximize the likelihood there are collisions between beads and/or between beads and the wall. In a cyclical bead beating protocol, this means that the bead velocity is high enough, and the distance between beads is short enough, that the maximum distance a bead can travel during one cycle is greater than the distance between beads or between the walls of the tube (so that the beads in the tube are able to collide with another bead or a wall). One way to think about this for a cyclical process would be as follows.

Assuming a tube with volume of beads+liquid, V (m³), filled with N beads that are uniformly dispersed, one can assume that the centers of each bead have a separation, r (meters), given by the equation

$$r = \frac{1}{{n}^{\frac{1}{3}}}$$

(this is equation is based off the assumption that each particle takes up a uniform per particle volume) where n (number of beads/m³) is the particle density of beads in the tube (n=N/V). [40]

From this distance it is possible to determine how far away the edges of the beads are in this situation by subtracting from this distance 2 times the radius of the beads, R (meters). Next, we can determine the maximum distance L (meters) a bead is able to travel during one cycle of bead beating, (the part of the bead beating before the beads substantially change direction) at velocity, vb (m/s), and frequency (Hz) using the equation

$$L = {v}_{b} \cdot \frac{1}{f r e q u e n c y} .$$

In this case, vb (m/s) is the total velocity of the beads if collisions with the walls of the container are of interest, or inter-bead velocity if bead-to-bead collisions are to be considered.

If L is much larger than r, distance between the beads, or distance between the walls of the tubes, then we know the bead is able to collide with another bead and/or a wall of the tube. For example, using 161 beads 1.4 mm in diameter in 0.996 mL of total volumes of beads and solution, the average distance between beads is about 4.36*10⁻⁴ m or 0.436 mm. With 4.5 m/s velocity and 35 Hz cycle frequency, beads would be able to travel an L=0.129 m (12.9 cm) distance per cycle if they were not constrained by walls and collisions. Under these conditions, clearly the distance between beads and the tube dimensions are much smaller than the travel distance, therefore beads will collide and bead beating can proceed. Even when relative velocities of beads are much less than the total velocities (e.g. 5% relative velocities was estimated in Gers et al, (2010). Numerical modelling of grinding in a stirred media mill: Hydrodynamics and collision characteristic. Chemical Engineering Science. 65. 2052-2064. 10.1016/j.ces.2009.12.003.), the relative velocity of beads would be 0.225 m/s traveled distance L would be 28.5 mm, still larger than the spacing between beads.

Therefore, when frequency of bead beating cycles and velocity of the beads can be estimated, in preferred embodiments the distance L that beads can travel in a bead beating cycle should be larger than at least one of the a) distance between beads r and b) the distance between the walls of the bead beating container.

Thus, the above calculations can serve as a valuable starting point and reasonable level of experimentation may be used for the any necessary fine-tuning of conditions as follows.

To calculate the crushed volume (V_{crushed} in m³) for a characteristic compartment type, the following steps are followed:
1) Calculate the cross-sectional area of the spherical cap:

$${A}_{cross section} = π {x}^{2}$$

$${A}_{cross section} = π ( 2 R \frac{1}{2} {M}_{d} - {( \frac{1}{2} {M}_{d} )}^{2} )$$

2) Calculate the volume of the cylinder of radius x and height M_{d}.

$$V = {A}_{cross section} {M}_{d}$$

$$V = {M}_{d} π ( 2 R \frac{1}{2} {M}_{d} - {( \frac{1}{2} {M}_{d} )}^{2} )$$

Calculate the two cap volumes:

$${V}_{cap} = \frac{π {( \frac{1}{2} {M}_{d} )}^{2}}{3} ( 3 R - \frac{1}{2} {M}_{d} )$$

$$2 {V}_{cap} = \frac{2 π {( \frac{1}{2} {M}_{d} )}^{2}}{3} ( 3 R - \frac{1}{2} {M}_{d} )$$

3) Finally, crushed volume for a given compartment (V_{crushed,compartment}) with size M_{d} is the volume calculated in (3) subtracted from the cylinder approximation from step (2):

$${V}_{crushed . compartment} = {M}_{d} π ( 2 R \frac{1}{2} {M}_{d} - {( \frac{1}{2} {M}_{d} )}^{2} ) - \frac{2 π {( \frac{1}{2} {M}_{d} )}^{2}}{3} ( 3 R - \frac{1}{2} {M}_{d} )$$

As described above, for a given compartment, the effective crushed volume of the solution in a bead beating protocol (V_{effective,compartment}) given a set of bead beating conditions (bead number, bead collision frequency per bead freq_coll (s⁻¹), beating duration, bead size) can be estimated with the following equation:

$${V}_{effective , compartment} [ {m}^{3} ] = \frac{beads [ \# ] * {freq}_{coll} [ {s}^{- 1} ] * duration [ s ] * {V}_{crushed , compartment} [ {m}^{3} ]}{{E}_{adj , compartment}}$$

In determining the Veffective compartment the relative hardness of target compartments is accounted for with a variable E_adj, compartment (for either microbe or host) based on the Young's modulus of that compartment. For a compartment with a Young's modulus greater than or equal to 1e7 Pa, as is the case for many microbes, E_adj is 100. For a compartment with a Young's modulus less than 1e5 Pa, as is the case for many host cells, E_adj is equal to 1.

The Veffective compartment is determined for Stokes number {Gers, 2010 #328}

$$St = \frac{R \cdot ρ \cdot Δ U}{9 μ} ,$$

where R is the diameter of the beads (m), p is the density of the beads in

$$\frac{k g}{{m}^{3}} , Δ U$$

is the relative velocity of the beads (in other words, the velocity at which they are approaching each other) in

$$\frac{m}{s} ,$$

and μ is the viscosity of the particle suspension in Pa·s. ΔU is taken to be 10% of the total bead velocity. The total bead velocity can be provided by the instrument or calculated using manufacturer's instructions by multiplying the instrument frequency and rotor dimensions by the equation: ν=dπƒ, where d is the rotor diameter, ƒ is the frequency. Furthermore, if only speed is controlled by the user of the instrument, the same approach may be used to estimate frequency from the speed. When beat beating is performed by a repetitive movement, the frequency of this repetition can be used as the frequency parameter f.

For example, in the MEM example for 1.4 mm beads in a suspension with viscosity of water, with collision velocity to be

$$Δ U = 0.225 \frac{m}{s} ,$$

density of beads

$${ρ}_{=} 4500 \frac{k g}{{m}^{3}} ,$$

R=1.4·10⁻³ m, and μ=1·10⁻³ Pa·s, we estimate that St=158. This indicates that viscous forces do not dominate this collision. However, the same calculation performed on a high viscosity mucus sample (e.g. saliva sample with high viscosity mucus), where maintaining

$$ρ = 4500 \frac{k g}{{m}^{3}}$$

and adjusting the viscosity such that μ=0.1 Pa·s, one estimates that St=1.58. The number falling to around 1 indicates that viscous forces significantly cushion the bead collisions, and predicts bead beating to be less effective in such viscous samples. When 0.1 mm beads are used in the same experiment, Stokes number is about 11 in the non-viscous solution and 0.011 in the viscous solution. Larger beads have higher Stokes number and can be used to disrupt more viscous samples. Preferably, for such highly viscous samples, reducing their viscosity (e.g. by addition of DTT as we have done with some saliva samples) would significantly increase Stokes number and would facilitate bead beating. Note that higher Stokes number does not always mean better, as excessive bead beating could lead to undesirable effects such as degradation of microbial compartments.

Above, the frequency of collisions (freq_coll, s−1) per bead can be estimated by taking the product of the bead beating frequency (set by the user, in Hz) and the number of collisions per bead beating cycle per bead (estimated to be 300). It should be understood that the collision frequency (freq_coll, s−1) per bead is the effective collision frequency, which determines the crushed volume per bead beating cycle. Beads may undergo multiple collisions per cycle. Beads may also undergo various types of collisions. For example, when upon collision beads slide along each other's surface, the increased crushed volume V_{crushed, sliding} that is created by such collisions is reflected in the equation by increasing the effective collision frequency (freq_coll, s−1) proportionally to the ratio of the crushed volume of a sliding collision to the crushed volume of a direct non-sliding collision, given by V_{crushed, sliding}/V_{crushed}.

The percentage of the total sample volume that is bead beaten for a given compartment (BB%_{compartment}) can be estimated by comparing V_{effective,compartment} with Vₛₐₘₚₗₑ, or the total sample volume in the bead tube. This percentage is useful for comparisons of different bead beating conditions because it does not saturate−a value far above 100%, for example 1,000%, indicates efficient disruption.

$$\% {BB}_{c o m p a r t m e n t} = \frac{{V}_{effective , compartment}}{{V}_{s a m p l e}}$$

The protocol is expected to optimize bead beating frequency, duration, and bead size such that the larger host compartments can be preferentially targeted for disruption while smaller microbial compartments are preserved.

Also the protocol is expected to result in selective crushing of compartments based on difference in elastic modulus preferentially targeting host compartments (typically having an elastic modulus equal to or lower than 10\hat{ }5 Pa) while preserving microbial compartment having an elastic modulus equal to or higher than 10\hat{ }7 Pa. this will apply even for large microbial compartments (having an Md higher than 5 um).

In selecting the appropriate lysis matrix bead material type, a material with a Young's modulus of at least 10 GPa, preferably at least 50 GPa is selected for the effective lysis of animal compartments. Examples include but are not limited to:

| Material | Young's modulus (GPa) |
|---|---|
| Silica | 66.3-74.8 |
| Zirconia | ~200 |
| Zirconium | 94.5 |
| Stainless steel | 190-203 |
| Zirconium silicate (Zircon) | 400 +/− 15.3 or 336 +/− 8.7 |
| | (depending on plane |
| | for tetragonal zircon) |
| Yttria-stabilized zirconium | ~205 |
| oxide (Zirconium oxide is | |
| the same as zirconia) | |

Selection of lysis beads preferably is directed the use of spherical beads, or nearly-spherical beads which minimize the number of edges. Garnett beads, or beads of angled, jagged, or sharp shapes, are less preferred. Selection of lysis beads preferably is directed the use of fragmentation resistant beads.

To preferentially preserve microbial compartment (with Md of up to 5 um) and lyse animal compartments (with Md of 8 um or greater), the % BBₐₙᵢₘₐₗ should exceed 200%, ideally 1000%, while maintaining % BB_{microbial} to be less than 50%, ideally, less than 10%, as will be understood by a skilled person and will understand upon reading of the present disclosure and of the Following Examples.

### Example 4 Bacterial Loads and Bacterial NA Detected with of MEM in Comparison with Known Host-Depletion Methods

The MEM sample processing of the disclosure was tested in comparison with existing methodologies for ability to perform microbial compartments and microbial nucleic acid isolation and analysis (see FIG. 3A (7), (8) and (9)).

In particular To quantify how MEM affects microbial community composition and relative abundances of individual taxa, frozen mouse fecal samples were first use. For validation, fecal samples were chosen instead of a contrived community to characterize microbial impacts on a wider range of unique taxa and on a continuum of abundances. Additionally, contrived communities still require an extracted control due to variation in extraction kit efficiency. Mouse fecal samples do not typically require host-depletion because they have low levels of host contamination (more than 90% of the DNA biomass originates from non-host cells). Thus, the high biomass of microbial cells in such samples makes them ideal for characterizing the impact of different host-depletion methods on the microbial community composition.

Beads were selected for MEM processing to selectively target host cells vs microbial compartments possibly present in the sample. As a consequence the beads were chosen that are more than an order of magnitude larger (1.4 mm) than beads usually typically used for microbial lysis (0.1 mm) to create high mechanical shear stress on the large host cells while leaving small bacterial cells intact in accordance with MEM processing. Next, Benzonase was added to degrade accessible extracellular nucleic acids including NA from dead lysed microbes. Proteinase K further lyses host cells and degrades host histones for DNA release.

To compare host-depletion by MEM with existing methods, we selected three published methods that utilize different cell-lysis approaches: MolYsis, QIAamp, and lyPMA. All host-depletion methods are composed of two main steps: selective lysis followed by NA removal. QIAamp lyses cells lacking a cell wall through a weak detergent, saponin.

In particular, three stool pellets from three different mice were freshly collected by gently handling the mice. Pellets were transferred to clean microfuge tubes with sterile tweezers. Samples were stored on ice for up to 30 min before being processed in the laboratory. A total of 1 mL of saline was added to each stool pellet and the samples were homogenized by pipetting. Homogenized stool samples were diluted 3-fold in saline and 100 μL from each diluted stool sample was processed with various host-depletion methodologies. Samples for MEM treatment were placed into 2-mL 1.4-mm ceramic bead-beating tubes (Lysing Matrix D from MP Biomedical, Cat #116913050-CF) with a maximum volume of 400 μL.

For solid sample types (stool and intestinal tissue), up to 400 μL of saline (0.9% NaCl, autoclaved) was added into the bead-beating tube. Samples were homogenized using FastPrep-24 (MP Biomedical Cat #116004500) for 30 sec at 4.5 m/sec and then immediately placed on ice. A total of 150 μL of homogenized tissue was removed and placed into a clean microfuge tube containing 10 μL of buffer (100 mM Tris+40 mM MgCl₂, pH 8.0 and 0.22 μm sterile filtered), 33 μL of saline (0.9% NaCl, autoclaved), 2 μL of Benzonase Nuclease HC (EMD Millipore Cat #71205), and 5 μL of Proteinase K (NEB Cat #P8107S).

Samples were mixed lightly by manually pipetting up and down 5-10 times and spun briefly to pool (1,000×g for 5 seconds). Tubes were placed on a dry block incubator for 15 min at 37° C. with shaking at 600 rpm. Samples were then pelleted at 10,000×g for 2 min and the supernatant was removed and discarded. Pellets were resuspended in 150 μL of PrimeStore MTM (Longhorn), a transport medium, to inactivate residual enzymatic activity and stored at −80° C. until nucleic-acid extraction.

A first set of results on bacterial loads obtained by MEM and the tested known methodologies is reported in in FIG. 5A. in particular FIG. 5B shows bacterial loads from mouse stool samples treated with five different host-depletion methods. QIAamp lyses cells lacking a cell wall through a weak detergent, saponin. MolYsis selectively lysis the more fragile mammalian cells through exposure to a weak concentration of guanidinium. lyPMA lyses mammalian cells through osmotic lysis and uses photochemistry to render DNA accessible to PMA non-amplifiable. Loads were normalized to the control (no host depletion) stool samples (N=3; error bars are 95% CI centered on the mean).

Elutions from the sample treated to provide the results shown in FIG. 5A were sequenced as described in “Quant-Seq”. in particular, nucleic acids were isolated following Qiagen's AllPrep PowerViral DNA/RNA Kit (Cat #28000-50). Samples were homogenized in 0.1 mm glass beads for 1 min at 6 m/s using FastPrep-24 (MP Biomedical Cat #116004500) to ensure complete microbial lysis. Microbial characterization and quantification was performed using the quantitative sequencing (“Quant-Seq”) pipeline described previously. [41]

The results reported in FIG. 5B, show Empirical cumulative distribution function (ECDF) of 16S rRNA gene amplicon sequencing results from mouse stool samples normalized to the control stool samples (N=3). Curves shifted to the left of the control indicate a greater percentage of taxa with lower abundance than the control samples following host depletion.

In view of the results shown in FIGS. 5A and 5B, on homogenized stool samples, similar losses in microbial recovery were observed across all five host depletion protocols compared to a control, untreated sample (see FIG. 5A). MEM induced on average 29% (SD: 3.8%) bacterial loss, which falls within the expected fraction of 10-50% of dead microbial cells in stool [74]. To characterize how MEM and the other host-depletion methods affect the microbiome at a taxonomic level, we next performed quantitative 16S rRNA gene sequencing [42] on the mouse fecal samples (N=3). By comparing paired host-depleted and control samples, we found that lyPMA and QIAamp induced the most total bacterial losses whereas MolYsis and QIAamp induced the least uniform bacterial losses, with some taxa dropping more than 100-fold (FIG. 5A and FIG. 5B).

Accordingly the data confirmed that MEM induced minimal losses in the microbial community; more than 90% of genera showed no significant difference in relative abundance between MEM and control samples (paired t-test, two-sided, P=0.05). Additionally, all taxa that were detected in the control samples were also detected in the MEM-treated samples, whereas MolYsis and QIAamp host-depletion methods resulted in some taxa drop-out.

In particular, the data discussed in this Example support the conclusion that because MEM selectively lyses host cells based on cell size differences, MEM introduces lower bacteria bias compared with chemical lysis alternatives (MolYsis and QIAamp) where degree of lysis may differ based on bacterial cell wall/membrane structures. Additionally, MolYsis showed increased bacterial recovery, likely due to the additional mutanolysis step.[43, 44]

The results obtained with this detection and analysis methodologies for microbial compartments and/or NA isolation and are expected to be representative of performance with other methodologies such as those reported in Example 1 and Example 2 in view of the impact of the MEM enrichment demonstrated in these examples in other methodologies as will be understood by a skilled person.

### Example 5 Host NA Detected with of MEM in Comparison with Known Host-Depletion Methods

The MEM sample processing of the disclosure was tested in comparison with existing methodologies also for ability to perform host NA isolation and analysis (see FIG. 3 (5), and (6)).

In order to determine how effectively MEM and the other host-depletion methods removed host material, the amount of host DNA remaining after each host-depletion method was quantified on three additional sample types: liquid, soft tissue, and hard-tissue samples in which the host DNA made up as much as 99.9% of the total biomass.

In particular mouse intestinal mucosal scrapings were also retrieved as a representative of soft tissue and remaining mouse genomes were quantified (N=3; biologic replicates from one mouse). The mouse intestinal scrapings isolate the epithelial layer with mucosa-associated bacteria as will be understood by a skilled person.

Rat colonic sections were used as a representative hard tissue (including connective tissue, muscle, and mucosa) and remaining rat genomes were quantified (N=3; biologic replicates from one rat). Rat colonic sections are more anatomically similar to a human intestinal biopsy.

Human saliva samples were used as a representative liquid tissue. Healthy adult volunteers were instructed to pool saliva in their mouths and spit 2 mL of saliva, ignoring bubbles when estimating volume, into a 15 mL conical tube through a plastic funnel. Prior to undergoing MEM, saliva samples underwent a DTT (dithiothreitol) pre-treatment for MEM+DTT condition. Saliva was mixed at a 1:1 ratio with fresh DTT (10 mM DTT in 1×PBS, Sigma Aldrich Cat #43815), vortexed briefly, and incubated for 1 min at room temperature before undergoing host-depletion processing. Remaining human genomes in fresh human saliva were quantified after treatment with each host-depletion method and in untreated controls (N=4 biological replicates for Control with N=2-3 technical replicates. N=4 biological replicates for MEM with N=3 technical replicates for one biological replicate. N=3 technical replicates for MEM+DTT).

In both processed mouse fecal sample and human saliva sample the remaining host DNA in the disrupted sample was quantified through ddPCR. Host load present in extracted DNA was characterized by droplet digital PCR (ddPCR) of a single-copy gene. For human saliva, the gene EIF5B was amplified based on primers found from literature (F: GCCAAACTTCAGCCTTCTCTTC (SEQ ID: 1) and R: CTCTGGCAACATTTCACACTACA (SEQ ID: 2)). For samples originating from rodents, the gene Cyp8b1 was amplified based on primers found from literature (F: GGCTGGCTTCCTGAGCTTATT (SEQ ID: 3) and R: ACTTCCTGAACAGCTCATCGG (SEQ ID: 4). Samples were amplified on the C-1000 thermocycler (Bio-Rad Cat #1851196) and quantified using the QX200 droplet digital PCR system (Bio-rad Cat #1864001). The concentrations of the components in the ddPCR mix used in this study were as follows: 1×QX200 ddPCR EvaGreen SuperMix (Bio-Rad Cat #1864035), 500 nM forward primer, and 500 nM reverse primer for a total reaction volume of 25 μL. Thermocycling was performed as follows: 95° C. for 5 min, 40 cycles of 95° C. for 30 sec, 60° C. for 30 sec, and 68° C. for 30 sec, followed by a dye-stabilization step at 4° C. for 5 min and 90° C. for 5 min. All ramp rates were 2° C. per sec. Reported genomes remaining refers to the abundance of this single-copy gene present in 1 μL of elution.

The results of these tests are reported in FIGS. 5C, FIG. 5D and FIG. 5E as discussed below,

In saliva, all methodologies enabled some host removal. Following MEM treatment, over 40-fold depletion of host was achieved (FIG. 5C). The addition of DTT pre-treatment, which was added due to the high mucin content of saliva, slightly increased host removal by MEM in some participants (FIG. 15D-E). The results shown in FIG. 5D for the existing methods, indicated that even if PMA appeared similarly effective at host removal as MEM with the DTT pretreatment, was difficult to use predictably because the stoichiometric nature of the method can result in large microbial losses when host levels are lower than expected (FIG. 40). Additionally, MolYsis showed increased bacterial recovery, likely due to the additional mutanolysis step.

The illustration of FIG. 5E show that the mucosal scraping samples were efficiently host-depleted by MEM and some of the published methods. MEM, MolYsis, and QIAamp all showed around 1000-fold depletion of host with QIAamp showing slightly greater host-removal (MEM had an average 1,600-fold depletion [SD: 170]). lyPMA performed poorly on the soft tissue sample because this method relies on UV-activated crosslinking making it incompatible with opaque sample types.

The illustration of FIG. 5E shows the result of MEM processing in hard tissue in comparison with the MolYsis, and QIAamp. For this set of experiments lyPMA was excluded from the hard-tissue experiment due to its poor performance on soft tissue (see FIG. 5D). The illustration of FIG. 5E shows that MEM was the only method that worked on the solid-tissue sample type. MEM treatment resulted in almost complete removal of the host DNA (3,600-fold removal; SD: 1,500), whereas MolYsis and QIAamp host DNA levels after treatment were similar to the control as would be understood by a skilled person.

A host removal after MEM treatment of the same order of magnitude is expected in similar sample types, such as other bodily fluids or soft tissue types from various animals. In particular it is expected that extraction with a bacterial enzymatic lysis step may improve bacterial recovery after MEM, as seen with MolYsis. It is also expected that any bias MEM induced to specific bacterial and archaea taxa can be extrapolated to most other bacterial and archaea taxa as will be understood by a skilled person upon reading of the present disclosure.

### Example 6 Shotgun Sequencing of MEM-Treated Saliva and Stool Shows Enrichment of Microbial Reads and Unbiased Species Identification

The utility of MEM for applications of shotgun metagenomics as a methodology suitable in microbial analysis and microbial NA analysis (see Example 2 Figure A1 (9)) when directed to a target plurality of microbes and/or microbial NA) was investigated.

Due to biomass limitations, accurate characterization of microbial communities through shotgun sequencing of control (not host-depleted) samples is not feasible on intestinal biopsies. Thus, we first used saliva and stool samples to investigate potential biases associated with MEM treatment within the microbial fraction.

Stool samples were processed as described in Example 4 for control and MEM samples. Saliva samples were processed as described in Example 5 for control, MEM, and MEM+DTT samples. Extracted DNA was prepared for sequencing using Illumina DNA Prep (Cat #20018704). A maximum input of 500 ng of DNA was used for library prep. Finished libraries were quantified through Qubit's dsDNA High Sensitivity assay and a High Sensitivity D1000 TapeStation Chip (Agilent Cat #5067-5585, #5067-5584). If additional peaks were seen at 45 bp or 120 bp, indicating the presence of primer dimers or adapter dimers, we performed an additional clean-up step with AMPureXP beads (Beckman Coulter, Cat #A63880) at a ratio of 0.8:1 of beads to library volume. For quantification, finalized libraries were amplified on the CFX-96 qPCR (Bio-Rad Cat #1855196) with primers targeting the Illumina adapter sequence (F: AAT GAT ACG GCG ACC ACC GA (Seq ID: 5) and R: CAA GCA GAA GAC GGC ATA CGA (Seq ID: 6)).

Libraries were diluted 1:40,000 in NF water prior to amplification to fall within the range of KAPA standards concentrations (Roche Cat #07960387001) for quantification. The concentrations of the components in the qPCR mix used were as follows: 1× SsoFast EvaGreen Supermix (Bio-Rad Cat #1725201), 125 nM forward primer, and 125 nM reverse primer for a total reaction volume of 10 uL. Thermocycling was performed as follows: 95° C. for 5 min, 40 cycles of 95° C. for 30 sec and 60° C. for 45 sec, followed by a melt-curve step at 95° C. for 15 sec, 50° C. for 15 sec, 70° C. for 1 sec, and 95° C. for 5 sec. Pooled samples were quantified through Qubit's dsDNA High Sensitivity assay and a High Sensitivity D1000 TapeStation Chip before submitting the samples for sequencing. Sequencing was performed by Fulgent Genetics using the Illumina NovaSeq6000 platform.

Samples were demultiplexed on the NovaSeq6000 and raw fastq files for read 1 and read 2 were provided along with fastqc files for each sample. Sequencing data was processed using the KneadData v0.10.0[22]. Through KneadData, quality control (QC) and host removal were performed with Trimmomatic v0.39[23]

Human saliva samples were aligned to KneadData's default human reference genome (a combination of hg38 human genome reference (GenBank assembly accession #GCA_000001405.29) and small contaminant sequences) and aligned reads were removed. Samples acquired from mice stool were processed using the reference genome GRCm39 constructed from C57BL/6J mouse-strains (GenBank assembly accession #GCA_000001635.9). After bioinformatic host removal, the percentages of host reads were calculated by dividing reads remaining after host filtering by the total reads that passed QC.

The results of these experiments are illustrated in FIGS. 6A to 6E.

In particular FIG. 6A, show results of experiments where the percentages of non-host reads in control and MEM-treated mouse stool samples were calculated bioinformatically through alignment to a mouse reference genome (N=3; error bars are 95% CI centered on the mean) The results reported in FIG. 6A confirmed that MEM treatment enabled reliable reduction in host reads in mouse stool.

FIG. 6B, show results of experiments where species-level taxon relative abundances were plotted for control and MEM-treated mouse stool and overlaid on a dashed line showing 1:1 correlation. The results reported in FIG. 6B show that there was a high correlation between the relative abundances of bacterial taxa in control and MEM-treated samples (R²=0.93 for stool). For stool, a high correlation between the relative abundance of species in control vs MEM-treated samples showed that MEM did not substantially alter microbiome composition.

FIG. 6C, show results of experiments where Shotgun sequencing performed on control and MEM-treated fresh human saliva. The results reported in FIG. 6C show that the percentages of non-host reads were calculated bioinformatically through alignment to a human reference genome. One saliva sample was evenly split nine ways for this comparison (N=3). DTT pre-treatment in saliva improved host removal roughly 10-fold.

FIG. 6D, show results of experiments where species-level taxon relative abundances were plotted for control and MEM-treated fresh human saliva and overlaid on a dashed line showing 1:1 correlation. An additional DTT pre-treatment was performed prior to MEM treatment for a subset of MEM-treated samples (MEM+DTT) (N=3). The results reported in FIG. 6D show that there was a high correlation between the relative abundances of bacterial taxa in control and MEM-treated samples (R²=0.90 for both MEM and MEM+DTT in saliva; with R²=0.93 for taxa above 0.1% relative abundance). For saliva, a high correlation was observed in the relative abundances of microbial taxa between the control sample and both the MEM and MEM+DTT treatments (FIG. 6D,). The correlation was less pronounced for low-abundance taxa with enrichment of specific species in MEM-treated samples and was investigated more quantitatively by comparing the coefficient of variation (CV) across samples for low-abundance species as shown in FIG. 6E below).

FIG. 6E, show results of experiments where Coefficient of variation was plotted against relative species abundance and colored based on treatment types the taxa were detected in. Each point represents a species; grey, dark-blue and light-blue points indicate taxa that were present in all three treatments (control, MEM, and MEM+DTT). The results reported in FIG. 6E show that MEM/MEM+DTT only (red points) indicate the 10 taxa found only in the MEM-treated samples. Control only (orange point) indicates the single taxon that was found only in the control samples, which was identified as Haemophilus. MEM-treated saliva samples had lower CV (50% CV, 95% CI), indicating better replicability compared with untreated controls. Additionally, MEM improved quantification of low-abundance species (>0.05% relative abundance), enabling detection of an additional 10 species that were undetected in the control.

The above results confirm that MEM treatment enabled reliable reduction in host reads across mouse stool and human saliva samples (FIG. 6A, 6C). DTT pre-treatment in saliva improved host removal roughly 10-fold (FIG. 6C).

In general following comparison of the results of shotgun sequencing between the control and MEM-treated samples. There was a high correlation between the relative abundances of bacterial taxa in control and MEM-treated samples for both stool and saliva (R²=0.93 for stool and R²=0.90 for both MEM and MEM+DTT in saliva; with R²=0.93 for taxa above 0.1% relative abundance). For stool, a high correlation between the relative abundance of species in control vs MEM-treated samples showed that MEM did not substantially alter microbiome composition (FIG. 6B,). For saliva, we observed a high correlation in the relative abundances of microbial taxa between the control sample and both the MEM and MEM+DTT treatments (FIG. 6D). The correlation was less pronounced for low-abundance taxa with enrichment of specific species in MEM-treated samples and was investigated more quantitatively by comparing the coefficient of variation (CV) across samples for low-abundance species (FIG. 6E). MEM-treated saliva samples had lower CV (50% CV, 95% CI), indicating better replicability compared with untreated controls. Additionally, MEM improved quantification of low-abundance species (>0.05% relative abundance), enabling detection of an additional 10 species that were undetected in the control.

Additional experiments further confirmed these taxa were not introduced during MEM processing (FIG. 41).

These shotgun-sequencing experiments with mouse fecal and human saliva samples demonstrated that MEM treatment introduced minimal microbial biases (more than 98% of microbial species experienced less than a 4-fold loss in relative abundance) while detecting additional microbial taxa at equivalent sequencing depths.

In view of the above, it would be apparent to a skilled person that these shotgun-sequencing experiments with mouse fecal and human saliva samples demonstrated that MEM treatment introduced minimal microbial biases (more than 98% of microbial species experienced less than a 4-fold loss in relative abundance) while detecting additional microbial taxa at equivalent sequencing depths.

A skilled person would also understand upon reading of the present disclosure that shotgun sequencing after MEM treatment is expected to be feasible on similar sample types where bacterial load is high. Similar results are expected in host and non-host reads for any Illumina sequencing platforms. It is also expected that the additional non-host reads gained from MEM+DTT to be dependent on individual samples (i.e. not all saliva samples may see this effect).

### Example 7: Analysis of Microbial Enrichment in Paired Human Intestinal Biopsies Processed with and without MEM

To assess the impact of MEM on intestinal microbes, 8 ascending colon biopsies, designated as 10 cm distal to the ileocecal valve, were collected from a single field of view (5 cm diameter) for five different participants.

All activities related to enrollment of participants, collection of samples, and sample analysis were approved by the University of Chicago IRB and performed under IRB protocols #15573A and #13-1080. De-identified samples were received at Caltech and analyzed under Caltech IRB protocol #21-1083. Adults scheduled for routine colon cancer screenings via colonoscopy at the University of Chicago Medicine (UCM) were screened for diagnosis and eligibility criteria for enrollment in the study on a weekly basis. Exclusion criteria included: participants with chronic infectious diseases such as human immunodeficiency virus (HIV) or hepatitis C (HCV); active, untreated Clostridium difficile infection; active infection with severe acute respiratory syndrome coronavirus 2 (SARS-CoV-2); intravenous or illicit drug use such as cocaine, heroin, non-prescription methamphetamines; active use of blood thinners; severe comorbid diseases; participants on active cancer treatment; and participants who were pregnant. Approaching prospective participants was at the discretion of their treating physician and was not done in cases that would put participants at any increased risk, regardless of reason. Participants were approached the day of their procedure and informed, written consent was obtained before any samples were acquired.

Biopsies were collected in a total of 2-3 passages with 3-4 biopsies per passage using a pair of 2.8-mm biopsy forceps. Biopsies from the same passage were stored together on ice in a dry microfuge tube for an average of 28 min (ranging from 15 min to 36 min). After samples were transferred to the lab, biopsies from the same passage were split into control and MEM groups for a total of 4 biopsies per condition with evenly sized biopsies present in each group. Biopsy size ranged from 0.2 mg to 4.8 mg with an average weight of 2.49 mg. Non-host-depleted biopsies were processed individually by adding 150 μL of PrimeStore MTM inactivation buffer (Longhorn) to each biopsy and vortexing briefly before storing at −80° C. until DNA extraction. Depleted samples were processed individually at University of Chicago before shipment on dry ice to Caltech for DNA extraction.

Samples were processed with MEM and extracted as described in Example 1 (FIG. 5A) and the results of the analysis are reported in FIGS. 7A to 7E.

In particular, in FIG. 7A sampling graphic illustrates collection from four participants (CT15, CT17, CT18, and CT19) each with eight ascending colon biopsies. Four biopsies from each participant were MEM-treated and four were untreated controls. From each of four participants, we obtained eight mucosal biopsies; four biopsies from each participant were assigned to the MEM-treatment group and four were untreated controls (FIG. 7A). Due to concerns regarding contaminant DNA in samples with low bacterial loads,[45-48] the background bacterial signal associated with MEM was also characterized as well as the processing methods through quantitative 16S rRNA gene sequencing of MEM processing blanks.

FIG. 7B, show results of experiments where host DNA was quantified for each biopsy using ddPCR of a single-copy host primer as described in Example 6 (see FIG. 6C). Human genomes remaining refers to the abundance of this single-copy gene present in 1 μL of elution (* indicate measurement was below limit of blank [LoB] defined as LoB=mean_{blank}+1.645[SD_{blank}] based on three processing blanks). The results reported in FIG. 7B show that MEM removed host DNA more than 2,000-fold across all 16 biopsies, with most biopsies having host levels comparable to a processing blank after MEM treatment. (FIG. 7B).

FIG. 7C, show results of experiments where Quant-Seq was performed on all biopsies as described in Example 5 (see experiments related to results of FIG. 5B) to determine how MEM affects the human intestinal microbiome at a community level, performed quantitative 16S rRNA gene sequencing. [42] was performed. In particular, principal coordinates analysis (PCA) on microbial genus-level relative abundances were performed to visualize microbial population variation.

The results reported in FIG. 7C show that roughly 93% of genera remained in MEM-treated and control biopsies after computationally removing taxa found at higher absolute abundances in the blanks, giving us confidence most detected taxa were not background contaminants. To further confirm that MEM did not introduce additional contamination, a strong agreement was found between taxon abundances in MEM and control biopsies. PCA of sequencing results showed that any differences in microbial relative abundances introduced by MEM were less than the differences observed between participants (FIG. 7C). Analysis of sequencing results revealed minimal changes in relative abundances of most taxa after MEM treatment, with roughly 88% of taxa having no significant differences in relative abundances from the controls (Mann Whitney U test, two-sided P=0.05). For taxa present at greater than 1% relative abundance, more than 95% of taxa had no significant differences between MEM and control samples.

FIG. 7D shows results of experiments where Log 2-fold differences in relative abundances of the taxa between control and MEM-treated biopsies were detected and plotted with a standard normal distribution overlaid in black. The results reported in FIG. 7D show that the log 2-fold difference in taxa between control and MEM-treated samples approximated a normal distribution (Kolmogorov-Smirnov test against normal distribution, statistic=0.074 P=0.11).

FIG. 7E shows relative abundance of taxa measured in control vs MEM-treated biopsies were plotted and overlaid on a dashed line showing 1:1 correlation. Highlighted in gray are taxa that were below the assay limit of quantification (LOQ). Highlighted in orange are taxa with greater than 4-fold changes between control and MEM biopsies. The results reported in FIG. 7E show that there was a linear correlation in relative taxon abundances between the control and MEM-treated samples.

In view of the above, a skilled person will understand that the speed and simplicity of MEM enables over 1,000-fold host removal while introducing minimal biases in microbial relative abundances when used in a clinical setting on human intestinal biopsies.

The above exemplified MEM is expected to work on biopsies that were immediately processed or on biopsies that were processed an hour after collection. MEM is also expected to induce larger losses to anaerobic microbes if samples are not processed under anaerobic conditions, as will be understood by a skilled person upon reading of the present disclosure.

### Example 8: Shotgun Sequencing of MEM-Treated Human Intestinal Biopsies

To investigate whether MEM enables detection and characterization of additional microbial species, pathways, and genes from human intestinal biopsies, paired control and MEM-treated were shotgun-sequenced at a depth of above 100 million reads (N=2 for each condition)

A set of experiments was performed to determine with MEM analysis whether mucosa-associated microbes vary along the GI tract has been challenging to determine due to the low number of microbial reads that could be recovered from mucosal biopsies. [49]

Human intestinal biopsies were obtained and processed with MEM and extraction as described in Example 7. Four biopsies from participant CT18 (two MEM-treated and two control) were shotgun-sequenced. Shotgun prep and sequencing was performed as described in Example 6. In particular, paired control and MEM-treated biopsies from CT18 (see Example 7 and FIG. 7A). were shotgun-sequenced at a depth of above 100 million reads (N=2 for each condition).

Analysis for species assignment was performed by processing sequencing data using the KneadData v0.10.0[22]. Through KneadData, quality control (QC) and host removal were performed with Trimmomatic v0.39[23]. Human derived sample types were aligned to KneadData's default human reference genome (a combination of hg38 human genome reference (GenBank assembly accession #GCA_000001405.29) and small contaminant sequences) and aligned reads were removed. After bioinformatic host removal, the percentages of host reads were calculated by dividing reads remaining after host filtering by the total reads that passed QC. To assign species, non-host reads from read 1 and read 2 were then concatenated and processed using the Metaphlan 3.0 workflow outlined in bioBakery.[22] under default settings (Database: mpa_v30_CHOCOPhlAn_201901)[22]. Analysis for pathways and genes was performed by processing with HUMAnN 3.0. Non-host read 1 and read 2 outputted from KneadData were concatenated and processed using the HUMAnN 3.0 workflow outlined in bioBakery under default settings.[22] Taxonomic profiles obtained from MetaPhlan (see “Marker Gene Analyses”) were merged within patients and used as taxonomic inputs using the “--taxonomic-profile” flag in HUMAnN. Reported pathway abundances and gene abundances were normalized to relative abundances and concatenated. The results are reported in FIGS. 8A to 84F.

In particular, in the illustration of FIG. 84A the number of microbial species, pathways, and genes identified in each sample were plotted (N=2). A roughly a 100-fold increase was observed in the number of organisms that could be detected, a 700-fold increase in the number of pathways detected, and over a 400-fold increase in the total number of genes detected in MEM-treated samples compared with the control samples (FIG. 8A). When comparing only completed pathways, defined as above 90% complete, no complete pathways were detected in either of the control biopsies. An average of 1.5 (SD: 1.5) species and 728 (SD: 107) genes were detected in the control biopsies, whereas an average of 137.5 (SD: 21.5) species and 300,641 (SD: 6,922) genes were detected in the MEM-treated biopsies. MEM treatment enabled shotgun-sequencing classification of microbes down to a relative abundance of 0.005%, whereas in control biopsies a minimum relative abundance of 10% was required to detect microbes at similar sequencing depths. MEM-treated biopsies could detect genes down to a relative abundance of 10⁻¹⁰, whereas in control biopsies genes could only be detected when present at a minimum relative abundance of 10⁻⁵.

FIG. 8B reports results of experiments where relative abundance of genes was compared. For the top 5,000 abundant genes, the log 2 fold-change in relative abundances between the two MEM-treated biopsies and the two control biopsies were plotted. The results reported in FIG. 8B, show that MEM treatment improved reproducibility of detecting the most abundant genes. In the control biopsies, a high percentage of the genes (98%) were detected in only one sample, whereas for the MEM-treated samples only 3% of the genes were detected in one biological replicate but not the other.

FIGS. 8C to 8F, report results of experiments directed to test whether MEM would enable characterization of microbial variation (at the taxon-, pathway-, and gene-level) cross sectionally across individuals and longitudinally across the GI tract of a single individual. In particular, for longitudinal sampling, a total of five participants were sampled 12 times from 4 different locations during a routine colonoscopy. The 4 locations sampled were the terminal ileum, ascending colon (designated as 10 cm distal to the ileocecal valve), descending colon, and rectum. From a single field of view (5 cm diameter) from each location, 3 biopsies were collected in one passage with 2.8 mm biopsy forceps and stored dry on ice in a microfuge. For participant CT14, only one rectal sample was obtained. On average, biopsies were 2.5 mg with a minimum size of 0.1 mg and a maximum of 5.9 mg. All biopsies were then processed individually in the laboratory at University of Chicago before shipment on dry ice to Caltech for DNA extraction. Time between specimen collection and processing ranged from 10 min to 52 min. Samples were processed individually in the laboratory at University of Chicago before shipment on dry ice to Caltech for DNA extraction. Samples were processed with MEM and extracted as described in Example 3 (see FIG. 5a)C. Quant-seq was performed as described in Example 3 (see FIG. 5B). Shotgun prep and sequencing was performed as described in Example 6 at an average read depth of 25 million, producing an average of 2 million non-host reads.

FIG. 8C shows a sampling graphic for collection of 12 biopsies from each of 5 participants (3 biopsies taken from 4 separate regions of the GI tract). Biopsies within one region were sampled within one field of view (5-cm diameter).

FIG. 8D reports results of experiments where all 60 biopsies were processed with MEM, followed by 16S rRNA gene sequencing (for genera) and shotgun sequencing (for species, pathways, and genes). The number of features for genera, species, pathways, and genes were grouped based on whether they were present in at least one biopsy sample from only one participant (1/5), two participants (2/5), three participants (3/5), four participants (4/5), or from all participants (5/5). Analysis was performed on annotated genes, and separately on all genes (annotated+unannotated). The results reported in FIG. 8D show that about half (91 of 187) of the microbial species identified were unique to an individual. These unique species ranged in relative abundance from 10% to 0.01%. As was observed previously, pathways and genes appeared more conserved across participants compared with taxonomy (genera and species). As was observed previously, pathways and genes appeared more conserved across participants compared with taxonomy (genera and species), [50, 51].

FIGS. 8E to 8F reports results of experiments where PCA was performed on all 60 longitudinal samples grouped by participant (CT7, CT8, CT12, CT13, CT14).

In particular, in the experiments reported in FIG. 8E, it was first tested whether microbial variation between GI sites is present at the genus-level. PCA on relative abundance of 16S rRNA gene sequencing genera assignments. In particular for each participant sample, a quantitative[42] 16S rRNA gene sequencing was used to quantify genus-level microbial changes longitudinally along the GI tract. The results reported in FIG. 8E show that microbial taxa from the proximal colon (terminal ileum [TI] and ascending colon [AC]) and taxa from the distal colon (descending colon [DC] and rectum [R]) showed some clustering by location in most participants.

FIG. 8F reports results of experiments where for each participant sample, shotgun sequencing was used to test whether the observed variation in taxa along the GI tract extended to the species, pathway, or gene levels. PCA on relative abundance of shotgun-sequencing species assignments. Clustering between the TI/AC vs the DC/R was seen in some participants across species, namely in participants CT7, CT12, CT13, and CT14. There appeared to be minimal clustering between the TI/AC vs the DC/R at the pathway and gene-level (FIG. 42). Additionally, there is high variation within regions for some individuals, which may be attributed to read depth limitations. For example, for one DC sample from CT13 no microbial marker genes were identified due to the minimal number of non-host reads so it is missing from the PCA plots.

In view of the above it would be apparent to a skilled person upon reading of the disclosure, that the shotgun sequencing of MEM-treated human intestinal biopsies enabled characterization of high- and low-abundance microbial species, pathways, and genes. The exemplary characterization of this Example documented longitudinal shifts in the mucosal microbiome along the lower human GI tract at the level of species, pathways, and genes.

It is expected that other forms of shotgun analysis to also be compatible with MEM-treated samples, such as k-mer based species assignments including Kraken and/or other marker gene analysis software and/or other gene and pathway assignments. It is also expected other shotgun sequencing prep methodologies and sequencing to be compatible with MEM-treated samples, including long-read sequencing if long-read compatible extraction kits are employed. It is further expected additional sequencing depth will enable additional species, gene, and/or pathways identifications as will be understood by a skilled person upon reading of the present disclosure.

### Example 9: MAG Construction with MEM-Treated Human Intestinal Biopsies Performed from Shotgun Metagenomic Sequencing

To determine whether a single microbial strain varies along the GI tract, a set of experiments was performed to assemble microbial genomes from MEM-treated intestinal biopsies shotgun metagenomic data.

In particular, metagenome-assembled genomes (MAGs) were constructed after processing with MEM, with two control and two MEM-treated biopsies with similar bacterial loads from participant CT18 (see Example 7 FIG. 7A, and Example 8, FIG. 8A).

In particular, shotgun sequencing data generated in Example 8 (see FIG. 4A) was utilized for this analysis. The results are reported in FIGS. 9A to 9D.

In particular, FIG. 9A reports the results of experiments where two control and two MEM-treated biopsies from the same participant (CT18) and intestinal region (ascending colon) were shotgun-sequenced. Number of non-host reads were determined after alignment to a human reference genome. In the experiments of FIG. 9A, both control and MEM-treated biopsies were sequenced to measure the additional information MEM treatment can help yield at equivalent sequencing depths and processing steps. After processing, host reads were removed bioinformatically and ˜10% of reads were identified as non-host in MEM-treated samples whereas less than 0.01% of reads were identified as non-host in the untreated controls.

With respect to the results reported in FIGS. 9B to 9D the analysis was performed as previously described. [52] Sequencing data was processed using the metagenomic workflow[24],[25] outlined in anvi'o[26, 27] v7.1 (https://anvio.org). QC filtering of short reads was performed using the Illumina-utils library [28] v2.12. Host reads were removed by alignment to the hg38 human genome reference (GenBank assembly accession #GCA_000001405.29). Assembly was performed on each sample individually using MEGAHIT[29] v1.2.9 unless co-assembly was explicitly stated as in FIG. 8A to 8F, with default setting except setting a minimum contig length of 1000 bp. Short reads generated from each sample were then aligned to contigs generated from all assemblies using Bowtie2[30] v2.3.5. Contigs were processed using anvi'o to generate a contig databases with the command “anvi-gen-contigs-database” with default settings and with Prodigal [31] v2.6.3 to identify open reading frames. Single-copy core genes were detected with “anvi-run-hmm” to (bacteria n=71 and archaea n=76, modified from Lee, 2019[32], ribosomal RNAs (rRNAs) (n=12, modified from [5]) using HMMer[33, 34] v3.3.2. Genes were annotated using using ‘anvi-run-ncbi-cogs’ for NCBI's Clusters of Orthologous Groups (COGs) database[35] and ‘anvi-run-kegg-kofams’ from the KOfam HMM database of KEGG orthologs (KOs)[36]. BAM files were profiled with “anvi-profile” and merged with “anvi-merge” for samples originating from the same participant. Automatic binning was performed by CONCOCT[37] v1.1.0 by specifying a maximum number of bins based on the estimated number of bacterial genomes computed from each sample's contigs. The maximum number of bins was set to 1/3 the number of expected genomes to limit the likelihood of fragmentation. Bins generated with CONCOCT were imported in the anvi'o profile database and were then manually refined and summarized to obtain fasta files of individual MAGs. Once manual binning of all samples from the same participant was complete, MAGs above 50% complete were dereplicated to generate a unique list of genomes using anvi'o and pyani v0.2.11. Representative genomes were chosen based on quality scores and clustered based on >95% ANI. The final list of MAGs were taxonomically assigned with GTDB-Tk (Genome Taxonomy Database Toolkit; v2.1.0[38, 39]) using classify_wf with default settings.

In particular FIG. 9B reports results of experiments where contigs were constructed from co-assembly of the two samples from each condition and the distribution of contig lengths was plotted. The number of prokaryotic genes identified in these contigs is shown. We first tried to reconstruct MAGs from the control samples, however, the assembly of the short reads from non-host-depleted samples and our subsequent attempts to bin the resulting contigs into MAGs were unsuccessful because these assemblies suffered from remarkably short contigs. Co-assembly was then performed on MEM-treated samples and resulted in substantially more and longer contigs compared with the control samples, with contig lengths of up to 833 kbp In the experiments reported in FIG. 9B, automatic binning and manual refinement steps resulted in a total of 34 high-quality bacterial MAGs (more than 90% complete and less than 5% redundant) and more than 70 medium-quality MAGs (more than 50% complete and less than 10% redundant), demonstrating how MEM treatment of human intestinal biopsies makes it possible to reconstruct MAGs from these samples.

FIG. 9C reports results of experiments performed to confirm that the MAGs reconstructed from MEM-treated samples were accurate representations of the untreated biopsies, we assessed the uniformity of coverage of the control reads when mapped back onto the MAGs. To perform this analysis, we chose a MAG resolved to Alistipes putredinis, a known gut microbe that had the highest detection in the control samples. Control samples showed an even distribution of reads among the 29 contigs present, indicating that this MAG was also present in the control samples, but sequencing depth limitations prevented the reconstruction of a genome. Bar heights represent mean coverage and are scaled independently for each sample.

FIG. 9D reports results from co-assembly of MEM biopsies, 34 high-quality MAGs (>90% complete, <5% redundant) and more than 70 medium-quality MAGs (more than 50% complete and less than 10% redundant) were constructed de novo. Heatmap shows the percentage of each genome that is covered at least 1× by the sample (i.e., detection or breath of coverage), with a maximum of 3.7% in control samples and 99.999% in MEM samples. For the 34 high-quality MAGs, detection was computed, which reports the proportion of nucleotides in a given reference sequence that are covered by at least one short read in a given metagenome. Thus, detection is an extremely effective way to be able to discuss the presence of a given population in a given sample, independent of read coverage, and by avoiding false positives due to non-specific read recruitment. The results reported in FIG. 9D shows that the average detection for MEM1, MEM2, Cntrl1, and Cntrl2 were 99.8% (SD: 0.7%), 97.3% (SD: 6.4%), 0.8% (SD: 0.7%), and 1.2% (SD: 1.1%) respectively across all MAGs. Overall, we observed a higher detection of all 34 high-quality MAGs in MEM-treated samples compared to control samples.

To assess whether any of these MAGs were contaminated, a taxonomic classification on each genome was performed.[38, 39] With a threshold of 95% average nucleotide identity (ANI), 33 MAGs were successfully classified. We compared the size of each classified MAG with the matching reference genomes in GTDB and found high agreement with current microbial databases (R²=0.8057728, P=2.2×10⁻¹⁶) indicating that the MAGs constructed from MEM-treated samples were not artifacts. One Fusobacterium MAG matched closely with a published fecal-derived MAG at 86.85% ANI but GTDB was unable to assign species-level taxonomy. Because all MAGs were constructed in the same manner and with similar quality metrics, it is likely that this Fusobacterium MAG is a novel taxon rather than contamination. These 34 MAGs spanned six bacterial phyla (FIG. 9D) and an archaeon (Methanobrevibacter smithii) MAG was constructed from patient CT12, demonstrating MEM-treated biopsies enabled genome reconstruction of archaea and a wide variety of bacteria. Taxonomy was assigned for each MAG and listed to the right along with completion/redundancy (C/R). The phylogenetic tree to the left of the heatmap highlights taxonomic grouping of each MAG.

In view of the above it is expected that using MEM additional MAGs will be constructed with improved sequencing depth. It is also expected that long-read sequencing to improve MAG construction from MEM-treated samples as will be understood by a skilled person upon reading of the present disclosure.

### Example 10: Interindividual and Intraindividual Bacterial Biodiversity Present Along GI Tract

Shotgun sequencing data obtained as described in Example 8 (see FIG. 8C to 8F) were analyzed to investigate how microbial genomes may vary across individuals and within individuals.

To determine whether MEM enables differentiation of population-level microbial differences across individuals within a single taxon, a total of six biopsies from participant CT12 were re-sequenced to a sequencing depth of roughly 250 million reads as described in Example 6. Assembly and binning were performed on each of the six biopsies individually and MAGs were dereplicated across samples as described in Example 9. The results are reported in FIGS. 10A to 10C.

In particular, FIG. 10A reports the results of experiment were gene-level analysis was performed on Phocaeicola vulgatus for all five participants. A MAG of Phocaeicola vulgatus, the most prevalent and abundant species found in all participants, from participant CT12 was constructed and annotated. Reads from all 60 intestinal biopsies taken from all five participants (from Example 8 FIGS. 8C to 8F) were then mapped onto this MAG to identify which genes were absent from the other participants' biopsies. In particular, in the experiments repotted in FIG. 10A a gene-resolved analysis of naturally occurring P. vulgatus populations through metagenomic read recruitment was performed, as described previously,[53] and revealed a large core genome, and differentially occurring genes across individuals. Samples were grouped by gene detection, defined as percentage of each gene with at least 1× coverage, and showed strong participant-dependent grouping but lacked grouping by GI location.

The results reported in FIG. 10A show that genes from biopsies taken across GI tract regions within participant CT12 appeared conserved (CT12 samples had an average gene detection of >96%). Some genes with high detection were only found in one or two patients (either CT12 only or CT12 and one other patient), which were defined as unique genes. To assess whether these genes were functionally distinct, genes were annotated with the Clusters of Orthologous Groups (COG) database to identify orthologous genes. Of the 287 genes unique to CT12, 100 of these were annotated by COG and corresponded to a wide range of functions. Of the gene clusters unique to two participants (i.e., CT12 and one other individual), about 30% were annotated. MEM treatment enables insights into functionally distinct microbial populations of the same taxon that occupy the same geographical location in the gut across individuals with similar health status.

FIG. 10B reports results of experiments directed to investigate whether MEM treatment could enable studies of microbial population genetics in low-biomass samples through single-nucleotide variants (SNVs) as a result of the increased depth of coverage. For this analysis, we analyzed MAGs from patient CT12 as reference genomes and mapped the paired-end reads from the terminal ileum and descending colon from CT12 onto these assembled genomes. Six MAGs had a mean coverage above 50× across all 6 samples (3 terminal ileum and 3 descending colon) and were selected for subsequent SNV analysis. SNV profiles were generated from the paired-end reads of each sample by comparing them with the reference sequence (MAG). It was investigated whether PCR errors were responsible for some of the SNVs observed in the data by preparing libraries for an additional three technical replicates from a single terminal ileum biopsy, with the expectation that differences in the SNV profiles of the technical replicates should be minimal.

The plot reported in FIG. 10B shows the ECDF of the occurrence of single-nucleotide variants (SNVs) in a MAG of Ruminococcus bromii and the deviation of these SNVs from the reference across three technical replicates. 1/3, 2/3, and 3/3 indicates the number of technical replicates that had an SNV at that location followed by the total number of SNVs in each of these categories. A black dashed line is drawn at 21% deviation from reference; above this value, all observed SNVs were present in all three technical replicates. By looking at nucleotide variations occurring in one, two, or all three replicates, we observed that a minimum deviation from the reference nucleotide of 21% for Ruminococcus bromii allowed for the selection of SNVs only and minimized the impact of PCR errors in the population structure analysis.

FIG. 10C reports results of experiments where nucleotide-level analysis was performed on MAGs with a mean coverage above 50× across all samples. Shown here is the fixation index from SNVs analyzed within the coding region of R. bromii with a minimum deviation from reference set at 21%. Samples were clustered based on fixation index and strong region-dependent groupings can be seen. DC, descending colon; TI, terminal ileum. Analyses of these data using fixation index showed that some taxa, such as R. bromii and Gemmiger formicilis, were composed of subpopulations that were distinct between the upper and lower intestinal tract. To assess whether these SNVs were functionally significant, codon-level and translated (amino acid) analyses of SNVs in R. bromii were performed and similar clustering of biopsies by location was detected. The results reported in FIG. 10C show that the recovery of SNVs afforded by the deeper sequencing and increased coverage of MAGs from biopsy samples allowed detection of the presence of subpopulation structures for some individual taxa along the lower GI tract of a single individual.

Accordingly, in view of the above. It can be concluded that MEM enables MAG construction and deep characterization of intestinal microbes from human biopsies as will be understood by a skilled person. In particular, a skilled person will understand upon reading of the present disclosure that MEM treatment enables insights into functionally distinct microbial populations of the same taxon that occupy the same geographical location in the gut across individuals with similar health status.

On this basis a pangenomic analysis is expected to be feasible on MEM-treated samples. Strain tracking is also expected to be feasible using SNV profiles from MEM-treated samples. PCR-free library preparation is also expected to be possibly preferred for high biomass samples for SNV analysis as will be understood by a skilled person upon reading of the present disclosure.

### Example 11: MEM Protocol Optimization

MEM protocol and extraction was performed as described in Example 4 (see FIG. 5A with the described modifications in this example. The relates results are reported in FIG. 11.

In particular, FIG. 11 panel A reported experiments where MEM protocol was tested with and without Proteinase K (PK) treatment on mammalian mouse cell culture. Host was quantified after MEM treatment and DNA extraction with single-copy mouse primers with qPCR as described in Example 5 (see FIG. 5C-5F)

FIG. 11 panel B reported experiments where MEM protocol was performed on mammalian mouse cell culture to compare the effectiveness of a homogenizer or vortexer in host cell disruption. A comparison between homogenizer (4.5 m/s for 30 sec) vs vortexer adapter (1 min at max speed) was performed and remaining host DNA after MEM treatment was quantified using qPCR on single-copy mouse primers as described in Example 5 (See FIGS. 5C to 1E). The results reported in FIG. 11 Panel B support the conclusion that concluded homogenizers are not necessary and this may be important for field work for future environmental samples. The results obtained in this example confirm the ones expected for the used configuration of beads and beads parameters according to the host depletion method of the present disclosure. Reference is made in this connection to the data reported in the table below.

| | | | | | |
|---|---|---|---|---|---|
| Radius of Bead (mm) | 0.7 | Frequency (Hz) | 16 | Bead Fill Volume % | 25.00% |
| Number of beads | 348 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 3E−10 | 0.04% |
| bacteria | 1 | 1E−15 | 1.00E+08 | 100 | 1E−09 | 0.15% |
| bacteria | 2 | 4E−15 | 1.00E+08 | 100 | 4E−09 | 0.59% |
| fungi | 5 | 3E−14 | 1.00E+08 | 100 | 3E−08 | 3.66% |
| fungi | 10 | 1E−13 | 1.00E+08 | 100 | 1E−07 | 14.62% |
| fungi | 20 | 4E−13 | 1.00E+08 | 100 | 4E−07 | 58.22% |
| virus | 0.1 | 1E−17 | 1.00E+09 | 100 | 1E−11 | 0.00% |
| archaea | 1 | 1E−15 | 1.00E+10 | 100 | 1E−09 | 0.15% |
| host | 8 | 7E−14 | 1.00E+04 | 1 | 7E−06 | 936.83% |
| host | 15 | 2E−13 | 1.00E+04 | 1 | 2E−05 | 3282.51% |
| host | 50 | 3E−12 | 1.00E+04 | 1 | 3E−04 | 35860.06% |

FIG. 11 Panel C reports experiments where MEM was performed on 3 rat biopsies split into two different reactions with 15-min or 30-min incubation at 37° C. Rat biopsies were obtained as described in Example 5 (see FIG. 5E). Host load was quantified through LINE1 transposon primers with qPCR. Minimal differences in host DNA removal with incubation of 15 or 30 minutes in nuclease. Final protocol used 15 minute incubation to minimize processing times.

In view of the above it is expected that the addition of proteinase K removes more host reads after shotgun sequencing. It is also expected some microbes, such as non-enveloped viruses, can possibly be lost during proteinase K treatment and therefore treatment can be optimized to the microbial target of interest. It is further expected that MEM will be effective on liquid samples when performed at a homogenizing speed of 1,000 rpm to 7,000 rpm. It is additionally expected that MEM incubation with Benzonase will be saturated by 15 minutes when working with ˜10 mg of tissue. It is therefore expected MEM incubation with Benzonase could range anywhere from 2 minutes to 60 minutes with similar host and microbial DNA content after extraction.

### Example 12: Impact of Host Depletion on Bacterial Phyla of Host Samples

FIGS. 12 and 13 shows a diagram illustrating the Impact of host depletion on specific bacterial phyla and in particular detection of Log 2 fold-change between relative abundance of genera within each phylum in treated and control samples from 16S rRNA gene sequencing data for FIG. 12, human saliva on paired MEM-treated and untreated controls, and FIG. 13B, human biopsies on paired MEM-treated and untreated controls. The histograms in FIGS. 12 and 13 are overlaid with a normal distribution (black line).

### Example 13: Correlation Between Bacterial Load and Non-Host Reads

Shotgun sequencing was performed on longitudinally sampled intestinal biopsies after processing with host depletion as described in Example 8 (see FIGS. 8 C-F).

The results reported in FIG. 14 show that toughly 25 million reads on average were obtained for each biopsy and all samples fit on a single NovaSeq6000 S1 flowcell. After host-filtering an average of 2 million reads were remaining with a range from 2E4 reads to 2E7 reads. The variability in non-host reads remaining had a strong correlation (Spearman, r=0.79) with the total microbial load as measured by digital PCR as described in Example 4 (see FIG. 5B).

This strong correlation indicated that our process was achieving a relatively uniform depletion across all samples. Additionally, the strong correlation indicates that the majority of non-human reads in our samples come from bacteria picked up by the 16S primers used for total microbial load quantification.

On this basis it is expected that bacterial load can be used to predict non-host reads after MEM treatment. It is also expected that MEM can achieve consistent host removal across samples from various individuals. It is further expected that the measured non-host reads percentage will be independent of sequencing depth. We expect if biopsies were not proteinase K treated there would be higher percentages of host reads.

### Example 14: Bacterial Loads of Longitudinal Biopsies

16S rRNA gene copies were quantified as a proxy for bacterial load for all biopsies as described in Example 5 (see FIG. 5A) and Example 8 (see FIGS. 8C to 8F).

Samples were plotted by participant and then by location. The results are reported in FIG. 15 which support the expectation that MEM can remove host nucleic acid independent of bacterial/archaeal loads. (see also Example 5 and Example 8)

### Example 15: Abundance of Participant Unique Species

From shotgun sequencing of 5 participants longitudinally as described in Example 8 (see FIG. 8C to 8F), unique species were defined as present in only one of the five participants.

The results did show relative abundances of some of these unique species are shown with the bars representing the average abundance across all 12 biopsies from one participant.

### Example 16: MAG of Fusobacterium

From two MEM-treated ascending biopsies from CT18 as described in Example 8 (see FIG. 4A-B) and Example 9, a MAG of Fusobacterium was constructed (completeness: 94%, redundancy: 1.4%). The results are shown in Figure S10.

### Example 17: Archaeon Methanobrevibacter smithii Found Along the Lower GI Tract

From shotgun sequencing as described in Example 8 (see FIG. 8C to 8F), it was detected that participant CT12 had low levels of Methanobrevibacter smithii present in the terminal ileum, descending colon, and rectal biopsies.

MAG construction was performed on co-assembly of all biopsies taken from the terminal ileum and descending colon, as described in Example 9, to reconstruct a full Methanobrevibacter smithii genome (completeness: 100%, redundancy: 0%). The results are shown in FIG. 17

### Example 18: Genes Driving Participant Unique Strains of Phocaeicola vulgatus

Sequencing data was obtained and analyzed as described in Example 10 (see FIG. 10A).

The results reported in FIG. 18 show that 100 annotated genes found in only CT12 were sorted based on COG20 Category and the number of genes in each category are shown. FIG. 18 reports result of the same analysis repeated for genes found in only CT12 and one other participant (labeled).

### Example 19: Fixation Index Across MAGs with Varying Deviation from Reference

Six MAGs with greater than 50× mean coverage were selected for SNPs analysis as described in Example 10 (see Figure B-C. Fixation index analysis was performed on each MAG for various thresholds of minimum departure from reference nucleotide as described in Example 10 (see Figure B). Clustering of fixation index by location can be seen for some MAGs (red indicates terminal ileum samples vs blue are descending colon). The results are shown in FIG. 19.

### Example 20: Ruminococcus bromii Strain Variants at the Nucleotide (SNV), Codon (SCV), and Amino Acid (AA) Level

SNVs present in R. bromii above the threshold of 21% deviation from reference were analyzed at the codon and translated-level to determine if SNVs may indicate a functional change as described in Example 10 (see FIG. 5C). The fixation index for each level of analysis were plotted and are shown in FIG. 20.

### Example 21: MEM Supports Both DNA and RNA Depletion in Lipidaceous Samples

The microbial enrichment method (MEM) supports a wide variety of tissue types. Here, we demonstrate the effectiveness of MEM in depleting both DNA and RNA from lipidaceous samples.

Mesenteric adipose tissue (MAT) was used as an exemplar of lipidaceous sample processing since this sample type has a high proportion of lipids relative to other sample types.

Mesenteric adipose tissue (MAT) was collected at Cedars-Sinai Hospital from Crohn's Disease (CD) patients during ileal resection related to disease. The sample was processed as follows.

MEM Treatment: 100 mg of human mesenteric adipose tissue (MAT) was resuspended in 800 ul of PBS in Lysing Matrix D bead beating tubes (MP Biomedicals, MP116913050). Samples were then homogenized through bead beating for various durations. In cases where an experiment required greater than 700 ul of homogenate, multiple tissue aliquots were homogenized in parallel and then pooled and split as needed. 183 ul of homogenized sample was mixed with 10 ul of buffer comprised of 100 mM Tris and 40 mM MgCl2, along with 5 ul of Proteinase K (NEB, P8107S) and 2 ul of nuclease Benzonase (Sigma-Aldrich, 71205-3). Samples were mixed by pipetting and incubated at 37 C with 600 RPM shaking for 15 minutes. Samples were then centrifuged at 10,000 g for 2 minutes and the supernatant was discarded. Remaining cell pellets were resuspended in 200 ul of lysis buffer.

DNA/RNA Isolation and Quantification: DNA and RNA were extracted from samples using Qiagen's AllPrep PowerViral DNA/RNA extraction kit (Qiagen, 28000-50) following the manufacturer's protocol (with the recommended addition of beta-mercaptoethanol for RNA capture). Notably, all samples were diluted in PM1 lysis buffer and bead beat across three 60 s cycles at 6.5 m/s using Lysing Matrix E beads (MP Biomedicals, 116914050-CF) to ensure complete homogenization prior to extraction. Processed samples were eluted in 100 ul of nuclease free water and split into DNA and RNA aliquots. RNA aliquots underwent DNA removal using the TURBO DNAse protocol (Thermo Fisher Scientific AM1907) by incubating 2 ul of DNAse with 25 ul of RNA elution and 3 ul of 10× Buffer and incubating at 37 C for 30 minutes. DNAse was then removed using the Zymo RNA Clean & Concentrator kit (Zymo R1017). 2 ul of cleaned RNA was then converted to cDNA using the High-Capacity cDNA Reverse Transcription Kit (Thermo Fisher Scientific 4368814). DNA and cDNA were quantified with qPCR using AccuStart II PCR SuperMix (Quantabio, 95137-100) with EvaGreen fluorophore (Biotium, 31000). Host loads were quantified using primers specific to mammalian ribosomal 18S gene (F: ACGGACCAGAGCGAAAGCAT (SEQ ID: 7), R: TGTCAATCCTGTCCGTGTCC (SEQ ID: 8)). Microbial loads were quantified using microbial 16S ribosomal gene specific primers targeting the V4 region (F: CAGCMGCCGCGGTAA (SEQ ID: 9), R: GGACTACHVGGGTWTCTAAT (SEQ ID: 10)).

Lipid Depletion: Lipid depletion was performed on a subset of samples using the following protocol. Samples were centrifuged at 12,000 g for 15 minutes at room temperature to separate the lipid phase from the aqueous phase. The lipid phase was then physically removed using an oliophilic swab (Puritan, 25-1506). Samples were then resuspended, and MEM conditions proceeded through enzyme treatment while control samples proceeded immediately to nucleic acid extraction.

The results reported in FIG. 21 shows MEM performance among sample processing replicates under the conditions a) untreated (Control), b) MEM treatment (+MEM), and c) lipid depletion (LD) with MEM treatment (+MEM/LD). Detailed descriptions of the protocols are explained below. Both DNA and RNA from host cells were effectively depleted following the MEM protocol, with slightly improved host nucleic acid depletion under LD conditions. Microbial nucleic acids were preserved through MEM processing in both +MEM and +MEM/LD conditions.

Tissue homogenization was optimized by bead beating MAT samples using various conditions. MAT samples were homogenized by bead beating with spherical 1.4 mm zirconium-silicate lysis beads (MP Biomedicals, MP116913050) at 4.5 m/s for 15, 30, or 120 seconds. Following bead beating, samples either proceeded directly to extraction (Control) or were treated by MEM prior to extraction (+MEM).

The results obtained in this example confirm the ones expected for the used configuration of beads and beads parameters according to the host depletion method of the present disclosure. Reference is made in this connection to the data reported in the tables below.
2R: 1.2 mm to 1.6 mm+one 4 mm bead
Number of beads: 30%
Frequency: 50 Hz
Time: 15 s

| | | | | | |
|---|---|---|---|---|---|
| Radius of Bead (mm) | 0.6 | Frequency (Hz) | 50 | Bead Fill Volume % | 30.00% |
| Number of beads | 663 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 4E−10 | 0.05% |
| bacteria | 1 | 9E−16 | 1.00E+08 | 100 | 1E−09 | 0.19% |
| bacteria | 2 | 4E−15 | 1.00E+08 | 100 | 6E−09 | 0.75% |
| fungi | 5 | 2E−14 | 1.00E+08 | 100 | 4E−08 | 4.67% |
| fungi | 10 | 9E−14 | 1.00E+08 | 100 | 1E−07 | 18.65% |
| fungi | 20 | 4E−13 | 1.00E+08 | 100 | 6E−07 | 74.17% |
| virus | 0.1 | 9E−18 | 1.00E+09 | 100 | 1E−11 | 0.00% |
| archaea | 1 | 9E−16 | 1.00E+10 | 100 | 1E−09 | 0.19% |
| host | 8 | 6E−14 | 1.00E+04 | 1 | 9E−06 | 1194.67% |
| host | 15 | 2E−13 | 1.00E+04 | 1 | 3E−05 | 4183.59% |
| host | 50 | 2E−12 | 1.00E+04 | 1 | 3E−04 | 45572.92% |

| | | | | | |
|---|---|---|---|---|---|
| Radius of Bead (mm) | 0.8 | Frequency (Hz) | 50 | Bead Fill Volume % | 30.00% |
| Number of beads | 280 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 2E−10 | 0.03% |
| bacteria | 1 | 1E−15 | 1.00E+08 | 100 | 8E−10 | 0.11% |
| bacteria | 2 | 5E−15 | 1.00E+08 | 100 | 3E−09 | 0.42% |
| fungi | 5 | 3E−14 | 1.00E+08 | 100 | 2E−08 | 2.63% |
| fungi | 10 | 1E−13 | 1.00E+08 | 100 | 8E−08 | 10.50% |
| fungi | 20 | 5E−13 | 1.00E+08 | 100 | 3E−07 | 41.84% |
| virus | 0.1 | 1E−17 | 1.00E+09 | 100 | 8E−12 | 0.00% |
| archaea | 1 | 1E−15 | 1.00E+10 | 100 | 8E−10 | 0.11% |
| host | 8 | 8E−14 | 1.00E+04 | 1 | 5E−06 | 672.75% |
| host | 15 | 3E−13 | 1.00E+04 | 1 | 2E−05 | 2358.22% |
| host | 50 | 3E−12 | 1.00E+04 | 1 | 2E−04 | 25817.87% |

a. R: 1.2 mm to 1.6 mm+one 4 min bead
b. Number of beads: 30%
C. Frequency: 50 Hz
d. Time: 1.20 s

| | | | | | |
|---|---|---|---|---|---|
| Radius of Bead (mm) | 0.6 | Frequency (Hz) | 50 | Bead Fill Volume % | 30.00% |
| Number of beads | 663 | Time (s) | 120 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 3E−09 | 0.37% |
| bacteria | 1 | 9E−16 | 1.00E+08 | 100 | 1E−08 | 1.50% |
| bacteria | 2 | 4E−15 | 1.00E+08 | 100 | 4E−08 | 5.99% |
| fungi | 5 | 2E−14 | 1.00E+08 | 100 | 3E−07 | 37.40% |
| fungi | 10 | 9E−14 | 1.00E+08 | 100 | 1E−06 | 149.17% |
| fungi | 20 | 4E−13 | 1.00E+08 | 100 | 4E−06 | 593.33% |
| virus | 0.1 | 9E−18 | 1.00E+09 | 100 | 1E−10 | 0.01% |
| archaea | 1 | 9E−16 | 1.00E+10 | 100 | 1E−08 | 1.50% |
| host | 8 | 6E−14 | 1.00E+04 | 1 | 7E−05 | 9557.33% |
| host | 15 | 2E−13 | 1.00E+04 | 1 | 3E−04 | 33468.75% |
| host | 50 | 2E−12 | 1.00E+04 | 1 | 3E−03 | 364583.33% |

| | | | | | |
|---|---|---|---|---|---|
| Radius of Bead (mm) | 0.8 | Frequency (Hz) | 50 | Bead Fill Volume % | 30.00% |
| Number of beads | 280 | Time (s) | 120 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 2E−09 | 0.21% |
| bacteria | 1 | 1E−15 | 1.00E+08 | 100 | 6E−09 | 0.84% |
| bacteria | 2 | 5E−15 | 1.00E+08 | 100 | 3E−08 | 3.37% |
| fungi | 5 | 3E−14 | 1.00E+08 | 100 | 2E−07 | 21.05% |
| fungi | 10 | 1E−13 | 1.00E+08 | 100 | 6E−07 | 84.02% |
| fungi | 20 | 5E−13 | 1.00E+08 | 100 | 3E−06 | 334.69% |
| virus | 0.1 | 1E−17 | 1.00E+09 | 100 | 6E−11 | 0.01% |
| archaea | 1 | 1E−15 | 1.00E+10 | 100 | 6E−09 | 0.84% |
| host | 8 | 8E−14 | 1.00E+04 | 1 | 4E−05 | 5382.00% |
| host | 15 | 3E−13 | 1.00E+04 | 1 | 1E−04 | 18865.72% |
| host | 50 | 3E−12 | 1.00E+04 | 1 | 2E−03 | 206542.97% |

Host cell DNA signal presented in FIG. 22 shows increasing host cell disruption as a function of time, as demonstrated by increasing signal in Control conditions and decreasing signal in +MEM conditions (where freed host DNA is degraded during MEM). Since RNA is substantially less stable than DNA, we hypothesize that reduced RNA signal over time in Control conditions is attributable to RNA degradation during bead beating. Bacterial DNA and RNA remains relatively stable across time points in both Control and +MEM conditions, which is expected given the large size of the lysis beads.

The strong performance of MEM in MAT samples demonstrates the protocol's efficacy in a wide range of tissue types. MAT samples contain a high proportion of lipids and otherwise have similar matrices to other tissue types. Additionally, the red color of the tissues processed in the experiments above indicate a high blood content, likely due to the Crohn's Disease origin of the samples. Together, we expect these experiments to be representative of “worst case scenario” tissue processing. That is, the large tissue masses (100 mg) used, alongside the high proportion of contaminants in the MAT matrix, comprise MEM processing conditions more likely to be inhibited than other tissue types or lower masses of samples. We therefore expect these experiments to serve as a proof of concept demonstrating the efficacy of MEM processing in a range of sample conditions. These include tissue masses ranging from less than 1 mg up to 100 mg, as well as additional samples types including other tissues types (e.g. mucosal scrapings, SI biopsies, blood) and/or non-tissue lipidacous samples (e.g. milk, cheese, yogurt).

### Example 22: Lysis Efficiency of Fungi with Different Bead Sizes

Testing lysis efficiency of a liquid culture of Saccharomyces boulardii was performed with two different lysis beads for 3 different durations. 200 uL of diluted S. boulardii culture was placed in three different bead beating tubes and 600 uL of PM1 (Lysis buffer from Qiagen AllPrep PowerViral DNA/RNA Extraction Kit) was added. The two bead beating tubes tested were: Lysis Matrix E are 0.1 mm (can range from 0.074-0.150 mm) glass beads (SiO2)+1.4 mm (can range from 1.2-1.6 mm) zirconium silicate (ZrO2 64%, SiO2 33%)+4 mm glass beads (SiO2). Lysis Matrix Y consists of 0.5 mm diameter yttria-stabilized zirconium oxide beads (ZrO2 95%, Y2O3 5%) beads.

All samples were tested by bead beating for either 1, 2, or 3 minutes (labeled as cycles) at speed of 6.0 m/s. Samples were extracted with Qiagen AllPrep PowerViral DNA/RNA Extraction Kit and qPCR was performed on elutions targeting the TEF (translation elongation factor) gene. TEF is a single-copy gene present in nearly all fungi, including S. boulardii that can be used to approximate the fungal DNA content present. Extraction and media blanks were included to confirm no contaminating fungal signal was detected.

The results obtained in this example confirm the ones expected for the used configuration of beads and beads parameters according to the host depletion method of the present disclosure. Reference is made in this connection to the data reported in the table below.
a. 2R: 0.5 mm beads
b. Number of beads: 30%
c. Frequency: 60 Hz
d. Time: 180 s

| | | | | | |
|---|---|---|---|---|---|
| Radius of Bead (mm) | 0.25 | Frequency (Hz) | 60 | Bead Fill Volume % | 30.00% |
| Number of beads | 9167 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 1E−16 | 1.00E+08 | 100 | 3E−08 | 3.89% |
| bacteria | 1 | 4E−16 | 1.00E+08 | 100 | 1E−07 | 15.53% |
| bacteria | 2 | 2E−15 | 1.00E+08 | 100 | 5E−07 | 62.04% |
| fungi | 5 | 1E−14 | 1.00E+08 | 100 | 3E−06 | 386.21% |
| fungi | 10 | 4E−14 | 1.00E+08 | 100 | 1E−05 | 1534.46% |
| fungi | 20 | 2E−13 | 1.00E+08 | 100 | 5E−05 | 6054.91% |
| virus | 0.1 | 4E−18 | 1.00E+09 | 100 | 1E−09 | 0.16% |
| archaea | 1 | 4E−16 | 1.00E+10 | 100 | 1E−07 | 15.53% |
| host | 8 | 2E−14 | 1.00E+04 | 1 | 7E−04 | 98471.12% |
| host | 15 | 9E−14 | 1.00E+04 | 1 | 3E−03 | 342921.60% |
| host | 50 | 9E−13 | 1.00E+04 | 1 | 3E−02 | 3628800.00% |

e. 2R: 0.5 mm beads
f. Number of beads: 30%
g. Frequency: 60 Hz
h. Time: 60 s

| | | | | | |
|---|---|---|---|---|---|
| Radius of Bead (mm) | 0.25 | Frequency (Hz) | 60 | Bead Fill Volume % | 30.00% |
| Number of beads | 9167 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 1E−16 | 1.00E+08 | 100 | 1E−08 | 1.30% |
| bacteria | 1 | 4E−16 | 1.00E+08 | 100 | 4E−08 | 5.18% |
| bacteria | 2 | 2E−15 | 1.00E+08 | 100 | 2E−07 | 20.68% |
| fungi | 5 | 1E−14 | 1.00E+08 | 100 | 1E−06 | 128.74% |
| fungi | 10 | 4E−14 | 1.00E+08 | 100 | 4E−06 | 511.49% |
| fungi | 20 | 2E−13 | 1.00E+08 | 100 | 2E−05 | 2018.30% |
| virus | 0.1 | 4E−18 | 1.00E+09 | 100 | 4E−10 | 0.05% |
| archaea | 1 | 4E−16 | 1.00E+10 | 100 | 4E−08 | 5.18% |
| host | 8 | 2E−14 | 1.00E+04 | 1 | 2E−04 | 32823.71% |
| host | 15 | 9E−14 | 1.00E+04 | 1 | 9E−04 | 114307.20% |
| host | 50 | 9E−13 | 1.00E+04 | 1 | 9E−03 | 1209600.00% |

The results reported in FIG. 23 show that from this experiment, fungal DNA recovery was and therefore lysis was high under the given bead beating parameters, therefore matching the predicted outcomes. Improved fungal DNA recovery was noted with increased bead beating times. For mechanical disruption, we expect beads from 0.1 mm to 1 mm to lyse some fungi.

### Example 23 Benzonase Time Series and Proteinase K Optional Step

The effect was evaluated of differing enzyme inputs and incubation times on host depletion and microbial recovery using the MEM protocol. Human mesenteric adipose tissue was homogenized by bead beating at 4.5 m/s for 30 s using 1.4 mm zirconium-silicate beads (MP Biomedicals, MP116913050). Samples were then split and a) proceeded directly to extraction (Control), b) mixed with 2 ul of nuclease benzonase (+Bz −Pk), or c) mixed with 2 ul of nuclease benzonase and 5 ul of Proteinase K (+Bz +Pk) and incubated at 37 C with 600 RPM shaking for either 2 minutes or 15 minutes. After incubation, samples were centrifuged at 10,000 g for 2 minutes and resuspended in 200 ul of PM1 lysis buffer (Qiagen, 32500). Samples were extracted using Qiagen's AllPrep PowerViral DNA/RNA extraction kit (Qiagen, 32500) and eluted in 100 ul of nuclease free water and quantified using RT/qPCR. For RNA measurements, 25 ul of elution was mixed with 2 ul of TURBO DNAse and 3 ul of 10×DNAse buffer and incubated at 37 C for 30 minutes to remove DNA. DNAse was then removed using the Zymo RNA Clean & Concentrator kit (Zymo R1017). 2 ul of cleaned RNA template was then converted to cDNA using the High-Capacity cDNA Reverse Transcription Kit (Thermo Fisher Scientific 4368814). DNA and cDNA were quantified with qPCR using AccuStart II PCR SuperMix (Quantabio, 95137-100) with EvaGreen fluorophore (Biotium, 31000). Host loads were quantified using primers specific to mammalian ribosomal 18S gene (F: ACGGACCAGAGCGAAAGCAT (SEQ ID: 7), R: TGTCAATCCTGTCCGTGTCC (SEQ ID: 8). [5] Microbial loads were quantified using microbial 16S ribosomal gene specific primers targeting the V4 region (F: CAGCMGCCGCGGTAA (SEQ ID: 9), R: GGACTACHVGGGTWTCTAAT (SEQ ID: 10)).

The results reported in FIG. 24 show that varying enzyme input and incubation time yielded variable microbial recovery and host NA depletion. For example, incubating with benzonase but not proteinase produced relatively low host RNA depletion, likely because the ribosomal targets were being protected from nuclease degradation by riboproteins. The same samples yield reduced microbial recovery with proteinase treatment, likely for similar reasons. We conclude that these variable results are suitable for different applications. For example, in some applications where high microbial recovery is necessary and host RNA depletion is non-essential, it is likely sufficient to forego addition of proteinase K or long incubation. Alternatively, if maximum removal of host DNA and RNA is desired, longer incubation times and the addition of proteinase K would be beneficial.

### Example 24 Effect of Excess Bead Beating

The impact of non-specific bacterial cell lysis was characterized when bead beating using large (1.4 mm) zirconium silicate beads across high, mid, and low bacterial loads. Escherichia coli were innoculated in 2 mL of brain heart infusion (BHI) broth and cultured to stationary phase overnight at 37 C with 220 RPM shaking. Cultures were diluted and incubated until they reached exponential phase (as determined by taking serial spectrophotometer measurements until cultures reached an OD of 0.2). Once cultures reached exponential phase, they were transferred to centrifuge tubes and washed twice with PBS by centrifuging at 10,000 g for 2 minutes, removing the supernatant, and resuspending cell pellets with 1 mL of PBS.

Washed cells were then quantified using a spectrophotometer and diluted to 1e5 cells/mL (low), 1e7 cells/mL (mid), and 1e9 cells/mL (high) loads. Low, mid, and high load samples were then split and bead beat for either 0, 1, 2, or 3 cycles at 4.5 m/s for 1 minute using bead beating tubes with a mixture of 1.4 mm zirconium silicate beads (MP Biomedicals, 116913050-CF). Samples were then MEM treated by mixing 183 ul of bead beat sample with 10 ul of buffer comprised of 100 mM Tris and 40 mM MgCl2, along with 5 ul of Proteinase K (NEB, P8107S) and 2 ul of nuclease Benzonase (Sigma-Aldrich, 71205-3).

Samples were then incubated at 37 C for 15 minutes at 600 RPM shaking and then recovered by centrifuging at 10,000 g for 2 minutes, removing the supernatant, and resuspending cell pellets with 800 ul of lysis buffer. DNA and RNA from each sample was then recovered using Qiagen's AllPrep PowerViral DNA/RNA extraction kit (Qiagen, 32500) and eluted in 100 ul of nuclease free water. DNA loads were quantified with qPCR using AccuStart II PCR SuperMix (Quantabio, 95137-100) with EvaGreen fluorophore (Biotium, 31000) and using microbial 16S ribosomal gene specific primers targeting the V4 region (F: CAGCMGCCGCGGTAA (SEQ ID: 9), R: GGACTACHVGGGTWTCTAAT (SEQ ID: 10)).

The results obtained in this example confirm the ones expected for the used configuration of beads and beads parameters according to the host depletion method of the present disclosure. Reference is made in this connection to the data reported in the tables below.

| | | | | | |
|---|---|---|---|---|---|
| Radius of Bead (mm) | 0.6 | Frequency (Hz) | 40 | Bead Fill Volume % | 25.00% |
| Number of beads | 553 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 3E−09 | 0.37% |
| bacteria | 1 | 9E−16 | 1.00E+08 | 100 | 1E−08 | 1.50% |
| bacteria | 2 | 4E−15 | 1.00E+08 | 100 | 4E−08 | 5.99% |
| fungi | 5 | 2E−14 | 1.00E+08 | 100 | 3E−07 | 37.40% |
| fungi | 10 | 9E−14 | 1.00E+08 | 100 | 1E−06 | 149.17% |
| fungi | 20 | 4E−13 | 1.00E+08 | 100 | 4E−06 | 593.33% |
| virus | 0.1 | 9E−18 | 1.00E+09 | 100 | 1E−10 | 0.01% |
| archaea | 1 | 9E−16 | 1.00E+10 | 100 | 1E−08 | 1.50% |
| host | 8 | 6E−14 | 1.00E+04 | 1 | 7E−05 | 9557.33% |
| host | 15 | 2E−13 | 1.00E+04 | 1 | 3E−04 | 33468.75% |
| host | 50 | 2E−12 | 1.00E+04 | 1 | 3E−03 | 364583.33% |

2R: 1.2 mm to 1.6 mm
Number of beads: 25%
Frequency: 40 Hz

| | | | | | |
|---|---|---|---|---|---|
| Radius of Bead (mm) | 0.8 | Frequency (Hz) | 40 | Bead Fill Volume % | 25.00% |
| Number of beads | 233 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 2E−09 | 0.21% |
| bacteria | 1 | 1E−15 | 1.00E+08 | 100 | 6E−09 | 0.84% |
| bacteria | 2 | 5E−15 | 1.00E+08 | 100 | 3E−08 | 3.37% |
| fungi | 5 | 3E−14 | 1.00E+08 | 100 | 2E−07 | 21.05% |
| fungi | 10 | 1E−13 | 1.00E+08 | 100 | 6E−07 | 84.02% |
| fungi | 20 | 5E−13 | 1.00E+08 | 100 | 3E−06 | 334.69% |
| virus | 0.1 | 1E−17 | 1.00E+09 | 100 | 6E−11 | 0.01% |
| archaea | 1 | 1E−15 | 1.00E+10 | 100 | 6E−09 | 0.84% |
| host | 8 | 8E−14 | 1.00E+04 | 1 | 4E−05 | 5382.00% |
| host | 15 | 3E−13 | 1.00E+04 | 1 | 1E−04 | 18865.72% |
| host | 50 | 3E−12 | 1.00E+04 | 1 | 2E−03 | 206542.97% |

The results reported in FIG. 25 show that across low, mid, and high-load sample conditions, we found minimal losses to bead beating. This is concordant with our expectations that large (1.4 mm) beads would have minimal off-target lysis for small (˜1 um) bacterial cells. It is expected that these results to translate to other microbial cells with similar physical characteristics to Escherichia coli (e.g. other bacterial species found in the human microbiome).

Similarly, it is expected expect other cell types, including mammalian and fungal cell types, to follow similar trends. That is, specific lysis activity is expected through bead beating. For example, it is expected expect that bead beating large fungal cells with proportionately larger beads (i.e. >1.4 mm) over several cycles would yield low off-target lysis of those fungal cells across varying cell loads. In addition, it is expected expect these results to hold across different sample matrices, including homogenized tissue matrices. This is because increasing the viscosity of the sample matrix (to exceed the viscosity of PBS presented above) serves to reduce the impact of colliding beads against off-target cells. It is expected that increasing bead beating cycles using tissue samples with varying microbial loads would have a minimal effect on microbial cell lysis.

### Example 25: DTT Optional Step

FIG. 26: Effect of DTT in saliva on efficiency of host removal. Two healthy patients donated stimulated saliva and were split to be processed with and without MEM (with and without DTT). For MEM, 200 uL saliva was mixed with 200 uL saline (0.9% NaCl) and then bead beat in Lysing Matrix D (1.4 mm zirconium silicate) for 30 seconds at 4.5 m/s. For MEM+DTT, 200 uL saliva was mixed with 200 uL 10 mM DTT and then bead beat in Lysing Matrix D for 30 seconds at 4.5 m/s. For both MEM and MEM+DTT, 183 uL of homogenate was removed and incubated with 2 uL Benzonase+5 uL Proteinase K+10 uL 100 mM Tris+40 mM MgCl₂ (pH 8.0 and 0.22 μm sterile filtered) for 15 minutes at 37 C at 600 rpm before centrifuging at 10,000×g for 2 minutes.

The supernatant was then removed and the pellet was extracted using Qiagen AllPrep PowerViral DNA/RNA Extraction Kit. For the Control, 200 uL saliva was directly extracted using Qiagen AllPrep PowerViral DNA/RNA Extraction Kit. ddPCR was performed on the elutions using primers targeting the 16S rRNA gene and a single-copy host gene. Host loads are shown in panel A using the loads from the single-copy primer to estimate the number of human genomes remaining in the elution. Bacterial loads normalized to the Control samples are shown in panel B. Lxqimit of blank [LoB] defined as LoB=mean_{blank}+1.645[SD_{blank}] based on three processing blanks).

The results reported in FIG. 26 show that DTT can improve host removal with no impact on microbial recovery but works in a sample dependent manner, possibly due to mucus levels present in the sample. Host removal in Patient 1's saliva was unaffected by DTT while host removal in Patient 2's saliva was improved with DTT. Since it causes no detrimental effect to the microbial community, we recommend DTT treatment for samples with potentially high viscosity, such as saliva, aspirates, etc., and/or samples containing viscosity above 10 mPa-seconds due to the presence of mucus.

### Example 26 Small Host Fragments

FIG. 27: A human colonic biopsy was collected and processed with MEM treatment. MEM treatment consisted of: Placing the biopsy in 400 uL saline (0.9% NaCl) then bead beat in Lysing Matrix D (1.4 mm zirconium silicate) for 30 seconds at 4.5 m/s. 183 uL of homogenate was removed and incubated with 2 uL Benzonase+5 uL Proteinase K+10 uL 100 mM Tris+40 mM MgCl₂ (pH 8.0 and 0.22 μm sterile filtered) for 15 minutes at 37 C at 600 rpm before centrifuging at 10,000×g for 2 minutes. The supernatant was then removed, and the pellet was extracted using Qiagen AllPrep PowerViral DNA/RNA Extraction Kit. The elution was prepped for shotgun sequencing and sequenced using a 2×150 bp NovaSeq6000 at a depth of roughly 250M reads. Insert size was calculated by joining R1 and R2 through BBtools merge.sh. The distribution of insert size was plotted above with a stacked barplot. An expected average insert size is 300-350 bp. Inserts above 300 bp are not shown as no read overlap is detectable.

The results reported in FIG. 27 show that shotgun sequencing after MEM treatment generates a large population of libraries with very small inserts (i.e. between 30-100 bp). When we look at the identity of these small inserts, they appear to be almost exclusively host derived. This may be due to the presence of partly degraded host DNA. Therefore if those fragments can be removed the host depletion during sequencing can be improved as will be understood by a skilled person.

An exemplary method to remove partly degraded host DNA (i.e. defined as below 5000 bp) is through SPRI bead clean-ups. To test this, rat scrapings (RS) that were previously MEM treated (following the same protocol as the above biopsies) were SPRI cleaned (AMPure XP beads were utilized) at various ratios of beads to elution (see legend) to remove partly degraded host DNA. A ratio of 1.8:1 beads:elution will remove fragments below −20 bp. A ratio of 0.9 beads:elution will remove fragments below −100 bp. A ratio of 0.6 beads:elution will remove fragments below 300 bp. Host and bacterial DNA content in samples were then quantified using qPCR targeting a LINE1 transposon gene in rat (ORF2A; 50 bp amplicon) as a proxy for host DNA content and 16S rRNA gene (200 bp amplicon) as a proxy for bacterial/archaea DNA content. A transposon gene was used due to its high abundance in the rat genome, making it a sensitive marker for host DNA content.

The results reported in FIG. 28 show additional removal of ORF2A host signal with bead clean-up above 100 bp, with minimal impact on 16S bacterial signal. However, there is some additional signal present in the qPCR using ORF2A primers. We would expect additional removal of host signal may be possible by increasing the cutoff size of the SPRI beads. Size cutoffs should be decided based on the extraction method utilized. For long read sequencing, this size cutoff may be much larger than for the mechanical shearing methods and column extraction we utilized in this experiment.

### Example 27 Validation on Pancreatic Samples

FIG. 29 MEM was tested on mouse pancreatic tissue. Roughly 50 mg of mouse pancreatic tissue was obtained from the tumors of mice with pancreatic cancer. Pancreatic tissue from 9 different mice were collected, typically from the head of the pancreas as this is the most common tumor location. After sample collection, the pancreatic tumor was homogenized with a pipette tip and MEM samples were then placed into Lysis Matrix D (1.4 mm zirconium silicate beads) with 400 uL saline and then processed with Benzonase and Proteinase K as described in Example 7. Control samples were extracted immediately with Qiagen AllPrep PowerViral DNA/RNA Extraction Kit. qPCR was performed on elutions with primers targeting a single-copy mouse primer to quantify host genomes remaining. Cqs were converted to copies per mg of tissue using an approximated standard curve and by normalizing to input mass.

The results reported in FIG. 29 show that MEM treatment consistently removed host ˜10,000-fold in pancreatic tissue. Therefore, we show MEM applicability in soft organ tissue types. We expect MEM to work on other organs, such as the brain, lungs, liver, kidney, spleen, thyroid, and heart. We expect MEM to work with up to 50 mg of sample using the above protocol.

### Example 28: mRNA Fixation Applications of MEM

FIG. 30 To study the transcriptomic profiles of bacteria cells, the sample can be treated with different embodiments of MEM. In this experiment, known transcriptomic changes in ptsG, ptsH, and gapA were induced in E. coli cells by growing them in media containing either Glucose or Pyruvate as the only carbon source.

As shown in FIG. 30, the expected increase of these transcripts in the glucose growth condition was observed in the control, with No MEM condition. Additionally, it is shown that some changes in transcript levels can be captured by embodiments of MEM with and without fixation steps (changes in ptsG and ptsH), but that an embodiment of MEM including a fixation step expands our ability to capture transcriptomic changes in the treated bacteria (changes in gapA, ptsG, and ptsH can all be seen in the Fix MEM 2 min condition). All mRNA levels were normalized to 16S levels in order to account for different amounts of total RNA extracted from the samples. This normalization enables us to capture fold changes in mRNA that are similar to what has been documented previously (4-fold or 2 Cq increases for gapA and 2.4-4.8-fold or 1.3-2.3 Cq increases for ptsG and ptsH in the glucose growth conditions). Absolute transcript levels for gapA, 16S, ptsG, and ptsH are shown in part A, and normalized transcript levels of gapA, ptsG, and ptsH are shown in B.

### Example 29 Inactivation of Benzonase

FIG. 31 a method was identified that can inactivate the nucleases added during the MEM during microbial lysis. To test this, we added 2 uL of Benzonase into inactivation (PrimeStore MTM) buffer and a sample of DNA/RNA from Neisseria gonorrhoeae. We left the sample to incubate for 24 hours at room temperature to quantify any residual DNase or RNase activity in Benzonase. After 24 hours, samples were extracted with Qiagen AllPrep PowerViral DNA/RNA Extraction Kit. The original spike-in of N. gonorrhoeae was extracted with the same method to quantify any losses to the kit (labeled Control). The N gonorrhoeae DNA and RNA in the elutions were then quantified with qPCR and RT-qPCR respectively targeting a 16S rRNA gene. qPCR, or DNA load, are plotted on the left and RT-qPCR, or RNA load, are plotted on the right.

The results reported in FIG. 3l show that no DNA or RNA losses were detected when exposed to PrimeStore inactivated Benzonase. Additionally, PrimeStore was compatible with the NA extraction kit. We expect lower concentrations of Benzonase to have DNase and RNase activity inhibited as well. We expect samples stored for longer time periods (on the order of a week at room temperature or 6 months at −80 C) will also have stable DNA and RNA loads.

Additionally 100 mg aliquots of human mesenteric adipose tissue (MAT) were prepared under aerobic conditions. Sample aliquots were homogenized under either aerobic or anaerobic conditions by suspending samples in 800 ul of PBS along with 1.4 mm zirconium-silicate beads (MP Biomedicals, MP116913050). Samples were then vortexed at maximum speed for 2 minutes and sample homogenates either a) proceeded immediately to DNA/RNA extraction (No MEM), or b) MEM treated prior to DNA/RNA extraction (MEM). For anaerobic processing conditions, both MEM and No MEM conditions remained under anaerobic conditions until DNA/RNA extraction. MEM treatment consisted of combining 183 ul of homogenate with 10 ul of 100 mM Tris+40 mM MgCl₂, 5 ul of Proteinase K, and 2 ul of nuclease Benzonase. MEM samples were then mixed and incubated at 37 C with 600 RPM shaking for 15 minutes. After incubation, MEM samples were centrifuged at 10,000 g for 2 minutes and resuspended in 200 ul of PM1 lysis buffer (Qiagen, 32500). All samples were extracted using Qiagen's AllPrep PowerViral DNA/RNA extraction kit (Qiagen, 32500) and eluted in 100 ul of nuclease free water. DNA was quantified with qPCR using AccuStart II PCR SuperMix (Quantabio, 95137-100) with EvaGreen fluorophore (Biotium, 31000). Host loads were quantified using primers specific to mammalian ribosomal 18S gene (F: ACGGACCAGAGCGAAAGCAT (SEQ ID: 7), R: TGTCAATCCTGTCCGTGTCC (SEQ ID: 8)) and microbial loads were quantified using microbial 16S ribosomal gene specific primers targeting the V4 region (F: CAGCMGCCGCGGTAA (SEQ ID: 9), R: GGACTACHVGGGTWTCTAAT SEQ ID: 10)). loads were quantified using microbial 16S ribosomal gene specific primers targeting the V4 region (F: CAGCMGCCGCGGTAA (SEQ ID: 9), R: GGACTACHVGGGTWTCTAAT (SEQ ID: 10)). loads were quantified using microbial 16S ribosomal gene specific primers targeting the V4 region (F: CAGCMGCCGCGGTAA (SEQ ID: 9), R: GGACTACHVGGGTWTCTAAT (SEQ ID: 10)).

The results shown in FIG. 32 indicate that MEM processing yielded similar results between aerobic and anaerobic conditions. In particular, No MEM and MEM host and microbe signals were similar across aerobic and anaerobic conditions, with differences being attributable to variability among samples. These results emphasize the ease with which MEM processing can be accomplished. Notably, MEM was completed in under 30 minutes within a highly-confined anaerobic chamber. We consider the simple homogenization and stepwise addition of enzymes a feature of the MEM protocol and expect that the ease of the protocol will support similarly difficult processing conditions (e.g. processing that requires BSL2 or higher).

### Example 30 Validation of Method on Flash Frozen Samples

Validation has been performed on paired fresh and flash frozen saliva samples to confirm MEM compatibility on flash frozen liquid sample types. One human saliva sample was split 6 different ways. 3 of the samples were flash frozen immediately after collection by placing it on an ethanol dry ice bath. After freezing, samples were re-thawed and processed using the MEM treatment and extracted as described in Example 6. The other 3 saliva samples were MEM treated immediately after collection and extracted as described in Example 5. 16S rRNA gene sequencing and analysis was performed on all elutions as described in Example 1D.

The results reported in FIG. 33 show Relative abundances of taxa present in the flash frozen (x-axis) vs the fresh (y-axis) saliva samples were plotted against each other. High correlation for almost all taxa (or all taxa above 0.1% relative abundance) was present between the flash frozen and fresh samples. This shows feasibility of MEM treatment on both fresh and flash frozen samples.

We expect other flash freezing processes to yield similar results, such as flash freezing in liquid nitrogen. We expect additional cryoprotectants can be added to the flash freezing process to increase preservation of specific microbes of interest. We expect other liquid samples would also be compatible with flash freezing.

Validation has been performed on paired control (untreated), fresh, and flash frozen duodenal biopsy samples to confirm MEM compatibility on flash frozen solid tissue sample types. Six human duodenal biopsies were collected during endoscopy from one field of view (5 cm diameter). Two of the biopsies were flash frozen immediately after collection by placing it on an ethanol dry ice bath (labeled Flash Frozen MEM). After freezing, samples were re-thawed and processed using the MEM treatment and extracted as described in Example 6. Two other biopsies were MEM treated immediately after collection and extracted as described in Example 6. The last two biopsies were extracted immediately following the extraction protocol in Example 6 but no MEM treatment. Quant-Seq was performed on all biopsies as described in Example 4 (see FIG. 5B. Principal coordinates analysis (PCA) on microbial genus-level relative abundances were performed to visualize microbial population variation.

The results reported in FIG. 34 show PCA of sequencing results showed that any differences in microbial relative abundances introduced by MEM were less than the differences observed between participants. This shows feasibility of MEM treatment on both fresh and flash frozen duodenal biopsies.

It is expected that other flash freezing processes to yield similar results, such as flash freezing in liquid nitrogen. It is also expected that additional cryoprotectants can be added to the flash freezing process to increase preservation of specific microbes of interest. It is further expected that other solid tissue samples would also be compatible with flash freezing. It is additionally expected that expect MEM to be effective on other samples with low bacterial load.

In additional experiments A culture of Saccharomyces cerevisiae var. boulardii CNCM I-745 was inoculated in liquid YPD broth (BD 242820) in aerobic conditions at 30 C. At approximately 3, 24, and 48 hours, 500 uL of the cell culture was removed from culture. The cell concentration was normalized to an OD600 of 1.0 with saline and promptly split into two volumes; one to be treated with host depletion (+) and one without host depletion (−). The host depletion (+) volume underwent the host depletion protocol as previously described, while the host depletion (−) sample was volume adjusted to match the (+) sample. Finally, liquid samples were diluted and plated onto YPD plates with 1.5% agar and incubated for 48 hours before colonies were counted.

The results reported in FIG. 35 show Spread plating of S. boulardii before and after host depletion at different time points after culture inoculation. Colony counts of S. boulardii are plotted on the y axis (log 10 CFU/mL) for samples that were processed before (−) and after (+) host depletion at exponential growth phase (3 hours after culture inoculation) and stationary phases (24 hours and 48 hours after the initial inoculation). Each bar is calculated from the average of four plating replicates, except for the (−) host depletion condition at stationary (48 hr) growth, where only one plate was counted. Error bars depict standard deviation.

In a further set of experiments A ten-taxa whole cell mix (ATCC MSA2010) was purchased as a lyophilized powder and resuspended in 1 mL PBS. On the day of the experiment, 100 uL of this MSA2010 taxa mix was diluted in 900 uL of saline to create the 10× dilution condition, and was serial diluted further 100× for the 1000× dilution condition. Following dilution, samples were either treated with or without host depletion as previously described. Samples that did not undergo host depletion were dilution matched with the addition of saline in the place of host depletion reagents.

Immediately following host depletion, all samples were first extracted via mechanical disruption using Lysis Matrix E (MP Bio 116914050-CF) for three cycles of 60 seconds at 6.5 m/s. Following bead beating, samples were extracted using the ZymoBIOMICs MagBead DNA/RNA extraction kit (ZYMO R2135). The resulting elution was analyzed for DNA, while 20 uL was taken for subsequent DNase treatment, clean-up, and RT.

Following extraction, 20 uL of the elution was taken and treated with TURBO DNase (Invitrogen AM2238) and subsequently cleaned up with RNA Clean & Concentrator kit (ZYMO R1017). cDNA library was then synthesized using a High-Capacity cDNA Reverse Transcriptase Kit (Applied Biosystems 4368814). Following cDNA library synthesis, samples were cleaned up by adding 20 uL of cDNA library and 36 uL of AMPure XP beads (Beckman Coulter A63881). After incubating at room temperature for 5 minutes, samples were placed on a magnetic tube rack for separation and washed with 200 uL of 70% ethanol for two cycles. Following a 5 minute drying step, samples were resuspended in 20 uL of nuclease-free water.

The results reported in FIG. 36. 18S RNA and DNA of a fungal community before and after host depletion. The Cq of 18S RNA (first two columns) and 18S DNA (third and fourth columns) before (−HD) and after (+HD) host depletion is plotted on the y axis for a complex ten-taxa mix (MSA2010) that has been either 10× diluted (solid circle) or 1000× diluted (solid upside down triangle) from stock. HD negative controls, or samples with no fungal cells before (−HD) and after (+HD) host depletion are depicted as empty squares. RT negative controls, or samples with no fungal DNA/RNA with (+RT) and without (−RT) reverse transcriptase are depicted as empty upside down triangles. Each data point is an extraction replicate averaged from two qPCR replicates. The horizontal dotted line at 40 Cq indicates the number of cycles used in the qPCR protocol. ND, not detected.

In an additional set of experiments was performed to detect community composition of a ten-taxa fungal mix before and after host depletion.

Extracted DNA was amplified and sequenced using barcoded universal primers and protocol modified to reduce amplification of off target DNA and primer-dimer formation (cite Jacob). Amplification was monitored in a CFX96 RT-PCR machine (Bio-Rad) and samples were removed once fluorescence measurements reached early stationary phase.

The 18S rRNA gene sequence was amplified in duplicate with the following PCR reaction components: 1× AccuStart mastermix, 1× Evagreen, 500 nM forward and reverse primers. PCR cycling conditions were as follows: 94 C for 5 min, up to 40 cycles of 94 C for 30 s, 58.3 C for 50 s and 72 C for 1 min.

The ITS rRNA gene sequence was amplified in duplicate with the following PCR reaction components: 1×KAPA HiFi mastermix, 1× Evagreen, 500 nM forward and reverse primers. PCR cycling conditions were as follows: 94 C for 3 min, up to 40 cycles of 95 C for 30 s, 61.4 C for 30 s and 72 C for 2 min.

Reactions were pooled together and quantified with Kapa library quantification kit (Kapa Biosystems, KK4824, Wilmington, MA, USA) before equimolar sample pooling. Libraries were concentrated and cleaned using AMpureXP beads (Beckman Coulter, Brea, CA USA). Sequencing was performed using the Illumina MiSeq platform with v2 chemistry.

Raw reads were first converted to FASTQ format and demultiplexed using Illumina's “bcl2fastq” program (v1.8.4). Primer sequences and adapters were then removed using cutadapt (v4.4). Denoising and dereplication was then performed using DADA2 (v1.16). Taxonomical classification of amplicon sequence variants was performed using a naïve Bayes classifier from QIIME2 (v2023.5) and sci-kit learn. Training data for the taxonomical classifier was obtained by performing in silico PCR on NCBI's RefSeq database.

The results reported in FIG. 37 shows the detect community composition of a ten-taxa fungal mix before and after host depletion. The ratio of the relative abundances of each fungal taxa after and before host depletion (+HD/−HD) are plotted on the y axis for each fungal taxon in MSA2010 for 18S. Each condition is calculated from the average of three extraction replicates. Error bars depict the standard deviation. The dotted horizontal line depicts no change in community composition before and after host depletion. Samples were prepared and extracted as stated above. The primers used for analysis targeted the ITS and 18S regions of the fungal genome.

### Example 31: SNVs Detectable Across Locations in the Lower GI Tract within an Individual

A set of experiments was performed to investigate whether MEM treatment could enable studies of microbial population genetics in low-biomass samples through single-nucleotide variants (SNVs) as a result of the increased depth of coverage. For this analysis, we analyzed MAGs from patient CT12 as reference genomes and mapped the paired-end reads from the terminal ileum and descending colon from CT12 onto these assembled genomes.

Six MAGs had a mean coverage above 50× across all 6 samples (3 terminal ileum and 3 descending colon) and were selected for subsequent SNV analysis (FIG. 19). SNV profiles were generated from the paired-end reads of each sample by comparing them with the reference sequence (MAG).

It was investigated whether PCR errors can be responsible for some of the SNVs observed in our data by preparing libraries for an additional three technical replicates from a single terminal ileum biopsy (FIG. 10B), with the expectation that differences in the SNV profiles of the technical replicates should be minimal. By looking at nucleotide variations occurring in one, two, or all three replicates, we observed that a minimum deviation from the reference nucleotide of 21% for Ruminococcus bromii (FIG. 10B) allowed for the selection of SNVs only and minimized the impact of PCR errors in the population structure analysis. Analyses of these data using fixation index showed that some taxa, such as R. bromii (FIG. 6C) and Gemmiger formicilis (FIG. 19), were composed of subpopulations that were distinct between the upper and lower intestinal tract. To assess whether these SNVs were functionally significant, codon-level and translated (amino acid) analyses of SNVs in R. bromii were performed and similar clustering of biopsies by location was detected (FIG. 20). The recovery of SNVs afforded by the deeper sequencing and increased coverage of MAGs from biopsy samples allowed us to detect the presence of subpopulation structures for some individual taxa along the lower GI tract of a single individual.

### Example C1 Individual Cells

The set of beads radius and beads parameters expected for bacteria, archaea, fungi, and virus with Md below 1 um:
2R: 1.0 mm to 1.8 mm
Number of beads: 5-60% by volume of the tube (before packing)
Frequency: 16 Hz to 150 Hz
Time: 15 s to 300 s

As shown in the following tables:

| | | | | | |
|---|---|---|---|---|---|
| Radius of Bead (mm) | 0.5 | Frequency (Hz) | 16 | Bead Fill Volume % | 5.00% |
| Number of beads | 191 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 3E−11 | 0.00% |
| bacteria | 1 | 8E−16 | 1.00E+08 | 100 | 1E−10 | 0.01% |
| bacteria | 2 | 3E−15 | 1.00E+08 | 100 | 4E−10 | 0.06% |
| fungi | 5 | 2E−14 | 1.00E+08 | 100 | 3E−09 | 0.36% |
| fungi | 10 | 8E−14 | 1.00E+08 | 100 | 1E−08 | 1.43% |
| fungi | 20 | 3E−13 | 1.00E+08 | 100 | 4E−08 | 5.68% |
| virus | 0.1 | 8E−18 | 1.00E+09 | 100 | 1E−12 | 0.00% |
| archaea | 1 | 8E−16 | 1.00E+10 | 100 | 1E−10 | 0.01% |
| host | 8 | 5E−14 | 1.00E+04 | 1 | 7E−07 | 91.67% |
| host | 15 | 2E−13 | 1.00E+04 | 1 | 2E−06 | 320.76% |
| host | 50 | 2E−12 | 1.00E+04 | 1 | 3E−05 | 3480.00% |

| | | | | | |
|---|---|---|---|---|---|
| Radius of Bead (mm) | 0.7 | Frequency (Hz) | 83 | Bead Fill Volume % | 32.50% |
| Number of beads | 452 | Time (s) | 158 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 5E−09 | 0.65% |
| bacteria | 1 | 1E−15 | 1.00E+08 | 100 | 2E−08 | 2.60% |
| bacteria | 2 | 4E−15 | 1.00E+08 | 100 | 8E−08 | 10.39% |
| fungi | 5 | 3E−14 | 1.00E+08 | 100 | 5E−07 | 64.87% |
| fungi | 10 | 1E−13 | 1.00E+08 | 100 | 2E−06 | 258.88% |
| fungi | 20 | 4E−13 | 1.00E+08 | 100 | 8E−06 | 1030.56% |
| virus | 0.1 | 1E−17 | 1.00E+09 | 100 | 2E−10 | 0.03% |
| archaea | 1 | 1E−15 | 1.00E+10 | 100 | 2E−08 | 2.60% |
| host | 8 | 7E−14 | 1.00E+04 | 1 | 1E−04 | 16584.01% |
| host | 15 | 2E−13 | 1.00E+04 | 1 | 4E−04 | 58108.07% |
| host | 50 | 3E−12 | 1.00E+04 | 1 | 5E−03 | 634807.08% |

| | | | | | |
|---|---|---|---|---|---|
| Radius of Bead (mm) | 0.9 | Frequency (Hz) | 150 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−08 | 2.50% |
| bacteria | 1 | 1E−15 | 1.00E+08 | 100 | 7E−08 | 10.00% |
| bacteria | 2 | 6E−15 | 1.00E +08 | 100 | 3E−07 | 39.97% |
| fungi | 5 | 4E−14 | 1.00E+08 | 100 | 2E−06 | 249.54% |
| fungi | 10 | 1E−13 | 1.00E+08 | 100 | 7E−06 | 996.30% |
| fungi | 20 | 6E−13 | 1.00E+08 | 100 | 3E−05 | 3970.37% |
| virus | 0.1 | 1E−17 | 1.00E+09 | 100 | 7E−10 | 0.10% |
| archaea | 1 | 1E−15 | 1.00E+10 | 100 | 7E−08 | 10.00% |
| host | 8 | 9E−14 | 1.00E+04 | 1 | 5E−04 | 63810.37% |
| host | 15 | 3E−13 | 1.00E+04 | 1 | 2E−03 | 223750.00% |
| host | 50 | 3E−12 | 1.00E+04 | 1 | 2E−02 | 2453703.70% |

### Example C2 Individual Cells

The set of beads radius and beads parameters expected for bacteria, archaea, fungi, and virus with Md between 1-2 um:
2R: 1.0 mm to 1.3 mm
Number of beads: 5-30% by volume of the tube (before packing)
Frequency: 16 Hz to 60 Hz
Time: 15 s to 60 s

As shown in the tables below:

| | | | | | |
|---|---|---|---|---|---|
| Radius of Bead (mm) | 0.5 | Frequency (Hz) | 16 | Bead Fill Volume % | 5.00% |
| Number of beads | 191 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 3E−11 | 0.00% |
| bacteria | 1 | 8E−16 | 1.00E+08 | 100 | 1E−10 | 0.01% |
| bacteria | 2 | 3E−15 | 1.00E+08 | 100 | 4E−10 | 0.06% |
| fungi | 5 | 2E−14 | 1.00E+08 | 100 | 3E−09 | 0.36% |
| fungi | 10 | 8E−14 | 1.00E+08 | 100 | 1E−08 | 1.43% |
| fungi | 20 | 3E−13 | 1.00E+08 | 100 | 4E−08 | 5.68% |
| virus | 0.1 | 8E−18 | 1.00E+09 | 100 | 1E−12 | 0.00% |
| archaea | 1 | 8E−16 | 1.00E+10 | 100 | 1E−10 | 0.01% |
| host | 8 | 5E−14 | 1.00E+04 | 1 | 7E−07 | 91.67% |
| host | 15 | 2E−13 | 1.00E+04 | 1 | 2E−06 | 320.76% |
| host | 50 | 2E−12 | 1.00E+04 | 1 | 3E−05 | 3480.00% |

| | | | | | |
|---|---|---|---|---|---|
| Radius of Bead (mm) | 0.65 | Frequency (Hz) | 60 | Bead Fill Volume % | 30.00% |
| Number of beads | 522 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−09 | 0.19% |
| bacteria | 1 | 1E−15 | 1.00E+08 | 100 | 6E−09 | 0.77% |
| bacteria | 2 | 4E−15 | 1.00E+08 | 100 | 2E−08 | 3.06% |
| fungi | 5 | 3E−14 | 1.00E+08 | 100 | 1E−07 | 19.12% |
| fungi | 10 | 1E−13 | 1.00E+08 | 100 | 6E−07 | 76.29% |
| fungi | 20 | 4E−13 | 1.00E+08 | 100 | 2E−06 | 303.60% |
| virus | 0.1 | 1E−17 | 1.00E+09 | 100 | 6E−11 | 0.01% |
| archaea | 1 | 1E−15 | 1.00E+10 | 100 | 6E−09 | 0.77% |
| host | 8 | 7E−14 | 1.00E+04 | 1 | 4E−05 | 4887.79% |
| host | 15 | 2E−13 | 1.00E+04 | 1 | 1E−04 | 17121.71% |
| host | 50 | 2E−12 | 1.00E+04 | 1 | 1E−03 | 186800.18% |

### Example C3 Individual Cells

The set of beads radius and beads parameters expected for bacteria, archaea, fungi, and virus with Md between 1-2 um:
2R: 1.3 mm to 1.8 mm
Number of beads: 5-60% by volume of the tube (before packing)
Frequency: 13 Hz to 100 Hz
Time: 15 s to 1300 s

As shown in the following tables:

| | | | | | |
|---|---|---|---|---|---|
| Radius of beads (mm) | 0.65 | Frequency (Hz) | 16 | Bead fill Volume % | 5.00% |
| Number of beads | 87 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 2E−11 | 0.00% |
| bacteria | 1 | 1E−15 | 1.00E+08 | 100 | 6E−11 | 0.01% |
| bacteria | 2 | 4E−15 | 1.00E+08 | 100 | 3E−10 | 0.03% |
| fungi | 5 | 3E−14 | 1.00E+08 | 100 | 2E−09 | 0.21% |
| fungi | 10 | 1E−13 | 1.00E+08 | 100 | 6E−09 | 0.85% |
| fungi | 20 | 4E−13 | 1.00E+08 | 100 | 3E−08 | 3.37% |
| virus | 0.1 | 1E−17 | 1.00E+09 | 100 | 6E−13 | 0.00% |
| archaea | 1 | 1E−15 | 1.00E+10 | 100 | 6E−11 | 0.01% |
| host | 8 | 7E−14 | 1.00E+04 | 1 | 4E−07 | 54.31% |
| host | 15 | 2E−13 | 1.00E+04 | 1 | 1E−06 | 190.24% |
| host | 50 | 2E−12 | 1.00E+04 | 1 | 2E−05 | 2075.56% |

| | | | | | |
|---|---|---|---|---|---|
| Radius of Bead (mm) | 0.825 | Frequency (Hz) | 38 | Bead Fill Volume % | 17.50% |
| Number of beads | 149 | Time (s) | 38 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 2E−10 | 0.03% |
| bacteria | 1 | 1E−15 | 1.00E+08 | 100 | 8E−10 | 0.11% |
| bacteria | 2 | 5E−15 | 1.00E+08 | 100 | 3E−09 | 0.44% |
| fungi | 5 | 3E−14 | 1.00E+08 | 100 | 2E−08 | 2.74% |
| fungi | 10 | 1E−13 | 1.00E+08 | 100 | 8E−08 | 10.95% |
| fungi | 20 | 5E−13 | 1.00E+08 | 100 | 3E−07 | 43.61% |
| virus | 0.1 | 1E−17 | 1.00E+09 | 100 | 8E−12 | 0.00% |
| archaea | 1 | 1E−15 | 1.00E+10 | 100 | 8E−10 | 0.11% |
| host | 8 | 8E−14 | 1.00E+04 | 1 | 5E−06 | 701.20% |
| host | 15 | 3E−13 | 1.00E+04 | 1 | 2E−05 | 2458.15% |
| host | 50 | 3E−12 | 1.00E+04 | 1 | 2E−04 | 26924.20% |

| | | | | | |
|---|---|---|---|---|---|
| Radius of Bead (mm) | 0.9 | Frequency (Hz) | 100 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 1E−08 | 1.67% |
| bacteria | 1 | 1E−15 | 1.00E+08 | 100 | 5E−08 | 6.66% |
| bacteria | 2 | 6E−15 | 1.00E+08 | 100 | 2E−07 | 26.65% |
| fungi | 5 | 4E−14 | 1.00E+08 | 100 | 1E−06 | 166.36% |
| fungi | 10 | 1E−13 | 1.00E+08 | 100 | 5E−06 | 664.20% |
| fungi | 20 | 6E−13 | 1.00E+08 | 100 | 2E−05 | 2646.91% |
| virus | 0.1 | 1E−17 | 1.00E+09 | 100 | 5E−10 | 0.07% |
| archaea | 1 | 1E−15 | 1.00E+10 | 100 | 5E−08 | 6.66% |
| host | 8 | 9E−14 | 1.00E+04 | 1 | 3E−04 | 42540.25% |
| host | 15 | 3E−13 | 1.00E+04 | 1 | 1E−03 | 149166.67% |
| host | 50 | 3E−12 | 1.00E+04 | 1 | 1E−02 | 1635802.47% |

### Example C4: Individual Cells

The set of beads radius and beads parameters expected for bacteria, archaea, fungi, and virus with Md above 2 um:
2R: 1.2 mm to 1.6 mm
Number of beads: 5-30% by volume of the tube (before packing)
Frequency: 16 Hz to 35 Hz
Time: 15 s to 60 s

As shown in the following tables:

| | | | | | |
|---|---|---|---|---|---|
| Radius of Bead (mm) | 0.6 | Frequency (Hz) | 16 | Bead Fill Volume % | 5.00% |
| Number of beads | 111 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 2E−11 | 0.00% |
| bacteria | 1 | 9E−16 | 1.00E+08 | 100 | 7E−11 | 0.01% |
| bacteria | 2 | 4E−15 | 1.00E+08 | 100 | 3E−10 | 0.04% |
| fungi | 5 | 2E−14 | 1.00E+08 | 100 | 2E−09 | 0.25% |
| fungi | 10 | 9E−14 | 1.00E+08 | 100 | 7E−09 | 0.99% |
| fungi | 20 | 4E−13 | 1.00E+08 | 100 | 3E−08 | 3.96% |
| virus | 0.1 | 9E−18 | 1.00E+09 | 100 | 7E−13 | 0.00% |
| archaea | 1 | 9E−16 | 1.00E+10 | 100 | 7E−11 | 0.01% |
| host | 8 | 6E−14 | 1.00E+04 | 1 | 5E−07 | 63.72% |
| host | 15 | 2E−13 | 1.00E+04 | 1 | 2E−06 | 223.13% |
| host | 50 | 2E−12 | 1.00E+04 | 1 | 2E−05 | 2430.56% |

### Example C5: Individual Cells

The set of beads radius and beads parameters expected for bacteria, archaea, fungi, and virus with Md below 10 um:
2R: 1.2 mm to 1.6 mm
Number of beads: 5-30% by volume of the tube (before packing)
Frequency: 35 Hz to 60 Hz
Time: 15 s to 60 s

As shown in the following tables:

| | | | | | |
|---|---|---|---|---|---|
| Radius of Bead (mm) | 0.7 | Frequency (Hz) | 38 | Bead Fill Volume % | 17.50% |
| Number of beads | 244 | Time (s) | 38 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 3E−10 | 0.04% |
| bacteria | 1 | 1E−15 | 1.00E+08 | 100 | 1E−09 | 0.15% |
| bacteria | 2 | 4E−15 | 1.00E+08 | 100 | 5E−09 | 0.61% |
| fungi | 5 | 3E−14 | 1.00E+08 | 100 | 3E−08 | 3.81% |
| fungi | 10 | 1E−13 | 1.00E+08 | 100 | 1E−07 | 15.20% |
| fungi | 20 | 4E−13 | 1.00E+08 | 100 | 5E−07 | 60.49% |
| virus | 0.1 | 1E−17 | 1.00E+09 | 100 | 1E−11 | 0.00% |
| archaea | 1 | 1E−15 | 1.00E+10 | 100 | 1E−09 | 0.15% |
| host | 8 | 7E−14 | 1.00E+04 | 1 | 7E−06 | 973.42% |
| host | 15 | 2E−13 | 1.00E+04 | 1 | 3E−05 | 3410.73% |
| host | 50 | 3E−12 | 1.00E+04 | 1 | 3E−04 | 37260.84% |

| | | | | | |
|---|---|---|---|---|---|
| Radius of Bead (mm) | 0.8 | Frequency (Hz) | 60 | Bead Fill Volume % | 30.00% |
| Number of beads | 280 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 9E−10 | 0.13% |
| bacteria | 1 | 1E−15 | 1.00E+08 | 100 | 4E−09 | 0.51% |
| bacteria | 2 | 5E−15 | 1.00E+08 | 100 | 2E−08 | 2.02% |
| fungi | 5 | 3E−14 | 1.00E+08 | 100 | 9E−08 | 12.63% |
| fungi | 10 | 1E−13 | 1.00E+08 | 100 | 4E−07 | 50.41% |
| fungi | 20 | 5E−13 | 1.00E+08 | 100 | 2E−06 | 200.81% |
| virus | 0.1 | 1E−17 | 1.00E+09 | 100 | 4E−11 | 0.01% |
| archaea | 1 | 1E−15 | 1.00E+10 | 100 | 4E−09 | 0.51% |
| host | 8 | 8E−14 | 1.00E+04 | 1 | 2E−05 | 3229.20% |
| host | 15 | 3E−13 | 1.00E+04 | 1 | 8E−05 | 11319.43% |
| host | 50 | 3E−12 | 1.00E+04 | 1 | 9E−04 | 123925.78% |

### Example C6 Individual Cells

The set of beads radius and beads parameters expected for bacteria, archaea, fungi, and virus with Md above 2 um:
2R: 1.4 mm to 1.8 mm
Number of beads: 5-50% by volume of the tube (before packing.)
Frequency: 16 Hz to 40 Hz
Time: 15 s to 60 s

As shown in the following table:

| | | | | | |
|---|---|---|---|---|---|
| Radius of Bead (mm) | 0.7 | Frequency (Hz) | 16 | Bead Fill Volume % | 5.00% |
| Number of beads | 70 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−11 | 0.00% |
| bacteria | 1 | 1E−15 | 1.00E+08 | 100 | 6E−11 | 0.01% |
| bacteria | 2 | 4E−15 | 1.00E+08 | 100 | 2E−10 | 0.03% |
| fungi | 5 | 3E−14 | 1.00E+08 | 100 | 1E−09 | 0.18% |
| fungi | 10 | 1E−13 | 1.00E+08 | 100 | 5E−09 | 0.73% |
| fungi | 20 | 4E−13 | 1.00E+08 | 100 | 2E−08 | 2.91% |
| virus | 0.1 | 1E−17 | 1.00E+09 | 100 | 6E−13 | 0.00% |
| archaea | 1 | 1E−15 | 1.00E+10 | 100 | 6E−11 | 0.01% |
| host | 8 | 7E−14 | 1.00E+04 | 1 | 4E−07 | 46.84% |
| host | 15 | 2E−13 | 1.00E+04 | 1 | 1E−06 | 164.13% |
| host | 50 | 3E−12 | 1.00E+04 | 1 | 1E−05 | 1793.00% |

### Example C7 Individual Cells

The set of beads radius and beads parameters expected for bacteria, archaea, fungi, and virus with Md less than 10 um:
2R: 1.4 mm to 1.8 mm
Number of beads: 5-50% by volume of the tube (before packing)
Frequency: 40 Hz to 70 Hz
Time: 15 s to 60 s

As shown in the following tables:

| | | | | | |
|---|---|---|---|---|---|
| Radius of Bead (mm) | 0.8 | Frequency (Hz) | 58 | Bead Fill Volume % | 27.50% |
| Number of beads | 256 | Time (s) | 38 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 5E−10 | 0.07% |
| bacteria | 1 | 1E−15 | 1.00E+08 | 100 | 2E−09 | 0.28% |
| bacteria | 2 | 5E−15 | 1.00E+08 | 100 | 8E−09 | 1.12% |
| fungi | 5 | 3E−14 | 1.00E+08 | 100 | 5E−08 | 6.99% |
| fungi | 10 | 1E−13 | 1.00E+08 | 100 | 2E−07 | 27.92% |
| fungi | 20 | 5E−13 | 1.00E+08 | 100 | 8E−07 | 111.21% |
| virus | 0.1 | 1E−17 | 1.00E+09 | 100 | 2E−11 | 0.00% |
| archaea | 1 | 1E−15 | 1.00E+10 | 100 | 2E−09 | 0.28% |
| host | 8 | 8E−14 | 1.00E+04 | 1 | 1E−05 | 1788.39% |
| host | 15 | 3E−13 | 1.00E+04 | 1 | 5E−05 | 6268.92% |
| host | 50 | 3E−12 | 1.00E+04 | 1 | 5E−04 | 68632.51% |

The set of beads radius and beads parameters expected for bacteria, archaea, fungi and virus with Md less than or equal to 5 um:
2R: 1.4 mm to 1.8 mm
Number of beads: 5-50% by volume of the tube (before packing)
Frequency: 70 Hz to 100 Hz
Time: 15 s to 0 s

As shown in the following table:

| | | | | | |
|---|---|---|---|---|---|
| Radius of Bead (mm) | 0.9 | Frequency (Hz) | 100 | Bead Fill Volume % | 50.00% |
| Number of beads | 327 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−09 | 0.28% |
| bacteria | 1 | 1E−15 | 1.00E+08 | 100 | 8E−09 | 1.11% |
| bacteria | 2 | 6E−15 | 1.00E+08 | 100 | 3E−08 | 4.44% |
| fungi | 5 | 4E−14 | 1.00E+08 | 100 | 2E−07 | 27.73% |
| fungi | 10 | 1E−13 | 1.00E+08 | 100 | 8E−07 | 110.70% |
| fungi | 20 | 6E−13 | 1.00E+08 | 100 | 3E−06 | 441.15% |
| virus | 0.1 | 1E−17 | 1.00E+09 | 100 | 8E−11 | 0.01% |
| archaea | 1 | 1E−15 | 1.00E+10 | 100 | 8E−09 | 1.11% |
| host | 8 | 9E−14 | 1.00E+04 | 1 | 5E−05 | 7090.04% |
| host | 15 | 3E−13 | 1.00E+04 | 1 | 2E−04 | 24861.11% |
| host | 50 | 3E−12 | 1.00E+04 | 1 | 2E−03 | 272633.74% |

### Saliva Samples

### Example C8 Saliva Samples

The set of beads radius and beads parameters expected for bacteria, archaea, fungi, and virus with Md below 1 um:
2R: 1.0 mm to 1.8 mm
Number of beads: 10-60% by volume of the tube (before packing)
Frequency: 30 Hz to 150 Hz
Time: 30 s to 300 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 2E−10 | 0.03% |
| bacteria | 1 | 8E−16 | 1.00E+08 | 100 | 8E−10 | 0.11% |
| bacteria | 2 | 3E−15 | 1.00E+08 | 100 | 3E−09 | 0.43% |
| fungi | 5 | 2E−14 | 1.00E+08 | 100 | 2E−08 | 2.69% |
| fungi | 10 | 8E−14 | 1.00E+08 | 100 | 8E−08 | 10.73% |
| fungi | 20 | 3E−13 | 1.00E+08 | 100 | 3E−07 | 42.62% |
| virus | 0.1 | 8E−18 | 1.00E+09 | 100 | 8E−12 | 0.00% |
| archaea | 1 | 8E−16 | 1.00E+10 | 100 | 8E−10 | 0.11% |
| host | 8 | 5E−14 | 1.00E+04 | 1 | 5E−06 | 687.51% |
| host | 15 | 2E−13 | 1.00E+04 | 1 | 2E−05 | 2405.70% |
| host | 50 | 2E−12 | 1.00E+04 | 1 | 2E−04 | 26100.00% |

| | | | | | |
|---|---|---|---|---|---|
| Radius of Bead (mm) | 0.7 | Frequency (Hz) | 90 | Bead Fill Volume % | 35.00% |
| Number of beads | 487 | Time (s) | 165 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 6E−09 | 0.80% |
| bacteria | 1 | 1E−15 | 1.00E+08 | 100 | 2E−08 | 3.18% |
| bacteria | 2 | 4E−15 | 1.00E+08 | 100 | 1E−07 | 12.72% |
| fungi | 5 | 3E−14 | 1.00E+08 | 100 | 6E−07 | 79.36% |
| fungi | 10 | 1E−13 | 1.00E+08 | 100 | 2E−06 | 316.70% |
| fungi | 20 | 4E−13 | 1.00E+08 | 100 | 9E−06 | 1260.73% |
| virus | 0.1 | 1E−17 | 1.00E+09 | 100 | 2E−10 | 0.03% |
| archaea | 1 | 1E−15 | 1.00E+10 | 100 | 2E−08 | 3.18% |
| host | 8 | 7E−14 | 1.00E+04 | 1 | 2E−04 | 20288.13% |
| host | 15 | 2E−13 | 1.00E+04 | 1 | 5E−04 | 71086.80% |
| host | 50 | 3E−12 | 1.00E+04 | 1 | 6E−03 | 776594.39% |

| | | | | | |
|---|---|---|---|---|---|
| Radius of Bead (mm) | 0.9 | Frequency (Hz) | 150 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−08 | 2.50% |
| bacteria | 1 | 1E−15 | 1.00E+08 | 100 | 7E−08 | 10.00% |
| bacteria | 2 | 6E−15 | 1.00E+08 | 100 | 3E−07 | 39.97% |
| fungi | 5 | 4E−14 | 1.00E+08 | 100 | 2E−06 | 249.54% |
| fungi | 10 | 1E−13 | 1.00E+08 | 100 | 7E−06 | 996.30% |
| fungi | 20 | 6E−13 | 1.00E+08 | 100 | 3E−05 | 3970.37% |
| virus | 0.1 | 1E−17 | 1.00E+09 | 100 | 7E−10 | 0.10% |
| archaea | 1 | 1E−15 | 1.00E+10 | 100 | 7E−08 | 10.00% |
| host | 8 | 9E−14 | 1.00E+04 | 1 | 5E−04 | 63810.37% |
| host | 15 | 3E−13 | 1.00E+04 | 1 | 2E−03 | 223750.00% |
| host | 50 | 3E−12 | 1.00E+04 | 1 | 2E−02 | 2453703.70% |

### Example C9 Saliva Samples

The set of beads radius and beads parameters expected for bacteria, archaea, fungi, and virus with Md between 1-2 um:
2R: 1.0 mm to 1.3 mm
Number of beads: 10-30% by volume of the tube (before packing)
Frequency: 30 Hz to 60 Hz
Time: 30 s to 0 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 50 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 2E−10 | 0.03% |
| bacteria | 1 | 8E−16 | 1.00E+08 | 100 | 8E−10 | 0.11% |
| bacteria | 2 | 3E−15 | 1.00E+08 | 100 | 3E−09 | 0.43% |
| fungi | 5 | 2E−14 | 1.00E+08 | 100 | 2E−08 | 2.69% |
| fungi | 10 | 8E−14 | 1.00E+08 | 100 | 8E−08 | 10.73% |
| fungi | 20 | 3E−13 | 1.00E+08 | 100 | 3E−07 | 42.62% |
| virus | 0.1 | 8E−18 | 1.00E+09 | 100 | 8E−12 | 0.00% |
| archaea | 1 | 8E−16 | 1.00E+10 | 100 | 8E−10 | 0.11% |
| host | 8 | 5E−14 | 1.00E+04 | 1 | 5E−06 | 687.51% |
| host | 15 | 2E−13 | 1.00E+04 | 1 | 2E−05 | 2405.70% |
| host | 50 | 2E−12 | 1.00E+04 | 1 | 2E−04 | 26100.00% |

| | | | | | |
|---|---|---|---|---|---|
| Radius of Bead (mm) | 0.575 | Frequency (Hz) | 45 | Bead Fill Volume % | 20.00% |
| Number of beads | 502 | Time (s) | 45 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 7E−10 | 0.09% |
| bacteria | 1 | 9E−16 | 1.00E+08 | 100 | 3E−09 | 0.37% |
| bacteria | 2 | 4E−15 | 1.00E+08 | 100 | 1E−08 | 1.47% |
| fungi | 5 | 2E−14 | 1.00E+08 | 100 | 7E−08 | 9.16% |
| fungi | 10 | 9E−14 | 1.00E+08 | 100 | 3E−07 | 36.54% |
| fungi | 20 | 4E−13 | 1.00E+08 | 100 | 1E−06 | 145.29% |
| virus | 0.1 | 9E−18 | 1.00E+09 | 100 | 3E−11 | 0.00% |
| archaea | 1 | 9E−16 | 1.00E+10 | 100 | 3E−09 | 0.37% |
| host | 8 | 6E−14 | 1.00E+04 | 1 | 2E−05 | 2341.00% |
| host | 15 | 2E−13 | 1.00E+04 | 1 | 6E−05 | 8196.53% |
| host | 50 | 2E−12 | 1.00E+04 | 1 | 7E−04 | 89208.51% |

| | | | | | |
|---|---|---|---|---|---|
| Radius of Bead (mm) | 0.65 | Frequency (Hz) | 60 | Bead Fill Volume % | 30.00% |
| Number of beads | 522 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−09 | 0.19% |
| bacteria | 1 | 1E−15 | 1.00E+08 | 100 | 6E−09 | 0.77% |
| bacteria | 2 | 4E−15 | 1.00E+08 | 100 | 2E−08 | 3.06% |
| fungi | 5 | 3E−14 | 1.00E+08 | 100 | 1E−07 | 19.12% |
| fungi | 10 | 1E−13 | 1.00E+08 | 100 | 6E−07 | 76.29% |
| fungi | 20 | 4E−13 | 1.00E+08 | 100 | 2E−06 | 303.60% |
| virus | 0.1 | 1E−17 | 1.00E+09 | 100 | 6E−11 | 0.01% |
| archaea | 1 | 1E−15 | 1.00E+10 | 100 | 6E−09 | 0.77% |
| host | 8 | 7E−14 | 1.00E+04 | 1 | 4E−05 | 4887.79% |
| host | 15 | 2E−13 | 1.00E+04 | 1 | 1E−04 | 17121.71% |
| host | 50 | 2E−12 | 1.00E+04 | 1 | 1E−03 | 186800.18% |

### Example C10 Saliva Samples

The set of beads radius and beads parameters expected for bacteria, archaea, fungi, and virus with Md between 1-2 um:
2R: 1.3 mm to 1.8 mm
Number of beads: 10-60% by volume of the tube (before packing)
Frequency: 30 Hz to 100 Hz
Time: 30 s to 300 s

As shown in the following tables:

| | | | | | |
|---|---|---|---|---|---|
| Radius of Bead (mm) | 0.65 | Frequency (Hz) | 30 | Bead Fill Volume % | 10.00% |
| Number of beads | 174 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−10 | 0.02% |
| bacteria | 1 | 1E−15 | 1.00E+08 | 100 | 5E−10 | 0.06% |
| bacteria | 2 | 4E−15 | 1.00E+08 | 100 | 2E−09 | 0.26% |
| fungi | 5 | 3E−14 | 1.00E+08 | 100 | 1E−08 | 1.59% |
| fungi | 10 | 1E−13 | 1.00E+08 | 100 | 5E−08 | 6.36% |
| fungi | 20 | 4E−13 | 1.00E+08 | 100 | 2E−07 | 25.30% |
| virus | 0.1 | 1E−17 | 1.00E+09 | 100 | 5E−12 | 0.00% |
| archaea | 1 | 1E−15 | 1.00E+10 | 100 | 5E−10 | 0.06% |
| host | 8 | 7E−14 | 1.00E+04 | 1 | 3E−06 | 407.32% |
| host | 15 | 2E−13 | 1.00E+04 | 1 | 1E−05 | 1426.81% |
| host | 50 | 2E−12 | 1.00E+04 | 1 | 1E−04 | 15566.68% |

| | | | | | |
|---|---|---|---|---|---|
| Radius of Bead (mm) | 0.775 | Frequency (Hz) | 65 | Bead Fill Volume % | 35.00% |
| Number of beads | 359 | Time (s) | 165 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 4E−09 | 0.47% |
| bacteria | 1 | 1E−15 | 1.00E+08 | 100 | 1E−08 | 1.87% |
| bacteria | 2 | 5E−15 | 1.00E+08 | 100 | 6E−08 | 7.49% |
| fungi | 5 | 3E−14 | 1.00E+08 | 100 | 4E−07 | 46.77% |
| fungi | 10 | 1E−13 | 1.00E+08 | 100 | 1E−06 | 186.69% |
| fungi | 20 | 5E−13 | 1.00E+08 | 100 | 6E−06 | 743.52% |
| virus | 0.1 | 1E−17 | 1.00E+09 | 100 | 1E−10 | 0.02% |
| archaea | 1 | 1E−15 | 1.00E+10 | 100 | 1E−08 | 1.87% |
| host | 8 | 8E−14 | 1.00E+04 | 1 | 9E−05 | 11958.21% |
| host | 15 | 3E−13 | 1.00E+04 | 1 | 3E−04 | 41913.58% |
| host | 50 | 3E−12 | 1.00E+04 | 1 | 3E−03 | 458650.26% |

| | | | | | |
|---|---|---|---|---|---|
| Radius of Bead (mm) | 0.9 | Frequency (Hz) | 100 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 1E−08 | 1.67% |
| bacteria | 1 | 1E−15 | 1.00E+08 | 100 | 5E−08 | 6.66% |
| bacteria | 2 | 6E−15 | 1.00E+08 | 100 | 2E−07 | 26.65% |
| fungi | 5 | 4E−14 | 1.00E+08 | 100 | 1E−06 | 166.36% |
| fungi | 10 | 1E−13 | 1.00E+08 | 100 | 5E−06 | 664.20% |
| fungi | 20 | 6E−13 | 1.00E+08 | 100 | 2E−05 | 2646.91% |
| virus | 0.1 | 1E−17 | 1.00E+09 | 100 | 5E−10 | 0.07% |
| archaea | 1 | 1E−15 | 1.00E+10 | 100 | 5E−08 | 6.66% |
| host | 8 | 9E−14 | 1.00E+04 | 1 | 3E−04 | 42540.25% |
| host | 15 | 3E−13 | 1.00E+04 | 1 | 1E−03 | 149166.67% |
| host | 50 | 3E−12 | 1.00E+04 | 1 | 1E−02 | 1635802.47% |

### Example C10 Saliva Samples

The set of beads radius and beads parameters expected for bacteria, archaea, fungi, and virus with Md above 2 um:
2R: 1.2 mm to 16 min
Number of beads: 10-30% by volume of the tube (before packing)
Frequency: 30 Hz to 40 Hz
Time: 30 s to 60 s

As shown in the following tables:

| | | | | | |
|---|---|---|---|---|---|
| Radius of Bead (mm) | 0.6 | Frequency (Hz) | 30 | Bead Fill Volume % | 10.00% |
| Number of beads | 221 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 1E−10 | 0.02% |
| bacteria | 1 | 9E−16 | 1.00E+08 | 100 | 6E−10 | 0.07% |
| bacteria | 2 | 4E−15 | 1.00E+08 | 100 | 2E−09 | 0.30% |
| fungi | 5 | 2E−14 | 1.00E+08 | 100 | 1E−08 | 1.87% |
| fungi | 10 | 9E−14 | 1.00E+08 | 100 | 6E−08 | 7.46% |
| fungi | 20 | 4E−13 | 1.00E+08 | 100 | 2E−07 | 29.67% |
| virus | 0.1 | 9E−18 | 1.00E+09 | 100 | 6E−12 | 0.00% |
| archaea | 1 | 9E−16 | 1.00E+10 | 100 | 6E−10 | 0.07% |
| host | 8 | 6E−14 | 1.00E+04 | 1 | 4E−06 | 477.87% |
| host | 15 | 2E−13 | 1.00E+04 | 1 | 1E−05 | 1673.44% |
| host | 50 | 2E−12 | 1.00E+04 | 1 | 1E−04 | 18229.17% |

### Example C11 Saliva Samples

The set of beads radius and beads parameters expected for bacteria, archaea, fungi, and virus with Md less than or equal to 10 um:
2R: 1.2 mm to 1.6 mm
Number of beads: 10-30% by volume of the tube (before packing)
Frequency: 40 Hz to 60 Hz
Time: 30 s to 60 s

As shown in the following tables:

| | | | | | |
|---|---|---|---|---|---|
| Radius of Bead (mm) | 0.7 | Frequency (Hz) | 45 | Bead Fill Volume % | 20.00% |
| Number of beads | 278 | Time (s) | 45 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 5E−10 | 0.06% |
| bacteria | 1 | 1E−15 | 1.00E+08 | 100 | 2E−09 | 0.25% |
| bacteria | 2 | 4E−15 | 1.00E+08 | 100 | 7E−09 | 0.99% |
| fungi | 5 | 3E−14 | 1.00E+08 | 100 | 5E−08 | 6.18% |
| fungi | 10 | 1E−13 | 1.00E+08 | 100 | 2E−07 | 24.68% |
| fungi | 20 | 4E−13 | 1.00E+08 | 100 | 7E−07 | 98.24% |
| virus | 0.1 | 1E−17 | 1.00E+09 | 100 | 2E−11 | 0.00% |
| archaea | 1 | 1E−15 | 1.00E+10 | 100 | 2E−09 | 0.25% |
| host | 8 | 7E−14 | 1.00E+04 | 1 | 1E−05 | 1580.89% |
| host | 15 | 2E−13 | 1.00E+04 | 1 | 4E−05 | 5539.23% |
| host | 50 | 3E−12 | 1.00E+04 | 1 | 5E−04 | 60513.85% |

| | | | | | |
|---|---|---|---|---|---|
| Radius of Bead (mm) | 0.8 | Frequency (Hz) | 60 | Bead Fill Volume % | 30.00% |
| Number of beads | 280 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 9E−10 | 0.13% |
| bacteria | 1 | 1E−15 | 1.00E+08 | 100 | 4E−09 | 0.51% |
| bacteria | 2 | 5E−15 | 1.00E+08 | 100 | 2E−08 | 2.02% |
| fungi | 5 | 3E−14 | 1.00E+08 | 100 | 9E−08 | 12.63% |
| fungi | 10 | 1E−13 | 1.00E+08 | 100 | 4E−07 | 50.41% |
| fungi | 20 | 5E−13 | 1.00E+08 | 100 | 2E−06 | 200.81% |
| virus | 0.1 | 1E−17 | 1.00E+09 | 100 | 4E−11 | 0.01% |
| archaea | 1 | 1E−15 | 1.00E+10 | 100 | 4E−09 | 0.51% |
| host | 8 | 8E−14 | 1.00E+04 | 1 | 2E−05 | 3229.20% |
| host | 15 | 3E−13 | 1.00E+04 | 1 | 8E−05 | 11319.43% |
| host | 50 | 3E−12 | 1.00E+04 | 1 | 9E−04 | 123925.78% |

### Example C12 Saliva Samples

The set of beads radius and beads parameters expected for bacteria, archaea, fungi, and virus with Md above 2 um:
2R: 1.4 mm to 1.8 mm
Number of beads: 10-50% by volume of the tube (before packing)
Frequency: 30 Hz to 50 Hz
Time: 30 s to 60 s

As shown in the following tables:

| | | | | | |
|---|---|---|---|---|---|
| Radius of Bead (mm) | 0.7 | Frequency (Hz) | 30 | Bead Fill Volume % | 10.00% |
| Number of beads | 139 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−10 | 0.01% |
| bacteria | 1 | 1E−15 | 1.00E+08 | 100 | 4E−10 | 0.06% |
| bacteria | 2 | 4E−15 | 1.00E+08 | 100 | 2E−09 | 0.22% |
| fungi | 5 | 3E−14 | 1.00E+08 | 100 | 1E−08 | 1.37% |
| fungi | 10 | 1E−13 | 1.00E+08 | 100 | 4E−08 | 5.48% |
| fungi | 20 | 4E−13 | 1.00E+08 | 100 | 2E−07 | 21.83% |
| virus | 0.1 | 1E−17 | 1.00E+09 | 100 | 4E−12 | 0.00% |
| archaea | 1 | 1E−15 | 1.00E+10 | 100 | 4E−10 | 0.06% |
| host | 8 | 7E−14 | 1.00E+04 | 1 | 3E−06 | 351.31% |
| host | 15 | 2E−13 | 1.00E+04 | 1 | 9E−06 | 1230.94% |
| host | 50 | 3E−12 | 1.00E+04 | 1 | 1E−04 | 13447.52% |

### Example C13 Saliva Samples

The set of beads radius and beads parameters expected for bacteria, archaea, fungi, and virus with Md less than or equal to 10 um:
2R: 1.4 mm to 1.8 min
Number of beads: 10-50% by volume of the tube (before packing)
Frequency: 50 Hz to 80 Hz
Time: 30 s to 60 s

As shown in the following tables:

| | | | | | |
|---|---|---|---|---|---|
| Radius of Bead (mm) | 0.8 | Frequency (Hz) | 65 | Bead Fill Volume % | 30.00% |
| Number of beads | 280 | Time (s) | 45 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 8E−10 | 0.10% |
| bacteria | 1 | 1E−15 | 1.00E+08 | 100 | 3E−09 | 0.41% |
| bacteria | 2 | 5E−15 | 1.00E+08 | 100 | 1E−08 | 1.64% |
| fungi | 5 | 3E−14 | 1.00E+08 | 100 | 8E−08 | 10.26% |
| fungi | 10 | 1E−13 | 1.00E+08 | 100 | 3E−07 | 40.96% |
| fungi | 20 | 5E−13 | 1.00E+08 | 100 | 1E−06 | 163.16% |
| virus | 0.1 | 1E−17 | 1.00E+09 | 100 | 3E−11 | 0.00% |
| archaea | 1 | 1E−15 | 1.00E+10 | 100 | 3E−09 | 0.41% |
| host | 8 | 8E−14 | 1.00E+04 | 1 | 2E−05 | 2623.73% |
| host | 15 | 3E−13 | 1.00E+04 | 1 | 7E−05 | 9197.04% |
| host | 50 | 3E−12 | 1.00E+04 | 1 | 8E−04 | 100689.70% |

### Example C14 Saliva Samples

The set of beads radius and beads parameters expected for bacteria, archaea, fungi, and virus with Md less than or equal to 5 um:
2R: 1.4 mm to 1.8 mm
Number of beads: 10-50% by volume of the tube (before packing)
Frequency: 80 Hz to 100 Hz
Time: 30 s to 60 s

As shown in the following tables:

| | | | | | |
|---|---|---|---|---|---|
| Radius of Bead (mm) | 0.9 | Frequency (Hz) | 100 | Bead Fill Volume % | 50.00% |
| Number of beads | 327 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−09 | 0.28% |
| bacteria | 1 | 1E−15 | 1.00E+08 | 100 | 8E−09 | 1.11% |
| bacteria | 2 | 6E−15 | 1.00E+08 | 100 | 3E−08 | 4.44% |
| fungi | 5 | 4E−14 | 1.00E+08 | 100 | 2E−07 | 27.73% |
| fungi | 10 | 1E−13 | 1.00E+08 | 100 | 8E−07 | 110.70% |
| fungi | 20 | 6E−13 | 1.00E+08 | 100 | 3E−06 | 441.15% |
| virus | 0.1 | 1E−17 | 1.00E+09 | 100 | 8E−11 | 0.01% |
| archaea | 1 | 1E−15 | 1.00E+10 | 100 | 8E−09 | 1.11% |
| host | 8 | 9E−14 | 1.00E+04 | 1 | 5E−05 | 7090.04% |
| host | 15 | 3E−13 | 1.00E+04 | 1 | 2E−04 | 24861.11% |
| host | 50 | 3E−12 | 1.00E+04 | 1 | 2E−03 | 272633.74% |

### Intestinal Biopsies (Medium Solid)

### Example C15 Intestinal Biopsies (Medium Solid)

The set of beads radius and beads parameters expected for bacteria, archaea, fungi, and virus with Md below 1 um:
2R: 1.0 mm to 1.8 mm
Number of beads: 10-60% by volume of the tube (before packing)
Frequency: 30 Hz to 150 Hz
Time: 30 s to 300 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 2E−10 | 0.03% |
| bacteria | 1 | 8E−16 | 1.00E+08 | 100 | 8E−10 | 0.11% |
| bacteria | 2 | 3E−15 | 1.00E+08 | 100 | 3E−09 | 0.43% |
| fungi | 5 | 2E−14 | 1.00E+08 | 100 | 2E−08 | 2.69% |
| fungi | 10 | 8E−14 | 1.00E+08 | 100 | 8E−08 | 10.73% |
| fungi | 20 | 3E−13 | 1.00E+08 | 100 | 3E−07 | 42.62% |
| virus | 0.1 | 8E−18 | 1.00E+09 | 100 | 8E−12 | 0.00% |
| archaea | 1 | 8E−16 | 1.00E+10 | 100 | 8E−10 | 0.11% |
| host | 8 | 5E−14 | 1.00E+04 | 1 | 5E−06 | 687.51% |
| host | 15 | 2E−13 | 1.00E+04 | 1 | 2E−05 | 2405.70% |
| host | 50 | 2E−12 | 1.00E+04 | 1 | 2E−04 | 26100.00% |

| | | | | | |
|---|---|---|---|---|---|
| Radius of Bead (mm) | 0.7 | Frequency (Hz) | 90 | Bead Fill Volume % | 35.00% |
| Number of beads | 487 | Time (s) | 165 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 6E−09 | 0.80% |
| bacteria | 1 | 1E−15 | 1.00E+08 | 100 | 2E−08 | 3.18% |
| bacteria | 2 | 4E−15 | 1.00E+08 | 100 | 1E−07 | 12.72% |
| fungi | 5 | 3E−14 | 1.00E+08 | 100 | 6E−07 | 79.36% |
| fungi | 10 | 1E−13 | 1.00E+08 | 100 | 2E−06 | 316.70% |
| fungi | 20 | 4E−13 | 1.00E+08 | 100 | 9E−06 | 1260.73% |
| virus | 0.1 | 1E−17 | 1.00E+09 | 100 | 2E−10 | 0.03% |
| archaea | 1 | 1E−15 | 1.00E+10 | 100 | 2E−08 | 3.18% |
| host | 8 | 7E−14 | 1.00E+04 | 1 | 2E−04 | 20288.13% |
| host | 15 | 2E−13 | 1.00E+04 | 1 | 5E−04 | 71086.80% |
| host | 50 | 3E−12 | 1.00E+04 | 1 | 6E−03 | 776594.39% |

| | | | | | |
|---|---|---|---|---|---|
| Radius of Bead (mm) | 0.9 | Frequency (Hz) | 150 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−08 | 2.50% |
| bacteria | 1 | 1E−15 | 1.00E+08 | 100 | 7E−08 | 10.00% |
| bacteria | 2 | 6E−15 | 1.00E+08 | 100 | 3E−07 | 39.97% |
| fungi | 5 | 4E−14 | 1.00E+08 | 100 | 2E−06 | 249.54% |
| fungi | 10 | 1E−13 | 1.00E+08 | 100 | 7E−06 | 996.30% |
| fungi | 20 | 6E−13 | 1.00E+08 | 100 | 3E−05 | 3970.37% |
| virus | 0.1 | 1E−17 | 1.00E+09 | 100 | 7E−10 | 0.10% |
| archaea | 1 | 1E−15 | 1.00E+10 | 100 | 7E−08 | 10.00% |
| host | 8 | 9E−14 | 1.00E+04 | 1 | 5E−04 | 63810.37% |
| host | 15 | 3E−13 | 1.00E+04 | 1 | 2E−03 | 223750.00% |
| host | 50 | 3E−12 | 1.00E+04 | 1 | 2E−02 | 2453703.70% |

### Example C16 Intestinal Biopsies (Medium Solid)

The set of beads radius and beads parameters expected for bacteria, archaea, fungi, and virus with Md between 1-2 um:
2R: 1.0 mm to 1.3 mm
Number of beads: 10-30% by volume of the tube (before packing)
Frequency: 30 Hz to 60 Hz
Time: 30 s to 60 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 2E−10 | 0.03% |
| bacteria | 1 | 8E−16 | 1.00E+08 | 100 | 8E−10 | 0.11% |
| bacteria | 2 | 3E−15 | 1.00E+08 | 100 | 3E−09 | 0.43% |
| fungi | 5 | 2E−14 | 1.00E+08 | 100 | 2E−08 | 2.69% |
| fungi | 10 | 8E−14 | 1.00E+08 | 100 | 8E−08 | 10.73% |
| fungi | 20 | 3E−13 | 1.00E+08 | 100 | 3E−07 | 42.62% |
| virus | 0.1 | 8E−18 | 1.00E+09 | 100 | 8E−12 | 0.00% |
| archaea | 1 | 8E−16 | 1.00E+10 | 100 | 8E−10 | 0.11% |
| host | 8 | 5E−14 | 1.00E+04 | 1 | 5E−06 | 687.51% |
| host | 15 | 2E−13 | 1.00E+04 | 1 | 2E−05 | 2405.70% |
| host | 50 | 2E−12 | 1.00E+04 | 1 | 2E−04 | 26100.00% |

| | | | | | |
|---|---|---|---|---|---|
| Radius of Bead (mm) | 0.575 | Frequency (Hz) | 45 | Bead Fill Volume % | 20.00% |
| Number of beads | 502 | Time (s) | 45 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 7E−10 | 0.09% |
| bacteria | 1 | 9E−16 | 1.00E+08 | 100 | 3E−09 | 0.37% |
| bacteria | 2 | 4E−15 | 1.00E+08 | 100 | 1E−08 | 1.47% |
| fungi | 5 | 2E−14 | 1.00E+08 | 100 | 7E−08 | 9.16% |
| fungi | 10 | 9E−14 | 1.00E+08 | 100 | 3E−07 | 36.54% |
| fungi | 20 | 4E−13 | 1.00E+08 | 100 | 1E−06 | 145.29% |
| virus | 0.1 | 9E−18 | 1.00E+09 | 100 | 3E−11 | 0.00% |
| archaea | 1 | 9E−16 | 1.00E+10 | 100 | 3E−09 | 0.37% |
| host | 8 | 6E−14 | 1.00E+04 | 1 | 2E−05 | 2341.00% |
| host | 15 | 2E−13 | 1.00E+04 | 1 | 6E−05 | 8196.53% |
| host | 50 | 2E−12 | 1.00E+04 | 1 | 7E−04 | 89208.51% |

| | | | | | |
|---|---|---|---|---|---|
| Radius of Bead (mm) | 0.65 | Frequency (Hz) | 60 | Bead Fill Volume % | 30.00% |
| Number of beads | 522 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−09 | 0.19% |
| bacteria | 1 | 1E−15 | 1.00E+08 | 100 | 6E−09 | 0.77% |
| bacteria | 2 | 4E−15 | 1.00E+08 | 100 | 2E−08 | 3.06% |
| fungi | 5 | 3E−14 | 1.00E+08 | 100 | 1E−07 | 19.12% |
| fungi | 10 | 1E−13 | 1.00E+08 | 100 | 6E−07 | 76.29% |
| fungi | 20 | 4E−13 | 1.00E+08 | 100 | 2E−06 | 303.60% |
| virus | 0.1 | 1E−17 | 1.00E+09 | 100 | 6E−11 | 0.01% |
| archaea | 1 | 1E−15 | 1.00E+10 | 100 | 6E−09 | 0.77% |
| host | 8 | 7E−14 | 1.00E+04 | 1 | 4E−05 | 4887.79% |
| host | 15 | 2E−13 | 1.00E+04 | 1 | 1E−04 | 17121.71% |
| host | 50 | 2E−12 | 1.00E+04 | 1 | 1E−03 | 186800.18% |

### Example C17 Intestinal Biopsies (Medium Solid)

The set of beads radius and beads parameters expected for bacteria, archaea, fungi, and virus with Md between 1-2 um:
2R: 1.3 mm to 1.8 nm
Number of beads: 10-60% by volume of the tube (before packing)
Frequency: 30 Hz to 100 Hz
Time: 30 s to 300 s

As shown in the following tables:

| | | | | | |
|---|---|---|---|---|---|
| Radius of Bead (mm) | 0.65 | Frequency (Hz) | 30 | Bead Fill Volume % | 10.00% |
| Number of beads | 174 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−10 | 0.02% |
| bacteria | 1 | 1E−15 | 1.00E+08 | 100 | 5E−10 | 0.06% |
| bacteria | 2 | 4E−15 | 1.00E+08 | 100 | 2E−09 | 0.26% |
| fungi | 5 | 3E−14 | 1.00E+08 | 100 | 1E−08 | 1.59% |
| fungi | 10 | 1E−13 | 1.00E+08 | 100 | 5E−08 | 6.36% |
| fungi | 20 | 4E−13 | 1.00E+08 | 100 | 2E−07 | 25.30% |
| virus | 0.1 | 1E−17 | 1.00E+09 | 100 | 5E−12 | 0.00% |
| archaea | 1 | 1E−15 | 1.00E+10 | 100 | 5E−10 | 0.06% |
| host | 8 | 7E−14 | 1.00E+04 | 1 | 3E−06 | 407.32% |
| host | 15 | 2E−13 | 1.00E+04 | 1 | 1E−05 | 1426.81% |
| host | 50 | 2E−12 | 1.00E+04 | 1 | 1E−04 | 15566.68% |

| | | | | | |
|---|---|---|---|---|---|
| Radius of Bead (mm) | 0.775 | Frequency (Hz) | 65 | Bead Fill Volume % | 35.00% |
| Number of beads | 359 | Time (s) | 165 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 4E−09 | 0.47% |
| bacteria | 1 | 1E−15 | 1.00E+08 | 100 | 1E−08 | 1.87% |
| bacteria | 2 | 5E−15 | 1.00E+08 | 100 | 6E−08 | 7.49% |
| fungi | 5 | 3E−14 | 1.00E+08 | 100 | 4E−07 | 46.77% |
| fungi | 10 | 1E−13 | 1.00E+08 | 100 | 1E−06 | 186.69% |
| fungi | 20 | 5E−13 | 1.00E+08 | 100 | 6E−06 | 743.52% |
| virus | 0.1 | 1E−17 | 1.00E+09 | 100 | 1E−10 | 0.02% |
| archaea | 1 | 1E−15 | 1.00E+10 | 100 | 1E−08 | 1.87% |
| host | 8 | 8E−14 | 1.00E+04 | 1 | 9E−05 | 11958.21% |
| host | 15 | 3E−13 | 1.00E+04 | 1 | 3E−04 | 41913.58% |
| host | 50 | 3E−12 | 1.00E+04 | 1 | 3E−03 | 458650.26% |

| | | | | | |
|---|---|---|---|---|---|
| Radius of Bead (mm) | 0.9 | Frequency (Hz) | 100 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 1E−08 | 1.67% |
| bacteria | 1 | 1E−15 | 1.00E+08 | 100 | 5E−08 | 6.66% |
| bacteria | 2 | 6E−15 | 1.00E+08 | 100 | 2E−07 | 26.65% |
| fungi | 5 | 4E−14 | 1.00E+08 | 100 | 1E−06 | 166.36% |
| fungi | 10 | 1E−13 | 1.00E+08 | 100 | 5E−06 | 664.20% |
| fungi | 20 | 6E−13 | 1.00E+08 | 100 | 2E−05 | 2646.91% |
| virus | 0.1 | 1E−17 | 1.00E+09 | 100 | 5E−10 | 0.07% |
| archaea | 1 | 1E−15 | 1.00E+10 | 100 | 5E−08 | 6.66% |
| host | 8 | 9E−14 | 1.00E+04 | 1 | 3E−04 | 42540.25% |
| host | 15 | 3E−13 | 1.00E+04 | 1 | 1E−03 | 149166.67% |
| host | 50 | 3E−12 | 1.00E+04 | 1 | 1E−02 | 1635802.47% |

### Example C18 Intestinal Biopsies (Medium Solid)

The set of beads radius and beads parameters expected for bacteria, archaea, fungi, and virus with Md above 2 um:
2R: 1.2 mm to 1.6 mm
Number of beads: 10-30% by volume of the tube (before packing)
Frequency: 30 Hz to 40 Hz
Time: 30 s to 60 s

As shown in the table below:

| | | | | | |
|---|---|---|---|---|---|
| Radius of Bead (mm) | 0.6 | Frequency (Hz) | 30 | Bead Fill Volume % | 10.00% |
| Number of beads | 221 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 1E−10 | 0.02% |
| bacteria | 1 | 9E−16 | 1.00E+08 | 100 | 6E−10 | 0.07% |
| bacteria | 2 | 4E−15 | 1.00E+08 | 100 | 2E−09 | 0.30% |
| fungi | 5 | 2E−14 | 1.00E+08 | 100 | 1E−08 | 1.87% |
| fungi | 10 | 9E−14 | 1.00E+08 | 100 | 6E−08 | 7.46% |
| fungi | 20 | 4E−13 | 1.00E+08 | 100 | 2E−07 | 29.67% |
| virus | 0.1 | 9E−18 | 1.00E+09 | 100 | 6E−12 | 0.00% |
| archaea | 1 | 9E−16 | 1.00E+10 | 100 | 6E−10 | 0.07% |
| host | 8 | 6E−14 | 1.00E+04 | 1 | 4E−06 | 477.87% |
| host | 15 | 2E−13 | 1.00E+04 | 1 | 1E−05 | 1673.44% |
| host | 50 | 2E−12 | 1.00E+04 | 1 | 1E−04 | 18229.17% |

### Example C19 Intestinal Biopsies (Medium Solid)

The set of beads radius and beads parameters expected for bacteria, archaea, fungi, and virus with Md less than or equal to 10 um:
2R: 1.2 mm to 1.6 mm
Number of beads: 10-30% by volume of the tube (before packing)
Frequency: 40 Hz to 60 Hz
Time: 30 s to 60 s

As shown in the tables below:

| | | | | | |
|---|---|---|---|---|---|
| Radius of Bead (mm) | 0.7 | Frequency (Hz) | 45 | Bead Fill Volume % | 20.00% |
| Number of beads | 278 | Time (s) | 45 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 5E−10 | 0.06% |
| bacteria | 1 | 1E−15 | 1.00E+08 | 100 | 2E−09 | 0.25% |
| bacteria | 2 | 4E−15 | 1.00E+08 | 100 | 7E−09 | 0.99% |
| fungi | 5 | 3E−14 | 1.00E+08 | 100 | 5E−08 | 6.18% |
| fungi | 10 | 1E−13 | 1.00E+08 | 100 | 2E−07 | 24.68% |
| fungi | 20 | 4E−13 | 1.00E+08 | 100 | 7E−07 | 98.24% |
| virus | 0.1 | 1E−17 | 1.00E+09 | 100 | 2E−11 | 0.00% |
| archaea | 1 | 1E−15 | 1.00E+10 | 100 | 2E−09 | 0.25% |
| host | 8 | 7E−14 | 1.00E+04 | 1 | 1E−05 | 1580.89% |
| host | 15 | 2E−13 | 1.00E+04 | 1 | 4E−05 | 5539.23% |
| host | 50 | 3E−12 | 1.00E+04 | 1 | 5E−04 | 60513.85% |

| | | | | | |
|---|---|---|---|---|---|
| Radius of Bead (mm) | 0.8 | Frequency (Hz) | 60 | Bead Fill Volume % | 30.00% |
| Number of beads | 280 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 9E−10 | 0.13% |
| bacteria | 1 | 1E−15 | 1.00E+08 | 100 | 4E−09 | 0.51% |
| bacteria | 2 | 5E−15 | 1.00E+08 | 100 | 2E−08 | 2.02% |
| fungi | 5 | 3E−14 | 1.00E+08 | 100 | 9E−08 | 12.63% |
| fungi | 10 | 1E−13 | 1.00E+08 | 100 | 4E−07 | 50.41% |
| fungi | 20 | 5E−13 | 1.00E+08 | 100 | 2E−06 | 200.81% |
| virus | 0.1 | 1E−17 | 1.00E+09 | 100 | 4E−11 | 0.01% |
| archaea | 1 | 1E−15 | 1.00E+10 | 100 | 4E−09 | 0.51% |
| host | 8 | 8E−14 | 1.00E+04 | 1 | 2E−05 | 3229.20% |
| host | 15 | 3E−13 | 1.00E+04 | 1 | 8E−05 | 11319.43% |
| host | 50 | 3E−12 | 1.00E+04 | 1 | 9E−04 | 123925.78% |

### Example C20 Intestinal Biopsies (Medium Solid)

The set of beads radius and beads parameters expected for bacteria, archaea, fungi, and virus with Md above 2 um:
2R: 1.4 mm to 1.8 mm
Number of beads: 10-50%7 by volume of the tube (before packing)
Frequency: 30 Hz to 50 Hz
Time: 30 s to 60 s

As shown in the following table:

| | | | | | |
|---|---|---|---|---|---|
| Radius of Bead (mm) | 0.7 | Frequency (Hz) | 30 | Bead Fill Volume % | 10.00% |
| Number of beads | 139 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−10 | 0.01% |
| bacteria | 1 | 1E−15 | 1.00E+08 | 100 | 4E−10 | 0.06% |
| bacteria | 2 | 4E−15 | 1.00E+08 | 100 | 2E−09 | 0.22% |
| fungi | 5 | 3E−14 | 1.00E+08 | 100 | 1E−08 | 1.37% |
| fungi | 10 | 1E−13 | 1.00E+08 | 100 | 4E−08 | 5.48% |
| fungi | 20 | 4E−13 | 1.00E+08 | 100 | 2E−07 | 21.83% |
| virus | 0.1 | 1E−17 | 1.00E+09 | 100 | 4E−12 | 0.00% |
| archaea | 1 | 1E−15 | 1.00E+10 | 100 | 4E−10 | 0.06% |
| host | 8 | 7E−14 | 1.00E+04 | 1 | 3E−06 | 351.31% |
| host | 15 | 2E−13 | 1.00E+04 | 1 | 9E−06 | 1230.94% |
| host | 50 | 3E−12 | 1.00E+04 | 1 | 1E−04 | 13447.52% |

### Example C21 Intestinal Biopsies (Medium Solid)

The set of beads radius and beads parameters expected for bacteria, archaea, fungi, and virus with Md less than or equal to 10 um:
2R: 1.4 mm to 1.8 mm
Number of beads: 10-50% by volume of the tube (before packing)
Frequency: 50 Hz to 80 Hz
Time: 30 s to 60 s

As shown in the following tables:

| | | | | | |
|---|---|---|---|---|---|
| Radius of Bead (mm) | 0.8 | Frequency (Hz) | 65 | Bead Fill Volume % | 30.00% |
| Number of beads | 280 | Time (s) | 45 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 8E−10 | 0.10% |
| bacteria | 1 | 1E−15 | 1.00E+08 | 100 | 3E−09 | 0.41% |
| bacteria | 2 | 5E−15 | 1.00E+08 | 100 | 1E−08 | 1.64% |
| fungi | 5 | 3E−14 | 1.00E+08 | 100 | 8E−08 | 10.26% |
| fungi | 10 | 1E−13 | 1.00E+08 | 100 | 3E−07 | 40.96% |
| fungi | 20 | 5E−13 | 1.00E+08 | 100 | 1E−06 | 163.16% |
| virus | 0.1 | 1E−17 | 1.00E+09 | 100 | 3E−11 | 0.00% |
| archaea | 1 | 1E−15 | 1.00E+10 | 100 | 3E−09 | 0.41% |
| host | 8 | 8E−14 | 1.00E+04 | 1 | 2E−05 | 2623.73% |
| host | 15 | 3E−13 | 1.00E+04 | 1 | 7E−05 | 9197.04% |
| host | 50 | 3E−12 | 1.00E+04 | 1 | 8E−04 | 100689.70% |

### Example C22 Intestinal Biopsies (Medium Solid)

The set of beads radius and beads parameters expected for bacteria, archaea, fungi, and virus with Md less than or equal to 5 um:
2R: 1.4 mm to 1.8 mm
Number of beads: 10-50% by volume of the tube (before packing)
Frequency: 80 Hz to 100 Hz
Time: 30 s to 60 s

As shown in the following tables:

| | | | | | |
|---|---|---|---|---|---|
| Radius of Bead (mm) | 0.9 | Frequency (Hz) | 100 | Bead Fill Volume % | 50.00% |
| Number of beads | 327 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−09 | 0.28% |
| bacteria | 1 | 1E−15 | 1.00E+08 | 100 | 8E−09 | 1.11% |
| bacteria | 2 | 6E−15 | 1.00E+08 | 100 | 3E−08 | 4.44% |
| fungi | 5 | 4E−14 | 1.00E+08 | 100 | 2E−07 | 27.73% |
| fungi | 10 | 1E−13 | 1.00E+08 | 100 | 8E−07 | 110.70% |
| fungi | 20 | 6E−13 | 1.00E+08 | 100 | 3E−06 | 441.15% |
| virus | 0.1 | 1E−17 | 1.00E+09 | 100 | 8E−11 | 0.01% |
| archaea | 1 | 1E−15 | 1.00E+10 | 100 | 8E−09 | 1.11% |
| host | 8 | 9E−14 | 1.00E+04 | 1 | 5E−05 | 7090.04% |
| host | 15 | 3E−13 | 1.00E+04 | 1 | 2E−04 | 24861.11% |
| host | 50 | 3E−12 | 1.00E+04 | 1 | 2E−03 | 272633.74% |

### Glandular Tissue (Soft Solid Samples)

### Example C23 Glandular Tissue (Soft Solid Samples)

The set of beads radius and beads parameters expected for bacteria, archaea, fungi, and virus with Md below 1 um:
2R: 1.0 mm to 1.8 mm
Number of beads: 10-60% by volume of the tube (before packing)
Frequency: 30 Hz to 150 Hz
Time: 20 s to 300 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 20 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 1E−10 | 0.02% |
| bacteria | 1 | 8E−16 | 1.00E+08 | 100 | 5E−10 | 0.07% |
| bacteria | 2 | 3E−15 | 1.00E+08 | 100 | 2E−09 | 0.29% |
| fungi | 5 | 2E−14 | 1.00E+08 | 100 | 1E−08 | 1.79% |
| fungi | 10 | 8E−14 | 1.00E+08 | 100 | 5E−08 | 7.15% |
| fungi | 20 | 3E−13 | 1.00E+08 | 100 | 2E−07 | 28.42% |
| virus | 0.1 | 8E−18 | 1.00E+09 | 100 | 5E−12 | 0.00% |
| archaea | 1 | 8E−16 | 1.00E+10 | 100 | 5E−10 | 0.07% |
| host | 8 | 5E−14 | 1.00E+04 | 1 | 3E−06 | 458.34% |
| host | 15 | 2E−13 | 1.00E+04 | 1 | 1E−05 | 1603.80% |
| host | 50 | 2E−12 | 1.00E+04 | 1 | 1E−04 | 17400.00% |

| | | | | | |
|---|---|---|---|---|---|
| Radius of Bead (mm) | 0.7 | Frequency (Hz) | 90 | Bead Fill Volume % | 35.00% |
| Number of beads | 487 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 7E−09 | 0.87% |
| bacteria | 1 | 1E−15 | 1.00E+08 | 100 | 3E−08 | 3.47% |
| bacteria | 2 | 4E−15 | 1.00E+08 | 100 | 1E−07 | 13.87% |
| fungi | 5 | 3E−14 | 1.00E+08 | 100 | 6E−07 | 86.58% |
| fungi | 10 | 1E−13 | 1.00E+08 | 100 | 3E−06 | 345.49% |
| fungi | 20 | 4E−13 | 1.00E+08 | 100 | 1E−05 | 1375.35% |
| virus | 0.1 | 1E−17 | 1.00E+09 | 100 | 3E−10 | 0.03% |
| archaea | 1 | 1E−15 | 1.00E+10 | 100 | 3E−08 | 3.47% |
| host | 8 | 7E−14 | 1.00E+04 | 1 | 2E−04 | 22132.51% |
| host | 15 | 2E−13 | 1.00E+04 | 1 | 6E−04 | 77549.23% |
| host | 50 | 3E−12 | 1.00E+04 | 1 | 6E−03 | 847193.88% |

| | | | | | |
|---|---|---|---|---|---|
| Radius of Bead (mm) | 0.9 | Frequency (Hz) | 150 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−08 | 2.50% |
| bacteria | 1 | 1E−15 | 1.00E+08 | 100 | 7E−08 | 10.00% |
| bacteria | 2 | 6E−15 | 1.00E+08 | 100 | 3E−07 | 39.97% |
| fungi | 5 | 4E−14 | 1.00E+08 | 100 | 2E−06 | 249.54% |
| fungi | 10 | 1E−13 | 1.00E+08 | 100 | 7E−06 | 996.30% |
| fungi | 20 | 6E−13 | 1.00E+08 | 100 | 3E−05 | 3970.37% |
| virus | 0.1 | 1E−17 | 1.00E+09 | 100 | 7E−10 | 0.10% |
| archaea | 1 | 1E−15 | 1.00E+10 | 100 | 7E−08 | 10.00% |
| host | 8 | 9E−14 | 1.00E+04 | 1 | 5E−04 | 63810.37% |
| host | 15 | 3E−13 | 1.00E+04 | 1 | 2E−03 | 223750.00% |
| host | 50 | 3E−12 | 1.00E+04 | 1 | 2E−02 | 2453703.70% |

### Example C24 Glandular Tissue (Soft Solid Samples)

The set of beads radius and beads parameters expected for bacteria, archaea, fungi, and virus with Md between 1-2 um:
2R: 1.0 mm to 1.3 mm
Number of beads: 10-30% by volume of the tube (before packing)
Frequency: 30 Hz to 60 Hz
Time: 20 s to 60 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 20 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 1E−10 | 0.02% |
| bacteria | 1 | 8E−16 | 1.00E+08 | 100 | 5E−10 | 0.07% |
| bacteria | 2 | 3E−15 | 1.00E+08 | 100 | 2E−09 | 0.29% |
| fungi | 5 | 2E−14 | 1.00E+08 | 100 | 1E−08 | 1.79% |
| fungi | 10 | 8E−14 | 1.00E+08 | 100 | 5E−08 | 7.15% |
| fungi | 20 | 3E−13 | 1.00E+08 | 100 | 2E−07 | 28.42% |
| virus | 0.1 | 8E−18 | 1.00E+09 | 100 | 5E−12 | 0.00% |
| archaea | 1 | 8E−16 | 1.00E+10 | 100 | 5E−10 | 0.07% |
| host | 8 | 5E−14 | 1.00E+04 | 1 | 3E−06 | 458.34% |
| host | 15 | 2E−13 | 1.00E+04 | 1 | 1E−05 | 1603.80% |
| host | 50 | 2E−12 | 1.00E+04 | 1 | 1E−04 | 17400.00% |

| | | | | | |
|---|---|---|---|---|---|
| Radius of Bead (mm) | 0.575 | Frequency (Hz) | 45 | Bead Fill Volume % | 20.00% |
| Number of beads | 502 | Time (s) | 40 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 6E−10 | 0.08% |
| bacteria | 1 | 9E−16 | 1.00E+08 | 100 | 2E−09 | 0.33% |
| bacteria | 2 | 4E−15 | 1.00E+08 | 100 | 1E−08 | 1.31% |
| fungi | 5 | 2E−14 | 1.00E+08 | 100 | 6E−08 | 8.14% |
| fungi | 10 | 9E−14 | 1.00E+08 | 100 | 2E−07 | 32.48% |
| fungi | 20 | 4E−13 | 1.00E+08 | 100 | 1E−06 | 129.15% |
| virus | 0.1 | 9E−18 | 1.00E+09 | 100 | 2E−11 | 0.00% |
| archaea | 1 | 9E−16 | 1.00E+10 | 100 | 2E−09 | 0.33% |
| host | 8 | 6E−14 | 1.00E+04 | 1 | 2E−05 | 2080.89% |
| host | 15 | 2E−13 | 1.00E+04 | 1 | 5E−05 | 7285.81% |
| host | 50 | 2E−12 | 1.00E+04 | 1 | 6E−04 | 79296.46% |

| | | | | | |
|---|---|---|---|---|---|
| Radius of Bead (mm) | 0.65 | Frequency (Hz) | 60 | Bead Fill Volume % | 30.00% |
| Number of beads | 522 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−09 | 0.19% |
| bacteria | 1 | 1E−15 | 1.00E+08 | 100 | 6E−09 | 0.77% |
| bacteria | 2 | 4E−15 | 1.00E+08 | 100 | 2E−08 | 3.06% |
| fungi | 5 | 3E−14 | 1.00E+08 | 100 | 1E−07 | 19.12% |
| fungi | 10 | 1E−13 | 1.00E+08 | 100 | 6E−07 | 76.29% |
| fungi | 20 | 4E−13 | 1.00E+08 | 100 | 2E−06 | 303.60% |
| virus | 0.1 | 1E−17 | 1.00E+09 | 100 | 6E−11 | 0.01% |
| archaea | 1 | 1E−15 | 1.00E+10 | 100 | 6E−09 | 0.77% |
| host | 8 | 7E−14 | 1.00E+04 | 1 | 4E−05 | 4887.79% |
| host | 15 | 2E−13 | 1.00E+04 | 1 | 1E−04 | 17121.71% |
| host | 50 | 2E−12 | 1.00E+04 | 1 | 1E−03 | 186800.18% |

### Example C25 Glandular Tissue (Soft Solid Samples)

The set of beads radius and beads parameters expected for bacteria, archaea, fungi, and virus with Md between 1-2 um:
2R: 1.3 mm to 1.8 mm
Number of beads: 10-60% by volume of the tube (before packing)
Frequency: 30 Hz to 100 Hz
Time: 20 s to 300 s

As shown in the following tables:

| | | | | | |
|---|---|---|---|---|---|
| Radius of Bead (mm) | 0.65 | Frequency (Hz) | 30 | Bead Fill Volume % | 10.00% |
| Number of beads | 174 | Time (s) | 20 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 8E−11 | 0.01% |
| bacteria | 1 | 1E−15 | 1.00E+08 | 100 | 3E−10 | 0.04% |
| bacteria | 2 | 4E−15 | 1.00E+08 | 100 | 1E−09 | 0.17% |
| fungi | 5 | 3E−14 | 1.00E+08 | 100 | 8E−09 | 1.06% |
| fungi | 10 | 1E−13 | 1.00E+08 | 100 | 3E−08 | 4.24% |
| fungi | 20 | 4E−13 | 1.00E+08 | 100 | 1E−07 | 16.87% |
| virus | 0.1 | 1E−17 | 1.00E+09 | 100 | 3E−12 | 0.00% |
| archaea | 1 | 1E−15 | 1.00E+10 | 100 | 3E−10 | 0.04% |
| host | 8 | 7E−14 | 1.00E+04 | 1 | 2E−06 | 271.54% |
| host | 15 | 2E−13 | 1.00E+04 | 1 | 7E−06 | 951.21% |
| host | 50 | 2E−12 | 1.00E+04 | 1 | 8E−05 | 10377.79% |

| | | | | | |
|---|---|---|---|---|---|
| Radius of Bead (mm) | 0.775 | Frequency (Hz) | 65 | Bead Fill Volume % | 35.00% |
| Number of beads | 359 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 4E−09 | 0.51% |
| bacteria | 1 | 1E−15 | 1.00E+08 | 100 | 2E−08 | 2.04% |
| bacteria | 2 | 5E−15 | 1.00E+08 | 100 | 6E−08 | 8.17% |
| fungi | 5 | 3E−14 | 1.00E+08 | 100 | 4E−07 | 51.02% |
| fungi | 10 | 1E−13 | 1.00E+08 | 100 | 2E−06 | 203.66% |
| fungi | 20 | 5E−13 | 1.00E+08 | 100 | 6E−06 | 811.11% |
| virus | 0.1 | 1E−17 | 1.00E+09 | 100 | 2E−10 | 0.02% |
| archaea | 1 | 1E−15 | 1.00E+10 | 100 | 2E−08 | 2.04% |
| host | 8 | 8E−14 | 1.00E+04 | 1 | 1E−04 | 13045.32% |
| host | 15 | 3E−13 | 1.00E+04 | 1 | 3E−04 | 45723.90% |
| host | 50 | 3E−12 | 1.00E+04 | 1 | 4E−03 | 500345.74% |

| | | | | | |
|---|---|---|---|---|---|
| Radius of Bead (mm) | 0.9 | Frequency (Hz) | 100 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 1E−08 | 1.67% |
| bacteria | 1 | 1E−15 | 1.00E+08 | 100 | 5E−08 | 6.66% |
| bacteria | 2 | 6E−15 | 1.00E+08 | 100 | 2E−07 | 26.65% |
| fungi | 5 | 4E−14 | 1.00E+08 | 100 | 1E−06 | 166.36% |
| fungi | 10 | 1E−13 | 1.00E+08 | 100 | 5E−06 | 664.20% |
| fungi | 20 | 6E−13 | 1.00E+08 | 100 | 2E−05 | 2646.91% |
| virus | 0.1 | 1E−17 | 1.00E+09 | 100 | 5E−10 | 0.07% |
| archaea | 1 | 1E−15 | 1.00E+10 | 100 | 5E−08 | 6.66% |
| host | 8 | 9E−14 | 1.00E+04 | 1 | 3E−04 | 42540.25% |
| host | 15 | 3E−13 | 1.00E+04 | 1 | 1E−03 | 149166.67% |
| host | 50 | 3E−12 | 1.00E+04 | 1 | 1E−02 | 1635802.47% |

### Example C26 Glandular Tissue (Soft Solid Samples)

The set of beads radius and beads parameters expected for bacteria, archaea, fungi, and virus with Md above 2 um:
2R: 1.2 mm to 1.6 mm
Number of beads: 10-30% by volume of the tube (before packing)
Frequency: 30 Hz to 40 Hz
Time: 20 s to 60 s

As shown in the following tables:

| | | | | | |
|---|---|---|---|---|---|
| Radius of Bead (mm) | 0.6 | Frequency (Hz) | 30 | Bead Fill Volume % | 10.00% |
| Number of beads | 221 | Time (s) | 20 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 9E−11 | 0.01% |
| bacteria | 1 | 9E−16 | 1.00E+08 | 100 | 4E−10 | 0.05% |
| bacteria | 2 | 4E−15 | 1.00E+08 | 100 | 1E−09 | 0.20% |
| fungi | 5 | 2E−14 | 1.00E+08 | 100 | 9E−09 | 1.25% |
| fungi | 10 | 9E−14 | 1.00E+08 | 100 | 4E−08 | 4.97% |
| fungi | 20 | 4E−13 | 1.00E+08 | 100 | 1E−07 | 19.78% |
| virus | 0.1 | 9E−18 | 1.00E+09 | 100 | 4E−12 | 0.00% |
| archaea | 1 | 9E−16 | 1.00E+10 | 100 | 4E−10 | 0.05% |
| host | 8 | 6E−14 | 1.00E+04 | 1 | 2E−06 | 318.58% |
| host | 15 | 2E−13 | 1.00E+04 | 1 | 8E−06 | 1115.63% |
| host | 50 | 2E−12 | 1.00E+04 | 1 | 9E−05 | 12152.78% |

### Example C27 Glandular Tissue (Soft Solid Samples)

The set of beads radius and beads parameters expected for bacteria, archaea, fungi, and virus with Md less than or equal to 10 um:
2R: 1.2 mm to 1.6 mm
Number of beads: 10-30% by volume of the tube (before packing)
Frequency: 40 Hz to 60 Hz
Time: 20 s to 60 s

As shown in the following tables:

| | | | | | |
|---|---|---|---|---|---|
| Radius of Bead (mm) | 0.7 | Frequency (Hz) | 45 | Bead Fill Volume % | 20.00% |
| Number of beads | 278 | Time (s) | 40 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 4E−10 | 0.06% |
| bacteria | 1 | 1E−15 | 1.00E+08 | 100 | 2E−09 | 0.22% |
| bacteria | 2 | 4E−15 | 1.00E+08 | 100 | 7E−09 | 0.88% |
| fungi | 5 | 3E−14 | 1.00E+08 | 100 | 4E−08 | 5.50% |
| fungi | 10 | 1E−13 | 1.00E+08 | 100 | 2E−07 | 21.94% |
| fungi | 20 | 4E−13 | 1.00E+08 | 100 | 7E−07 | 87.32% |
| virus | 0.1 | 1E−17 | 1.00E+09 | 100 | 2E−11 | 0.00% |
| archaea | 1 | 1E−15 | 1.00E+10 | 100 | 2E−09 | 0.22% |
| host | 8 | 7E−14 | 1.00E+04 | 1 | 1E−05 | 1405.24% |
| host | 15 | 2E−13 | 1.00E+04 | 1 | 4E−05 | 4923.76% |
| host | 50 | 3E−12 | 1.00E+04 | 1 | 4E−04 | 53790.09% |

| | | | | | |
|---|---|---|---|---|---|
| Radius of Bead (mm) | 0.8 | Frequency (Hz) | 60 | Bead Fill Volume % | 30.00% |
| Number of beads | 280 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 9E−10 | 0.13% |
| bacteria | 1 | 1E−15 | 1.00E+08 | 100 | 4E−09 | 0.51% |
| bacteria | 2 | 5E−15 | 1.00E+08 | 100 | 2E−08 | 2.02% |
| fungi | 5 | 3E−14 | 1.00E+08 | 100 | 9E−08 | 12.63% |
| fungi | 10 | 1E−13 | 1.00E+08 | 100 | 4E−07 | 50.41% |
| fungi | 20 | 5E−13 | 1.00E+08 | 100 | 2E−06 | 200.81% |
| virus | 0.1 | 1E−17 | 1.00E+09 | 100 | 4E−11 | 0.01% |
| archaea | 1 | 1E−15 | 1.00E+10 | 100 | 4E−09 | 0.51% |
| host | 8 | 8E−14 | 1.00E+04 | 1 | 2E−05 | 3229.20% |
| host | 15 | 3E−13 | 1.00E+04 | 1 | 8E−05 | 11319.43% |
| host | 50 | 3E−12 | 1.00E+04 | 1 | 9E−04 | 123925.78% |

### Example C28 Glandular Tissue (Soft Solid Samples)

The set of beads radius and beads parameters expected for bacteria, archaea, fungi, and virus with Md above 2 um:
2R: 1.4 mm to 1.8 mm
Number of beads: 10-50% by volume of the tube (before packing)
Frequency: 30 Hz to 50 Hz
Time: 20 s to 60 s

As shown in the following table:

| | | | | | |
|---|---|---|---|---|---|
| Radius of Bead (mm) | 0.7 | Frequency (Hz) | 30 | Bead Fill Volume % | 10.00% |
| Number of beads | 139 | Time (s) | 20 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 7E−11 | 0.01% |
| bacteria | 1 | 1E−15 | 1.00E+08 | 100 | 3E−10 | 0.04% |
| bacteria | 2 | 4E−15 | 1.00E+08 | 100 | 1E−09 | 0.15% |
| fungi | 5 | 3E−14 | 1.00E+08 | 100 | 7E−09 | 0.92% |
| fungi | 10 | 1E−13 | 1.00E+08 | 100 | 3E−08 | 3.66% |
| fungi | 20 | 4E−13 | 1.00E+08 | 100 | 1E−07 | 14.55% |
| virus | 0.1 | 1E−17 | 1.00E+09 | 100 | 3E−12 | 0.00% |
| archaea | 1 | 1E−15 | 1.00E+10 | 100 | 3E−10 | 0.04% |
| host | 8 | 7E−14 | 1.00E+04 | 1 | 2E−06 | 234.21% |
| host | 15 | 2E−13 | 1.00E+04 | 1 | 6E−06 | 820.63% |
| host | 50 | 3E−12 | 1.00E+04 | 1 | 7E−05 | 8965.01% |

### Example C29 Glandular Tissue (Soft Solid Samples)

The set of beads radius and beads parameters expected for bacteria, archaea, fungi, and virus with Md less than or equal to 10 um:
2R: 1.4 mm to 1.8 mm
Number of beads: 10-50% by volume of the tube (before packing)
Frequency: 50 Hz to 70 Hz
Time: 20 s to 60 s

As shown in the following tables:

| | | | | | |
|---|---|---|---|---|---|
| Radius of Bead (mm) | 0.8 | Frequency (Hz) | 65 | Bead Fill Volume % | 30.00% |
| Number of beads | 280 | Time (s) | 40 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 7E−10 | 0.09% |
| bacteria | 1 | 1E−15 | 1.00E+08 | 100 | 3E−09 | 0.37% |
| bacteria | 2 | 5E−15 | 1.00E+08 | 100 | 1E−08 | 1.46% |
| fungi | 5 | 3E−14 | 1.00E+08 | 100 | 7E−08 | 9.12% |
| fungi | 10 | 1E−13 | 1.00E+08 | 100 | 3E−07 | 36.41% |
| fungi | 20 | 5E−13 | 1.00E+08 | 100 | 1E−06 | 145.03% |
| virus | 0.1 | 1E−17 | 1.00E+09 | 100 | 3E−11 | 0.00% |
| archaea | 1 | 1E−15 | 1.00E+10 | 100 | 3E−09 | 0.37% |
| host | 8 | 8E−14 | 1.00E+04 | 1 | 2E−05 | 2332.20% |
| host | 15 | 3E−13 | 1.00E+04 | 1 | 6E−05 | 8175.15% |
| host | 50 | 3E−12 | 1.00E+04 | 1 | 7E−04 | 89501.95% |

### Example C30 Glandular Tissue (Soft Solid Samples)

The set of beads radius and beads parameters expected for bacteria, archaea, fungi, and virus with Md less than or equal to 5 um:
2R: 1.4 mm to 1.8 mm
Number of beads: 10-50% by volume of the tube (before packing)
Frequency: 70 Hz to 100 Hz
Time: 20 s to 60 s

As shown in the following tables:

| | | | | | |
|---|---|---|---|---|---|
| Radius of Bead (mm) | 0.9 | Frequency (Hz) | 100 | Bead Fill Volume % | 50.00% |
| Number of beads | 327 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−09 | 0.28% |
| bacteria | 1 | 1E−15 | 1.00E+08 | 100 | 8E−09 | 1.11% |
| bacteria | 2 | 6E−15 | 1.00E+08 | 100 | 3E−08 | 4.44% |
| fungi | 5 | 4E−14 | 1.00E+08 | 100 | 2E−07 | 27.73% |
| fungi | 10 | 1E−13 | 1.00E+08 | 100 | 8E−07 | 110.70% |
| fungi | 20 | 6E−13 | 1.00E+08 | 100 | 3E−06 | 441.15% |
| virus | 0.1 | 1E−17 | 1.00E+09 | 100 | 8E−11 | 0.01% |
| archaea | 1 | 1E−15 | 1.00E+10 | 100 | 8E−09 | 1.11% |
| host | 8 | 9E−14 | 1.00E+04 | 1 | 5E−05 | 7090.04% |
| host | 15 | 3E−13 | 1.00E+04 | 1 | 2E−04 | 24861.11% |
| host | 50 | 3E−12 | 1.00E+04 | 1 | 2E−03 | 272633.74% |

### Adipose (Soft Solid Samples)

### Example C31 Adipose (Soft Solid Samples)

The set of beads radius and beads parameters expected for bacteria, archaea, fungi, and virus with Md below 1 um:
2R: 1.0 mm to 1.8 mm
Number of beads: 10-60% by volume of the tube (before packing)
Frequency: 30 Hz to 150 Hz
Time: 15 s to 300 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 1E−10 | 0.01% |
| bacteria | 1 | 8E−16 | 1.00E+08 | 100 | 4E−10 | 0.05% |
| bacteria | 2 | 3E−15 | 1.00E+08 | 100 | 2E−09 | 0.22% |
| fungi | 5 | 2E−14 | 1.00E+08 | 100 | 1E−08 | 1.35% |
| fungi | 10 | 8E−14 | 1.00E+08 | 100 | 4E−08 | 5.36% |
| fungi | 20 | 3E−13 | 1.00E+08 | 100 | 2E−07 | 21.31% |
| virus | 0.1 | 8E−18 | 1.00E+09 | 100 | 4E−12 | 0.00% |
| archaea | 1 | 8E−16 | 1.00E+10 | 100 | 4E−10 | 0.05% |
| host | 8 | 5E−14 | 1.00E+04 | 1 | 3E−06 | 343.76% |
| host | 15 | 2E−13 | 1.00E+04 | 1 | 9E−06 | 1202.85% |
| host | 50 | 2E−12 | 1.00E+04 | 1 | 1E−04 | 13050.00% |

| | | | | | |
|---|---|---|---|---|---|
| Radius of Bead (mm) | 0.7 | Frequency (Hz) | 90 | Bead Fill Volume % | 35.00% |
| Number of beads | 487 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 7E−09 | 0.87% |
| bacteria | 1 | 1E−15 | 1.00E+08 | 100 | 3E−08 | 3.47% |
| bacteria | 2 | 4E−15 | 1.00E+08 | 100 | 1E−07 | 13.87% |
| fungi | 5 | 3E−14 | 1.00E+08 | 100 | 6E−07 | 86.58% |
| fungi | 10 | 1E−13 | 1.00E+08 | 100 | 3E−06 | 345.49% |
| fungi | 20 | 4E−13 | 1.00E+08 | 100 | 1E−05 | 1375.35% |
| virus | 0.1 | 1E−17 | 1.00E+09 | 100 | 3E−10 | 0.03% |
| archaea | 1 | 1E−15 | 1.00E+10 | 100 | 3E−08 | 3.47% |
| host | 8 | 7E−14 | 1.00E+04 | 1 | 2E−04 | 22132.51% |
| host | 15 | 2E−13 | 1.00E+04 | 1 | 6E−04 | 77549.23% |
| host | 50 | 3E−12 | 1.00E+04 | 1 | 6E−03 | 847193.88% |

| | | | | | |
|---|---|---|---|---|---|
| Radius of Bead (mm) | 0.9 | Frequency (Hz) | 150 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−08 | 2.50% |
| bacteria | 1 | 1E−15 | 1.00E+08 | 100 | 7E−08 | 10.00% |
| bacteria | 2 | 6E−15 | 1.00E+08 | 100 | 3E−07 | 39.97% |
| fungi | 5 | 4E−14 | 1.00E+08 | 100 | 2E−06 | 249.54% |
| fungi | 10 | 1E−13 | 1.00E+08 | 100 | 7E−06 | 996.30% |
| fungi | 20 | 6E−13 | 1.00E+08 | 100 | 3E−05 | 3970.37% |
| virus | 0.1 | 1E−17 | 1.00E+09 | 100 | 7E−10 | 0.10% |
| archaea | 1 | 1E−15 | 1.00E+10 | 100 | 7E−08 | 10.00% |
| host | 8 | 9E−14 | 1.00E+04 | 1 | 5E−04 | 63810.37% |
| host | 15 | 3E−13 | 1.00E+04 | 1 | 2E−03 | 223750.00% |
| host | 50 | 3E−12 | 1.00E+04 | 1 | 2E−02 | 2453703.70% |

### Example C32 Adipose (Soft Solid Samples)

The set of beads radius and beads parameters expected for bacteria, archaea, fungi, and virus with Md between 1-2 um:
2R: 1.0 mm to 1.3 mm
Number of beads: 10-30% by volume of the tube (before packing)
Frequency: 30 Hz to 60 Hz
Time: 15 s to 60 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 1E−10 | 0.01% |
| bacteria | 1 | 8E−16 | 1.00E+08 | 100 | 4E−10 | 0.05% |
| bacteria | 2 | 3E−15 | 1.00E+08 | 100 | 2E−09 | 0.22% |
| fungi | 5 | 2E−14 | 1.00E+08 | 100 | 1E−08 | 1.35% |
| fungi | 10 | 8E−14 | 1.00E+08 | 100 | 4E−08 | 5.36% |
| fungi | 20 | 3E−13 | 1.00E+08 | 100 | 2E−07 | 21.31% |
| virus | 0.1 | 8E−18 | 1.00E+09 | 100 | 4E−12 | 0.00% |
| archaea | 1 | 8E−16 | 1.00E+10 | 100 | 4E−10 | 0.05% |
| host | 8 | 5E−14 | 1.00E+04 | 1 | 3E−06 | 343.76% |
| host | 15 | 2E−13 | 1.00E+04 | 1 | 9E−06 | 1202.85% |
| host | 50 | 2E−12 | 1.00E+04 | 1 | 1E−04 | 13050.00% |

| | | | | | |
|---|---|---|---|---|---|
| Radius of Bead (mm) | 0.575 | Frequency (Hz) | 45 | Bead Fill Volume % | 20.00% |
| Number of beads | 502 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 5E−10 | 0.06% |
| bacteria | 1 | 9E−16 | 1.00E+08 | 100 | 2E−09 | 0.24% |
| bacteria | 2 | 4E−15 | 1.00E+08 | 100 | 7E−09 | 0.98% |
| fungi | 5 | 2E−14 | 1.00E+08 | 100 | 5E−08 | 6.11% |
| fungi | 10 | 9E−14 | 1.00E+08 | 100 | 2E−07 | 24.36% |
| fungi | 20 | 4E−13 | 1.00E+08 | 100 | 7E−07 | 96.86% |
| virus | 0.1 | 9E−18 | 1.00E+09 | 100 | 2E−11 | 0.00% |
| archaea | 1 | 9E−16 | 1.00E+10 | 100 | 2E−09 | 0.24% |
| host | 8 | 6E−14 | 1.00E+04 | 1 | 1E−05 | 1560.67% |
| host | 15 | 2E−13 | 1.00E+04 | 1 | 4E−05 | 5464.35% |
| host | 50 | 2E−12 | 1.00E+04 | 1 | 4E−04 | 59472.34% |

| | | | | | |
|---|---|---|---|---|---|
| Radius of Bead (mm) | 0.65 | Frequency (Hz) | 60 | Bead Fill Volume % | 30.00% |
| Number of beads | 522 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−09 | 0.19% |
| bacteria | 1 | 1E−15 | 1.00E+08 | 100 | 6E−09 | 0.77% |
| bacteria | 2 | 4E−15 | 1.00E+08 | 100 | 2E−08 | 3.06% |
| fungi | 5 | 3E−14 | 1.00E+08 | 100 | 1E−07 | 19.12% |
| fungi | 10 | 1E−13 | 1.00E+08 | 100 | 6E−07 | 76.29% |
| fungi | 20 | 4E−13 | 1.00E+08 | 100 | 2E−06 | 303.60% |
| virus | 0.1 | 1E−17 | 1.00E+09 | 100 | 6E−11 | 0.01% |
| archaea | 1 | 1E−15 | 1.00E+10 | 100 | 6E−09 | 0.77% |
| host | 8 | 7E−14 | 1.00E+04 | 1 | 4E−05 | 4887.79% |
| host | 15 | 2E−13 | 1.00E+04 | 1 | 1E−04 | 17121.71% |
| host | 50 | 2E−12 | 1.00E+04 | 1 | 1E−03 | 186800.18% |

### Example C33 Adipose (Soft Solid Samples)

The set of beads radius and beads parameters expected for bacteria, archaea, fungi, and virus with Md between 1-2 um:
2R: 1.3 mm to 1.8 mm
Number of beads: 10-60% by volume of the tube (before packing)
Frequency: 30 Hz to 100 Hz
Time: 15 s to 300 s

As shown in the following tables:

| | | | | | |
|---|---|---|---|---|---|
| Radius of Bead (mm) | 0.65 | Frequency (Hz) | 30 | Bead Fill Volume % | 10.00% |
| Number of beads | 174 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 6E−11 | 0.01% |
| bacteria | 1 | 1E−15 | 1.00E+08 | 100 | 2E−10 | 0.03% |
| bacteria | 2 | 4E−15 | 1.00E+08 | 100 | 1E−09 | 0.13% |
| fungi | 5 | 3E−14 | 1.00E+08 | 100 | 6E−09 | 0.80% |
| fungi | 10 | 1E−13 | 1.00E+08 | 100 | 2E−08 | 3.18% |
| fungi | 20 | 4E−13 | 1.00E+08 | 100 | 9E−08 | 12.65% |
| virus | 0.1 | 1E−17 | 1.00E+09 | 100 | 2E−12 | 0.00% |
| archaea | 1 | 1E−15 | 1.00E+10 | 100 | 2E−10 | 0.03% |
| host | 8 | 7E−14 | 1.00E+04 | 1 | 2E−06 | 203.66% |
| host | 15 | 2E−13 | 1.00E+04 | 1 | 5E−06 | 713.40% |
| host | 50 | 2E−12 | 1.00E+04 | 1 | 6E−05 | 7783.34% |

| | | | | | |
|---|---|---|---|---|---|
| Radius of Bead (mm) | 0.775 | Frequency (Hz) | 65 | Bead Fill Volume % | 35.00% |
| Number of beads | 359 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 4E−09 | 0.51% |
| bacteria | 1 | 1E−15 | 1.00E+08 | 100 | 2E−08 | 2.04% |
| bacteria | 2 | 5E−15 | 1.00E+08 | 100 | 6E−08 | 8.17% |
| fungi | 5 | 3E−14 | 1.00E+08 | 100 | 4E−07 | 51.02% |
| fungi | 10 | 1E−13 | 1.00E+08 | 100 | 2E−06 | 203.66% |
| fungi | 20 | 5E−13 | 1.00E+08 | 100 | 6E−06 | 811.11% |
| virus | 0.1 | 1E−17 | 1.00E+09 | 100 | 2E−10 | 0.02% |
| archaea | 1 | 1E−15 | 1.00E+10 | 100 | 2E−08 | 2.04% |
| host | 8 | 8E−14 | 1.00E+04 | 1 | 1E−04 | 13045.32% |
| host | 15 | 3E−13 | 1.00E+04 | 1 | 3E−04 | 45723.90% |
| host | 50 | 3E−12 | 1.00E+04 | 1 | 4E−03 | 500345.74% |

| | | | | | |
|---|---|---|---|---|---|
| Radius of Bead (mm) | 0.9 | Frequency (Hz) | 100 | Bead Fill Volume % | 60.00% |
| Number of beads | 393 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 1E−08 | 1.67% |
| bacteria | 1 | 1E−15 | 1.00E+08 | 100 | 5E−08 | 6.66% |
| bacteria | 2 | 6E−15 | 1.00E+08 | 100 | 2E−07 | 26.65% |
| fungi | 5 | 4E−14 | 1.00E+08 | 100 | 1E−06 | 166.36% |
| fungi | 10 | 1E−13 | 1.00E+08 | 100 | 5E−06 | 664.20% |
| fungi | 20 | 6E−13 | 1.00E+08 | 100 | 2E−05 | 2646.91% |
| virus | 0.1 | 1E−17 | 1.00E+09 | 100 | 5E−10 | 0.07% |
| archaea | 1 | 1E−15 | 1.00E+10 | 100 | 5E−08 | 6.66% |
| host | 8 | 9E−14 | 1.00E+04 | 1 | 3E−04 | 42540.25% |
| host | 15 | 3E−13 | 1.00E+04 | 1 | 1E−03 | 149166.67% |
| host | 50 | 3E−12 | 1.00E+04 | 1 | 1E−02 | 1635802.47% |

### Example C34 Adipose (Soft Solid Samples)

The set of beads radius and beads parameters expected for bacteria, archaea, fungi, and virus with Md above 2 um:
2R: 1.2 mm to 1.6 mm
Number of beads: 10-30% by volume of the tube (before packing)
Frequency: 30 Hz to 40 Hz
Time: 15 s to 60 s

As shown in the following tables:

| | | | | | |
|---|---|---|---|---|---|
| Radius of Bead (mm) | 0.6 | Frequency (Hz) | 30 | Bead Fill Volume % | 10.00% |
| Number of beads | 221 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 7E−11 | 0.01% |
| bacteria | 1 | 9E−16 | 1.00E+08 | 100 | 3E−10 | 0.04% |
| bacteria | 2 | 4E−15 | 1.00E+08 | 100 | 1E−09 | 0.15% |
| fungi | 5 | 2E−14 | 1.00E+08 | 100 | 7E−09 | 0.93% |
| fungi | 10 | 9E−14 | 1.00E+08 | 100 | 3E−08 | 3.73% |
| fungi | 20 | 4E−13 | 1.00E+08 | 100 | 1E−07 | 14.83% |
| virus | 0.1 | 9E−18 | 1.00E+09 | 100 | 3E−12 | 0.00% |
| archaea | 1 | 9E−16 | 1.00E+10 | 100 | 3E−10 | 0.04% |
| host | 8 | 6E−14 | 1.00E+04 | 1 | 2E−06 | 238.93% |
| host | 15 | 2E−13 | 1.00E+04 | 1 | 6E−06 | 836.72% |
| host | 50 | 2E−12 | 1.00E+04 | 1 | 7E−05 | 9114.58% |

### Example C35 Adipose (Soft Solid Samples)

The set of beads radius and beads parameters expected for bacteria, archaea, fungi, and virus with Md less than or equal to 10 um:
2R: 1.2 mm to 1.6 mm
Number of beads: 10-30% by volume of the tube (before packing)
Frequency: 40 Hz to 60 Hz
Time: 15 s to 60 s

As shown in the following tables:

| | | | | | |
|---|---|---|---|---|---|
| Radius of Bead (mm) | 0.7 | Frequency (Hz) | 45 | Bead Fill Volume % | 20.00% |
| Number of beads | 278 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 3E−10 | 0.04% |
| bacteria | 1 | 1E−15 | 1.00E+08 | 100 | 1E−09 | 0.17% |
| bacteria | 2 | 4E−15 | 1.00E+08 | 100 | 5E−09 | 0.66% |
| fungi | 5 | 3E−14 | 1.00E+08 | 100 | 3E−08 | 4.12% |
| fungi | 10 | 1E−13 | 1.00E+08 | 100 | 1E−07 | 16.45% |
| fungi | 20 | 4E−13 | 1.00E+08 | 100 | 5E−07 | 65.49% |
| virus | 0.1 | 1E−17 | 1.00E+09 | 100 | 1E−11 | 0.00% |
| archaea | 1 | 1E−15 | 1.00E+10 | 100 | 1E−09 | 0.17% |
| host | 8 | 7E−14 | 1.00E+04 | 1 | 8E−06 | 1053.93% |
| host | 15 | 2E−13 | 1.00E+04 | 1 | 3E−05 | 3692.82% |
| host | 50 | 3E−12 | 1.00E+04 | 1 | 3E−04 | 40342.57% |

| | | | | | |
|---|---|---|---|---|---|
| Radius of Bead (mm) | 0.8 | Frequency (Hz) | 60 | Bead Fill Volume % | 30.00% |
| Number of beads | 280 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 9E−10 | 0.13% |
| bacteria | 1 | 1E−15 | 1.00E+08 | 100 | 4E−09 | 0.51% |
| bacteria | 2 | 5E−15 | 1.00E+08 | 100 | 2E−08 | 2.02% |
| fungi | 5 | 3E−14 | 1.00E+08 | 100 | 9E−08 | 12.63% |
| fungi | 10 | 1E−13 | 1.00E+08 | 100 | 4E−07 | 50.41% |
| fungi | 20 | 5E−13 | 1.00E+08 | 100 | 2E−06 | 200.81% |
| virus | 0.1 | 1E−17 | 1.00E+09 | 100 | 4E−11 | 0.01% |
| archaea | 1 | 1E−15 | 1.00E+10 | 100 | 4E−09 | 0.51% |
| host | 8 | 8E−14 | 1.00E+04 | 1 | 2E−05 | 3229.20% |
| host | 15 | 3E−13 | 1.00E+04 | 1 | 8E−05 | 11319.43% |
| host | 50 | 3E−12 | 1.00E+04 | 1 | 9E−04 | 123925.78% |

### Example C36 Adipose (Soft Solid Samples)

The set of beads radius and beads parameters expected for bacteria, archaea, fungi, and virus with Md above 2 um:
2R: 1.4 mm to 1.8 mm
Number of beads: 10-50% by volume of the tube (before packing)
Frequency: 30 Hz to 45 Hz
Time: 15 s to 60 s

As shown in the following table:

| | | | | | |
|---|---|---|---|---|---|
| Radius of Bead (mm) | 0.7 | Frequency (Hz) | 30 | Bead Fill Volume % | 10.00% |
| Number of beads | 139 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 5E−11 | 0.01% |
| bacteria | 1 | 1E−15 | 1.00E+08 | 100 | 2E−10 | 0.03% |
| bacteria | 2 | 4E−15 | 1.00E+08 | 100 | 8E−10 | 0.11% |
| fungi | 5 | 3E−14 | 1.00E+08 | 100 | 5E−09 | 0.69% |
| fungi | 10 | 1E−13 | 1.00E+08 | 100 | 2E−08 | 2.74% |
| fungi | 20 | 4E−13 | 1.00E+08 | 100 | 8E−08 | 10.92% |
| virus | 0.1 | 1E−17 | 1.00E+09 | 100 | 2E−12 | 0.00% |
| archaea | 1 | 1E−15 | 1.00E+10 | 100 | 2E−10 | 0.03% |
| host | 8 | 7E−14 | 1.00E+04 | 1 | 1E−06 | 175.65% |
| host | 15 | 2E−13 | 1.00E+04 | 1 | 5E−06 | 615.47% |
| host | 50 | 3E−12 | 1.00E+04 | 1 | 5E−05 | 6723.76% |

### Example C37 Adipose (Soft Solid Samples)

The set of beads radius and beads parameters expected for bacteria, archaea, fungi, and virus with Md less than or equal to 10 um:
2R: 1.4 mm to 1.8 mm
Number of beads: 10-50% by volume of the tube (before packing)
Frequency: 45 Hz to 80 Hz
Time: 15 s to 60 s

As shown in the following tables:

| | | | | | |
|---|---|---|---|---|---|
| Radius of Bead (mm) | 0.8 | Frequency (Hz) | 65 | Bead Fill Volume % | 35.00% |
| Number of beads | 326 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 6E−10 | 0.08% |
| bacteria | 1 | 1E−15 | 1.00E+08 | 100 | 2E−09 | 0.32% |
| bacteria | 2 | 5E−15 | 1.00E+08 | 100 | 1E−08 | 1.28% |
| fungi | 5 | 3E−14 | 1.00E+08 | 100 | 6E−08 | 7.98% |
| fungi | 10 | 1E−13 | 1.00E+08 | 100 | 2E−07 | 31.86% |
| fungi | 20 | 5E−13 | 1.00E+08 | 100 | 1E−06 | 126.90% |
| virus | 0.1 | 1E−17 | 1.00E+09 | 100 | 2E−11 | 0.00% |
| archaea | 1 | 1E−15 | 1.00E+10 | 100 | 2E−09 | 0.32% |
| host | 8 | 8E−14 | 1.00E+04 | 1 | 2E−05 | 2040.68% |
| host | 15 | 3E−13 | 1.00E+04 | 1 | 5E−05 | 7153.25% |
| host | 50 | 3E−12 | 1.00E+04 | 1 | 6E−04 | 78314.21% |

### Example C38 Adipose (Soft Solid Samples)

The set of beads radius and beads parameters expected for bacteria, archaea, fungi, and virus with Md less than or equal to 5 um:
2R: 1.4 mm to 1.8 mm
Number of beads: 10-50% by volume of the tube (before packing)
Frequency: 80 Hz to 100 Hz
Time: 15 s to 60 s

As shown in the following tables:

| | | | | | |
|---|---|---|---|---|---|
| Radius of Bead (mm) | 0.9 | Frequency (Hz) | 100 | Bead Fill Volume % | 50.00% |
| Number of beads | 327 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−09 | 0.28% |
| bacteria | 1 | 1E−15 | 1.00E+08 | 100 | 8E−09 | 1.11% |
| bacteria | 2 | 6E−15 | 1.00E+08 | 100 | 3E−08 | 4.44% |
| fungi | 5 | 4E−14 | 1.00E+08 | 100 | 2E−07 | 27.73% |
| fungi | 10 | 1E−13 | 1.00E+08 | 100 | 8E−07 | 110.70% |
| fungi | 20 | 6E−13 | 1.00E+08 | 100 | 3E−06 | 441.15% |
| virus | 0.1 | 1E−17 | 1.00E+09 | 100 | 8E−11 | 0.01% |
| archaea | 1 | 1E−15 | 1.00E+10 | 100 | 8E−09 | 1.11% |
| host | 8 | 9E−14 | 1.00E+04 | 1 | 5E−05 | 7090.04% |
| host | 15 | 3E−13 | 1.00E+04 | 1 | 2E−04 | 24861.11% |
| host | 50 | 3E−12 | 1.00E+04 | 1 | 2E−03 | 272633.74% |

### Whole Insect Organisms (Hard Solid Samples)

### Example C38 Whole Insect Organisms (Hard Solid Samples)

The set of beads radius and beads parameters expected for bacteria, archaea, fungi, and virus with Md below 1 um:
2R: 1.0 mm to 1.8 mm
Number of beads: 10-60% by volume of the tube (before packing)
Frequency: 30 Hz to 150 Hz
Time: 60 s to 300 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 4E−10 | 0.05% |
| bacteria | 1 | 8E−16 | 1.00E+08 | 100 | 2E−09 | 0.22% |
| bacteria | 2 | 3E−15 | 1.00E+08 | 100 | 6E−09 | 0.86% |
| fungi | 5 | 2E−14 | 1.00E+08 | 100 | 4E−08 | 5.38% |
| fungi | 10 | 8E−14 | 1.00E+08 | 100 | 2E−07 | 21.46% |
| fungi | 20 | 3E−13 | 1.00E+08 | 100 | 6E−07 | 85.25% |
| virus | 0.1 | 8E−18 | 1.00E+09 | 100 | 2E−11 | 0.00% |
| archaea | 1 | 8E−16 | 1.00E+10 | 100 | 2E−09 | 0.22% |
| host | 8 | 5E−14 | 1.00E+04 | 1 | 1E−05 | 1375.03% |
| host | 15 | 2E−13 | 1.00E+04 | 1 | 4E−05 | 4811.40% |
| host | 50 | 2E−12 | 1.00E+04 | 1 | 4E−04 | 52200.00% |

| | | | | | |
|---|---|---|---|---|---|
| Radius of Bead (mm) | 0.7 | Frequency (Hz) | 90 | Bead Fill Volume % | 35.00% |
| Number of beads | 487 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 7E−09 | 0.87% |
| bacteria | 1 | 1E−15 | 1.00E+08 | 100 | 3E−08 | 3.47% |
| bacteria | 2 | 4E−15 | 1.00E+08 | 100 | 1E−07 | 13.87% |
| fungi | 5 | 3E−14 | 1.00E+08 | 100 | 6E−07 | 86.58% |
| fungi | 10 | 1E−13 | 1.00E+08 | 100 | 3E−06 | 345.49% |
| fungi | 20 | 4E−13 | 1.00E+08 | 100 | 1E−05 | 1375.35% |
| virus | 0.1 | 1E−17 | 1.00E+09 | 100 | 3E−10 | 0.03% |
| archaea | 1 | 1E−15 | 1.00E+10 | 100 | 3E−08 | 3.47% |
| host | 8 | 7E−14 | 1.00E+04 | 1 | 2E−04 | 22132.51% |
| host | 15 | 2E−13 | 1.00E+04 | 1 | 6E−04 | 77549.23% |
| host | 50 | 3E−12 | 1.00E+04 | 1 | 6E−03 | 847193.88% |

| | | | | | |
|---|---|---|---|---|---|
| Radius of Bead (mm) | 0.8 | Frequency (Hz) | 150 | Bead Fill Volume % | 60.00% |
| Number of beads | 560 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 2E−08 | 3.16% |
| bacteria | 1 | 1E−15 | 1.00E+08 | 100 | 9E−08 | 12.65% |
| bacteria | 2 | 5E−15 | 1.00E+08 | 100 | 4E−07 | 50.58% |
| fungi | 5 | 3E−14 | 1.00E+08 | 100 | 2E−06 | 315.75% |
| fungi | 10 | 1E−13 | 1.00E+08 | 100 | 9E−06 | 1260.35% |
| fungi | 20 | 5E−13 | 1.00E+08 | 100 | 4E−05 | 5020.31% |
| virus | 0.1 | 1E−17 | 1.00E+09 | 100 | 9E−10 | 0.13% |
| archaea | 1 | 1E−15 | 1.00E+10 | 100 | 9E−08 | 12.65% |
| host | 8 | 8E−14 | 1.00E+04 | 1 | 6E−04 | 80730.00% |
| host | 15 | 3E−13 | 1.00E+04 | 1 | 2E−03 | 282985.84% |
| host | 50 | 3E−12 | 1.00E+04 | 1 | 2E−02 | 3098144.53% |

### Example C39 Whole Insect Organisms (Hard Solid Samples)

The set of beads radius and beads parameters expected for bacteria, archaea, fungi, and virus with Md between 1-2 um:
2R: 1.0 mm to 1.3 mm
Number of beads: 10-30% by volume of the tube (before packing)
Frequency: 30 Hz to 60 Hz
Time: 15 s to 60 s

As shown in the following tables:

| | | | | | |
|---|---|---|---|---|---|
| Radius of Bead (mm) | 0.5 | Frequency (Hz) | 30 | Bead Fill Volume % | 10.00% |
| Number of beads | 382 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 1E−10 | 0.01% |
| bacteria | 1 | 8E−16 | 1.00E+08 | 100 | 4E−10 | 0.05% |
| bacteria | 2 | 3E−15 | 1.00E+08 | 100 | 2E−09 | 0.22% |
| fungi | 5 | 2E−14 | 1.00E+08 | 100 | 1E−08 | 1.35% |
| fungi | 10 | 8E−14 | 1.00E+08 | 100 | 4E−08 | 5.36% |
| fungi | 20 | 3E−13 | 1.00E+08 | 100 | 2E−07 | 21.31% |
| virus | 0.1 | 8E−18 | 1.00E+09 | 100 | 4E−12 | 0.00% |
| archaea | 1 | 8E−16 | 1.00E+10 | 100 | 4E−10 | 0.05% |
| host | 8 | 5E−14 | 1.00E+04 | 1 | 3E−06 | 343.76% |
| host | 15 | 2E−13 | 1.00E+04 | 1 | 9E−06 | 1202.85% |
| host | 50 | 2E−12 | 1.00E+04 | 1 | 1E−04 | 13050.00% |

| | | | | | |
|---|---|---|---|---|---|
| Radius of Bead (mm) | 0.575 | Frequency (Hz) | 45 | Bead Fill Volume % | 20.00% |
| Number of beads | 502 | Time (s) | 30 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 5E−10 | 0.06% |
| bacteria | 1 | 9E−16 | 1.00E+08 | 100 | 2E−09 | 0.24% |
| bacteria | 2 | 4E−15 | 1.00E+08 | 100 | 7E−09 | 0.98% |
| fungi | 5 | 2E−14 | 1.00E+08 | 100 | 5E−08 | 6.11% |
| fungi | 10 | 9E−14 | 1.00E+08 | 100 | 2E−07 | 24.36% |
| fungi | 20 | 4E−13 | 1.00E+08 | 100 | 7E−07 | 96.86% |
| virus | 0.1 | 9E−18 | 1.00E+09 | 100 | 2E−11 | 0.00% |
| archaea | 1 | 9E−16 | 1.00E+10 | 100 | 2E−09 | 0.24% |
| host | 8 | 6E−14 | 1.00E+04 | 1 | 1E−05 | 1560.67% |
| host | 15 | 2E−13 | 1.00E+04 | 1 | 4E−05 | 5464.35% |
| host | 50 | 2E−12 | 1.00E+04 | 1 | 4E−04 | 59472.34% |

| | | | | | |
|---|---|---|---|---|---|
| Radius of Bead (mm) | 0.65 | Frequency (Hz) | 60 | Bead Fill Volume % | 30.00% |
| Number of beads | 522 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 1E−09 | 0.19% |
| bacteria | 1 | 1E−15 | 1.00E+08 | 100 | 6E−09 | 0.77% |
| bacteria | 2 | 4E−15 | 1.00E+08 | 100 | 2E−08 | 3.06% |
| fungi | 5 | 3E−14 | 1.00E+08 | 100 | 1E−07 | 19.12% |
| fungi | 10 | 1E−13 | 1.00E+08 | 100 | 6E−07 | 76.29% |
| fungi | 20 | 4E−13 | 1.00E+08 | 100 | 2E−06 | 303.60% |
| virus | 0.1 | 1E−17 | 1.00E+09 | 100 | 6E−11 | 0.01% |
| archaea | 1 | 1E−15 | 1.00E+10 | 100 | 6E−09 | 0.77% |
| host | 8 | 7E−14 | 1.00E+04 | 1 | 4E−05 | 4887.79% |
| host | 15 | 2E−13 | 1.00E+04 | 1 | 1E−04 | 17121.71% |
| host | 50 | 2E−12 | 1.00E+04 | 1 | 1E−03 | 186800.18% |

### Example C40 Whole Insect Organisms (Hard Solid Samples)

The set of beads radius and beads parameters expected for bacteria, archaea, fungi, and virus with Md between 1-2 um:
2R: 1.3 mm to 1.8 mm
Number of beads: 10-60% by volume of the tube (before packing)
Frequency: 30 Hz to 100 Hz
Time: 15 s to 300 s

As shown in the following tables:

| | | | | | |
|---|---|---|---|---|---|
| Radius of Bead (mm) | 0.65 | Frequency (Hz) | 30 | Bead Fill Volume % | 10.00% |
| Number of beads | 174 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 6E−11 | 0.01% |
| bacteria | 1 | 1E−15 | 1.00E+08 | 100 | 2E−10 | 0.03% |
| bacteria | 2 | 4E−15 | 1.00E+08 | 100 | 1E−09 | 0.13% |
| fungi | 5 | 3E−14 | 1.00E+08 | 100 | 6E−09 | 0.80% |
| fungi | 10 | 1E−13 | 1.00E+08 | 100 | 2E−08 | 3.18% |
| fungi | 20 | 4E−13 | 1.00E+08 | 100 | 9E−08 | 12.65% |
| virus | 0.1 | 1E−17 | 1.00E+09 | 100 | 2E−12 | 0.00% |
| archaea | 1 | 1E−15 | 1.00E+10 | 100 | 2E−10 | 0.03% |
| host | 8 | 7E−14 | 1.00E+04 | 1 | 2E−06 | 203.66% |
| host | 15 | 2E−13 | 1.00E+04 | 1 | 5E−06 | 713.40% |
| host | 50 | 2E−12 | 1.00E+04 | 1 | 6E−05 | 7783.34% |

| | | | | | |
|---|---|---|---|---|---|
| Radius of Bead (mm) | 0.775 | Frequency (Hz) | 65 | Bead Fill Volume % | 35.00% |
| Number of beads | 359 | Time (s) | 180 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 4E−09 | 0.51% |
| bacteria | 1 | 1E−15 | 1.00E+08 | 100 | 2E−08 | 2.04% |
| bacteria | 2 | 5E−15 | 1.00E+08 | 100 | 6E−08 | 8.17% |
| fungi | 5 | 3E−14 | 1.00E+08 | 100 | 4E−07 | 51.02% |
| fungi | 10 | 1E−13 | 1.00E+08 | 100 | 2E−06 | 203.66% |
| fungi | 20 | 5E−13 | 1.00E+08 | 100 | 6E−06 | 811.11% |
| virus | 0.1 | 1E−17 | 1.00E+09 | 100 | 2E−10 | 0.02% |
| archaea | 1 | 1E−15 | 1.00E+10 | 100 | 2E−08 | 2.04% |
| host | 8 | 8E−14 | 1.00E+04 | 1 | 1E−04 | 13045.32% |
| host | 15 | 3E−13 | 1.00E+04 | 1 | 3E−04 | 45723.90% |
| host | 50 | 3E−12 | 1.00E+04 | 1 | 4E−03 | 500345.74% |

| | | | | | |
|---|---|---|---|---|---|
| Radius of Bead (mm) | 0.775 | Frequency (Hz) | 100 | Bead Fill Volume % | 60.00% |
| Number of beads | 615 | Time (s) | 300 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 2E−08 | 2.25% |
| bacteria | 1 | 1E−15 | 1.00E+08 | 100 | 7E−08 | 8.99% |
| bacteria | 2 | 5E−15 | 1.00E+08 | 100 | 3E−07 | 35.93% |
| fungi | 5 | 3E−14 | 1.00E+08 | 100 | 2E−06 | 224.28% |
| fungi | 10 | 1E−13 | 1.00E+08 | 100 | 7E−06 | 895.20% |
| fungi | 20 | 5E−13 | 1.00E+08 | 100 | 3E−05 | 3565.32% |
| virus | 0.1 | 1E−17 | 1.00E+09 | 100 | 7E−10 | 0.09% |
| archaea | 1 | 1E−15 | 1.00E+10 | 100 | 7E−08 | 8.99% |
| host | 8 | 8E−14 | 1.00E+04 | 1 | 4E−04 | 57342.08% |
| host | 15 | 3E−13 | 1.00E+04 | 1 | 2E−03 | 200984.19% |
| host | 50 | 3E−12 | 1.00E+04 | 1 | 2E−02 | 2199321.94% |

### Example C41 Whole Insect Organisms (Hard Solid Samples)

The set of beads radius and beads parameters expected for bacteria, archaea, fungi, and virus with Md above 2 um:
2R: 1.2 mm to 1.6 mm
Number of beads: 10-30% by volume of the tube (before packing)
Frequency: 30 Hz to 40 Hz
Time: 15 s to 60 s

As shown in the following tables:

| | | | | | |
|---|---|---|---|---|---|
| Radius of Bead (mm) | 0.6 | Frequency (Hz) | 30 | Bead Fill Volume % | 10.00% |
| Number of beads | 221 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 2E−16 | 1.00E+08 | 100 | 7E−11 | 0.01% |
| bacteria | 1 | 9E−16 | 1.00E+08 | 100 | 3E−10 | 0.04% |
| bacteria | 2 | 4E−15 | 1.00E+08 | 100 | 1E−09 | 0.15% |
| fungi | 5 | 2E−14 | 1.00E+08 | 100 | 7E−09 | 0.93% |
| fungi | 10 | 9E−14 | 1.00E+08 | 100 | 3E−08 | 3.73% |
| fungi | 20 | 4E−13 | 1.00E+08 | 100 | 1E−07 | 14.83% |
| virus | 0.1 | 9E−18 | 1.00E+09 | 100 | 3E−12 | 0.00% |
| archaea | 1 | 9E−16 | 1.00E+10 | 100 | 3E−10 | 0.04% |
| host | 8 | 6E−14 | 1.00E+04 | 1 | 2E−06 | 238.93% |
| host | 15 | 2E−13 | 1.00E+04 | 1 | 6E−06 | 836.72% |
| host | 50 | 2E−12 | 1.00E+04 | 1 | 7E−05 | 9114.58% |

### Example C42 Whole Insect Organisms (Hard Solid Samples)

The set of beads radius and beads parameters expected for bacteria, archaea, fungi, and virus with Md less than or equal to 10 um:
2R: 1.2 mm to 1.6 mm
Number of beads: 10-30% by volume of the tube (before packing)
Frequency: 40 Hz to 50 Hz
Time: 15 s to 60 s

As shown in the following tables:

| | | | | | |
|---|---|---|---|---|---|
| Radius of Bead (mm) | 0.7 | Frequency (Hz) | 45 | Bead Fill Volume % | 20.00% |
| Number of beads | 278 | Time (s) | 38 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 4E−10 | 0.05% |
| bacteria | 1 | 1E−15 | 1.00E+08 | 100 | 2E−09 | 0.21% |
| bacteria | 2 | 4E−15 | 1.00E+08 | 100 | 6E−09 | 0.83% |
| fungi | 5 | 3E−14 | 1.00E+08 | 100 | 4E−08 | 5.15% |
| fungi | 10 | 1E−13 | 1.00E+08 | 100 | 2E−07 | 20.56% |
| fungi | 20 | 4E−13 | 1.00E+08 | 100 | 6E−07 | 81.87% |
| virus | 0.1 | 1E−17 | 1.00E+09 | 100 | 2E−11 | 0.00% |
| archaea | 1 | 1E−15 | 1.00E+10 | 100 | 2E−09 | 0.21% |
| host | 8 | 7E−14 | 1.00E+04 | 1 | 1E−05 | 1317.41% |
| host | 15 | 2E−13 | 1.00E+04 | 1 | 3E−05 | 4616.03% |
| host | 50 | 3E−12 | 1.00E+04 | 1 | 4E−04 | 50428.21% |

| | | | | | |
|---|---|---|---|---|---|
| Radius of Bead (mm) | 0.8 | Frequency (Hz) | 60 | Bead Fill Volume % | 30.00% |
| Number of beads | 280 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 9E−10 | 0.13% |
| bacteria | 1 | 1E−15 | 1.00E+08 | 100 | 4E−09 | 0.51% |
| bacteria | 2 | 5E−15 | 1.00E+08 | 100 | 2E−08 | 2.02% |
| fungi | 5 | 3E−14 | 1.00E+08 | 100 | 9E−08 | 12.63% |
| fungi | 10 | 1E−13 | 1.00E+08 | 100 | 4E−07 | 50.41% |
| fungi | 20 | 5E−13 | 1.00E+08 | 100 | 2E−06 | 200.81% |
| virus | 0.1 | 1E−17 | 1.00E+09 | 100 | 4E−11 | 0.01% |
| archaea | 1 | 1E−15 | 1.00E+10 | 100 | 4E−09 | 0.51% |
| host | 8 | 8E−14 | 1.00E+04 | 1 | 2E−05 | 3229.20% |
| host | 15 | 3E−13 | 1.00E+04 | 1 | 8E−05 | 11319.43% |
| host | 50 | 3E−12 | 1.00E+04 | 1 | 9E−04 | 123925.78% |

### Example C43 Whole Insect Organisms (Hard Solid Samples)

The set of beads radius and beads parameters expected for bacteria, archaea, fungi, and virus with Md above 2 um:
2R: 1.4 mm to 1.8 mm
Number of beads: 10-50% by volume of the tube (before packing)
Frequency: 30 Hz to 50 Hz
Time: 15 s to 60 s

As shown in the following table:

| | | | | | |
|---|---|---|---|---|---|
| Radius of Bead (mm) | 0.7 | Frequency (Hz) | 30 | Bead Fill Volume % | 10.00% |
| Number of beads | 139 | Time (s) | 15 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 5E−11 | 0.01% |
| bacteria | 1 | 1E−15 | 1.00E+08 | 100 | 2E−10 | 0.03% |
| bacteria | 2 | 4E−15 | 1.00E+08 | 100 | 8E−10 | 0.11% |
| fungi | 5 | 3E−14 | 1.00E+08 | 100 | 5E−09 | 0.69% |
| fungi | 10 | 1E−13 | 1.00E+08 | 100 | 2E−08 | 2.74% |
| fungi | 20 | 4E−13 | 1.00E+08 | 100 | 8E−08 | 10.92% |
| virus | 0.1 | 1E−17 | 1.00E+09 | 100 | 2E−12 | 0.00% |
| archaea | 1 | 1E−15 | 1.00E+10 | 100 | 2E−10 | 0.03% |
| host | 8 | 7E−14 | 1.00E+04 | 1 | 1E−06 | 175.65% |
| host | 15 | 2E−13 | 1.00E+04 | 1 | 5E−06 | 615.47% |
| host | 50 | 3E−12 | 1.00E+04 | 1 | 5E−05 | 6723.76% |

### Example C44 Whole Insect Organisms (Hard Solid Samples)

The set of beads radius and beads parameters expected for bacteria, archaea, fungi, and virus with Md less than or equal to 10 um:
2R: 1.4 mm to 1.8 mm
Number of beads: 10-50% by volume of the tube (before packing)
Frequency: 50 Hz to 80 Hz
Time: 15 s to 60 s

As shown in the following tables:

| | | | | | |
|---|---|---|---|---|---|
| Radius of Bead (mm) | 0.8 | Frequency (Hz) | 65 | Bead Fill Volume % | 30.00% |
| Number of beads | 280 | Time (s) | 38 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 3E−16 | 1.00E+08 | 100 | 6E−10 | 0.09% |
| bacteria | 1 | 1E−15 | 1.00E+08 | 100 | 3E−09 | 0.34% |
| bacteria | 2 | 5E−15 | 1.00E+08 | 100 | 1E−08 | 1.37% |
| fungi | 5 | 3E−14 | 1.00E+08 | 100 | 6E−08 | 8.55% |
| fungi | 10 | 1E−13 | 1.00E+08 | 100 | 3E−07 | 34.13% |
| fungi | 20 | 5E−13 | 1.00E+08 | 100 | 1E−06 | 135.97% |
| virus | 0.1 | 1E−17 | 1.00E+09 | 100 | 3E−11 | 0.00% |
| archaea | 1 | 1E−15 | 1.00E+10 | 100 | 3E−09 | 0.34% |
| host | 8 | 8E−14 | 1.00E+04 | 1 | 2E−05 | 2186.44% |
| host | 15 | 3E−13 | 1.00E+04 | 1 | 6E−05 | 7664.20% |
| host | 50 | 3E−12 | 1.00E+04 | 1 | 6E−04 | 83908.08% |

### Example C45 Whole Insect Organisms (Hard Solid Samples)

The set of beads radius and beads parameters expected for bacteria, archaea, fungi, and virus with Md less than or equal to 5 um:
2R: 1.4 mm to 1.8 mm
Number of beads: 10-50% by volume of the tube (before packing)
Frequency: 80 Hz to 100 Hz.
Time: 15 s to 0 s

As shown in the following table:

| | | | | | |
|---|---|---|---|---|---|
| Radius of Bead (mm) | 0.9 | Frequency (Hz) | 100 | Bead Fill Volume % | 50.00% |
| Number of beads | 327 | Time (s) | 60 | Sample volume (micro L) | 750 |

| Compartment | lengthscale (Md, um) | V crushed, comp (m3) | moduli (Pa) | E_adj | V effective (m3) | % BB, comp |
|---|---|---|---|---|---|---|
| bacteria | 0.5 | 4E−16 | 1.00E+08 | 100 | 2E−09 | 0.28% |
| bacteria | 1 | 1E−15 | 1.00E+08 | 100 | 8E−09 | 1.11% |
| bacteria | 2 | 6E−15 | 1.00E+08 | 100 | 3E−08 | 4.44% |
| fungi | 5 | 4E−14 | 1.00E+08 | 100 | 2E−07 | 27.73% |
| fungi | 10 | 1E−13 | 1.00E+08 | 100 | 8E−07 | 110.70% |
| fungi | 20 | 6E−13 | 1.00E+08 | 100 | 3E−06 | 441.15% |
| virus | 0.1 | 1E−17 | 1.00E+09 | 100 | 8E−11 | 0.01% |
| archaea | 1 | 1E−15 | 1.00E+10 | 100 | 8E−09 | 1.11% |
| host | 8 | 9E−14 | 1.00E+04 | 1 | 5E−05 | 7090.04% |
| host | 15 | 3E−13 | 1.00E+04 | 1 | 2E−04 | 24861.11% |
| host | 50 | 3E−12 | 1.00E+04 | 1 | 2E−03 | 272633.74% |

In summary, provided herein are methods and systems to selectively deplete a biological sample of host compartments and/or host nucleic acid while enriching the sample microbial compartments and/or related microbial nucleic acid. Also provided herein compositions, methods and systems related to said host depletion and microbial enrichment methods and systems.

The examples set forth above are provided to give those of ordinary skill in the art a complete disclosure and description of how to make and use the embodiments of the compounds, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including webpages patents, patent applications, journal articles, abstracts, laboratory manuals, books, or other disclosures) in the Background, Summary, Detailed Description, and Examples is hereby incorporated herein by reference. All references cited in this disclosure, including references cited in any one of the Appendices, are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

### REFERENCES

1. Collier L, B. A., Sussman M Mahy B, Topley and Wilson's Microbiology and Microbial Infections. Virology. Vol. 1 (9th ed.) 1998: Collier LA (eds.).
2. Caspar D L, K. A., Physical principles in the construction of regular viruses. 1962: Cold Spring Harbor Symposia on Quantitative Biology.
3. Crick F H, W. J., Structure of small viruses. Nature, March 1956. 177 (4506): p. 473-75.
4. Krasner, R. B., The microbial challenge: a public health perspective. Mass: Jones & Bartlett Learning, 2014.
5. Available from: web page en.wikipedia.org/wiki/Viral_envelope at the filing date of the preesnt disclosure.
6. Fajardo, G. and H. Hornicke, Problems in estimating the extent of coprophagy in the rat. Br J Nutr, 1989. 62(3): p. 551-61.
7. Jastrzebski, D., Nature and Properties of Engineering Materials. Wiley International ed. ed. 1959: John Wiley & Sons, Inc.
8. Song, J. a. C. Y., Chemical Modifications to RNA: A New Layer of Gene Expression Regulation. ACS Chem Biol, 2017. 12(2): p. 316-325.
9. Jackman, J. E. a. J. D. A., Transfer RNA modifications: Nature's combinatorial chemistry playground. Wiley interdisciplinary reviews. RNA, 2013. 4(1): p. 35-48.
10. Yu, M. K., E. C. Fogarty, and A. M. Eren, The genetic and ecological landscape of plasmids in the human gut. 2022, bioRxiv.
11. Alberts, B. J., Alexander; Lewis, Julian; Raff, Martin; Roberts, Keith; Walter, Peter Molecular Biology of the Cell 4th ed. 2002: Garland Science.
12. Burden, D., Guide to disruption of biological sample. Random Primers, January 2012(12): p. 1-25.
13. Koressaar, T. and M. Remm, Enhancements and modifications of primer design program Primer3. Bioinformatics, 2007. 23(10): p. 1289-1291.
14. Untergasser, A., et al., Primer3—new capabilities and interfaces. Nucleic acids research, 2012.40(15): p. e115-e115.
15. Köressaar, T., et al., Primer3_masker: integrating masking of template sequence with primer design software. Bioinformatics, 2018. 34(11): p. 1937-1938.
16. Metzker, M. L., Sequencing technologies—the next generation. Nature Reviews Genetics, 2010. 11(1): p. 31-46.
17. Zymo Research: ZymoBIOMICS Microbial Community Standards. Available from: www.zymoresearch.com/collections/zymobiomics-microbialcommunity-standards.
18. Mancabelli, L., et al., Guideline for the analysis of the microbial communities of the human upper airways. J Oral Microbiol, 2022. 14(1): p. 2103282.
19. Kline, M. C., E. L. Romsos, and D. L. Duewer, Evaluating Digital PCR for the Quantification of Human Genomic DNA: Accessible Amplifiable Targets. Anal Chem, 2016. 88(4): p. 2132-9.
20. Zhang, X., T. Osaka, and S. Tsuneda, Bacterial metabolites directly modulate farnesoid X receptor activity. Nutr Metab (Lond), 2015. 12(1): p. 48.
21. Leite, G., et al., The duodenal microbiome is altered in small intestinal bacterial overgrowth. PLOS ONE, 2020. 15(7): p. e0234906.
22. Beghini, F., et al., Integrating taxonomic, functional, and strain-level profiling of diverse microbial communities with bioBakery 3. Elife, 2021. 10.
23. Bolger, A. M., M. Lohse, and B. Usadel, Trimmomatic: a flexible trimmer for Illumina sequence data. Bioinformatics, 2014. 30(15): p. 2114-20.
24. Shaiber, A., et al., Functional and genetic markers of niche partitioning among enigmatic members of the human oral microbiome. Genome Biol, 2020. 21(1): p. 292.
25. Koster, J. and S. Rahmann, Snakemake—a scalable bioinformatics workflow engine. Bioinformatics, 2012. 28(19): p. 2520-2.
26. Eren, A. M., et al., Anvi'o: an advanced analysis and visualization platform for 'omics data. PeerJ, 2015. 3: p. e1319.
27. Eren, A. M., et al., Community-led, integrated, reproducible multi-omics with anvi'o. Nat Microbiol, 2021. 6(1): p. 3-6.
28. Eren, A. M., et al., A filtering method to generate high quality short reads using illumina paired-end technology. PLoS One, 2013. 8(6): p. e66643.
29. Li, D., et al., MEGAHIT: an ultra-fast single-node solution for large and complex metagenomics assembly via succinct de Bruijn graph. Bioinformatics, 2015. 31(10): p. 1674-6.
30. Langmead, B. and S. L. Salzberg, Fast gapped-read alignment with Bowtie 2. Nat Methods, 2012. 9(4): p. 357-9.
31. Hyatt, D., et al., Prodigal: prokaryotic gene recognition and translation initiation site identification. BMC Bioinformatics, 2010. 11(1): p. 119.
32. Lee, M. D., GToTree: a user-friendly workflow for phylogenomics. Bioinformatics, 2019. 35(20): p. 4162-4164.
33. Finn, R. D., J. Clements, and S. R. Eddy, HMMER web server: interactive sequence similarity searching. Nucleic Acids Res, 2011. 39(Web Server issue): p. W29-37.
34. Eddy, S. R., Accelerated Profile HMM Searches. PLoS Comput Biol, 2011. 7(10): p. e1002195.
35. Tatusov, R. L., et al., The COG database: an updated version includes eukaryotes. BMC Bioinformatics, 2003. 4: p. 41.
36. Kanehisa, M. and S. Goto, KEGG: kyoto encyclopedia of genes and genomes. Nucleic Acids Res, 2000. 28(1): p. 27-30.
37. Alneberg, J., et al., Binning metagenomic contigs by coverage and composition. Nat Methods, 2014. 11(11): p. 1144-6.
38. Chaumeil, P. A., et al., GTDB-Tk: a toolkit to classify genomes with the Genome Taxonomy Database. Bioinformatics, 2019. 36(6): p. 1925-1927.
39. Chaumeil, P. A., et al., GTDB-Tk v2: memory friendly classification with the Genome Taxonomy Database. Bioinformatics, 2022: p. btac672.
40. Roux, S., et al., VirSorter: mining viral signal from microbial genomic data. PeerJ, 2015. 3: p. e985.
41. Barlow, J. T., et al., Quantitative sequencing clarifies the role of disruptor taxa, oral microbiota, and strict anaerobes in the human small-intestine microbiome. Microbiome, 2021. 9(1): p. 214.
42. Barlow, J. T., S. R. Bogatyrev, and R. F. Ismagilov, A quantitative sequencing framework for absolute abundance measurements of mucosal and lumenal microbial communities. Nat Commun, 2020. 11(1): p. 2590.
43. Bruggeling, C. E., et al., Optimized bacterial DNA isolation method for microbiome analysis of human tissues. Microbiologyopen, 2021. 10(3): p. e1191.
44. Cheng, W. Y., et al., High Sensitivity of Shotgun Metagenomic Sequencing in Colon Tissue Biopsy by Host DNA Depletion. Genomics Proteomics Bioinformatics, 2022.
45. Weyrich, L. S., et al., Laboratory contamination over time during low-biomass sample analysis. Mol Ecol Resour, 2019. 19(4): p. 982-996.
46. Salter, S. J., et al., Reagent and laboratory contamination can critically impact sequence—based microbiome analyses. BMC Biol, 2014. 12: p. 87.
47. Velasquez-Mejia, E. P., J. de la Cuesta-Zuluaga, and J. S. Escobar, Impact of DNA extraction, sample dilution, and reagent contamination on 16S rRNA gene sequencing of human feces. Appl Microbiol Biotechnol, 2018. 102(1): p. 403-411.
48. Liu, Y., et al., De novo identification of microbial contaminants in low microbial biomass microbiomes with Squeegee. Nat Commun, 2022. 13(1): p. 6799.
49. Vaga, S., et al., Compositional and functional differences of the mucosal microbiota along the intestine of healthy individuals. Sci Rep, 2020. 10(1): p. 14977.
50. Mehta, R. S., et al., Stability of the human faecal microbiome in a cohort of adult men. Nat Microbiol, 2018. 3(3): p. 347-355.
51. Human Microbiome Project, C., Structure, function and diversity of the healthy human microbiome. Nature, 2012. 486(7402): p. 207-14.
52. Wilson, M. R., et al., Clinical Metagenomic Sequencing for Diagnosis of Meningitis and Encephalitis. New England Journal of Medicine, 2019. 380(24): p. 2327-2340.
53. Delmont, T. O. and A. M. Eren, Linking pangenomes and metagenomes: the Prochlorococcus metapangenome. PeerJ, 2018. 6: p. e4320.

1. A method to deplete a biological sample of a host comportment compartment encapsulating a host nucleic acid, the host compartment having a host compartment elastic modulus and a host compartment diameter in at least one dimension, the method comprising
disrupting the biological sample by contacting the biological sample with a set of beads, each having a bead radius, in accordance with beads parameters,
the bead radius and beads parameters configured to selectively disrupt the host compartment with respect to a microbial compartment having a microbial compartment elastic modulus and a microbial compartment diameter in at least one dimension, the microbial compartment encapsulating a microbial nucleic acid, the microbial nucleic acid having a mass equal to or lower than the host nucleic acid,
the bead radius and beads parameters being obtained by
a) selecting the bead radius to determine, for each of the host compartment and the microbial compartment, a compartment crush volume as a function of compartment diameter and bead radius;
b) selecting the bead parameters including number of beads, bead collision frequency and bead beating time to determine, for each of the host compartment and the microbial compartment, a compartment effective crush volume as a function of said compartment crush volume, the compartment elastic modulus and said bead parameters;
c) determining, for each of the host compartment and the microbial compartment, a percentage of total sample volume that is bead beaten for a given compartment as a ratio between the compartment effective crush volume and a total volume of the biological sample, and
d) selecting the set of beads having the bead radius and the bead parameters to obtain a percentage of total sample volume of at least 100% for the given host compartment and a percentage of total sample volume of up to 50% for the given microbial compartment,
the contacting performed in a container for a time and under conditions according to the beads parameters to provide a disrupted biological sample depleted of the host compartment and comprising at least one disrupted host compartment and accessible host nucleic acid.

2. The method of claim 1, wherein and the host compartment has a host compartment diameter of 8 um or higher and the microbial compartment has a microbial compartment diameter not greater than 5 um.

3. The method of claim 1, wherein the microbial compartment has a microbial compartment diameter not greater than 3 um.

4. The method of claim 1, wherein the host compartment has an elastic modulus equal to or lower than 10\hat{ }5 Pa and the microbial compartment has an elastic modulus equal to or higher than 10\hat{ }7 Pa.

5. The method of claim 1, wherein the microbial compartment has a microbial compartment diameter higher than 5 um and an elastic modulus equal to or higher than 10\hat{ }8 Pa.

6. The method of claim 5, wherein the host compartment has an elastic modulus not greater than 10\hat{ }5 Pa.

7. The method of claim 1, wherein dimension and material of the beads are selected so that a Stokes number of the beads is maintained at a value of more than 3.

8. The method of claim 1, wherein the beads of the set of beads have an elastic modulus of at least 50 GPa.

9. The method of claim 1, wherein the beads of the set of beads have a substantially spherical shape.

10. The method of claim 1, wherein beads of the set of beads have a bead radius selected from 1.0 to 1.8 mm.

11. The method of claim 1, wherein the number of beads is selected from 5% to 50% by volume of the container, the bead collision frequency is from 4800 Hz to 45000 Hz, and the duration of the contacting is selected from 15 to 120 seconds, the bead collision frequency from 4800 Hz to 45000 Hz corresponding to a bead agitation frequency from 16 Hz to 150 Hz.

12. The method of claim 1, wherein the microbial compartment comprises bacterial cells having a bacterial cell diameter not greater than 5 um and a bacterial cell elastic modulus not lower than 10\hat{ }7 Pa, and the host compartments are animal cells having a diameter of 8 um or higher and an elastic modulus of lower than 10\hat{ }5 Pa.

13. The method of claim 1, wherein the microbial compartment comprises archaea cells having an archaea cell diameter not greater than 5 um and an archaea cell elastic modulus not greater than 10\hat{ }10 Pa, and the host compartments are animal cells having an animal cell diameter of 8 um or higher and an animal cell elastic modulus of equal or lower than 10\hat{ }5 Pa.

14. The method of a m claim 1, wherein the microbial compartment comprises a viral compartment having a viral compartment diameter not greater than 0.5 um and a viral compartment elastic modulus not greater than 10\hat{ }10 Pa, and the host compartment comprises animal cells having an animal cell diameter of 8 um or higher and an animal cell elastic modulus of equal or lower than 10\hat{ }5 Pa.

15. The method of claim 1, wherein that microbial compartment comprises a viral compartment having a viral compartment diameter not greater than 0.2 um and a viral compartment elastic modulus not greater than 10\hat{ }10 Pa, and the host compartment comprises animal cells having an animal cell diameter of 8 um or higher and an animal cell elastic modulus host of equal or lower than 10\hat{ }5 Pa.

16. The method of a m claim 1, wherein the microbial compartment comprises a fungal compartment having a fungal compartment diameter equal to or higher than 3 um and a fungal compartment elastic modulus not greater than 10\hat{ }8 Pa, and the host compartment comprises animal cells having an animal cell diameter of 8 um or higher and an animal cell elastic modulus of equal or lower than 10\hat{ }5 Pa.

17. The method of claim 5, wherein the microbial compartment is a human-associated microbe.

18. The method of claim 17, wherein the human-associated microbe is a human pathogen.

19. The method of claim 17, wherein the biological sample is a human sample.

20. The method of claim 1, wherein the biological sample is an isolated portion of a liquid or soft tissue of an individual, the liquid or soft tissue having an elastic modulus from 1 Pa to 20 kPa, and the biological sample, in combination with the set of beads, has a volume up to 70% of a volume of the container.

21. The method of claim 20, wherein the liquid tissue is selected from saliva, individual cells, swabs resuspended in liquid, and sputum.

22. The method of claim 20, wherein the soft tissue is selected from brain, glandular tissue, adipose tissue liver, uterine tissue, and lamina propria.

23. The method of claim 1, wherein the biological sample is an isolated portion of a medium softness tissue of an individual, the medium softness tissue having an elastic modulus from 20 kPa to 100 MPa, and the biological sample has a volume up to 10% of a volume of the container.

24. The method of claim 23, wherein the medium softness tissue is selected from muscle, heart, skin, mucosa, and adipose tissue.

25. The method of claim 1, wherein the biological sample is an isolated portion of a hard tissue of an individual, the hard tissue having an elastic modulus from 0.1 GPa to 500 GPa, and the biological sample is pre-treated to lower the elastic modulus of the hard tissue to 100 MPa or lower.

26. The method of claim 1, wherein the method further comprises treating the biological sample before the contacting to cleave bonds within a matrix of the sample or between a host cell and a matrix of the sample and decrease viscosity of the sample.

27. The method of claim 1, the method further comprising obtaining the set of beads by performing a) said selecting the bead radius, b) said selecting the bead parameters, c) said determining and d) said providing the selected said of beads.

28.-69. (canceled)
